SEQUENCE LISTING (495 sequences)

SEQ ID NO: 1 - protein, 132 aa, synthetic construct
QVQLVESGGGLVQPGDSLRLSCAASGFILDTYSVAWFRQAPGKEREGVSCISSRDGNTFYSDSVKGRFTISRDNAKNTVYLQMNSLKSEDTAVYTCAAGAQAHCTIFTSYFNSDYYRRYNYWGQGTQVTVSS

SEQ ID NO: 2 - protein, 118 aa, synthetic construct
QVQLVESGGGLVQPGGSLRLSCAASGFIFSDKVMSWVRQAPGKGREWVSTITPGGTATSYTESVKGRFTISRDNAKNTLYLQMNNLKPDDTALYYCRIGGPGGRYDNWGQGTQVTVSS

SEQ ID NO: 3 - protein, 121 aa, synthetic construct
QVQLVESGGGLVQAGGSLTLSCAASRSIFEINTMTMGWYRQAPGKQRELIAASRGATTNYADSVKGRFTISRDNAKNTVYLQMNSLKPEDTAVYYCSADRYGFGYGDNDYWGQGTQVTVSS

SEQ ID NO: 4 - protein, 130 aa, synthetic construct
QVQLVESGGGLVQPGGSLRLSCAASGFSLDYYHIGWFRQAPGKEREGISCISSSDGYTLYADSVKGRFTISRDNAKNTVYLQMNSLKPEDTAVYYCAASPRRLACAGSLYPPLSADFSSWGQGTQVTVSS

SEQ ID NO: 5 - protein, 120 aa, synthetic construct
QVQLVESGGGLVQTGGSLRLSCVASGAIVSAESMGWYRQAPGNQRELVAEIISGSKSNYGESVKGRFTISRDNAENTVYLQMNSVKPEDTAVYYCKRTERIWTNNPQVYWGQGTQVTVSS

SEQ ID NO: 6 - protein, 116 aa, synthetic construct
QVQLVESGGGLVQAGGSLRLSCAASGTFSSINLMGYYRQAPGKQREFVARDYTEGTTDYADSVKGRFTISRDNTKKTVFLQMNNLKPEDTAIYYCWLMVRAGDVYWGQGTQVTVSS

SEQ ID NO: 7 - protein, 118 aa, synthetic construct
QVQLVESGGGLVHTGGSLRLSCAASGSISGLNTMGWYRQAPGKQRELVATIISGTMTRYADSVKGRFTISKDNAKNTVYLQMNSLKPEDTAVYYCTFKEITRDSRSYWGQGTQVTVSS

SEQ ID NO: 8 - protein, 117 aa, synthetic construct
QVQLVESGGGLVQAGGSLRLSCAASGSSVSMNSMAWYRQAPGKQRELVALITPGDRINYADFVKGRFTISRDNAKNTVNLQMNNLKPEDTAVYYCNIGATRPPFGAWGQGTQVTVSS

SEQ ID NO: 9 - protein, 118 aa, synthetic construct
QVQLVESGGGLVQAGGSLRLSCVASGFIYSISTMGWYRQAPGKQRELVATITSGGNTNYADSVKGRFTISRDNAQNTVYLQMNNLKPEDTAIYYCNTAWRETIVSRVWGQGTQVTVSS

SEQ ID NO: 10 - protein, 121 aa, synthetic construct
QVQLVESGGGLVQAGGSLKLACVVSGRYFRINAMGWYRQAPGKQRDMVASISNDGSTNYGDVVKGRFTISRDNNKNTVYLQMNSLKPEDTAVYSCNVKALPFLSSNELSYWGQGTQVTVSS

SEQ ID NO: 11 - protein, 116 aa, synthetic construct
QVQLVESGGGLVQAGGSLKLSCAASGTFSSIALMGYYRQGPGKQREFVARVSIGGVTEYADSVKGRFTISGDNAKKTVDLQMNNLKPDDTGVYYCWRMEGAGDVYWGQGTQVTVSS

SEQ ID NO: 12 - protein, 119 aa, synthetic construct
QVQLVESGGGLVQAGGSLRLSCVASGIIFRIFSMGWYRQAPGKQRELVATITSGGNTNYAHSVKGRFTISRDDAKNTVNLQMNSLKPEDTAVYYCNVAIPSGIVDRSAWGQGTQVTVSS

SEQ ID NO: 13 - protein, 8 aa, synthetic construct
GFILDTYS

SEQ ID NO: 14 - protein, 8 aa, synthetic construct
ISSRDGNT

SEQ ID NO: 15 - protein, 25 aa, synthetic construct
AAGAQAHCTIFTSYFNSDYYRRYNY

SEQ ID NO: 16 - protein, 8 aa, synthetic construct
GFIFSDKV

SEQ ID NO: 17 - protein, 8 aa, synthetic construct
ITPGGTAT

SEQ ID NO: 18 - protein, 11 aa, synthetic construct
RIGGPGGRYDN

SEQ ID NO: 19 - protein, 10 aa, synthetic construct
RSIFEINTMT

SEQ ID NO: 20 - protein, 6 aa, synthetic construct
SRGATT

SEQ ID NO: 21 - protein, 14 aa, synthetic construct
SADRYGFGYGDNDY

SEQ ID NO: 22 - protein, 8 aa, synthetic construct
GFSLDYYH

SEQ ID NO: 23 - protein, 8 aa, synthetic construct
ISSSDGYT

SEQ ID NO: 24 - protein, 23 aa, synthetic construct
AASPRRLACAGSLYPPLSADFSS

SEQ ID NO: 25 - protein, 8 aa, synthetic construct
GAIVSAES

SEQ ID NO: 26 - protein, 7 aa, synthetic construct
IISGSKS

SEQ ID NO: 27 - protein, 14 aa, synthetic construct
KRTERIWTNNPQVY

SEQ ID NO: 28 - protein, 8 aa, synthetic construct
GTFSSINL

SEQ ID NO: 29 - protein, 7 aa, synthetic construct
DYTEGTT

SEQ ID NO: 30 - protein, 10 aa, synthetic construct
WLMVRAGDVY

SEQ ID NO: 31 - protein, 8 aa, synthetic construct
GSISGLNT

SEQ ID NO: 32 - protein, 7 aa, synthetic construct
IISGTMT

SEQ ID NO: 33 - protein, 12 aa, synthetic construct
TFKEITRDSRSY

SEQ ID NO: 34 - protein, 8 aa, synthetic construct
GSSVSMNS

SEQ ID NO: 35 - protein, 7 aa, synthetic construct
ITPGDRI

SEQ ID NO: 36 - protein, 11 aa, synthetic construct
NIGATRPPFGA

SEQ ID NO: 37 - protein, 8 aa, synthetic construct
GFIYSIST

SEQ ID NO: 38 - protein, 7 aa, synthetic construct
ITSGGNT

SEQ ID NO: 39 - protein, 12 aa, synthetic construct
NTAWRETIVSRV

SEQ ID NO: 40 - protein, 8 aa, synthetic construct
GRYFRINA

SEQ ID NO: 41 - protein, 7 aa, synthetic construct
ISNDGST

SEQ ID NO: 42 - protein, 15 aa, synthetic construct
NVKALPFLSSNELSY

SEQ ID NO: 43 - protein, 8 aa, synthetic construct
GTFSSIAL

SEQ ID NO: 44 - protein, 7 aa, synthetic construct
VSIGGVT

SEQ ID NO: 45 - protein, 10 aa, synthetic construct
WRMEGAGDVY

SEQ ID NO: 46 - protein, 8 aa, synthetic construct
GIIFRIFS

SEQ ID NO: 47 - protein, 7 aa, synthetic construct
ITSGGNT

SEQ ID NO: 48 - protein, 13 aa, synthetic construct
NVAIPSGIVDRSA

SEQ ID NO: 49 - protein, 132 aa, synthetic construct
EVQLVESGGGLVQPGGSLRLSCAASGFILDTYSVAWFRQAPGKEREGVSCISSRDGNTFYSDSVKGRFTISRDNAKNTVYLQMNSLRAEDTAVYTCAAGAQAHCTIFTSYFNSDYYRRYNYWGQGTLVTVSS

SEQ ID NO: 50 - protein, 118 aa, synthetic construct
EVQLVESGGGLVQPGGSLRLSCAASGFIFSDKVMSWVRQAPGKGREWVSTITPGGTATSYTESVKGRFTISRDNSKNTLYLQMNSLKADDTAVYYCRIGGPGGRYDNWGQGTLVTVSS

SEQ ID NO: 51 - protein, 121 aa, synthetic construct
EVQLVESGGGLVQPGGSLRLSCAASRSIFEINTMTMGWYRQAPGKQRELISASRGATTNYADSVKGRFTISRDNAKNTVYLQMNSLKAEDTAVYYCSADRYGFGYGDNDYWGQGTQVTVSS

SEQ ID NO: 52 - protein, 117 aa, synthetic construct
AVQLVDSGGGLVTAGDSLTLSCVASGRTLSPYTAGWFRQAPGREREFVALITARGDWTSYSDSVKGRFTISRDNAKNTIYLQMNSLEPEDTALYYCVRDLLGRDDYWGQGTQVTVSS

SEQ ID NO: 53 - protein, 119 aa, synthetic construct
HVQLVESGGGLVQTGGSLRLSCAASGRTLNNYVVAWFRQAPGKEREFVATMWWSGGSPWYSDNVKGRFTISRDNAKNTLYLQMNSLRPEDTAVYYCAATWVGTSEYRHWGQGTQVTVSS

SEQ ID NO: 54 - protein, 123 aa, synthetic construct
QVQLVESGGGLVQAGGSLRLSCAASGSIFAYHVMGWYRQAPGKQRELVALITSGGSTNYTDSVKGRFTISRDNAKNTVYLQMNSLKPEDTAVYYCAIGYYSGSYYWERSGDYWGQGTQVTVSS

SEQ ID NO: 55 - protein, 119 aa, synthetic construct
QVKLEESGGGLVQTGGSLRLSCAASGRTLNNYVVAWFRQAPGKEREFVATMWWSGGSPWYSDNVKGRFTISRDNAKNTLYLQMNSLRPEDTAVYYCAATWVGTSEYQHWGQGTQVTVSS

SEQ ID NO: 56 - protein, 119 aa, synthetic construct
QVKLEESGGGLVQAGGSLRLSCAASGRTFSDYTVAWFRQAPGSEREFVAASVWTDGKPYYVDSVRGRFTISRDNAKNTVWLQMDSLQPDDTAVYYCQALTGGSWILDYWGQGTPVTVSS

SEQ ID NO: 57 - protein, 123 aa, synthetic construct
QVKLEESGGGLVQAGDSLTLSCAASGRPLRMYNMGWFRQAPGKEREFVAFISWSGKTTYYSDAVKGRFVISRDNAKRTVYLQMDSLKPDDTGDYYCFMNVWADTSDSSADAYWGQGTQVTVSS

SEQ ID NO: 58 - protein, 122 aa, synthetic construct
EVQLVDSGGGLVQAGGSLRLSCAASGSTFSRYAMGWIRQAPGKEREFVAAISWSGGSTYYADSVKGRFTISRDVAKNTVSLQMNSLKPEDAGVYFCFGDIIRSGERSDYEYWGQGTRVTVTS

SEQ ID NO: 59 - protein, 120 aa, synthetic construct
QVQLVQSGGGLVLPGGSLRLSCAASGFTLSSYWMHWLRQAPGKGLEWVSAIKPESGITYYAESMKGRFTISRDNAKNTLYLQMNSLKSEDSALYYCVREDYDSAYVGDYWGKGTQVTVSS

SEQ ID NO: 60 - protein, 123 aa, synthetic construct
QVKLEESGGGFVQPGGSLRLSCAASGFTYSSYWMHWVRQAPGKGLEWVSGISTGGDTTDTYYADSVKGRFTISRDNAKNTLYLQMNSLKPEDTAVYYCVALNLWGTDLEHDYWGRGTQVTVSS

SEQ ID NO: 61 - protein, 123 aa, synthetic construct
HVQLVDSGGGLVRSGGSLRLSCVMSGGTLEYYAMAWFRQAPGKERETVAAITWSGGSTYYVDSVKGRFTISRDNAKNTLYLQMNSLKPEDTAVYYCAAQFVEVEILVRSYEYWGQGTQVTVST

SEQ ID NO: 62 - protein, 121 aa, synthetic construct
QVQLVDSGGGLVQPGGSLRLSCAASGFTFSSYWMYWVRQAPGKGLEWVSAINTDGDSTYYADSVKGRFTVSRDVAGNMIYLQMTNLKPEDTAVYTCAAIVTRSDGHQYDYWGRGTQVTVSV

SEQ ID NO: 63 - protein, 124 aa, synthetic construct
QVQLVESGGGSVQAGGSLRLSCAASGRIDSGYTMAWFRQAPGSEREFVAAVVGSDGRDYYIDSVRGRFTVSRDSAKNTVYLEMTGLKPEDTAVYYCAATNYYSDYLDHLSRGYWGQGTQVTVSS

SEQ ID NO: 64 - protein, 118 aa, synthetic construct
QVQLVDSGGRLVQAGDSLRLSCSASGFTFGSYWMFWVRQAPGKGLEWVSAIDTSGGHVYYGDSVNGRFTISRDNAKNTLYLQMNSLKPEDTALYYCARVENTWESIYWGQGTTVTVSS

SEQ ID NO: 65 - protein, 118 aa, synthetic construct
QVQLVESGGGLAQSGGSLELSCAASGHTLNSYAMGWFRQAPGKEREFVAAISRSGEKTYYADSVKGRFTISGDNAKNTAALQMGRLKPEDTAVYYCGAWNFVKNDAYWGQGTQVTVSA

SEQ ID NO: 66 - protein, 114 aa, synthetic construct
QVQLVESGGGLVQPGGSLRLSCAASGFTFSTYSMYWVRQAPGKGLEWVSAIDARGVNTYYADSVRGRFTVSRDNAKNTLYLQMNNLKLEDTALYYCGGWEFATWGRGTQVTVSS

SEQ ID NO: 67 - protein, 114 aa, synthetic construct
QVKLEESGGGLVQAGGSLRLSCAASGHTFSNSAMGWIRQAPGKEREFVAAISLNGGNTHYAESVKGRFAISRDNAKNTMYLQMNSLKPEDTAVYYCATLGFASWGQGTQVTVSS

SEQ ID NO: 68 - protein, 119 aa, synthetic construct
QVQLVESGGGLVQAGGSLRLSCAASGRTFSGYTMAWFRQAPGSEREFVAAAVGSDGSTYYVDSVKGRFTMSRDSAKNTVYLMMTSLQPVDSAVYFCVKLDSGAWSLAEWGQGTQVTVSS

SEQ ID NO: 69 - protein, 119 aa, synthetic construct
PVQLVESGGGLVQAGGSLRLSCAASGRTFSSYAMGWFRQAPGKEREFVAGVSWTGDNTGYADSVKGRFTISRDNFKSTVYLEMNSLKPEDTAVYFCAAWNWGHHEYTYWGRGTQVTVSS

SEQ ID NO: 70 - protein, 120 aa, synthetic construct
HVQLVESGGGLVQPGGSLRLSCSVSGRTIGSFVMGWFRQAPGKEREFVAAVNWRGSSTYYADSVRGRFTISRGNAATTMSLQMNSLKPEDAALYYCARWSWDIGADFGSWGQGTQVTVSS

SEQ ID NO: 71 - protein, 122 aa, synthetic construct
QVQLVESGGGLVQPGGSLRLSCAASGFTFSNYAMIWVRQAPGKGLEWVSSINANSDTTAYSDSVKGRFAISRDNARNTLYLQMNSLKPEDTAVYFCVSDSYIGGLYATYVYWGQGTQVTVSS

SEQ ID NO: 72 - protein, 121 aa, synthetic construct
QVKLEESGGGLVQAGGSLSLSCKASVRYFSTYAMGWFRQAPGKEREFVAAISWSGGSTYYADSVKGRFTISRDNAKNTVYLEMNSLKPEDTAVYYCAAPYGSSQNLEYDYWGQGTQVTVSS

SEQ ID NO: 73 - protein, 124 aa, synthetic construct
HVQLVDSGGGLVQAGGSLRLSCAASGFTFDDYAMSWVRQAPGKGLEWVSAISWNGGSTYYAESMKGRFTISRDNTKNTLYLQMNSLKPEDSAVYYCVPVSHSDSVCGSPYMDYWGKGTLVTVSS

SEQ ID NO: 74 - protein, 121 aa, synthetic construct
QVKLEESGGGLVHPGGSLTLSCVASGFNLTSDAVGWFSQAAPGKDLEPISCISRTESDTVQGRFAISRNSAKNTVYLQMNSLQPGDTAVYRCAADNAADRCSLSIYNYNLWGQGTQVTVSS

SEQ ID NO: 75 - protein, 115 aa, synthetic construct
QVKLEESGGGLVQAGGSLRLSCQVSGRTFASFAMAWFRQAPGKEREFVAGISWSGGFTSYADSVKGRFTISRDNAKHTVYLQMNSLKTEDTAMYYCGEWGGFSLLGQGTQVTVSS

SEQ ID NO: 76 - protein, 127 aa, synthetic construct
QVQLVESGGGLVQAGGSLRLSCAASGFTFDDYAIGWFRQAPGKEREGVSCIASSDGSTFYADSVKGRFTISSDNANNTVYLQMNNLNREDTAVYYCVAPCFWFDTVIAGTDPRYDYWGQGTQVTVSS

SEQ ID NO: 77 - protein, 129 aa, synthetic construct
QVQLVESGGGLVQAGGSLRLSCAASGFTLGYYAIGWFRQAPGKEREGISCISGRDGSTYYAGSVKGRFTISSDNAKNTVYLQMNSLKPVDTAVYYCAAVRGPIVSMDPDLCRPVEFDYRGQGTQVTVSS

SEQ ID NO: 78 - protein, 129 aa, synthetic construct
QVQLVESGGGLVQAGGSLRLSCATSGFTFEDYAIAWFRQAPGKEREAVSCTSKNDRMPYYAPSVKDRFTISTDNGKNTVYLQMNSLKPEDTAVYYCAATNGPAITLFPCHINYWLYDNWGPGTQVTVSS

SEQ ID NO: 79 - protein, 129 aa, synthetic construct
QVQLVESGGGLVQAGGSLRLSCATSGFTFEDYAIAWFRQAPGKEREAVSCTSKNDRMPYYAPSVKGRFTISTDNGKNTAYLQMNSLKPEDTAVYYCASADWRSPTPFPCGVSRSLYDHWGQGTQVTVSS

SEQ ID NO: 80 - protein, 112 aa, synthetic construct
QVQLVESGGGLVQAGGSLRLSCAASGNFLRFNAMGWFRQAPGKQRELVAMISSGGRTNYVDSVKGRFTVSRDNAKNTVTLQMNSLKPEDTAVYYCWSAPDYWGQGTQVTVSS

SEQ ID NO: 81 - protein, 120 aa, synthetic construct
QVQLVESGGGLVQAGGSLRLSCVVSGSFSSIDTVDWYRQAPGKQRELVATINRGGDTDYKDSVKGRFTISKDNAKNSVHLQMNNLKPEDTAVYTCAGSFTLATGDDFGSWGQGTQVTVSS

SEQ ID NO: 82 - protein, 117 aa, synthetic construct
QVQLVESGGGLVQAGGSLRLTCTASEQNFSADYMAWYRQAPGKERELIATISNSGRTHYVDSMAGRFTISRDNAKNTVYLQLDSLKPGDTAVYYCVAEPFSFRRRAWGRGTQVTVSS

SEQ ID NO: 83 - protein, 120 aa, synthetic construct
QVQLVESGGGLVQAGGSLRLTCTASEQNFSTDDMAWYRQAPGKERGLIATITNSGTTHYVDSMAGRFTISRDNAENTVYLQMNSLIPEDTAVYYCGESTTGWAECDFGCWGRRTQVTVSS

SEQ ID NO: 84 - protein, 119 aa, synthetic construct
QVQLVESGGGLVQPGGSLRLSCAASRSIFSINAMGWYRQAPGKQRELVATITNGGTTNYADSVKGRFTISRDNAENTVYLQMNSLKPEDTAVYYCNANSRYGVGWYNYWGQGTQVTVSS

SEQ ID NO: 85 - protein, 121 aa, synthetic construct
QVQLVESGGGLVQPGGSLRLSCAASGSIFSINPMGWYRQAPGKQRELVAAFTSGGTTNYADSVKGRFTISRDNAKNTVYLEMNSLKPEDTAVYYCNVRGGHCDPRYWREYWGQGTQVTVSS

SEQ ID NO: 86 - protein, 121 aa, synthetic construct
QVQLVESGGGLVQPGGSLRLSCAASGSIFSINPMGWYRQAPGKQRELVAAFTSGGTTNYADSVKGRFTISRDNAKNTVYLQMNSLKPEDTAAYYCNVRGGHYDPRYWREYWGQGTQVTVSS

SEQ ID NO: 87 - protein, 127 aa, synthetic construct
QVQLVESGGGLVQAGGSLRLSCAASGRPIDTYAMGWFRQAPGKEREFVAVVSWAGVYTYYADSVKGRFTISRDNAKNTVVLQVNRLKPEDTAVYYCAATKLPWNTIVMVQRSYCDYWSLGTHVTVSS

SEQ ID NO: 88 - protein, 127 aa, synthetic construct
QVQLVESGGGLVQAGGSRRLSCAASGRTISTYAMGWFRQAPGKEREFVAVVSWAGSYTYYADSVKGRFTISRDNAKNTVVLQVNSLKPEDTAVYYCAATKLPWNTSVMVKRSVYDYWGQGTQVTVSS

SEQ ID NO: 89 - protein, 124 aa, synthetic construct
QVQLVESGGGLVQAGDSLRLSCATSGRPLECYAMGWFRQAPGKEREFVAGISGNGVNTYYADSLKGRFTISRDNAKNTMNLQMNRLKPEDTAVYFCAATTVPVINLEISHMTYCGKGTLGTVSS

SEQ ID NO: 90 - protein, 127 aa, synthetic construct
QVQLVESGGGLVQAGDSLSLSCTSSGRRLEGYTMGWFRQPPGKEREFVAAISRNRVNTYYADSLKGRFTISRDNAKNTINLQMDRLKPEDTAVYYCAATNLPGITLLMSHMNYCDYWTLGTQVTVSS

SEQ ID NO: 91 - protein, 124 aa, synthetic construct
QVQLVESGGGLVQVGDSLNLSCTTSGPRLEGYTMGWFRQPPGKEREFVAVITRNRVNTYYANSLKGQFTFSKNNAKNTIILQMDRLKPEDTAVYFCAATTVPVINLQVSHINYWSKGTLVTVSS

SEQ ID NO: 92 - protein, 124 aa, synthetic construct
QVQLVESGGGLVQAGDSLSLSCTSSGRRLEGYTMGWFRQPPGKEREFVAAISRNRVNTYYADSLKGRFTISRDNAKNTINLQMDRLKPEDTAVYFCAATTVPVINLQVSHINYWGKGTLVTVSS

SEQ ID NO: 93 - protein, 124 aa, synthetic construct
QVQLVESGGGLVQAGDSLSLSCTSSGRRLEGYTMGWFRQPPGKEREFVAAISRNRVNTYYADSLKGRFTIPRDNAKNTINLQMDRLKPEDTAVYFCAATTVPVINLQVSHINYWGKGTLVTVSS

SEQ ID NO: 94 - protein, 124 aa, synthetic construct
QVQLVESGGGLVQVGGSLRLSCTASGRTFNLYAMGWFRQAPGEEREFVGVITWSGGSTGYADSVKGRFAISRDNAKNTVYLQMNNLKPEDTALYYCAAKVFPMATLDDDVYDYWGQGTQVTVSS

SEQ ID NO: 95 - protein, 128 aa, synthetic construct
QVQLVESGGGLVQAGATLRLSCAASGVTFSNYVMGWFRQAPGKEREFVATISWSGGSTYYADSVKGRFTISRDNAKNAVYLQMNSLKPEDTAVYYCAAESHRRNTIVIVTTPDEYDYWGQGTQVTVSS

SEQ ID NO: 96 - protein, 130 aa, synthetic construct
QVQLVESGGGLVQAGGSLRLSCAASGRTFSSYAMGWFRQAPGKEREFVAAISWSGGSTYYADSVKGRFTISRDNAKNTVYLQMNSLKPEDTAVYYCAADPSSGYNLFARTVVAFARYDYWGQGTQVTVSS

SEQ ID NO: 97 - protein, 122 aa, synthetic construct
QVQLVESGGGLVQAGGSLRLSCAASGRTFSSYAMGWFRQAPGKEREFVAAISWSGGSTYYADSVKGRFTIPRDSAKNAVYLQMNSLKPEDTATYCCAAHPAGAHGGLIYKNWGRGTHVTVSS

SEQ ID NO: 98 - protein, 121 aa, synthetic construct
QVQLVESGGGLVQAGGSLRLSCVAFGRTVSNYAVAWFRQAPGKERRFVAAINRSGTPYYGDSVKDRFTISRDSAKNAVYLQMNSLKPEDTATYSCAADPTGAHVGAIYKNWGQGTQVTVSS

SEQ ID NO: 99 - protein, 122 aa, synthetic construct
QVQLVESGGGLVQAGGSLRLSCVAFGRTFSNYAVAWFRQAPGKERRFVAAINRSGTPYYGDSVKGRFTISRDNAENTVYLQMNSLKPEDTAVYYCAADLRGSSWYFDGVDYWGKGTLVTVSS

SEQ ID NO: 100 - protein, 123 aa, synthetic construct
QVQLVESGGGLVQAGGSLRLSCAASGRTFSSYAMGWFRQAPGKEREFVAAISWSGGSTYYADSVKGRFTISRDNAKNTVYLQMNSLKPEDTAVYYCAADLRGSSWYFDGMDYWGKGTLVTVSS

SEQ ID NO: 101 - protein, 127 aa, synthetic construct
QVQLVESGGGLVQSGGSLRLSCAASGRTFSRYAMGWFRQAPGKEREFVAAITRSGTSTYYADSVKGRFTISRDNAKNTMYLQMNSLKPEDTAVYYCAAHEAQYSSRWSGTEKGYDYWGQGTQVTVSS

SEQ ID NO: 102 - protein, 130 aa, synthetic construct
QVQLVESGGGLVQAGGSLRLSCAASGRTFSSYAMGWFRQAPGKEREFVAAISWSGGSTYYADSVKGRFTTSRDNAKSTVYLQMNSLKPEDTAVYYCAADPSSGYNLFARTVVAFARYDYWGQGTQVTVSS

SEQ ID NO: 103 - protein, 8 aa, synthetic construct
GRTLSPYT

SEQ ID NO: 104 - protein, 8 aa, synthetic construct
ITARGDWT

SEQ ID NO: 105 - protein, 10 aa, synthetic construct
VRDLLGRDDY

SEQ ID NO: 106 - protein, 8 aa, synthetic construct
GRTLNNYV

SEQ ID NO: 107 - protein, 8 aa, synthetic construct
MWWSGGSP

SEQ ID NO: 108 - protein, 12 aa, synthetic construct
AATWVGTSEYRH

SEQ ID NO: 109 - protein, 8 aa, synthetic construct
GSIFAYHV

SEQ ID NO: 110 - protein, 7 aa, synthetic construct
ITSGGST

SEQ ID NO: 111 - protein, 17 aa, synthetic construct
AIGYYSGSYYWERSGDY

SEQ ID NO: 112 - protein, 12 aa, synthetic construct
AATWVGTSEYQH

SEQ ID NO: 113 - protein, 8 aa, synthetic construct
GRTFSDYT

SEQ ID NO: 114 - protein, 8 aa, synthetic construct
SVWTDGKP

SEQ ID NO: 115 - protein, 12 aa, synthetic construct
QALTGGSWILDY

SEQ ID NO: 116 - protein, 8 aa, synthetic construct
GRPLRMYN

SEQ ID NO: 117 - protein, 8 aa, synthetic construct
ISWSGKTT

SEQ ID NO: 118 - protein, 16 aa, synthetic construct
FMNVWADTSDSSADAY

SEQ ID NO: 119 - protein, 8 aa, synthetic construct
GSTFSRYA

SEQ ID NO: 120 - protein, 8 aa, synthetic construct
ISWSGGST

SEQ ID NO: 121 - protein, 15 aa, synthetic construct
FGDIIRSGERSDYEY

SEQ ID NO: 122 - protein, 8 aa, synthetic construct
GFTLSSYW

SEQ ID NO: 123 - protein, 8 aa, synthetic construct
IKPESGIT

SEQ ID NO: 124 - protein, 13 aa, synthetic construct
VREDYDSAYVGDY

SEQ ID NO: 125 - protein, 8 aa, synthetic construct
GFTYSSYW

SEQ ID NO: 126 - protein, 10 aa, synthetic construct
ISTGGDTTDT

SEQ ID NO: 127 - protein, 14 aa, synthetic construct
VALNLWGTDLEHDY

SEQ ID NO: 128 - protein, 8 aa, synthetic construct
GGTLEYYA

SEQ ID NO: 129 - protein, 8 aa, synthetic construct
ITWSGGST

SEQ ID NO: 130 - protein, 16 aa, synthetic construct
AAQFVEVEILVRSYEY

SEQ ID NO: 131 - protein, 8 aa, synthetic construct
GFTFSSYW

SEQ ID NO: 132 - protein, 8 aa, synthetic construct
INTDGDST

SEQ ID NO: 133 - protein, 14 aa, synthetic construct
AAIVTRSDGHQYDY

SEQ ID NO: 134 - protein, 8 aa, synthetic construct
GRIDSGYT

SEQ ID NO: 135 - protein, 8 aa, synthetic construct
VVGSDGRD

SEQ ID NO: 136 - protein, 17 aa, synthetic construct
AATNYYSDYLDHLSRGY

SEQ ID NO: 137 - protein, 8 aa, synthetic construct
GFTFGSYW

SEQ ID NO: 138 - protein, 8 aa, synthetic construct
IDTSGGHV

SEQ ID NO: 139 - protein, 11 aa, synthetic construct
ARVENTWESIY

SEQ ID NO: 140 - protein, 8 aa, synthetic construct
GHTLNSYA

SEQ ID NO: 141 - protein, 8 aa, synthetic construct
ISRSGEKT

SEQ ID NO: 142 - protein, 11 aa, synthetic construct
GAWNFVKNDAY

SEQ ID NO: 143 - protein, 8 aa, synthetic construct
GFTFSTYS

SEQ ID NO: 144 - protein, 8 aa, synthetic construct
IDARGVNT

SEQ ID NO: 145 - protein, 7 aa, synthetic construct
GGWEFAT

SEQ ID NO: 146 - protein, 8 aa, synthetic construct
GHTFSNSA

SEQ ID NO: 147 - protein, 8 aa, synthetic construct
ISLNGGNT

SEQ ID NO: 148 - protein, 7 aa, synthetic construct
ATLGFAS

SEQ ID NO: 149 - protein, 8 aa, synthetic construct
GRTFSGYT

SEQ ID NO: 150 - protein, 8 aa, synthetic construct
AVGSDGST

SEQ ID NO: 151 - protein, 12 aa, synthetic construct
VKLDSGAWSLAE

SEQ ID NO: 152 - protein, 8 aa, synthetic construct
GRTFSSYA

SEQ ID NO: 153 - protein, 8 aa, synthetic construct
VSWTGDNT

SEQ ID NO: 154 - protein, 12 aa, synthetic construct
AAWNWGHHEYTY

SEQ ID NO: 155 - protein, 8 aa, synthetic construct
GRTIGSFV

SEQ ID NO: 156 - protein, 8 aa, synthetic construct
VNWRGSST

SEQ ID NO: 157 - protein, 13 aa, synthetic construct
ARWSWDIGADFGS

SEQ ID NO: 158 - protein, 8 aa, synthetic construct
GFTFSNYA

SEQ ID NO: 159 - protein, 8 aa, synthetic construct
INANSDTT

SEQ ID NO: 160 - protein, 15 aa, synthetic construct
VSDSYIGGLYATYVY

SEQ ID NO: 161 - protein, 8 aa, synthetic construct
VRYFSTYA

SEQ ID NO: 162 - protein, 14 aa, synthetic construct
AAPYGSSQNLEYDY

SEQ ID NO: 163 - protein, 8 aa, synthetic construct
GFTFDDYA

SEQ ID NO: 164 - protein, 8 aa, synthetic construct
ISWNGGST

SEQ ID NO: 165 - protein, 17 aa, synthetic construct
VPVSHSDSVCGSPYMDY

SEQ ID NO: 166 - protein, 8 aa, synthetic construct
GFNLTSDA

SEQ ID NO: 167 - protein, 4 aa, synthetic construct
ISRT

SEQ ID NO: 168 - protein, 18 aa, synthetic construct
AADNAADRCSLSIYNYNL

SEQ ID NO: 169 - protein, 8 aa, synthetic construct
GRTFASFA

SEQ ID NO: 170 - protein, 8 aa, synthetic construct
ISWSGGFT

SEQ ID NO: 171 - protein, 8 aa, synthetic construct
GEWGGFSL

SEQ ID NO: 172 - protein, 8 aa, synthetic construct
IASSDGST

SEQ ID NO: 173 - protein, 20 aa, synthetic construct
VAPCFWFDTVIAGTDPRYDY

SEQ ID NO: 174 - protein, 8 aa, synthetic construct
GFTLGYYA

SEQ ID NO: 175 - protein, 8 aa, synthetic construct
ISGRDGST

SEQ ID NO: 176 - protein, 22 aa, synthetic construct
AAVRGPIVSMDPDLCRPVEFDY

SEQ ID NO: 177 - protein, 8 aa, synthetic construct
GFTFEDYA

SEQ ID NO: 178 - protein, 8 aa, synthetic construct
TSKNDRMP

SEQ ID NO: 179 - protein, 22 aa, synthetic construct
AATNGPAITLFPCHINYWLYDN

SEQ ID NO: 180 - protein, 22 aa, synthetic construct
ASADWRSPTPFPCGVSRSLYDH

SEQ ID NO: 181 - protein, 8 aa, synthetic construct
GNFLRFNA

SEQ ID NO: 182 - protein, 7 aa, synthetic construct
ISSGGRT

SEQ ID NO: 183 - protein, 6 aa, synthetic construct
WSAPDY

SEQ ID NO: 184 - protein, 8 aa, synthetic construct
GSFSSIDT

SEQ ID NO: 185 - protein, 7 aa, synthetic construct
INRGGDT

SEQ ID NO: 186 - protein, 14 aa, synthetic construct
AGSFTLATGDDFGS

SEQ ID NO: 187 - protein, 8 aa, synthetic construct
EQNFSADY

SEQ ID NO: 188 - protein, 7 aa, synthetic construct
ISNSGRT

SEQ ID NO: 189 - protein, 11 aa, synthetic construct
VAEPFSFRRRA

SEQ ID NO: 190 - protein, 8 aa, synthetic construct
EQNFSTDD

SEQ ID NO: 191 - protein, 7 aa, synthetic construct
ITNSGTT

SEQ ID NO: 192 - protein, 14 aa, synthetic construct
GESTTGWAECDFGC

SEQ ID NO: 193 - protein, 8 aa, synthetic construct
RSIFSINA

SEQ ID NO: 194 - protein, 7 aa, synthetic construct
ITNGGTT

SEQ ID NO: 195 - protein, 13 aa, synthetic construct
NANSRYGVGWYNY

SEQ ID NO: 196 - protein, 8 aa, synthetic construct
GSIFSINP

SEQ ID NO: 197 - protein, 7 aa, synthetic construct
FTSGGTT

SEQ ID NO: 198 - protein, 15 aa, synthetic construct
NVRGGHCDPRYWREY

SEQ ID NO: 199 - protein, 15 aa, synthetic construct
NVRGGHYDPRYWREY

SEQ ID NO: 200 - protein, 8 aa, synthetic construct
GRPIDTYA

SEQ ID NO: 201 - protein, 8 aa, synthetic construct
VSWAGVYT

SEQ ID NO: 202 - protein, 20 aa, synthetic construct
AATKLPWNTIVMVQRSYCDY

SEQ ID NO: 203 - protein, 8 aa, synthetic construct
GRTISTYA

SEQ ID NO: 204 - protein, 8 aa, synthetic construct
VSWAGSYT

SEQ ID NO: 205 - protein, 20 aa, synthetic construct
AATKLPWNTSVMVKRSVYDY

SEQ ID NO: 206 - protein, 8 aa, synthetic construct
GRPLECYA

SEQ ID NO: 207 - protein, 8 aa, synthetic construct
ISGNGVNT

SEQ ID NO: 208 - protein, 17 aa, synthetic construct
AATTVPVINLEISHMTY

SEQ ID NO: 209 - protein, 8 aa, synthetic construct
GRRLEGYT

SEQ ID NO: 210 - protein, 8 aa, synthetic construct
ISRNRVNT

SEQ ID NO: 211 - protein, 20 aa, synthetic construct
AATNLPGITLLMSHMNYCDY

SEQ ID NO: 212 - protein, 8 aa, synthetic construct
GPRLEGYT

SEQ ID NO: 213 - protein, 8 aa, synthetic construct
ITRNRVNT

SEQ ID NO: 214 - protein, 17 aa, synthetic construct
AATTVPVINLQVSHINY

SEQ ID NO: 215 - protein, 8 aa, synthetic construct
GRTFNLYA

SEQ ID NO: 216 - protein, 17 aa, synthetic construct
AAKVFPMATLDDDVYDY

SEQ ID NO: 217 - protein, 8 aa, synthetic construct
GVTFSNYV

SEQ ID NO: 218 - protein, 21 aa, synthetic construct
AAESHRRNTIVIVTTPDEYDY

SEQ ID NO: 219 - protein, 23 aa, synthetic construct
AADPSSGYNLFARTVVAFARYDY

SEQ ID NO: 220 - protein, 15 aa, synthetic construct
AAHPAGAHGGLIYKN

SEQ ID NO: 221 - protein, 8 aa, synthetic construct
GRTVSNYA

SEQ ID NO: 222 - protein, 7 aa, synthetic construct
INRSGTP

SEQ ID NO: 223 - protein, 15 aa, synthetic construct
AADPTGAHVGAIYKN

SEQ ID NO: 224 - protein, 8 aa, synthetic construct
GRTFSNYA

SEQ ID NO: 225 - protein, 16 aa, synthetic construct
AADLRGSSWYFDGVDY

SEQ ID NO: 226 - protein, 16 aa, synthetic construct
AADLRGSSWYFDGMDY

SEQ ID NO: 227 - protein, 8 aa, synthetic construct
GRTFSRYA

SEQ ID NO: 228 - protein, 8 aa, synthetic construct
ITRSGTST

SEQ ID NO: 229 - protein, 20 aa, synthetic construct
AAHEAQYSSRWSGTEKGYDY

SEQ ID NO: 230 - protein, 123 aa, synthetic construct
QVQLVESGGGLVQPGGSLRLSCAASGSIFAYHVMGWYRQAPGKQRELVALITSGGSTNYTDSVKGRFTISRDNSKNTVYLQMNSLRAEDTAVYYCAIGYYSGSYYWERSGDYWGQGTLVTVSS

SEQ ID NO: 231 - protein, 123 aa, synthetic construct
EVQLVESGGGLVQPGGSLRLSCAASGSIFAYHVMGWYRQAPGKQLELVSLITSGGSTNYTDSVKGRFTISRDNSKNTLYLQMNSLRAEDTAVYYCAIGYYSGSYYWERSGDYWGQGTQVTVSS

SEQ ID NO: 232 - protein, 123 aa, synthetic construct
QVQLVESGGGVVQPGGSLRLSCAASGSIFAYHVMGWYRQAPGKQRELVSLITSGGSTNYTDSVKGRFTISRDNAKNTLYLQMNSLRAEDTAVYYCAIGYYSGSYYWERSGDYWGQGTQVTVSS

SEQ ID NO: 233 - protein, 123 aa, synthetic construct
EVQLVESGGGLVQPGGSLRLSCAASGSIFAYHVMGWYRQAPGKQRELVALITSGGSTNYTDSVKGRFTISRDNAKNTLYLQMNSLRAEDTAVYYCAIGYYSGSYYWERSGDYWGQGTLVTVSS

SEQ ID NO: 234 - protein, 123 aa, synthetic construct
QVQLVESGGGVVQPGGSLRLSCAASGSIFAYHVMGWYRQAPGKQLELVALITSGGSTNYTDSVKGRFTISRDNAKNTLYLQMNSLRAEDTAVYYCAIGYYSGSYYWERSGDYWGQGTQVTVSS

SEQ ID NO: 235 - protein, 123 aa, synthetic construct
QVQLVESGGGLVQPGGSLRLSCAASGSIFAYHVMGWYRQAPGKQRELVALITSGGSTNYTDSVKGRFTISRDNAKNTVYLQMNSLRAEDTAVYYCAIGYYSGSYYWERSGDYWGQGTQVTVSS

SEQ ID NO: 236 - protein, 123 aa, synthetic construct
QVQLVESGGGVVQPGGSLRLSCAASGSIFAYHVMGWYRQAPGKQRELVSLITSGGSTNYTDSVKGRFTISRDNAKNTVYLQMNSLRAEDTAVYYCAIGYYSGSYYWERSGDYWGQGTQVTVSS

SEQ ID NO: 237 - protein, 123 aa, synthetic construct
QVQLVESGGGLVQPGGSLRLSCAASGSIFAYHVMGWYRQAPGKQRELVALITSGGSTNYTDSVKGRFTISRDNAKNTVYLQMNSLRAEDTAVYYCAIGYYSGSYYWERSGDYWGQGTLVTVSS

SEQ ID NO: 238 - protein, 123 aa, synthetic construct
QVQLVESGGGVVQPGGSLRLSCAASGSIFAYHVMGWYRQAPGKQRELVALITSGGSTNYTDSVKGRFTISRDNSKNTVYLQMNSLRAEDTAVYYCAIGYYSGSYYWERSGDYWGQGTLVTVSS

SEQ ID NO: 239 - protein, 123 aa, synthetic construct
QVQLVESGGGVVQPGGSLRLSCAASGSIFAYHVMGWYRQAPGKQRELVALITSGGSTNYTDSVKGRFTISRDNAKNTLYLQMNSLRAEDTAVYYCAIGYYSGSYYWERSGDYWGQGTLVTVSS

SEQ ID NO: 240 - protein, 123 aa, synthetic construct
EVQLVESGGGLVQPGGSLRLSCAASGSIFAYHVMGWYRQAPGKQRELVALITSGGSTNYTDSVKGRFTISRDNSKNTLYLQMNSLRAEDTAVYYCAIGYYSGSYYWERSGDYWGQGTQVTVSS

SEQ ID NO: 241 - protein, 123 aa, synthetic construct
EVQLVESGGGLVQPGGSLRLSCAASGSIFAYHVMGWYRQAPGKQLELVSLITSGGSTNYTDSVKGRFTISRDNSKNTVYLQMNSLRAEDTAVYYCAIGYYSGSYYWERSGDYWGQGTQVTVSS

SEQ ID NO: 242 - protein, 123 aa, synthetic construct
QVQLVESGGGLVQPGGSLRLSCAASGSIFAYHVMGWYRQAPGKQLELVALITSGGSTNYTDSVKGRFTISRDNSKNTVYLQMNSLRAEDTAVYYCAIGYYSGSYYWERSGDYWGQGTLVTVSS

SEQ ID NO: 243 - protein, 123 aa, synthetic construct
EVQLVESGGGLVQPGGSLRLSCAASGSIFAYHVMGWYRQAPGKQRELVALITSGGSTNYTDSVKGRFTISRDNSKNTVYLQMNSLRAEDTAVYYCAIGYYSGSYYWERSGDYWGQGTLVTVSS

SEQ ID NO: 244 - protein, 123 aa, synthetic construct
QVQLVESGGGLVQPGGSLRLSCAASGSIFAYHVMGWYRQAPGKQRELVALITSGGSTNYTDSVKGRFTISRDNSKNTVYLQMNSLRAEDTAVYYCAIGYYSGSYYWERSGDYWGQGTQVTVSS

SEQ ID NO: 245 - protein, 123 aa, synthetic construct
QVQLVESGGGVVQPGGSLRLSCAASGSIFAYHVMGWYRQAPGKQRELVSLITSGGSTNYTDSVKGRFTISRDNSKNTLYLQMNSLRAEDTAVYYCAIGYYSGSYYWERSGDYWGQGTLVTVSS

SEQ ID NO: 246 - protein, 123 aa, synthetic construct
QVQLVESGGGVVQPGGSLRLSCAASGSIFAYHVMGWYRQAPGKQLELVALITSGGSTNYTDSVKGRFTISRDNSKNTVYLQMNSLRAEDTAVYYCAIGYYSGSYYWERSGDYWGQGTLVTVSS

SEQ ID NO: 247 - protein, 123 aa, synthetic construct
EVQLVESGGGLVQPGGSLRLSCAASGSIFAYHVMGWYRQAPGKQRELVSLITSGGSTNYTDSVKGRFTISRDNSKNTVYLQMNSLRAEDTAVYYCAIGYYSGSYYWERSGDYWGQGTQVTVSS

SEQ ID NO: 248 - protein, 123 aa, synthetic construct
EVQLVESGGGVVQPGGSLRLSCAASGSIFAYHVMGWYRQAPGKQRELVALITSGGSTNYTDSVKGRFTISRDNSKNTLYLQMNSLRAEDTAVYYCAIGYYSGSYYWERSGDYWGQGTLVTVSS

SEQ ID NO: 249 - protein, 123 aa, synthetic construct
EVQLVESGGGVVQPGGSLRLSCAASGSIFAYHVMGWYRQAPGKQRELVALITSGGSTNYTDSVKGRFTISRDNSKNTVYLQMNSLRAEDTAVYYCAIGYYSGSYYWERSGDYWGQGTLVTVSS

SEQ ID NO: 250 - protein, 123 aa, synthetic construct
QVQLVESGGGLVQPGGSLRLSCAASGSIFAYHVMGWYRQAPGKQRELVALITSGGSTNYTDSVKGRFTISRDNSKNTLYLQMNSLRAEDTAVYYCAIGYYSGSYYWERSGDYWGQGTLVTVSS

SEQ ID NO: 251 - protein, 123 aa, synthetic construct
QVQLVESGGGLVQPGGSLRLSCAASGSIFAYHVMGWYRQAPGKQLELVSLITSGGSTNYTDSVKGRFTISRDNSKNTLYLQMNSLRAEDTAVYYCAIGYYSGSYYWERSGDYWGQGTLVTVSS

SEQ ID NO: 252 - protein, 123 aa, synthetic construct
EVQLVESGGGVVQPGGSLRLSCAASGSIFAYHVMGWYRQAPGKQRELVSLITSGGSTNYTDSVKGRFTISRDNSKNTVYLQMNSLRAEDTAVYYCAIGYYSGSYYWERSGDYWGQGTLVTVSS

SEQ ID NO: 253 - protein, 123 aa, synthetic construct
EVQLVESGGGLVQPGGSLRLSCAASGSIFAYHVMGWYRQAPGKQRELVSLITSGGSTNYTDSVKGRFTISRDNSKNTLYLQMNSLRAEDTAVYYCAIGYYSGSYYWERSGDYWGQGTLVTVSS

SEQ ID NO: 254 - protein, 123 aa, synthetic construct
QVQLVESGGGLVQPGGSLRLSCAASGSIFAYHVMGWYRQAPGKQRELVSLITSGGSTNYTDSVKGRFTISRDNAKNTLYLQMNSLRAEDTAVYYCAIGYYSGSYYWERSGDYWGQGTLVTVSS

SEQ ID NO: 255 - protein, 123 aa, synthetic construct
QVQLVESGGGLVQPGGSLRLSCAASGSIFAYHVMGWYRQAPGKQLELVALITSGGSTNYTDSVKGRFTISRDNSKNTVYLQMNSLRAEDTAVYYCAIGYYSGSYYWERSGDYWGQGTQVTVSS

SEQ ID NO: 256 - protein, 123 aa, synthetic construct
QVQLVESGGGVVQPGGSLRLSCAASGSIFAYHVMGWYRQAPGKQRELVSLITSGGSTNYTDSVKGRFTISRDNAKNTVYLQMNSLRAEDTAVYYCAIGYYSGSYYWERSGDYWGQGTLVTVSS

SEQ ID NO: 257 - protein, 123 aa, synthetic construct
QVQLVESGGGLVQPGGSLRLSCAASGSIFAYHVMGWYRQAPGKQRELVSLITSGGSTNYTDSVKGRFTISRDNSKNTVYLQMNSLRAEDTAVYYCAIGYYSGSYYWERSGDYWGQGTLVTVSS

SEQ ID NO: 258 - protein, 123 aa, synthetic construct
QVQLVESGGGLVQPGGSLRLSCAASGSIFAYHVMGWYRQAPGKQRELVSLITSGGSTNYTDSVKGRFTISRDNSKNTVYLQMNSLRAEDTAVYYCAIGYYSGSYYWERSGDYWGQGTQVTVSS

SEQ ID NO: 259 - protein, 124 aa, synthetic construct
SVQLVESEGGLVQAGGSLRLSCVASGRTVSSYAMGWFRQAPGKEREFVAAISWSGRSTYYADSMKGRFTISRDNAKNTVYLQMNNLRPEDTAVYYCATSRAVIPGRDWNYYEYSGQGTQVTVSS

SEQ ID NO: 260 - protein, 121 aa, synthetic construct
QVQLVESGGGLVQAGGSLRLSCAASGRTASAYVMGWFRQAPGKERDFVAGISGGAYYADSVKGRFTIARDNAKNTVYLQMNSLKPEDTAVYYCAAERGMRRLTESYQYDYWGQGTQVTVSS

SEQ ID NO: 261 - protein, 114 aa, synthetic construct
QVKLVESGGGLVQPGGSLRLSCVGSGIIFSASNLAWYRQALGKQRELVAGVTGGGSINYADSVKGRFTISRDNAKNTIYLQMNSLKPEDTAVYYCNARRSYSHWGQGTRVTVSS

SEQ ID NO: 262 - protein, 118 aa, synthetic construct
QVQLVESGGGLVQSGGSLRLSCTASGGFGMMYSMGWYRQAPGKQRELVAARTIDGSTNYADSVKDRFTVFRENNRNTVALEMDSLKPEDTAVYYCNAKPLNGRLTQYWGQGTQVTVSS

SEQ ID NO: 263 - protein, 119 aa, synthetic construct
QVHLVESGGGLVQAGGSLRLSCAASRIRFSINVMGWYRQAPGKQRELVATIAAGGTTNYADSVKGRFTISRDNAKNTVYLQMNSLKPDDTAVYYCNAVLSTLVLPSTYWGQGTQVTVSS

SEQ ID NO: 264 - protein, 121 aa, synthetic construct
QVHLVESGGGLVQPGGSLRLSCIASSERTFRSYTMGWFRQAPGKEREFVAAISWSHSSTYYADSVKGRFTISRDNAKNTVYLQMNNLKPEDTAVYYCAADLRLLPEEYDYYGQGTQVTVSS

SEQ ID NO: 265 - protein, 8 aa, synthetic construct
GRTVSSYA

SEQ ID NO: 266 - protein, 8 aa, synthetic construct
ISWSGRST

SEQ ID NO: 267 - protein, 17 aa, synthetic construct
ATSRAVIPGRDWNYYEY

SEQ ID NO: 268 - protein, 8 aa, synthetic construct
GRTASAYV

SEQ ID NO: 269 - protein, 5 aa, synthetic construct
ISGGA

SEQ ID NO: 270 - protein, 17 aa, synthetic construct
AAERGMRRLTESYQYDY

SEQ ID NO: 271 - protein, 8 aa, synthetic construct
GIIFSASN

SEQ ID NO: 272 - protein, 7 aa, synthetic construct
VTGGGSI

SEQ ID NO: 273 - protein, 8 aa, synthetic construct
NARRSYSH

SEQ ID NO: 274 - protein, 8 aa, synthetic construct
GGFGMMYS

SEQ ID NO: 275 - protein, 7 aa, synthetic construct
RTIDGST

SEQ ID NO: 276 - protein, 12 aa, synthetic construct
NAKPLNGRLTQY

SEQ ID NO: 277 - protein, 8 aa, synthetic construct
RIRFSINV

SEQ ID NO: 278 - protein, 7 aa, synthetic construct
IAAGGTT

SEQ ID NO: 279 - protein, 13 aa, synthetic construct
NAVLSTLVLPSTY

SEQ ID NO: 280 - protein, 9 aa, synthetic construct
SERTFRSYT

SEQ ID NO: 281 - protein, 8 aa, synthetic construct
ISWSHSST

SEQ ID NO: 282 - protein, 13 aa, synthetic construct
AADLRLLPEEYDY

SEQ ID NO: 283 - protein, 124 aa, synthetic construct
QVQLVESGGGLVQPGGSLRLSCVASGRTVSSYAMGWFRQAPGKEREFVAAISWSGRSTYYADSMKGRFTISRDNAKNTVYLQMNSLRAEDTAVYYCATSRAVIPGRDWNYYEYSGQGTQVTVSS

SEQ ID NO: 284 - protein, 124 aa, synthetic construct
QVQLVESGGGLVQPGGSLRLSCVASGRTVSSYAMGWFRQAPGKEREFVAAISWSGRSTYYADSMKGRFTISRDNAKNTVYLQMNSLRAEDTAVYYCATSRAVIPGRDWNYYEYSGQGTLVTVSS

SEQ ID NO: 285 - protein, 124 aa, synthetic construct
QVQLVESGGGLVQPGGSLRLSCVASGRTVSSYAMGWFRQAPGKEREFVAAISWSGRSTYYADSMKGRFTISRDNAKNTVYLQMNSLRAEDTAVYYCATSRAVIPGRDWNYYEYWGQGTQVTVSS

SEQ ID NO: 286 - protein, 124 aa, synthetic construct
QVQLVESGGGLVQPGGSLRLSCAASGRTVSSYAMGWFRQAPGKEREFVAAISWSGRSTYYADSMKGRFTISRDNAKNTVYLQMNSLRAEDTAVYYCATSRAVIPGRDWNYYEYSGQGTQVTVSS

SEQ ID NO: 287 - protein, 124 aa, synthetic construct
QVQLVESGGGLVQPGGSLRLSCAASGRTVSSYAMGWFRQAPGKEREFVAAISWSGRSTYYADSMKGRFTISRDNAKNTVYLQMNSLRAEDTAVYYCATSRAVIPGRDWNYYEYSGQGTLVTVSS

SEQ ID NO: 288 - protein, 124 aa, synthetic construct
QVQLVESGGGLVQPGGSLRLSCAASGRTVSSYAMGWFRQAPGKEREFVSAISWSGRSTYYADSMKGRFTISRDNAKNTLYLQMNSLRAEDTAVYYCATSRAVIPGRDWNYYEYSGQGTLVTVSS

SEQ ID NO: 289 - protein, 124 aa, synthetic construct
QVQLVESGGGLVQPGGSLRLSCAASGRTVSSYAMGWFRQAPGKEREFVSAISWSGRSTYYADSMKGRFTISRDNAKNTLYLQMNSLRAEDTAVYYCATSRAVIPGRDWNYYEYWGQGTLVTVSS

SEQ ID NO: 290 - protein, 124 aa, synthetic construct
QVQLVESGGGLVQPGGSLRLSCVASGRTVSSYAMGWFRQAPGKEREFVSAISWSGRSTYYADSMKGRFTISRDNAKNTLYLQMNSLRAEDTAVYYCATSRAVIPGRDWNYYEYWGQGTLVTVSS

SEQ ID NO: 291 - protein, 124 aa, synthetic construct
QVQLVESGGGLVQPGGSLRLSCAASGRTVSSYAMGWFRQAPGKEREFVSAISWSGRSTYYADSMKGRFTISRDNAKNTVYLQMNSLRAEDTAVYYCATSRAVIPGRDWNYYEYWGQGTLVTVSS

SEQ ID NO: 292 - protein, 114 aa, synthetic construct
QVKLVESGGGLVQPGGSLRLSCVGSGIIFSASNLAWYRQAPGKQRELVAGVTGGGSINYADSVKGRFTISRDNAKNTIYLQMNSLRPEDTAVYYCNARRSYSHWGQGTLVTVSS

SEQ ID NO: 293 - protein, 114 aa, synthetic construct
QVKLVESGGGLVQPGGSLRLSCVGSGIIFSASNLAWYRQAPGKQRELVAGVTGGGSINYADSVKGRFTISRDNAKNTIYLQMNSLRAEDTAVYYCNARRSYSHWGQGTRVTVSS

SEQ ID NO: 294 - protein, 114 aa, synthetic construct
QVKLVESGGGLVQPGGSLRLSCVGSGIIFSASNLAWYRQAPGKQRELVAGVTGGGSINYADSVKGRFTISRDNAKNTIYLQMNSLRAEDTAVYYCNARRSYSHWGQGTLVTVSS

SEQ ID NO: 295 - protein, 114 aa, synthetic construct
QVKLVESGGGLVQPGGSLRLSCVGSGIIFSASNLAWYRQAPGKQRELVAGVTGGGSINYADSVKGRFTISRDNSKNTIYLQMNSLRAEDTAVYYCNARRSYSHWGQGTLVTVSS

SEQ ID NO: 296 - protein, 114 aa, synthetic construct
QVQLVESGGGLVQPGGSLRLSCVGSGIIFSASNLAWYRQAPGKQRELVAGVTGGGSINYADSVKGRFTISRDNAKNTIYLQMNSLRAEDTAVYYCNARRSYSHWGQGTLVTVSS

SEQ ID NO: 297 - protein, 114 aa, synthetic construct
QVKLVESGGGLVQPGGSLRLSCAASGIIFSASNLAWYRQAPGKQRELVAGVTGGGSINYADSVKGRFTISRDNAKNTIYLQMNSLRAEDTAVYYCNARRSYSHWGQGTLVTVSS

SEQ ID NO: 298 - protein, 114 aa, synthetic construct
QVQLVESGGGLVQPGGSLRLSCAASGIIFSASNLAWYRQAPGKQRELVSGVTGGGSINYADSVKGRFTISRDNSKNTIYLQMNSLRAEDTAVYYCNARRSYSHWGQGTLVTVSS

SEQ ID NO: 299 - protein, 114 aa, synthetic construct
QVQLVESGGGLVQPGGSLRLSCAGSGIIFSASNLAWYRQAPGKQRELVAGVTGGGSINYADSVKGRFTISRDNAKNTIYLQMNSLRAEDTAVYYCNARRSYSHWGQGTLVTVSS

SEQ ID NO: 300 - protein, 114 aa, synthetic construct
QVQLVESGGGLVQPGGSLRLSCAASGIIFSASNLAWYRQAPGKQRELVAGVTGGGSINYADSVKGRFTISRDNAKNTIYLQMNSLRAEDTAVYYCNARRSYSHWGQGTLVTVSS

SEQ ID NO: 301 - protein, 114 aa, synthetic construct
QVQLVESGGGLVQPGGSLRLSCVASGIIFSASNLAWYRQAPGKQRELVAGVTGGGSINYADSVKGRFTISRDNAKNTIYLQMNSLRAEDTAVYYCNARRSYSHWGQGTLVTVSS

SEQ ID NO: 302 - protein, 114 aa, synthetic construct
QVQLVESGGGLVQPGGSLRLSCVGSGIIFSASNLAWYRQAPGKQRELVAGVTGGGSINYADSVKGRFTISRDNAKNTIYLQMNSLRAEDTAVYYCNARRSYSHWGQGTRVTVSS

SEQ ID NO: 303 - protein, 121 aa, synthetic construct
QVQLVESGGGLVQPGGSLRLSCAASGRTASAYVMGWFRQAPGKERDFVAGISGGAYYADSVKGRFTIARDNAKNTVYLQMNSLRAEDTAVYYCAAERGMRRLTESYQYDYWGQGTQVTVSS

SEQ ID NO: 304 - protein, 121 aa, synthetic construct
QVQLVESGGGLVQPGGSLRLSCAASGRTASAYVMGWFRQAPGKERDFVAGISGGAYYADSVKGRFTIARDNAKNTVYLQMNSLRAEDTAVYYCAAERGMRRLTESYQYDYWGQGTLVTVSS

SEQ ID NO: 305 - protein, 121 aa, synthetic construct
QVQLVESGGGLVQPGGSLRLSCAASGRTASAYVMGWFRQAPGKEREFVAGISGGAYYADSVKGRFTIARDNAKNTVYLQMNSLRAEDTAVYYCAAERGMRRLTESYQYDYWGQGTLVTVSS

SEQ ID NO: 306 - protein, 121 aa, synthetic construct
QVQLVESGGGLVQPGGSLRLSCAASGRTASAYVMGWFRQAPGKERDFVAGISGGAYYADSVKGRFTISRDNAKNTVYLQMNSLRAEDTAVYYCAAERGMRRLTESYQYDYWGQGTLVTVSS

SEQ ID NO: 307 - protein, 121 aa, synthetic construct
QVQLVESGGGLVQPGGSLRLSCAASGRTASAYVMGWFRQAPGKEREFVAGISGGAYYADSVKGRFTISRDNSKNTVYLQMNSLRAEDTAVYYCAAERGMRRLTESYQYDYWGQGTLVTVSS

SEQ ID NO: 308 - protein, 118 aa, synthetic construct
QVQLVESGGGLVQPGGSLRLSCAASGGFGMMYSMGWYRQAPGKQRELVAARTIDGSTNYADSVKDRFTISRENNRNTLYLQNDSLRAEDTAVYYCNAKPLNGRLTQYWGQGTQVTVSS

SEQ ID NO: 309 - protein, 118 aa, synthetic construct
QVQLVESGGGLVQPGGSLRLSCAASGGFGMMYSMGWYRQAPGKQRELVAARTIDGSTNYADSVKDRFTISRENNRNTLYLQMDSLRAEDTAVYYCNAKPLNGRLTQYWGQGTQVTVSS

SEQ ID NO: 310 - protein, 118 aa, synthetic construct
QVQLVESGGGLVQPGGSLRLSCAASGGFGMMYSMGWYRQAPGKQRELVAARTIDGSTNYADSVKDRFTVSRENNRNTLALQNDSLRAEDTAVYYCNAKPLNGRLTQYWGQGTQVTVSS

SEQ ID NO: 311 - protein, 118 aa, synthetic construct
QVQLVESGGGLVQPGGSLRLSCTASGGFGMMYSMGWYRQAPGKQRELVAARTIDGSTNYADSVKDRFTIFRENNRNTLYLQMDSLRAEDTAVYYCNAKPLNGRLTQYWGQGTQVTVSS

SEQ ID NO: 312 - protein, 118 aa, synthetic construct
QVQLVESGGGLVQPGGSLRLSCAASGGFGMMYSMGWYRQAPGKQRELVAARTIDGSTNYADSVKDRFTISRENNRNTVYLQNDSLRAEDTAVYYCNAKPLNGRLTQYWGQGTLVTVSS

SEQ ID NO: 313 - protein, 118 aa, synthetic construct
QVQLVESGGGLVQPGGSLRLSCTASGGFGMMYSMGWYRQAPGKQRELVAARTIDGSTNYADSVKDRFTVFRENNRNTVYLQNDSLRAEDTAVYYCNAKPLNGRLTQYWGQGTQVTVSS

SEQ ID NO: 314 - protein, 118 aa, synthetic construct
QVQLVESGGGLVQPGGSLRLSCTASGGFGMMYSMGWYRQAPGKQRELVAARTIDGSTNYADSVKDRFTVFRENNRNTVYLQMDSLRAEDTAVYYCNAKPLNGRLTQYWGQGTQVTVSS

SEQ ID NO: 315 - protein, 118 aa, synthetic construct
QVQLVESGGGLVQPGGSLRLSCTASGGFGMMYSMGWYRQAPGKQRELVAARTIDGSTNYADSVKDRFTVFRENNRNTVALQNDSLRAEDTAVYYCNAKPLNGRLTQYWGQGTQVTVSS

SEQ ID NO: 316 - protein, 118 aa, synthetic construct
QVQLVESGGGLVQPGGSLRLSCAASGGFGMMYSMGWYRQAPGKQRELVAARTIDGSTNYADSVKDRFTIFRENNRNTVALQNDSLRAEDTAVYYCNAKPLNGRLTQYWGQGTQVTVSS

SEQ ID NO: 317 - protein, 118 aa, synthetic construct
QVQLVESGGGLVQPGGSLRLSCTASGGFGMMYSMGWYRQAPGKQRELVAARTIDGSTNYADSVKDRFTVFRENNRNTVYLQMDSLRAEDTAVYYCNAKPLNGRLTQYWGQGTLVTVSS

SEQ ID NO: 318 - protein, 118 aa, synthetic construct
QVQLVESGGGLVQPGGSLRLSCAASGGFGMMYSMGWYRQAPGKQRELVAARTIDGSTNYADSVKDRFTVFRENNRNTVYLQMDSLRAEDTAVYYCNAKPLNGRLTQYWGQGTLVTVSS

SEQ ID NO: 319 - protein, 118 aa, synthetic construct
QVQLVESGGGLVQPGGSLRLSCAASGGFGMMYSMGWYRQAPGKQRELVAARTIDGSTNYADSVKDRFTVSRENNRNTVALQNDSLRAEDTAVYYCNAKPLNGRLTQYWGQGTLVTVSS

SEQ ID NO: 320 - protein, 118 aa, synthetic construct
QVQLVESGGGLVQPGGSLRLSCTASGGFGMMYSMGWYRQAPGKQRELVAARTIDGSTNYADSVKDRFTVSRENNRNTVYLQMDSLRAEDTAVYYCNAKPLNGRLTQYWGQGTLVTVSS

SEQ ID NO: 321 - protein, 116 aa, synthetic construct
QVQFVESGGGLVQAGGSLRLSCAASGITVSRNDMGWYRQAPGKQRELVAIMNRVGSTDTADSVKDRFTISRDNTKNTLYLQMNNLKPEDTAVYYCNALNTVITWPWGQGTQVTVSS

SEQ ID NO: 322 - protein, 122 aa, synthetic construct
QVQLVESGGGLVQAGGSLRLSCTASVHIISHYSMGWFRQAPGKERELVAAIPVSGRVPYYLESVKGRFTISRDNAKNRLYLQMNNLKAEDTAVYYCAAYPRKGLEGNEYEYWGQGTQVTVSS

SEQ ID NO: 323 - protein, 122 aa, synthetic construct
QVQLVESGGGSVQAGGSLRLSCVDSGHTLSTYAMGWFRQAPGKEREFVAAIARDGGATYLSGSAQGRATISRDNAKSTVYLQMNNLKPDDTAVYYCAASSMFSTAKRDYSYWGQGTQVTVSS

SEQ ID NO: 324 - protein, 122 aa, synthetic construct
QVHLVESGGTLVQAGGSLRLSCAASGRTFSTYAMGWFRQGPGKEREFVAVIDTTGSASAYAGSVRGRFTVSRDNTENTVYLQMNTLKPEDTAVYYCAAARRYSTAPGDYDYWGQGTQVTVSS

SEQ ID NO: 325 - protein, 122 aa, synthetic construct
QVQLVESGGGLVQAGGSLRLSCAASGSHFSIATMGWYRQAPGMERELVAVLSSSGRPTYADSVKGRFAIYRNNSMNIVELQMNGLKPEDTAVYVCKANLKRFFIEERYRDYWGQGTQVIVSS

SEQ ID NO: 326 - protein, 8 aa, synthetic construct
GITVSRND

SEQ ID NO: 327 - protein, 8 aa, synthetic construct
IMNRVGST

SEQ ID NO: 328 - protein, 10 aa, synthetic construct
NALNTVITWP

SEQ ID NO: 329 - protein, 8 aa, synthetic construct
VHIISHYS

SEQ ID NO: 330 - protein, 8 aa, synthetic construct
IPVSGRVP

SEQ ID NO: 331 - protein, 15 aa, synthetic construct
AAYPRKGLEGNEYEY

SEQ ID NO: 332 - protein, 8 aa, synthetic construct
GHTLSTYA

SEQ ID NO: 333 - protein, 8 aa, synthetic construct
IARDGGAT

SEQ ID NO: 334 - protein, 15 aa, synthetic construct
AASSMFSTAKRDYSY

SEQ ID NO: 335 - protein, 8 aa, synthetic construct
GRTFSTYA

SEQ ID NO: 336 - protein, 8 aa, synthetic construct
IDTTGSAS

SEQ ID NO: 337 - protein, 15 aa, synthetic construct
AAARRYSTAPGDYDY

SEQ ID NO: 338 - protein, 8 aa, synthetic construct
GSHFSIAT

SEQ ID NO: 339 - protein, 7 aa, synthetic construct
LSSSGRP

SEQ ID NO: 340 - protein, 16 aa, synthetic construct
KANLKRFFIEERYRDY

SEQ ID NO: 341 - protein, 116 aa, synthetic construct
QVQLVESGGGLIQPGGSLRLSCAASGITVSRNDMGWYRQAPGKQRELVAIMNRVGSTDTADSVKGRFTISRDNTKNSLYLQMNSLRAEDTAVYYCNALNTVITWPWGQGTQVTVSS

SEQ ID NO: 342 - protein, 116 aa, synthetic construct
QVQFVESGGGLIQPGGSLRLSCAASGITVSRNDMGWYRQAPGKQRELVAIMNRVGSTDTADSVKGRFTISRDNTKNSLYLQMNSLRAEDTAVYYCNALNTVITWPWGQGTQVTVSS

SEQ ID NO: 343 - protein, 116 aa, synthetic construct
QVQLVESGGGLVQPGGSLRLSCAASGITVSRNDMGWYRQAPGKQRELVAIMNRVGSTDTADSVKGRFTISRDNTKNSLYLQMNSLRAEDTAVYYCNALNTVITWPWGQGTQVTVSS

SEQ ID NO: 344 - protein, 116 aa, synthetic construct
QVQLVESGGGLIQPGGSLRLSCAASGITVSRNDMGWYRQAPGKQRELVAIMNRVGSTDTADSVKGRFTISRDNTKNTLYLQMNSLRAEDTAVYYCNALNTVITWPWGQGTQVTVSS

SEQ ID NO: 345 - protein, 116 aa, synthetic construct
QVQFVESGGGLVQPGGSLRLSCAASGITVSRNDMGWYRQAPGKQRELVAIMNRVGSTDTADSVKGRFTISRDNTKNTLYLQMNSLRAEDTAVYYCNALNTVITWPWGQGTQVTVSS

SEQ ID NO: 346 - protein, 5 aa, synthetic construct
TYSVA

SEQ ID NO: 347 - protein, 17 aa, synthetic construct
CISSRDGNTFYSDSVKG

SEQ ID NO: 348 - protein, 23 aa, synthetic construct
GAQAHCTIFTSYFNSDYYRRYNY

SEQ ID NO: 349 - protein, 5 aa, synthetic construct
DKVMS

SEQ ID NO: 350 - protein, 17 aa, synthetic construct
TITPGGTATSYTESVKG

SEQ ID NO: 351 - protein, 9 aa, synthetic construct
GGPGGRYDN

SEQ ID NO: 352 - protein, 7 aa, synthetic construct
INTMTMG

SEQ ID NO: 353 - protein, 15 aa, synthetic construct
ASRGATTNYADSVKG

SEQ ID NO: 354 - protein, 12 aa, synthetic construct
DRYGFGYGDNDY

SEQ ID NO: 355 - protein, 10 aa, synthetic construct
GFILDTYSVA

SEQ ID NO: 356 - protein, 10 aa, synthetic construct
CISSRDGNTF

SEQ ID NO: 357 - protein, 23 aa, synthetic construct
GAQAHCTIFTSYFNSDYYRRYNY

SEQ ID NO: 358 - protein, 10 aa, synthetic construct
GFIFSDKVMS

SEQ ID NO: 359 - protein, 10 aa, synthetic construct
TITPGGTATS

SEQ ID NO: 360 - protein, 9 aa, synthetic construct
GGPGGRYDN

SEQ ID NO: 361 - protein, 12 aa, synthetic construct
RSIFEINTMTMG

SEQ ID NO: 362 - protein, 8 aa, synthetic construct
ASRGATTN

SEQ ID NO: 363 - protein, 12 aa, synthetic construct
DRYGFGYGDNDY

SEQ ID NO: 364 - protein, 6 aa, synthetic construct
DTYSVA

SEQ ID NO: 365 - protein, 13 aa, synthetic construct
GVSCISSRDGNTF

SEQ ID NO: 366 - protein, 24 aa, synthetic construct
AAGAQAHCTIFTSYFNSDYYRRYN

SEQ ID NO: 367 - protein, 6 aa, synthetic construct
SDKVMS

SEQ ID NO: 368 - protein, 13 aa, synthetic construct
WVSTITPGGTATS

SEQ ID NO: 369 - protein, 10 aa, synthetic construct
RIGGPGGRYD

SEQ ID NO: 370 - protein, 8 aa, synthetic construct
EINTMTMG

SEQ ID NO: 371 - protein, 11 aa, synthetic construct
LISASRGATTN

SEQ ID NO: 372 - protein, 13 aa, synthetic construct
SADRYGFGYGDND

SEQ ID NO: 373 - protein, 7 aa, synthetic construct
GFILDTY

SEQ ID NO: 374 - protein, 6 aa, synthetic construct
SSRDGN

SEQ ID NO: 375 - protein, 23 aa, synthetic construct
GAQAHCTIFTSYFNSDYYRRYNY

SEQ ID NO: 376 - protein, 7 aa, synthetic construct
GFIFSDK

SEQ ID NO: 377 - protein, 6 aa, synthetic construct
TPGGTA

SEQ ID NO: 378 - protein, 9 aa, synthetic construct
GGPGGRYDN

SEQ ID NO: 379 - protein, 7 aa, synthetic construct
RSIFEIN

SEQ ID NO: 380 - protein, 4 aa, synthetic construct
RGAT

SEQ ID NO: 381 - protein, 12 aa, synthetic construct
DRYGFGYGDNDY

SEQ ID NO: 382 - protein, 119 aa, synthetic construct
QVQLVESGGGLVQPGGSLRLSCAASGRTLNNYVVAWFRQAPGKEREFVATMWWSGGSPWYSDNVKGRFTISRDNSKNTLYLQMNSLRAEDTAVYYCAATWVGTSEYRHWGQGTLVTVSS

SEQ ID NO: 383 - protein, 119 aa, synthetic construct
QVQLVESGGGLVQPGGSLRLSCAASGRTLNNYVVAWFRQAPGKEREFVSTMWWSGGSPWYSDNVKGRFTISRDNSKNTLYLQMNSLRAEDTAVYYCAATWVGTSEYRHWGQGTQVTVSS

SEQ ID NO: 384 - protein, 119 aa, synthetic construct
QVQLVESGGGLVQTGGSLRLSCAASGRTLNNYVVAWFRQAPGKEREFVATMWWSGGSPWYSDNVKGRFTISRDNSKNTLYLQMNSLRPEDTAVYYCAATWVGTSEYRHWGQGTLVTVSS

SEQ ID NO: 385 - protein, 119 aa, synthetic construct
EVQLVESGGGLVQTGGSLRLSCAASGRTLNNYVVAWFRQAPGKEREFVATMWWSGGSPWYSDNVKGRFTISRDNSKNTLYLQMNSLRAEDTAVYYCAATWVGTSEYRHWGQGTLVTVSS

SEQ ID NO: 386 - protein, 119 aa, synthetic construct
QVQLVESGGGLVQPGGSLRLSCAASGRTLNNYVVAWFRQAPGKEREFVATMWWSGGSPWYSDNVKGRFTISRDNSKNTLYLQMNSLRAEDTAVYYCAATWVGTSEYRHWGQGTQVTVSS

SEQ ID NO: 387 - protein, 119 aa, synthetic construct
QVQLVESGGGLVQPGGSLRLSCAASGRTLNNYVVAWFRQAPGKEREFVATMWWSGGSPWYSDNVKGRFTISRDNAKNTLYLQMNSLRAEDTAVYYCAATWVGTSEYRHWGQGTLVTVSS

SEQ ID NO: 388 - protein, 119 aa, synthetic construct
HVQLVESGGGLVQPGGSLRLSCAASGRTLNNYVVAWFRQAPGKEREFVATMWWSGGSPWYSDNVKGRFTISRDNSKNTLYLQMNSLRAEDTAVYYCAATWVGTSEYRHWGQGTLVTVSS

SEQ ID NO: 389 - protein, 119 aa, synthetic construct
EVQLVESGGGLVQPGGSLRLSCAASGRTLNNYVVAWFRQAPGKEREFVSTMWWSGGSPWYSDNVKGRFTISRDNSKNTLYLQMNSLRPEDTAVYYCAATWVGTSEYRHWGQGTLVTVSS

SEQ ID NO: 390 - protein, 119 aa, synthetic construct
QVQLVESGGGLVQTGGSLRLSCAASGRTLNNYVVAWFRQAPGKEREFVSTMWWSGGSPWYSDNVKGRFTISRDNSKNTLYLQMNSLRPEDTAVYYCAATWVGTSEYRHWGQGTLVTVSS

SEQ ID NO: 391 - protein, 119 aa, synthetic construct
EVQLVESGGGLVQPGGSLRLSCAASGRTLNNYVVAWFRQAPGKEREFVATMWWSGGSPWYSDNVKGRFTISRDNAKNTLYLQMNSLRPEDTAVYYCAATWVGTSEYRHWGQGTQVTVSS

SEQ ID NO: 392 - protein, 119 aa, synthetic construct
EVQLVESGGGLVQPGGSLRLSCAASGRTLNNYVVAWFRQAPGKEREFVSTMWWSGGSPWYSDNVKGRFTISRDNAKNTLYLQMNSLRAEDTAVYYCAATWVGTSEYRHWGQGTLVTVSS

SEQ ID NO: 393 - protein, 119 aa, synthetic construct
EVQLVESGGGLVQPGGSLRLSCAASGRTLNNYVVAWFRQAPGKEREFVATMWWSGGSPWYSDNVKGRFTISRDNSKNTLYLQMNSLRAEDTAVYYCAATWVGTSEYRHWGQGTQVTVSS

SEQ ID NO: 394 - protein, 119 aa, synthetic construct
QVQLVESGGGLVQTGGSLRLSCAASGRTLNNYVVAWFRQAPGKEREFVATMWWSGGSPWYSDNVKGRFTISRDNAKNTLYLQMNSLRPEDTAVYYCAATWVGTSEYRHWGQGTLVTVSS

SEQ ID NO: 395 - protein, 119 aa, synthetic construct
HVQLVESGGGLVQPGGSLRLSCAASGRTLNNYVVAWFRQAPGKEREFVSTMWWSGGSPWYSDNVKGRFTISRDNSKNTLYLQMNSLRAEDTAVYYCAATWVGTSEYRHWGQGTQVTVSS

SEQ ID NO: 396 - protein, 119 aa, synthetic construct
HVQLVESGGGLVQPGGSLRLSCAASGRTLNNYVVAWFRQAPGKEREFVATMWWSGGSPWYSDNVKGRFTISRDNAKNTLYLQMNSLRAEDTAVYYCAATWVGTSEYRHWGQGTLVTVSS

SEQ ID NO: 397 - protein, 119 aa, synthetic construct
EVQLVESGGGLVQPGGSLRLSCAASGRTLNNYVVAWFRQAPGKEREFVATMWWSGGSPWYSDNVKGRFTISRDNSKNTLYLQMNSLRAEDTAVYYCAATWVGTSEYRHWGQGTLVTVSS

SEQ ID NO: 398 - protein, 119 aa, synthetic construct
EVQLVESGGGLVQTGGSLRLSCAASGRTLNNYVVAWFRQAPGKEREFVSTMWWSGGSPWYSDNVKGRFTISRDNAKNTLYLQMNSLRAEDTAVYYCAATWVGTSEYRHWGQGTLVTVSS

SEQ ID NO: 399 - protein, 119 aa, synthetic construct
HVQLVESGGGLVQTGGSLRLSCAASGRTLNNYVVAWFRQAPGKEREFVATMWWSGGSPWYSDNVKGRFTISRDNSKNTLYLQMNSLRAEDTAVYYCAATWVGTSEYRHWGQGTQVTVSS

SEQ ID NO: 400 - protein, 119 aa, synthetic construct
HVQLVESGGGLVQPGGSLRLSCAASGRTLNNYVVAWFRQAPGKEREFVATMWWSGGSPWYSDNVKGRFTISRDNSKNTLYLQMNSLRPEDTAVYYCAATWVGTSEYRHWGQGTLVTVSS

SEQ ID NO: 401 - protein, 119 aa, synthetic construct
EVQLVESGGGLVQPGGSLRLSCAASGRTLNNYVVAWFRQAPGKEREFVSTMWWSGGSPWYSDNVKGRFTISRDNSKNTLYLQMNSLRAEDTAVYYCAATWVGTSEYRHWGQGTLVTVSS

SEQ ID NO: 402 - protein, 5 aa, synthetic construct
YHVMG

SEQ ID NO: 403 - protein, 16 aa, synthetic construct
LITSGGSTNYTDSVKG

SEQ ID NO: 404 - protein, 15 aa, synthetic construct
GYYSGSYYWERSGDY

SEQ ID NO: 405 - protein, 5 aa, synthetic construct
NYVVA

SEQ ID NO: 406 - protein, 17 aa, synthetic construct
TMWWSGGSPWYSDNVKG

SEQ ID NO: 407 - protein, 10 aa, synthetic construct
TWVGTSEYRH

SEQ ID NO: 408 - protein, 10 aa, synthetic construct
GSIFAYHVMG

SEQ ID NO: 409 - protein, 9 aa, synthetic construct
LITSGGSTN

SEQ ID NO: 410 - protein, 15 aa, synthetic construct
GYYSGSYYWERSGDY

SEQ ID NO: 411 - protein, 10 aa, synthetic construct
GRTLNNYVVA

SEQ ID NO: 412 - protein, 10 aa, synthetic construct
TMWWSGGSPW

SEQ ID NO: 413 - protein, 10 aa, synthetic construct
TWVGTSEYRH

SEQ ID NO: 414 - protein, 6 aa, synthetic construct
AYHVMG

SEQ ID NO: 415 - protein, 12 aa, synthetic construct
LVALITSGGSTN

SEQ ID NO: 416 - protein, 16 aa, synthetic construct
AIGYYSGSYYWERSGD

SEQ ID NO: 417 - protein, 6 aa, synthetic construct
NNYVVA

SEQ ID NO: 418 - protein, 13 aa, synthetic construct
FVATMWWSGGSPW

SEQ ID NO: 419 - protein, 11 aa, synthetic construct
AATWVGTSEYR

SEQ ID NO: 420 - protein, 7 aa, synthetic construct
GSIFAYH

SEQ ID NO: 421 - protein, 5 aa, synthetic construct
TSGGS

SEQ ID NO: 422 - protein, 15 aa, synthetic construct
GYYSGSYYWERSGDY

SEQ ID NO: 423 - protein, 7 aa, synthetic construct
GRTLNNY

SEQ ID NO: 424 - protein, 6 aa, synthetic construct
WWSGGS

SEQ ID NO: 425 - protein, 10 aa, synthetic construct
TWVGTSEYRH

SEQ ID NO: 426 - protein, 5 aa, synthetic construct
SYAMG

SEQ ID NO: 427 - protein, 17 aa, synthetic construct
AISWSGRSTYYADSMKG

SEQ ID NO: 428 - protein, 15 aa, synthetic construct
SRAVIPGRDWNYYEY

SEQ ID NO: 429 - protein, 5 aa, synthetic construct
ASNLA

SEQ ID NO: 430 - protein, 16 aa, synthetic construct
GVTGGGSINYADSVKG

SEQ ID NO: 431 - protein, 6 aa, synthetic construct
RRSYSH

SEQ ID NO: 432 - protein, 5 aa, synthetic construct
AYVMG

SEQ ID NO: 433 - protein, 14 aa, synthetic construct
GISGGAYYADSVKG

SEQ ID NO: 434 - protein, 15 aa, synthetic construct
ERGMRRLTESYQYDY

SEQ ID NO: 435 - protein, 5 aa, synthetic construct
MYSMG

SEQ ID NO: 436 - protein, 16 aa, synthetic construct
ARTIDGSTNYADSVKD

SEQ ID NO: 437 - protein, 10 aa, synthetic construct
KPLNGRLTQY

SEQ ID NO: 438 - protein, 10 aa, synthetic construct
GRTVSSYAMG

SEQ ID NO: 439 - protein, 10 aa, synthetic construct
AISWSGRSTY

SEQ ID NO: 440 - protein, 15 aa, synthetic construct
SRAVIPGRDWNYYEY

SEQ ID NO: 441 - protein, 10 aa, synthetic construct
GIIFSASNLA

SEQ ID NO: 442 - protein, 9 aa, synthetic construct
GVTGGGSIN

SEQ ID NO: 443 - protein, 6 aa, synthetic construct
RRSYSH

SEQ ID NO: 444 - protein, 10 aa, synthetic construct
GRTASAYVMG

SEQ ID NO: 445 - protein, 7 aa, synthetic construct
GISGGAY

SEQ ID NO: 446 - protein, 15 aa, synthetic construct
ERGMRRLTESYQYDY

SEQ ID NO: 447 - protein, 10 aa, synthetic construct
GGFGMMYSMG

SEQ ID NO: 448 - protein, 9 aa, synthetic construct
ARTIDGSTN

SEQ ID NO: 449 - protein, 10 aa, synthetic construct
KPLNGRLTQY

SEQ ID NO: 450 - protein, 6 aa, synthetic construct
SSYAMG

SEQ ID NO: 451 - protein, 13 aa, synthetic construct
FVSAISWSGRSTY

SEQ ID NO: 452 - protein, 16 aa, synthetic construct
ATSRAVIPGRDWNYYE

SEQ ID NO: 453 - protein, 6 aa, synthetic construct
SASNLA

SEQ ID NO: 454 - protein, 12 aa, synthetic construct
LVAGVTGGGSIN

SEQ ID NO: 455 - protein, 7 aa, synthetic construct
NARRSYS

SEQ ID NO: 456 - protein, 6 aa, synthetic construct
SAYVMG

SEQ ID NO: 457 - protein, 10 aa, synthetic construct
FVAGISGGAY

SEQ ID NO: 458 - protein, 16 aa, synthetic construct
AAERGMRRLTESYQYD

SEQ ID NO: 459 - protein, 6 aa, synthetic construct
MMYSMG

SEQ ID NO: 460 - protein, 12 aa, synthetic construct
LVAARTIDGSTN

SEQ ID NO: 461 - protein, 11 aa, synthetic construct
NAKPLNGRLTQ

SEQ ID NO: 462 - protein, 7 aa, synthetic construct
GRTVSSY

SEQ ID NO: 463 - protein, 6 aa, synthetic construct
SWSGRS

SEQ ID NO: 464 - protein, 15 aa, synthetic construct
SRAVIPGRDWNYYEY

SEQ ID NO: 465 - protein, 7 aa, synthetic construct
GIIFSAS

SEQ ID NO: 466 - protein, 5 aa, synthetic construct
TGGGS

SEQ ID NO: 467 - protein, 7 aa, synthetic construct
GRTASAY

SEQ ID NO: 468 - protein, 4 aa, synthetic construct
ISGG

SEQ ID NO: 469 - protein, 15 aa, synthetic construct
ERGMRRLTESYQYDY

SEQ ID NO: 470 - protein, 7 aa, synthetic construct
GGFGMMY

SEQ ID NO: 471 - protein, 5 aa, synthetic construct
TIDGS

SEQ ID NO: 472 - protein, 10 aa, synthetic construct
KPLNGRLTQY

SEQ ID NO: 473 - protein, 127 aa, synthetic construct
QVQLVESGGGLVQAGGSLRLSCATSGRPLESYAMGWFRQAPGKEREFVAAVSWNRGYTYYADSLKGRFTISRDNAKNTMVLQMNRLKPEDTAVYYCAATDFPWNTPVMGKRSLYEYWRWGTRTNVSS

SEQ ID NO: 474 - protein, 8 aa, synthetic construct
GRPLESYA

SEQ ID NO: 475 - protein, 8 aa, synthetic construct
VSWNRGYT

SEQ ID NO: 476 - protein, 20 aa, synthetic construct
AATDFPWNTPVMGKRSLYEY

SEQ ID NO: 477 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 478 - protein, 11 aa, synthetic construct
WGQGTQVIVSS

SEQ ID NO: 479 - protein, 5 aa, synthetic construct
RNDMG

SEQ ID NO: 480 - protein, 16 aa, synthetic construct
IMNRVGSTDTADSVKG

SEQ ID NO: 481 - protein, 8 aa, synthetic construct
LNTVITWP

SEQ ID NO: 482 - protein, 10 aa, synthetic construct
GITVSRNDMG

SEQ ID NO: 483 - protein, 9 aa, synthetic construct
IMNRVGSTD

SEQ ID NO: 484 - protein, 8 aa, synthetic construct
LNTVITWP

SEQ ID NO: 485 - protein, 6 aa, synthetic construct
SRNDMG

SEQ ID NO: 486 - protein, 12 aa, synthetic construct
LVAIMNRVGSTD

SEQ ID NO: 487 - protein, 9 aa, synthetic construct
NALNTVITW

SEQ ID NO: 488 - protein, 7 aa, synthetic construct
GITVSRN

SEQ ID NO: 489 - protein, 5 aa, synthetic construct
NRVGS

SEQ ID NO: 490 - protein, 8 aa, synthetic construct
LNTVITWP

SEQ ID NO: 491 - protein, 6 aa, synthetic construct
RRSYSH

SEQ ID NO: 492 - protein, 6 aa, synthetic construct
HHHHHH

SEQ ID NO: 493 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 494 - RNA, 250 nt, synthetic construct
  source 1..250: mol_type = other RNA
  misc_difference 1..250: note = This sequence may encompass 100-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 495 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0274983 A1
(43) Pub. Date: Sep. 17, 2026

(54) ANTI-CD38, ANTI-BCMA, ANTI-GPRC5D, AND ANTI-FCRH5 ANTIBODIES AND USES TO TREAT VARIOUS DISEASES SUCH AS CANCER

(71) Applicant: ModernaTX, Inc., Cambridge, MA (US)

(72) Inventors: Michael Deci, Cambridge, MA (US); Graham Farrington, Acton, MA (US); Ying Fu, Cambridge, MA (US); Maiko Obana, Cambridge, MA (US); Anuradha Yammanuru, Sharon, MA (US); Zhen Zhang, Brighton, MA (US)

(21) Appl. No.: 19/166,098

(22) PCT Filed: Mar. 25, 2024
(86) PCT No.: PCT/US2024/021287

§ 371 Date: Sep. 17, 2025

Related U.S. Application Data
(60) Provisional application No. 63/454,829, filed on Mar. 27, 2023.
(60) Provisional application No. 63/612,440, filed on Dec. 20, 2023.

(51) Int. Cl. C07K 16/28; A61K 48/00

(52) U.S. Cl. CPC C07K 16/2896; A61K 48/00; C07K 16/28; C07K 16/283; C07K 16/2878; C07K 2317/10; C07K 2317/24; C07K 2317/92

(57) ABSTRACT

Described herein are anti-CD38, anti-BCMA, anti-GPRC5D, and anti-FcRH5 antibodies and binding proteins including such binding domains, as well as nucleic acids encoding the same. The disclosure also features methods of using such antibodies and binding proteins to treat various diseases such as cancer.

### CROSS-REFERENCES TO RELATED APPLICATIONS

This application claims the benefit of priority of U.S. Provisional Appl. No. 63/454,829, filed Mar. 27, 2023, and U.S. Provisional Appl. No. 63/612,440, filed Dec. 20, 2023, the contents of both of which are incorporated by reference in their entirety herein.

### SEQUENCE LISTING

The instant application contains a sequence listing which has been submitted electronically in xml file format and is hereby incorporated by reference in its entirety. Said xml copy, created on Feb. 22, 2024, is named 45817-0157WO1_SL.xml and is 450,487 bytes in size.

### FIELD

The present disclosure relates generally to antibodies and binding molecules, that specifically bind tumor targeting antigens found on multiple myeloma cells (e.g., CD38, BCMA, GPRC5 D, and FcRH5) and nucleic acids encoding the same. The present disclosure further relates to methods of producing the disclosed antibodies, binding domains, proteins (e.g., purified anti-CD38, anti-BCMA, anti-GPRC5D, and anti-FcRH5 binding proteins or chimeric molecules comprising such binding proteins), and nucleic acid molecules encoding such binding proteins and/or chimeric molecules, as well as medical applications and treatments utilizing the disclosed antibodies, binding domains, proteins, and nucleic acid molecules.

### BACKGROUND

The following description of the background of the present technology is provided simply as an aid in understanding the present technology and is not admitted to describe or constitute prior art to the present technology.

Multiple myeloma (MM) is a malignancy of bone marrow plasma cells and is the second most common hematologic malignancy after non-Hodgkin's lymphoma. It is estimated that MM accounts for nearly 2% of all cancers with about 34,000 new cases in the US annually. The prevalence of MM has increased due to therapeutic advances with about 150,000 MM patients living in the US. The cause of MM is unknown, although some environmental and occupational exposures have been potentially associated, including pesticides and farming. There therefore remains a need for antibodies, binding domains, and related proteins that bind tumor-targeting antigens, including those that target MM (e.g., CD38, BCMA, GPRC5D, and FcRH5) to facilitate the reduction or elimination of such cells.

The following application describes the development and characterization of novel CD38-, BCMA-, GPRC5D-, and FcRH5-binding antibodies, binding domains, and related proteins, including single domain antibodies (sdAbs).

### SUMMARY OF THE INVENTION

The present disclosure provides, among other things, antibodies, binding domains, and related proteins that bind CD38, B-cell maturation antigen (BCMA), G protein-coupled receptor, class C, group 5, member D (GPRC5D), and Fc receptor-homolog 5 (FcRH5), and nucleic acids encoding the same. Such antibodies, binding domains, and binding proteins are useful for treating various diseases such as cancer.

In one aspect, the present disclosure provides a single-domain that specifically binds CD38 and comprises the following complementary determining regions (CDRs): (a) a CDR-1 comprising the amino acid sequence GFILDTYS (SEQ ID NO: 13), a CDR-2 comprising the amino acid sequence ISSRDGNT (SEQ ID NO: 14), and a CDR-3 comprising the amino acid sequence AAGAQAHCTIFTSYFNSDYYRRYNY (SEQ ID NO: 15); (b) a CDR-1 comprising the amino acid sequence GFIFSDKV (SEQ ID NO: 16), a CDR-2 comprising the amino acid sequence ITPGGTAT (SEQ ID NO: 17), and a CDR-3 comprising the amino acid sequence RIGGPGGRYDN (SEQ ID NO: 18); (c) a CDR-1 comprising the amino acid sequence RSIFEINTMT (SEQ ID NO: 19), a CDR-2 comprising the amino acid sequence SRGATT (SEQ ID NO: 20), and a CDR-3 comprising the amino acid sequence SADRYGFGYGDNDY (SEQ ID NO: 21); (d) a CDR-1 comprising the amino acid sequence GFSLDYYH (SEQ ID NO: 22), a CDR-2 comprising the amino acid sequence ISSSDGYT (SEQ ID NO: 23), and a CDR-3 comprising the amino acid sequence AASPRRLACAGSLYPPLSADFSS (SEQ ID NO: 24); (e) a CDR-1 comprising the amino acid sequence GAIVSAES (SEQ ID NO: 25), a CDR-2 comprising the amino acid sequence IISGSKS (SEQ ID NO: 26), and a CDR-3 comprising the amino acid sequence KRTERIWTNNPQVY (SEQ ID NO: 27); (f) a CDR-1 comprising the amino acid sequence GTFSSINL (SEQ ID NO: 28), a CDR-2 comprising the amino acid sequence DYTEGTT (SEQ ID NO: 29), and a CDR-3 comprising the amino acid sequence WLMVRAGDVY (SEQ ID NO: 30); (g) a CDR-1 comprising the amino acid sequence GSISGLNT (SEQ ID NO: 31), a CDR-2 comprising the amino acid sequence IISGTMT (SEQ ID NO: 32), and a CDR-3 comprising the amino acid sequence TFKEITRDSRSY (SEQ ID NO: 33); (h) a CDR-1 comprising the amino acid sequence GSSVSMNS (SEQ ID NO: 34), a CDR-2 comprising the amino acid sequence ITPGDRI (SEQ ID NO: 35), and a CDR-3 comprising the amino acid sequence NIGATRPPFGA (SEQ ID NO: 36); (i) a CDR-1 comprising the amino acid sequence GFIYSIST (SEQ ID NO: 37), a CDR-2 comprising the amino acid sequence ITSGGNT (SEQ ID NO: 38), and a CDR-3 comprising the amino acid sequence NTAWRETIVSRV (SEQ ID NO: 39); (i) a CDR-1 comprising the amino acid sequence GRYFRINA (SEQ ID NO: 40), a CDR-2 comprising the amino acid sequence ISNDGST (SEQ ID NO: 41), and a CDR-3 comprising the amino acid sequence NVKALPFLSSNELSY (SEQ ID NO: 42); (k) a CDR-1 comprising the amino acid sequence GTFSSIAL (SEQ ID NO: 43), a CDR-2 comprising the amino acid sequence VSIGGVT (SEQ ID NO: 44), and a CDR-3 comprising the amino acid sequence WRMEGAGDVY (SEQ ID NO: 45); or (1) a CDR-1 comprising the amino acid sequence GIIFRIFS (SEQ ID NO: 46), a CDR-2 comprising the amino acid sequence ITSGGNT (SEQ ID NO: 47), and a CDR-3 comprising the amino acid sequence NVAIPSGIVDRSA (SEQ ID NO: 48).

In some embodiments, the antibody comprises an amino acid sequence that is at 85% identical, at least 95% identical, or 100% identical to the amino acid sequence of any one of SEQ ID NOs: 52-102 and 473 and 230-258.

In one aspect, the present disclosure provides a single-domain that specifically binds BCMA and comprises the following complementary determining regions (CDRs): (a) a CDR-1 comprising the amino acid sequence GFILDTYS (SEQ ID NO: 13), a CDR-2 comprising the amino acid sequence ITARGDWT (SEQ ID NO: 104), and a CDR-3 comprising the amino acid sequence VRDLLGRDDY (SEQ ID NO: 105); (b) a CDR-1 comprising the amino acid sequence GRTLNNYV (SEQ ID NO: 106), a CDR-2 comprising the amino acid sequence MWWSGGSP (SEQ ID NO: 107), and a CDR-3 comprising the amino acid sequence AATWVGTSEYRH (SEQ ID NO: 108); (c) a CDR-1 comprising the amino acid sequence GSIFAYHV (SEQ ID NO: 109), a CDR-2 comprising the amino acid sequence ITSGGST (SEQ ID NO: 110), and a CDR-3 comprising the amino acid sequence AIGYYSGSYYWERSGDY (SEQ ID NO: 111); (d) a CDR-1 comprising the amino acid sequence GRTLNNYV (SEQ ID NO: 106), a CDR-2 comprising the amino acid sequence MWWSGGSP (SEQ ID NO: 107), and a CDR-3 comprising the amino acid sequence AATWVGTSEYQH (SEQ ID NO: 112); (e) a CDR-1 comprising the amino acid sequence GRTFSDYT (SEQ ID NO: 113), a CDR-2 comprising the amino acid sequence SVWTDGKP (SEQ ID NO: 114), and a CDR-3 comprising the amino acid sequence QALTGGSWILDY (SEQ ID NO: 115); (f) a CDR-1 comprising the amino acid sequence GRPLRMYN (SEQ ID NO: 116), a CDR-2 comprising the amino acid sequence ISWSGKTT (SEQ ID NO: 117), and a CDR-3 comprising the amino acid sequence FMNVWADTSDSSADAY (SEQ ID NO: 118); (g) a CDR-1 comprising the amino acid sequence GSTFSRYA (SEQ ID NO: 119), a CDR-2 comprising the amino acid sequence ISWSGGST (SEQ ID NO: 120), and a CDR-3 comprising the amino acid sequence FGDIIRSGERSDYEY (SEQ ID NO: 121); (h) a CDR-1 comprising the amino acid sequence GFTLSSYW (SEQ ID NO: 122), a CDR-2 comprising the amino acid sequence IKPESGIT (SEQ ID NO: 123), and a CDR-3 comprising the amino acid sequence VREDYDSAYVGDY (SEQ ID NO: 124); (i) a CDR-1 comprising the amino acid sequence GFTYSSYW (SEQ ID NO: 125), a CDR-2 comprising the amino acid sequence ISTGGDTTDT (SEQ ID NO: 126), and a CDR-3 comprising the amino acid sequence VALNLWGTDLEHDY (SEQ ID NO: 127); (j) a CDR-1 comprising the amino acid sequence GGTLEYYA (SEQ ID NO: 128), a CDR-2 comprising the amino acid sequence ITWSGGST (SEQ ID NO: 129), and a CDR-3 comprising the amino acid sequence AAQFVEVEILVRSYEY (SEQ ID NO: 130); (k) a CDR-1 comprising the amino acid sequence GFTFSSYW (SEQ ID NO: 131), a CDR-2 comprising the amino acid sequence INTDGDST (SEQ ID NO: 132), and a CDR-3 comprising the amino acid sequence AAIVTRSDGHQYDY (SEQ ID NO: 133); (1) a CDR-1 comprising the amino acid sequence GRIDSGYT (SEQ ID NO: 134), a CDR-2 comprising the amino acid sequence VVGSDGRD (SEQ ID NO: 135), and a CDR-3 comprising the amino acid sequence AATNYYSDYLDHLSRGY (SEQ ID NO: 136); (m) a CDR-1 comprising the amino acid sequence GFTFGSYW (SEQ ID NO: 137), a CDR-2 comprising the amino acid sequence IDTSGGHV (SEQ ID NO: 138), and a CDR-3 comprising the amino acid sequence ARVENTWESIY (SEQ ID NO: 139); (n) a CDR-1 comprising the amino acid sequence GHTLNSYA (SEQ ID NO: 140), a CDR-2 comprising the amino acid sequence ISRSGEKT (SEQ ID NO: 141), and a CDR-3 comprising the amino acid sequence GAWNFVKNDAY (SEQ ID NO: 142); (o) a CDR-1 comprising the amino acid sequence GFTFSTYS (SEQ ID NO: 143), a CDR-2 comprising the amino acid sequence IDARGVNT (SEQ ID NO: 144), and a CDR-3 comprising the amino acid sequence GGWEFAT SEQ ID NO: 145); (p) a CDR-1 comprising the amino acid sequence GHTFSNSA (SEQ ID NO: 146), a CDR-2 comprising the amino acid sequence ISLNGGNT (SEQ ID NO: 147), and a CDR-3 comprising the amino acid sequence ATLGFAS (SEQ ID NO: 148); (q) a CDR-1 comprising the amino acid sequence GRTFSGYT (SEQ ID NO: 149), a CDR-2 comprising the amino acid sequence AVGSDGST (SEQ ID NO: 150), and a CDR-3 comprising the amino acid sequence VKLDSGAWSLAE (SEQ ID NO: 151); (r) a CDR-1 comprising the amino acid sequence GRTFSSYA (SEQ ID NO: 152), a CDR-2 comprising the amino acid sequence VSWTGDNT (SEQ ID NO: 153), and a CDR-3 comprising the amino acid sequence AAWNWGHHEYTY (SEQ ID NO: 154); (s) a CDR-1 comprising the amino acid sequence GRTIGSFV (SEQ ID NO: 155), a CDR-2 comprising the amino acid sequence VNWRGSST (SEQ ID NO: 156), and a CDR-3 comprising the amino acid sequence ARWSWDIGADFGS (SEQ ID NO: 157); (t) a CDR-1 comprising the amino acid sequence GFTFSNYA (SEQ ID NO: 158), a CDR-2 comprising the amino acid sequence INANSDTT (SEQ ID NO: 159), and a CDR-3 comprising the amino acid sequence VSDSYIGGLYATYVY (SEQ ID NO: 160); (u) a CDR-1 comprising the amino acid sequence VRYFSTYA (SEQ ID NO: 161), a CDR-2 comprising the amino acid sequence ISWSGGST (SEQ ID NO: 120), and a CDR-3 comprising the amino acid sequence AAPYGSSQNLEYDY (SEQ ID NO: 162); (v) a CDR-1 comprising the amino acid sequence GFTFDDYA (SEQ ID NO: 163), a CDR-2 comprising the amino acid sequence ISWNGGST (SEQ ID NO: 164), and a CDR-3 comprising the amino acid sequence VPVSHSDSVCGSPYMDY (SEQ ID NO: 165); (w) a CDR-1 comprising the amino acid sequence GFNLTSDA (SEQ ID NO: 166), a CDR-2 comprising the amino acid sequence ISRT (SEQ ID NO: 167), and a CDR-3 comprising the amino acid sequence AADNAADRCSLSIYNYNL (SEQ ID NO: 168); (x) a CDR-1 comprising the amino acid sequence GRTFASFA (SEQ ID NO: 169), a CDR-2 comprising the amino acid sequence ISWSGGFT (SEQ ID NO: 170), and a CDR-3 comprising the amino acid sequence GEWGGFSL (SEQ ID NO: 171); (y) a CDR-1 comprising the amino acid sequence GFTFDDYA (SEQ ID NO: 163), a CDR-2 comprising the amino acid sequence IASSDGST (SEQ ID NO: 172), and a CDR-3 comprising the amino acid sequence VAPCFWFDTVIAGTDPRYDY (SEQ ID NO: 173); (z) a CDR-1 comprising the amino acid sequence GFTLGYYA (SEQ ID NO: 174), a CDR-2 comprising the amino acid sequence VVGSDGRD (SEQ ID NO: 135), and a CDR-3 comprising the amino acid sequence AATNYYSDYLDHLSRGY (SEQ ID NO: 136); (aa) a CDR-1 comprising the amino acid sequence GRPLRMYN (SEQ ID NO: 116), a CDR-2 comprising the amino acid sequence ISGRDGST (SEQ ID NO: 175), and a CDR-3 comprising the amino acid sequence AAVRGPIVSMDPDLCRPVEFDY (SEQ ID NO: 176); (ab) a CDR-1 comprising the amino acid sequence GFTFEDYA (SEQ ID NO: 177), a CDR-2 comprising the amino acid sequence TSKNDRMP (SEQ ID NO: 178), and a CDR-3 comprising the amino acid sequence AATNGPAITLFPCHINYWLYDN (SEQ ID NO: 179); (ac) a CDR-1 comprising the amino acid sequence GFTFEDYA (SEQ ID NO: 177), a CDR-2 comprising the amino acid sequence TSKNDRMP (SEQ ID NO: 178), and a CDR-3 comprising the amino acid sequence ASADWRSPTPFPCGVSRSLYDH (SEQ ID NO: 180); (ad) a CDR-1 comprising the amino acid sequence GNFLRFNA (SEQ ID NO: 181), a CDR-2 comprising the amino acid sequence ISSGGRT (SEQ ID NO: 182), and a CDR-3 comprising the amino acid sequence WSAPDY (SEQ ID NO: 183); (ae) a CDR-1 comprising the amino acid sequence GSFSSIDT (SEQ ID NO: 184), a CDR-2 comprising the amino acid sequence INRGGDT (SEQ ID NO: 185), and a CDR-3 comprising the amino acid sequence AGSFTLATGDDFGS (SEQ ID NO: 186); (af) a CDR-1 comprising the amino acid sequence EQNFSADY (SEQ ID NO: 187), a CDR-2 comprising the amino acid sequence ISNSGRT (SEQ ID NO: 188), and a CDR-3 comprising the amino acid sequence VAEPFSFRRRA (SEQ ID NO: 189); (ag) a CDR-1 comprising the amino acid sequence EQNFSTDD (SEQ ID NO: 190), a CDR-2 comprising the amino acid sequence ITNSGTT (SEQ ID NO: 191), and a CDR-3 comprising the amino acid sequence GESTTGWAECDFGC (SEQ ID NO: 192); (ah) a CDR-1 comprising the amino acid sequence RSIFSINA (SEQ ID NO: 193), a CDR-2 comprising the amino acid sequence ITNGGTT (SEQ ID NO: 194), and a CDR-3 comprising the amino acid sequence NANSRYGVGWYNY (SEQ ID NO: 195); (ai) a CDR-1 comprising the amino acid sequence GSIFSINP (SEQ ID NO: 196), a CDR-2 comprising the amino acid sequence FTSGGTT (SEQ ID NO: 197), and a CDR-3 comprising the amino acid sequence NVRGGHCDPRYWREY (SEQ ID NO: 198); (aj) a CDR-1 comprising the amino acid sequence GSIFSINP (SEQ ID NO: 196), a CDR-2 comprising the amino acid sequence FTSGGTT (SEQ ID NO: 197), and a CDR-3 comprising the amino acid sequence NVRGGHYDPRYWREY (SEQ ID NO: 199); (ak) a CDR-1 comprising the amino acid sequence GRPIDTYA (SEQ ID NO: 200), a CDR-2 comprising the amino acid sequence VSWAGVYT (SEQ ID NO: 201), and a CDR-3 comprising the amino acid sequence AATKLPWNTIVMVQRSYCDY (SEQ ID NO: 202); (al) a CDR-1 comprising the amino acid sequence GRTISTYA (SEQ ID NO: 203), a CDR-2 comprising the amino acid sequence VSWAGSYT (SEQ ID NO: 204), and a CDR-3 comprising the amino acid sequence AATKLPWNTSVMVKRSVYDY (SEQ ID NO: 205); (am) a CDR-1 comprising the amino acid sequence GRPLECYA (SEQ ID NO: 206), a CDR-2 comprising the amino acid sequence ISGNGVNT (SEQ ID NO: 207), and a CDR-3 comprising the amino acid sequence AATTVPVINLEISHMTY (SEQ ID NO: 208); (an) a CDR-1 comprising the amino acid sequence GRRLEGYT (SEQ ID NO: 209), a CDR-2 comprising the amino acid sequence ISRNRVNT (SEQ ID NO: 210), and a CDR-3 comprising the amino acid sequence AATNLPGITLLMSHMNYCDY (SEQ ID NO: 211); (ao) a CDR-1 comprising the amino acid sequence GPRLEGYT (SEQ ID NO: 212), a CDR-2 comprising the amino acid sequence ITRNRVNT (SEQ ID NO: 213), and a CDR-3 comprising the amino acid sequence AATTVPVINLQVSHINY (SEQ ID NO: 214); (ap) a CDR-1 comprising the amino acid sequence GRRLEGYT (SEQ ID NO: 209), a CDR-2 comprising the amino acid sequence ISRNRVNT (SEQ ID NO: 210), and a CDR-3 comprising the amino acid sequence AATTVPVINLQVSHINY (SEQ ID NO: 214); (aq) a CDR-1 comprising the amino acid sequence GRTFNLYA (SEQ ID NO: 215), a CDR-2 comprising the amino acid sequence ITWSGGST (SEQ ID NO: 129), and a CDR-3 comprising the amino acid sequence AAKVFPMATLDDDVYDY (SEQ ID NO: 216); (ar) a CDR-1 comprising the amino acid sequence GVTFSNYV (SEQ ID NO: 217), a CDR-2 comprising the amino acid sequence ISWSGGST (SEQ ID NO: 120), and a CDR-3 comprising the amino acid sequence AAESHRRNTIVIVTTPDEYDY (SEQ ID NO: 218); (as) a CDR-1 comprising the amino acid sequence GRTFSSYA (SEQ ID NO: 152), a CDR-2 comprising the amino acid sequence ISWSGGST (SEQ ID NO: 120), and a CDR-3 comprising the amino acid sequence AADPSSGYNLFARTVVAFARYDY (SEQ ID NO: 219); (at) a CDR-1 comprising the amino acid sequence GRTFSSYA (SEQ ID NO: 152), a CDR-2 comprising the amino acid sequence ISWSGGST (SEQ ID NO: 120), and a CDR-3 comprising the amino acid sequence AAHPAGAHGGLIYKN (SEQ ID NO: 220); (au) a CDR-1 comprising the amino acid sequence GRTVSNYA (SEQ ID NO: 221), a CDR-2 comprising the amino acid sequence INRSGTP (SEQ ID NO: 222), and a CDR-3 comprising the amino acid sequence AADLRGSSWYFDGVDY (SEQ ID NO: 225); (av) a CDR-1 comprising the amino acid sequence GRTFSSYA (SEQ ID NO: 152), a CDR-2 comprising the amino acid sequence ISWSGGST (SEQ ID NO: 120), and a CDR-3 comprising the amino acid sequence AADLRGSSWYFDGMDY (SEQ ID NO: 226); (aw) a CDR-1 comprising the amino acid sequence GRTFSRYA (SEQ ID NO: 227), a CDR-2 comprising the amino acid sequence ITRSGTST (SEQ ID NO: 228), and a CDR-3 comprising the amino acid sequence AAHEAQYSSRWSGTEKGYDY (SEQ ID NO: 229); or (ax) a CDR-1 comprising the amino acid sequence GRTFSSYA (SEQ ID NO: 152), a CDR-2 comprising the amino acid sequence ISWSGGST (SEQ ID NO: 120), and a CDR-3 comprising the amino acid sequence AADPSSGYNLFARTVVAFARYDY (SEQ ID NO: 219).

In some embodiments, the antibody comprises an amino acid sequence that is at least 85% identical, at least 95% identical, or 100% identical to the amino acid sequence of any one of SEQ ID NOs: 52-102 and 473 and 230-258.

In one aspect, the present disclosure provides a single-domain that specifically binds GPRC5D and comprises the following complementary determining regions (CDRs): (a) a CDR-1 comprising the amino acid sequence GRTVSSYA (SEQ ID NO: 265), a CDR-2 comprising the amino acid sequence ISWSGRST (SEQ ID NO: 266), and a CDR-3 comprising the amino acid sequence ATSRAVIPGRDWNYYEY (SEQ ID NO:267); (b) a CDR-1 comprising the amino acid sequence GRTASAYV (SEQ ID NO: 268), a CDR-2 comprising the amino acid sequence ISGGA (SEQ ID NO: 269), and a CDR-3 comprising the amino acid sequence AAERGMRRLTESYQYDY (SEQ ID NO: 270); (c) a CDR-1 comprising the amino acid sequence GIIFSASN (SEQ ID NO: 271), a CDR-2 comprising the amino acid sequence VTGGGSI (SEQ ID NO: 272), and a CDR-3 comprising the amino acid sequence NARRSYSH (SEQ ID NO: 273); (d) a CDR-1 comprising the amino acid sequence GGFGMMYS (SEQ ID NO: 274), a CDR-2 comprising the amino acid sequence RTIDGST (SEQ ID NO: 275), and a CDR-3 comprising the amino acid sequence NAKPLNGRLTQY (SEQ ID NO: 276); (e) a CDR-1 comprising the amino acid sequence RIRFSINV (SEQ ID NO: 277), a CDR-2 comprising the amino acid sequence IAAGGTT (SEQ ID NO: 278), and a CDR-3 comprising the amino acid sequence NAVLSTLVLPSTY (SEQ ID NO: 279); or (f) a CDR-1 comprising the amino acid sequence SERTFRSYT (SEQ ID NO: 280), a CDR-2 comprising the amino acid sequence ISWSHSST (SEQ ID NO: 281), and a CDR-3 comprising the amino acid sequence AADLRLLPEEYDY (SEQ ID NO: 282).

In some embodiments, the antibody comprises an amino acid sequence that is at least 85% identical, at least 95% identical, or 100% identical to the amino acid sequence of any one of SEQ ID NOs: 259-264 and 283-320.

In one aspect, the present disclosure provides a single-domain that specifically binds FcRH5 and comprises the following complementary determining regions (CDRs):); (a) a CDR-1 comprising the amino acid sequence GITVSRND (SEQ ID NO: 326), a CDR-2 comprising the amino acid sequence IMNRVGST (SEQ ID NO: 327), and a CDR-3 comprising the amino acid sequence NALNTVITWP (SEQ ID NO: 328); (b) a CDR-1 comprising the amino acid sequence VHIISHYS (SEQ ID NO: 329), a CDR-2 comprising the amino acid sequence IPVSGRVP (SEQ ID NO: 330), and a CDR-3 comprising the amino acid sequence AAYPRKGLEGNEYEY (SEQ ID NO: 331); (c) a CDR-1 comprising the amino acid sequence GHTLSTYA (SEQ ID NO: 332), a CDR-2 comprising the amino acid sequence IARDGGAT (SEQ ID NO: 333), and a CDR-3 comprising the amino acid sequence AASSMFSTAKRDYSY (SEQ ID NO: 334); (d) a CDR-1 comprising the amino acid sequence GRTFSTYA (SEQ ID NO: 335), a CDR-2 comprising the amino acid sequence IDTTGSAS (SEQ ID NO: 336), and a CDR-3 comprising the amino acid sequence AAARRYSTAPGDYDY (SEQ ID NO: 337); or (e) a CDR-1 comprising the amino acid sequence GSHFSIAT (SEQ ID NO: 338), a CDR-2 comprising the amino acid sequence LSSSGRP (SEQ ID NO: 339), and a CDR-3 comprising the amino acid sequence KANLKRFFIEERYRDY (SEQ ID NO: 340).

In some embodiments, the antibody comprises an amino acid sequence that is at least 85% identical, at least 95% identical, or 100% identical to the amino acid sequence of any one of SEQ ID NOs: 321-325 and 341-345.

In some embodiments, the antibody binds CD38, BCMA, GPRC5D, or FcRH5 with a K_{D} of 20 nM or less. In some embodiments, the antibody binds CD38, BCMA, GPRC5D, or FcRH5 with a K_{D} of 10 nM or less, optionally wherein the antibody binds CD38, BCMA, GPRC5D, or FcRH5 with a K_{D} of 5 nM or less, optionally wherein the antibody binds CD38, BCMA, GPRC5D, or FcRH5 with a K_{D} of 1 nM or less, optionally wherein the antibody binds CD38, BCMA, GPRC5D, or FcRH5 with a K_{D} of 0.5 nM or less, optionally wherein the antibody binds CD38, BCMA, GPRC5D, or FcRH5 with a K_{D} of 0.1 nM or less, optionally wherein the antibody binds CD38, BCMA, GPRC5D, or FcRH5 with a K_{D} of 0.05 nM or less, optionally wherein the antibody binds CD38, BCMA, GPRC5D, or FcRH5 with a K_{D} of 0.01 nM or less

In some embodiments, the present disclosure provides a conjugate comprising a biologic and a single-domain antibody described herein. In some embodiments, the biologic and the single-domain antibody of the conjugate are covalently bound to one another as part of a single polypeptide chain. In some embodiments, the biologic and the single-domain antibody of the conjugate are connected via a chemical linker.

In some embodiments, the biologic of the conjugate is selected from an antibody, a cytokine, a growth factor, an enzyme, a polypeptide, a protein, a carbohydrate, and a nucleic acid. In some embodiments, the conjugate, when administered to a human subject, possesses a longer circulating half-life relative to the corresponding biologic that is not conjugated to the single-domain antibody.

In one aspect, the present disclosure provides an antibody or binding protein that specifically binds to CD38 and comprises the following complementarity-determining regions (CDRs): (a) a CDR-1 comprising the amino acid sequence GFILDTYS (SEQ ID NO: 13), a CDR-2 comprising the amino acid sequence ISSRDGNT (SEQ ID NO: 14), and a CDR-3 comprising the amino acid sequence AAGAQAHCTIFTSYFNSDYYRRYNY (SEQ ID NO: 15); (b) a CDR-1 comprising the amino acid sequence GFIFSDKV (SEQ ID NO: 16), a CDR-2 comprising the amino acid sequence ITPGGTAT (SEQ ID NO: 17), and a CDR-3 comprising the amino acid sequence RIGGPGGRYDN (SEQ ID NO: 18); (c) a CDR-1 comprising the amino acid sequence RSIFEINTMT (SEQ ID NO: 19), a CDR-2 comprising the amino acid sequence SRGATT (SEQ ID NO: 20), and a CDR-3 comprising the amino acid sequence SADRYGFGYGDNDY (SEQ ID NO: 21); (d) a CDR-1 comprising the amino acid sequence GFSLDYYH (SEQ ID NO: 22), a CDR-2 comprising the amino acid sequence ISSSDGYT (SEQ ID NO: 23), and a CDR-3 comprising the amino acid sequence AASPRRLACAGSLYPPLSADFSS (SEQ ID NO: 24); (e) a CDR-1 comprising the amino acid sequence GAIVSAES (SEQ ID NO: 25), a CDR-2 comprising the amino acid sequence IISGSKS (SEQ ID NO: 26), and a CDR-3 comprising the amino acid sequence KRTERIWTNNPQVY (SEQ ID NO: 27); (f) a CDR-1 comprising the amino acid sequence GTFSSINL (SEQ ID NO: 28), a CDR-2 comprising the amino acid sequence DYTEGTT (SEQ ID NO: 29), and a CDR-3 comprising the amino acid sequence WLMVRAGDVY (SEQ ID NO: 30); (g) a CDR-1 comprising the amino acid sequence GSISGLNT (SEQ ID NO: 31), a CDR-2 comprising the amino acid sequence IISGTMT (SEQ ID NO: 32), and a CDR-3 comprising the amino acid sequence TFKEITRDSRSY (SEQ ID NO: 33); (h) a CDR-1 comprising the amino acid sequence GSSVSMNS (SEQ ID NO: 34), a CDR-2 comprising the amino acid sequence ITPGDRI (SEQ ID NO: 35), and a CDR-3 comprising the amino acid sequence NIGATRPPFGA (SEQ ID NO: 36); (i) a CDR-1 comprising the amino acid sequence GFIYSIST (SEQ ID NO: 37), a CDR-2 comprising the amino acid sequence ITSGGNT (SEQ ID NO: 38), and a CDR-3 comprising the amino acid sequence NTAWRETIVSRV (SEQ ID NO: 39); (j) a CDR-1 comprising the amino acid sequence GRYFRINA (SEQ ID NO: 40), a CDR-2 comprising the amino acid sequence ISNDGST (SEQ ID NO: 41), and a CDR-3 comprising the amino acid sequence NVKALPFLSSNELSY (SEQ ID NO: 42); (k) a CDR-1 comprising the amino acid sequence GTFSSIAL (SEQ ID NO: 43), a CDR-2 comprising the amino acid sequence VSIGGVT (SEQ ID NO: 44), and a CDR-3 comprising the amino acid sequence WRMEGAGDVY (SEQ ID NO: 45); or (1) a CDR-1 comprising the amino acid sequence GIIFRIFS (SEQ ID NO: 46), a CDR-2 comprising the amino acid sequence ITSGGNT (SEQ ID NO: 47), and a CDR-3 comprising the amino acid sequence NVAIPSGIVDRSA (SEQ ID NO: 48).

In some embodiments, the antibody or binding protein of the present disclosure comprises an amino acid sequence that is at least 85% identical, at least 95% identical, or 100% identical to the amino acid sequence of any one of SEQ ID NOs: 1-12 and 49-51.

In one aspect, the present disclosure provides an antibody or binding protein that binds to BCMA and comprises the following complementarity-determining regions (CDRs): (a) a CDR-1 comprising the amino acid sequence GFILDTYS (SEQ ID NO: 13), a CDR-2 comprising the amino acid sequence ITARGDWT (SEQ ID NO: 104), and a CDR-3 comprising the amino acid sequence VRDLLGRDDY (SEQ ID NO: 105); (b) a CDR-1 comprising the amino acid sequence GRTLNNYV (SEQ ID NO: 106), a CDR-2 comprising the amino acid sequence MWWSGGSP (SEQ ID NO: 107), and a CDR-3 comprising the amino acid sequence AATWVGTSEYRH (SEQ ID NO: 108); (c) a CDR-1 comprising the amino acid sequence GSIFAYHV (SEQ ID NO: 109), a CDR-2 comprising the amino acid sequence ITSGGST (SEQ ID NO: 110), and a CDR-3 comprising the amino acid sequence AIGYYSGSYYWERSGDY (SEQ ID NO: 111); (d) a CDR-1 comprising the amino acid sequence GRTLNNYV (SEQ ID NO: 106), a CDR-2 comprising the amino acid sequence MWWSGGSP (SEQ ID NO: 107), and a CDR-3 comprising the amino acid sequence AATWVGTSEYQH (SEQ ID NO: 112); (e) a CDR-1 comprising the amino acid sequence GRTFSDYT (SEQ ID NO: 113), a CDR-2 comprising the amino acid sequence SVWTDGKP (SEQ ID NO: 114), and a CDR-3 comprising the amino acid sequence QALTGGSWILDY (SEQ ID NO: 115); (f) a CDR-1 comprising the amino acid sequence GRPLRMYN (SEQ ID NO: 116), a CDR-2 comprising the amino acid sequence ISWSGKTT (SEQ ID NO: 117), and a CDR-3 comprising the amino acid sequence FMNVWADTSDSSADAY (SEQ ID NO: 118); (g) a CDR-1 comprising the amino acid sequence GSTFSRYA (SEQ ID NO: 119), a CDR-2 comprising the amino acid sequence ISWSGGST (SEQ ID NO: 120), and a CDR-3 comprising the amino acid sequence FGDIIRSGERSDYEY (SEQ ID NO: 121); (h) a CDR-1 comprising the amino acid sequence GFTLSSYW (SEQ ID NO: 122), a CDR-2 comprising the amino acid sequence IKPESGIT (SEQ ID NO: 123), and a CDR-3 comprising the amino acid sequence VREDYDSAYVGDY (SEQ ID NO: 124); (i) a CDR-1 comprising the amino acid sequence GFTYSSYW (SEQ ID NO: 125), a CDR-2 comprising the amino acid sequence ISTGGDTTDT (SEQ ID NO: 126), and a CDR-3 comprising the amino acid sequence VALNLWGTDLEHDY (SEQ ID NO: 127); (j) a CDR-1 comprising the amino acid sequence GGTLEYYA (SEQ ID NO: 128), a CDR-2 comprising the amino acid sequence ITWSGGST (SEQ ID NO: 129), and a CDR-3 comprising the amino acid sequence AAQFVEVEILVRSYEY (SEQ ID NO: 130); (k) a CDR-1 comprising the amino acid sequence GFTFSSYW (SEQ ID NO: 131), a CDR-2 comprising the amino acid sequence INTDGDST (SEQ ID NO: 132), and a CDR-3 comprising the amino acid sequence AAIVTRSDGHQYDY (SEQ ID NO: 133); (1) a CDR-1 comprising the amino acid sequence GRIDSGYT (SEQ ID NO: 134), a CDR-2 comprising the amino acid sequence VVGSDGRD (SEQ ID NO: 135), and a CDR-3 comprising the amino acid sequence AATNYYSDYLDHLSRGY (SEQ ID NO: 136); (m) a CDR-1 comprising the amino acid sequence GFTFGSYW (SEQ ID NO: 137), a CDR-2 comprising the amino acid sequence IDTSGGHV (SEQ ID NO: 138), and a CDR-3 comprising the amino acid sequence ARVENTWESIY (SEQ ID NO: 139); (n) a CDR-1 comprising the amino acid sequence GHTLNSYA (SEQ ID NO: 140), a CDR-2 comprising the amino acid sequence ISRSGEKT (SEQ ID NO: 141), and a CDR-3 comprising the amino acid sequence GAWNFVKNDAY (SEQ ID NO: 142); (o) a CDR-1 comprising the amino acid sequence GFTFSTYS (SEQ ID NO: 143), a CDR-2 comprising the amino acid sequence IDARGVNT (SEQ ID NO: 144), and a CDR-3 comprising the amino acid sequence GGWEFAT SEQ ID NO: 145); (p) a CDR-1 comprising the amino acid sequence GHTFSNSA (SEQ ID NO: 146), a CDR-2 comprising the amino acid sequence ISLNGGNT (SEQ ID NO: 147), and a CDR-3 comprising the amino acid sequence ATLGFAS (SEQ ID NO: 148); (q) a CDR-1 comprising the amino acid sequence GRTFSGYT (SEQ ID NO: 149), a CDR-2 comprising the amino acid sequence AVGSDGST (SEQ ID NO: 150), and a CDR-3 comprising the amino acid sequence VKLDSGAWSLAE (SEQ ID NO: 151); (r) a CDR-1 comprising the amino acid sequence GRTFSSYA (SEQ ID NO: 152), a CDR-2 comprising the amino acid sequence VSWTGDNT (SEQ ID NO: 153), and a CDR-3 comprising the amino acid sequence AAWNWGHHEYTY (SEQ ID NO: 154); (s) a CDR-1 comprising the amino acid sequence GRTIGSFV (SEQ ID NO: 155), a CDR-2 comprising the amino acid sequence VNWRGSST (SEQ ID NO: 156), and a CDR-3 comprising the amino acid sequence ARWSWDIGADFGS (SEQ ID NO: 157); (t) a CDR-1 comprising the amino acid sequence GFTFSNYA (SEQ ID NO: 158), a CDR-2 comprising the amino acid sequence INANSDTT (SEQ ID NO: 159), and a CDR-3 comprising the amino acid sequence VSDSYIGGLYATYVY (SEQ ID NO: 160); (u) a CDR-1 comprising the amino acid sequence VRYFSTYA (SEQ ID NO: 161), a CDR-2 comprising the amino acid sequence ISWSGGST (SEQ ID NO: 120), and a CDR-3 comprising the amino acid sequence AAPYGSSQNLEYDY (SEQ ID NO: 162); (v) a CDR-1 comprising the amino acid sequence GFTFDDYA (SEQ ID NO: 163), a CDR-2 comprising the amino acid sequence ISWNGGST (SEQ ID NO: 164), and a CDR-3 comprising the amino acid sequence VPVSHSDSVCGSPYMDY (SEQ ID NO: 165); (w) a CDR-1 comprising the amino acid sequence GFNLTSDA (SEQ ID NO: 166), a CDR-2 comprising the amino acid sequence ISRT (SEQ ID NO: 167), and a CDR-3 comprising the amino acid sequence AADNAADRCSLSIYNYNL (SEQ ID NO: 168); (x) a CDR-1 comprising the amino acid sequence GRTFASFA (SEQ ID NO: 169), a CDR-2 comprising the amino acid sequence ISWSGGFT (SEQ ID NO: 170), and a CDR-3 comprising the amino acid sequence GEWGGFSL (SEQ ID NO: 171); (y) a CDR-1 comprising the amino acid sequence GFTFDDYA (SEQ ID NO: 163), a CDR-2 comprising the amino acid sequence IASSDGST (SEQ ID NO: 172), and a CDR-3 comprising the amino acid sequence VAPCFWFDTVIAGTDPRYDY (SEQ ID NO: 173); (z) a CDR-1 comprising the amino acid sequence GFTLGYYA (SEQ ID NO: 174), a CDR-2 comprising the amino acid sequence VVGSDGRD (SEQ ID NO: 135), and a CDR-3 comprising the amino acid sequence AATNYYSDYLDHLSRGY (SEQ ID NO: 136); (aa) a CDR-1 comprising the amino acid sequence GRPLRMYN (SEQ ID NO: 116), a CDR-2 comprising the amino acid sequence ISGRDGST (SEQ ID NO: 175), and a CDR-3 comprising the amino acid sequence AAVRGPIVSMDPDLCRPVEFDY (SEQ ID NO: 176); (ab) a CDR-1 comprising the amino acid sequence GFTFEDYA (SEQ ID NO: 177), a CDR-2 comprising the amino acid sequence TSKNDRMP (SEQ ID NO: 178), and a CDR-3 comprising the amino acid sequence AATNGPAITLFPCHINYWLYDN (SEQ ID NO: 179); (ac) a CDR-1 comprising the amino acid sequence GFTFEDYA (SEQ ID NO: 177), a CDR-2 comprising the amino acid sequence TSKNDRMP (SEQ ID NO: 178), and a CDR-3 comprising the amino acid sequence ASADWRSPTPFPCGVSRSLYDH (SEQ ID NO: 180); (ad) a CDR-1 comprising the amino acid sequence GNFLRFNA (SEQ ID NO: 181), a CDR-2 comprising the amino acid sequence ISSGGRT (SEQ ID NO: 182), and a CDR-3 comprising the amino acid sequence WSAPDY (SEQ ID NO: 183); (ae) a CDR-1 comprising the amino acid sequence GSFSSIDT (SEQ ID NO: 184), a CDR-2 comprising the amino acid sequence INRGGDT (SEQ ID NO: 185), and a CDR-3 comprising the amino acid sequence AGSFTLATGDDFGS (SEQ ID NO: 186); (af) a CDR-1 comprising the amino acid sequence EQNFSADY (SEQ ID NO: 187), a CDR-2 comprising the amino acid sequence ISNSGRT (SEQ ID NO: 188), and a CDR-3 comprising the amino acid sequence VAEPFSFRRRA (SEQ ID NO: 189); (ag) a CDR-1 comprising the amino acid sequence EQNFSTDD (SEQ ID NO: 190), a CDR-2 comprising the amino acid sequence ITNSGTT (SEQ ID NO: 191), and a CDR-3 comprising the amino acid sequence GESTTGWAECDFGC (SEQ ID NO: 192); (ah) a CDR-1 comprising the amino acid sequence RSIFSINA (SEQ ID NO: 193), a CDR-2 comprising the amino acid sequence ITNGGTT (SEQ ID NO: 194), and a CDR-3 comprising the amino acid sequence NANSRYGVGWYNY (SEQ ID NO: 195); (ai) a CDR-1 comprising the amino acid sequence GSIFSINP (SEQ ID NO: 196), a CDR-2 comprising the amino acid sequence FTSGGTT (SEQ ID NO: 197), and a CDR-3 comprising the amino acid sequence NVRGGHCDPRYWREY (SEQ ID NO: 198); (aj) a CDR-1 comprising the amino acid sequence GSIFSINP (SEQ ID NO: 196), a CDR-2 comprising the amino acid sequence FTSGGTT (SEQ ID NO: 197), and a CDR-3 comprising the amino acid sequence NVRGGHYDPRYWREY (SEQ ID NO: 199); (ak) a CDR-1 comprising the amino acid sequence GRPIDTYA (SEQ ID NO: 200), a CDR-2 comprising the amino acid sequence VSWAGVYT (SEQ ID NO: 201), and a CDR-3 comprising the amino acid sequence AATKLPWNTIVMVQRSYCDY (SEQ ID NO: 202); (al) a CDR-1 comprising the amino acid sequence GRTISTYA (SEQ ID NO: 203), a CDR-2 comprising the amino acid sequence VSWAGSYT (SEQ ID NO: 204), and a CDR-3 comprising the amino acid sequence AATKLPWNTSVMVKRSVYDY (SEQ ID NO: 205); (am) a CDR-1 comprising the amino acid sequence GRPLECYA (SEQ ID NO: 206), a CDR-2 comprising the amino acid sequence ISGNGVNT (SEQ ID NO: 207), and a CDR-3 comprising the amino acid sequence AATTVPVINLEISHMTY (SEQ ID NO: 208); (an) a CDR-1 comprising the amino acid sequence GRRLEGYT (SEQ ID NO: 209), a CDR-2 comprising the amino acid sequence ISRNRVNT (SEQ ID NO: 210), and a CDR-3 comprising the amino acid sequence AATNLPGITLLMSHMNYCDY (SEQ ID NO: 211); (ao) a CDR-1 comprising the amino acid sequence GPRLEGYT (SEQ ID NO: 212), a CDR-2 comprising the amino acid sequence ITRNRVNT (SEQ ID NO: 213), and a CDR-3 comprising the amino acid sequence AATTVPVINLQVSHINY (SEQ ID NO: 214); (ap) a CDR-1 comprising the amino acid sequence GRRLEGYT (SEQ ID NO: 209), a CDR-2 comprising the amino acid sequence ISRNRVNT (SEQ ID NO: 210), and a CDR-3 comprising the amino acid sequence AATTVPVINLQVSHINY (SEQ ID NO: 214); (aq) a CDR-1 comprising the amino acid sequence GRTFNLYA (SEQ ID NO: 215), a CDR-2 comprising the amino acid sequence ITWSGGST (SEQ ID NO: 129), and a CDR-3 comprising the amino acid sequence AAKVFPMATLDDDVYDY (SEQ ID NO: 216); (ar) a CDR-1 comprising the amino acid sequence GVTFSNYV (SEQ ID NO: 217), a CDR-2 comprising the amino acid sequence ISWSGGST (SEQ ID NO: 120), and a CDR-3 comprising the amino acid sequence AAESHRRNTIVIVTTPDEYDY (SEQ ID NO: 218); (as) a CDR-1 comprising the amino acid sequence GRTFSSYA (SEQ ID NO: 152), a CDR-2 comprising the amino acid sequence ISWSGGST (SEQ ID NO: 120), and a CDR-3 comprising the amino acid sequence AADPSSGYNLFARTVVAFARYDY (SEQ ID NO: 219); (at) a CDR-1 comprising the amino acid sequence GRTFSSYA (SEQ ID NO: 152), a CDR-2 comprising the amino acid sequence ISWSGGST (SEQ ID NO: 120), and a CDR-3 comprising the amino acid sequence AAHPAGAHGGLIYKN (SEQ ID NO: 220); (au) a CDR-1 comprising the amino acid sequence GRTVSNYA (SEQ ID NO: 221), a CDR-2 comprising the amino acid sequence INRSGTP (SEQ ID NO: 222), and a CDR-3 comprising the amino acid sequence AADLRGSSWYFDGVDY (SEQ ID NO: 225); (av) a CDR-1 comprising the amino acid sequence GRTFSSYA (SEQ ID NO: 152), a CDR-2 comprising the amino acid sequence ISWSGGST (SEQ ID NO: 120), and a CDR-3 comprising the amino acid sequence AADLRGSSWYFDGMDY (SEQ ID NO: 226); (aw) a CDR-1 comprising the amino acid sequence GRTFSRYA (SEQ ID NO: 227), a CDR-2 comprising the amino acid sequence ITRSGTST (SEQ ID NO: 228), and a CDR-3 comprising the amino acid sequence AAHEAQYSSRWSGTEKGYDY (SEQ ID NO: 229); or (ax) a CDR-1 comprising the amino acid sequence GRTFSSYA (SEQ ID NO: 152), a CDR-2 comprising the amino acid sequence ISWSGGST (SEQ ID NO: 120), and a CDR-3 comprising the amino acid sequence AADPSSGYNLFARTVVAFARYDY (SEQ ID NO: 219).

In some embodiments, the antibody or binding protein of the present disclosure comprises an amino acid sequence that is at least 85% identical, at least 95% identical, or 100% identical to the amino acid sequence of any one of SEQ ID NOs: 52-102 and 473 and 230-258.

In one aspect, the present disclosure provides an antibody or binding protein that binds to GPRC5D and comprises the following complementarity-determining regions (CDRs): (a) a CDR-1 comprising the amino acid sequence GRTVSSYA (SEQ ID NO: 265), a CDR-2 comprising the amino acid sequence ISWSGRST (SEQ ID NO: 266), and a CDR-3 comprising the amino acid sequence ATSRAVIPGRDWNYYEY (SEQ ID NO:267); (b) a CDR-1 comprising the amino acid sequence GRTASAYV (SEQ ID NO: 268), a CDR-2 comprising the amino acid sequence ISGGA (SEQ ID NO: 269), and a CDR-3 comprising the amino acid sequence AAERGMRRLTESYQYDY (SEQ ID NO: 270); (c) a CDR-1 comprising the amino acid sequence GIIFSASN (SEQ ID NO: 271), a CDR-2 comprising the amino acid sequence VTGGGSI (SEQ ID NO: 272), and a CDR-3 comprising the amino acid sequence NARRSYSH (SEQ ID NO: 273); (d) a CDR-1 comprising the amino acid sequence GGFGMMYS (SEQ ID NO: 274), a CDR-2 comprising the amino acid sequence RTIDGST (SEQ ID NO: 275), and a CDR-3 comprising the amino acid sequence NAKPLNGRLTQY (SEQ ID NO: 276); (e) a CDR-1 comprising the amino acid sequence RIRFSINV (SEQ ID NO: 277), a CDR-2 comprising the amino acid sequence IAAGGTT (SEQ ID NO: 278), and a CDR-3 comprising the amino acid sequence NAVLSTLVLPSTY (SEQ ID NO: 279); or (f) a CDR-1 comprising the amino acid sequence SERTFRSYT (SEQ ID NO: 280), a CDR-2 comprising the amino acid sequence ISWSHSST (SEQ ID NO: 281), and a CDR-3 comprising the amino acid sequence AADLRLLPEEYDY (SEQ ID NO: 282).

In some embodiments, the antibody or binding protein of the present disclosure comprises an amino acid sequence that is at least 85% identical, at least 95% identical, or 100% identical to the amino acid sequence of any one of SEQ ID NOs: 259-264 and 283-320.

In one aspect, the present disclosure provides an antibody or binding protein that binds to FcRH5 and comprises the following complementarity-determining regions (CDRs): (a) a CDR-1 comprising the amino acid sequence GITVSRND (SEQ ID NO: 326), a CDR-2 comprising the amino acid sequence IMNRVGST (SEQ ID NO: 327), and a CDR-3 comprising the amino acid sequence NALNTVITWP (SEQ ID NO: 328); (b) a CDR-1 comprising the amino acid sequence VHIISHYS (SEQ ID NO: 329), a CDR-2 comprising the amino acid sequence IPVSGRVP (SEQ ID NO: 330), and a CDR-3 comprising the amino acid sequence AAYPRKGLEGNEYEY (SEQ ID NO: 331); (c) a CDR-1 comprising the amino acid sequence GHTLSTYA (SEQ ID NO: 332), a CDR-2 comprising the amino acid sequence IARDGGAT (SEQ ID NO: 333), and a CDR-3 comprising the amino acid sequence AASSMFSTAKRDYSY (SEQ ID NO: 334); (d) a CDR-1 comprising the amino acid sequence GRTFSTYA (SEQ ID NO: 335), a CDR-2 comprising the amino acid sequence IDTTGSAS (SEQ ID NO: 336), and a CDR-3 comprising the amino acid sequence AAARRYSTAPGDYDY (SEQ ID NO: 337); or (e) a CDR-1 comprising the amino acid sequence GSHFSIAT (SEQ ID NO: 338), a CDR-2 comprising the amino acid sequence LSSSGRP (SEQ ID NO: 339), and a CDR-3 comprising the amino acid sequence KANLKRFFIEERYRDY (SEQ ID NO: 340).

In some embodiments, the antibody or binding protein of the present disclosure comprises an amino acid sequence that is at least 85% identical, at least 95% identical, or 100% identical to the amino acid sequence of any one of SEQ ID NOs: 321-325 and 341-345.

In some embodiments, the antibody or binding protein is a single-domain antibody.

In some embodiments, the antibody or binding protein binds CD38, BCMA, GPRC5D, or FcRH5 with a K_{D} of 20 nM or less. In some embodiments, the antibody or binding protein binds CD38, BCMA, GPRC5D, or FcRH5 with a K_{D} of 10 nM or less, optionally wherein the antibody or binding protein binds CD38, BCMA, GPRC5D, or FcRH5 with a K_{D} of 5 nM or less, optionally wherein the antibody or binding protein binds CD38, BCMA, GPRC5D, or FcRH5 with a K_{D} of 1 nM or less, optionally wherein the antibody or binding protein binds CD38, BCMA, GPRC5D, or FcRH5 with a K_{D} of 0.5 nM or less, optionally wherein the antibody or binding protein binds CD38, BCMA, GPRC5D, or FcRH5 with a K_{D} of 0.1 nM or less, optionally wherein the antibody or binding protein binds CD38, BCMA, GPRC5D, or FcRH5 with a K_{D} of 0.05 nM or less, optionally wherein the antibody or binding protein binds CD38, BCMA, GPRC5D, or FcRH5 with a K_{D} of 0.01 nM or less.

In some embodiments, the antibody or binding protein is a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, an antibody-like protein scaffold, a domain antibody, a Fv fragment, a Fab fragment, a F(ab′) 2 molecule, a tandem scFv (taFv), or a fusion protein. In some embodiments, the fusion protein comprises a biologic conjugated, either directly or indirectly, to the antibody or binding protein.

In some embodiments, the antibody or binding protein is a multi-specific antibody comprising at least one binding domain that specifically binds to an antigen other than CD38, BCMA, GPRC5D, or FcRH5, e.g., an antigen present on the same tumor cell or on a different cell.

In one aspect, the present disclosure provides a conjugate comprising a biologic and an antibody or binding protein disclosed herein. In some embodiments, the biologic and the antibody or binding protein are covalently bound to one another as part of a single polypeptide chain. In some embodiments, the biologic and the antibody or binding protein are connected via a chemical linker. In some embodiments, the biologic is selected from an antibody, a cytokine, a growth factor, an enzyme, a polypeptide, a protein, a carbohydrate, and a nucleic acid. In some embodiments, the conjugate, when administered to a human subject, possesses a longer circulating half-life relative to the corresponding biologic that is not conjugated to the single-domain antibody.

In one aspect, the present disclosure provides a nucleic acid encoding an antibody or binding protein or a conjugate disclosed herein. In some embodiments, the nucleic acid is an mRNA. In some embodiments, the nucleic acid molecule comprises, in the 5′-to-3′ direction: (a) a 5′ cap structure; (b) a 5′ untranslated region (UTR); (c) an open reading frame encoding a protein sequence comprising the binding domain of the single-domain antibody, conjugate, antibody, antigen binging fragment, or binding protein, wherein the open reading frame consists of nucleosides is selected from the group consisting of (i) uridine or a modified uridine, (ii) cytidine or a modified cytidine, (iii) adenosine or a modified adenosine, and (iv) guanosine or a modified guanosine; (d) a 3′ UTR; and (e) a 3′ tailing sequence of linked nucleosides. In some embodiments, the open reading frame of nucleosides is selected from the group consisting of (i) a modified uridine, (ii) cytidine, (iii) adenosine, and (iv) guanosine. In one embodiment, the open reading frame of nucleosides comprises a modified uridines.

In some embodiments, the modified uridine is 1-methylpseudouridine, pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine, 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 5-methyl-2-thio-uridine, 1-methyl-4-thio-pseudouridine, 4-thio-1-methyl-pseudouridine, 3-methylpseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropyl) pseudouridine, 5-(isopentenylaminomethyl) uridine, 5-(isopentenylaminomethyl)-2-thio-uridine, α-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, or 5-[3-(1-E-propenylamino) uridine. In some embodiments, the modified uridine is 1-methylpseudouridine.

In some embodiments, the modified cytidine is 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, or 2′-OH-ara-cytidine.

In some embodiments, the modified adenosine is 2-amino-purine, 2, 6-diaminopurine, 2-amino-6-halo-purine, 6-halo-purine, 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, α-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 8-azido-adenosine, 2′-F-ara-adenosine, 2′-F-adenosine, 2′-OH-ara-adenosine, or N6-(19-amino-pentaoxanonadecyl)-adenosine.

In some embodiments, the modified guanosine is inosine, 1-methyl-inosine, wyosine, methylwyosine, 4-demethyl-wyosine, isowyosine, wybutosine, peroxywybutosine, hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2, N2,7-dimethyl-guanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1,2′-O-dimethyl-inosine, 2′-O-ribosylguanosine, 1-thio-guanosine, O6-methyl-guanosine, 2′-F-ara-guanosine, or 2′-F-guanosine.

In some embodiments, the 3′ tailing sequence of linked nucleosides is a poly-adenylate (poly A) tail or a poly A-G quartet. In some embodiments, the nucleic acid of claim 29, wherein the 3′ tailing sequence of linked nucleosides is a poly A tail. In some embodiments, the nucleic acid of any one of claims 22-29, wherein the 5′ cap structure is Cap0, Cap1, ARCA, inosine, 1-methyl-guanosine, 2′fluoroguanosine, 7-deaza-guanosine, 8-oxo-guanosine, 2-amino-guanosine, LNA-guanosine, or 2-azidoguanosine.

In one aspect, the present disclosure provides a pharmaceutical composition comprising an antibody binding protein, conjugate, or nucleic acid disclosed herein. In one embodiment, the pharmaceutical composition further comprises one or more pharmaceutically acceptable carriers, diluents, excipients, or any combination thereof. In one embodiment, the pharmaceutical composition comprises a plurality of lipid nanoparticles encapsulating the nucleic acid. In one embodiment, the plurality of lipid nanoparticles has a mean particle size of from 80 nm to 160 nm. In one embodiment, the plurality of lipid nanoparticles has a polydispersity index (PDI) of from 0.02 to 0.2 and/or a lipid: nucleic acid ratio of from 10 to 20. In one embodiment, the lipid nanoparticles comprise a neutral lipid, a cationic lipid, a polyethyleneglycol (PEG) lipid, and/or a sterol. In one embodiment, the neutral lipid is 1,2-distearoyl-sn-glycero-3-phosphocholine. In one embodiment, the cationic lipid is a compound of Formula (I). In one embodiment, the PEG lipid is PEG 2000 dimyristoyl glycerol.

In one embodiment, the sterol of the pharmaceutical composition is cholesterol, adosterol, agosterol A, atheronals, avenasterol, azacosterol, blazein, cerevisterol, colestolone, cycloartenol, daucosterol, 7-dehydrocholesterol, 5-dehydroepisterol, 7-dehydrositosterol, 20a,22R-dihydroxycholesterol, dinosterol, epibrassicasterol, episterol, ergosterol, ergosterol, fecosterol, fucosterol, fungisterol, ganoderenic acid, ganoderic acid, ganoderiol, ganodermadiol, 7α-hydroxycholesterol, 22R-hydroxycholesterol, 27-hydroxycholesterol, inotodiol, lanosterol, lathosterol, lichesterol, lucidadiol, lumisterol, oxycholesterol, oxysterol, parkeol, saringosterol, spinasterol, sterol ester, trametenolic acid, zhankuic acid, or zymosterol. In one embodiment, the sterol is cholesterol.

In one aspect, the present disclosure provides a host cell comprising an antibody binding protein, conjugate, or nucleic acid disclosed herein. In one embodiment, the host cell is a eukaryotic cell. In one embodiment, the eukaryotic cell is a mammalian cell. In one embodiment, the mammalian cell is a CHO cell or HEK cell.

In one aspect, the present disclosure provides a method of treating cancer, comprising administering to a subject in need thereof to an antibody binding protein, conjugate, nucleic acid, pharmaceutical composition, or host cell disclosed herein.

In one aspect, the present disclosure provides a kit comprising (i) an antibody binding protein, conjugate, nucleic acid, pharmaceutical composition, or host cell disclosed herein, and (ii) a package insert instructing a user of the kit to administer the antibody binding protein, conjugate, nucleic acid, or pharmaceutical composition to a subject in need thereof.

In another aspect, the disclosure features a binding molecule comprising a VHH that specifically binds (i) CD38 and comprises a VHH CDR1, VHH CDR2, and VHH CDR3 of any one of the VHH sequences of SEQ ID NOs.: 1 to 12; (ii) BCMA and comprises a VHH CDR1, VHH CDR2, and VHH CDR3 of any one of the VHH sequences of SEQ ID NOs.: 52 to 102 and 473; (iii) GPRC5D and comprises a VHH CDR1, VHH CDR2, and VHH CDR3 of any one of the VHH sequences of SEQ ID NOs.: 259 to 264; or (iv) FcRH5 and comprises a VHH CDR1, VHH CDR2, and VHH CDR3 of any one of the VHH sequences of SEQ ID NOs.: 321-325.

In some instances, the VHH CDR1, VHH CDR2, and VHH CDR3 are based on any one of the Kabat, Chothia, enhanced Chothia, Contact, Aho, AbM, or IMGT definitions. In some instances, VHH specifically binds (i) CD38 and comprises the VHH CDR1, VHH CDR2, and VHH CDR3 of any one of the VHHs of Table 5 or Table A; (ii) BCMA and comprises the VHH CDR1, VHH CDR2, and VHH CDR3 of any one of the VHHs of Table 9 or Table B; (iii) GPRC5D and comprises the VHH CDR1, VHH CDR2, and VHH CDR3 of any one of the VHHs of Table 12 or Table C; or (iv) FcRH5 and comprises the VHH CDR1, VHH CDR2, and VHH CDR3 of any one of the VHHs of Table 16 or Table D.

In some instances, the VHH is humanized. In some cases, 1, 2, 3, 4, 5, 6, 7, 8, 9, 10, 11, 12, 13, 14, or 15 of the llama VHH sequence is substituted. In some cases, one, two, three, four, five, six, or seven of positions 14, 37, 44, 45, 47, 92, and 117 (numbering based on Kabat numbering) are substituted for humanization. In other cases, one, two, three, four, five, six, or seven of positions 14, 37, 44, 45, 47, 92, and 117 (numbering based on Kabat numbering) are not substituted during humanization. In some cases, positions 37 and/or 47 (numbering based on Kabat numbering) are not substituted during humanization. In some cases, positions 44 and/or 45 (numbering based on Kabat numbering) are substituted for humanization. In some cases, position 37 of the VHH is F, Y, V, I, or L; position 44 of the VHH is E, G, Q, or D; position 45 of the VHH is R or L; and/or position 47 of the VHH is G, F, W, L, T, P, A, or M, wherein the positions are based on Kabat numbering.

In some instances, a VHH that specifically binds (i) CD38 comprises an amino acid sequence that is at least 85%, at least 90%, at least 95%, at least 96%, at least 97%, at least 98%, or at least 99% identical to any one VHH of Table 4; (ii) BCMA comprises an amino acid sequence that is at least 85%, at least 90%, at least 95%, at least 96%, at least 97%, at least 98%, or at least 99% identical to any one VHH of Table 8; (iii) GPRC5D comprises an amino acid sequence that is at least 85%, at least 90%, at least 95%, at least 96%, at least 97%, at least 98%, or at least 99% identical to any one VHH of Table 11; or (iv) FcRH5 and comprises an amino acid sequence that is at least 85%, at least 90%, at least 95%, at least 96%, at least 97%, at least 98%, or at least 99% identical to any one VHH of Table 15.

In other instances, a VHH that specifically binds (i) CD38 comprises an amino acid sequence that is at least 95%, at least 96%, at least 97%, at least 98%, at least 99% or 100% identical to any one VHH of Table 7; (ii) BCMA comprises an amino acid sequence that is at least 95%, at least 96%, at least 97%, at least 98%, at least 99% or 100% identical to any one VHH of Table 10; (iii) GPRC5D comprises an amino acid sequence that is at least 95%, at least 96%, at least 97%, at least 98%, at least 99% or 100% identical to any one VHH of Table 14; or (iv) FcRH5 comprises an amino acid sequence that is at least 95%, at least 96%, at least 97%, at least 98%, at least 99% or 100% identical to any one VHH of Table 18.

In some instances, the VHH binds to cell surface expressed CD38, BCMA, GPRC5D, or FcRH5 with an EC50 of 0.1 nM to 10 nM, 0.1 nM to 7 nM, 0.1 nM to 5 nM, or 0.1 nM to 3 nM.

In certain instances, the VHH is attached to a peptide linker or a chemical linker. In some cases, the linker is a peptide linker. In certain cases, the peptide linker is a glycine linker, a serine linker, or a glycine-serine linker. In one instance, the linker is a peptide linker that is (G₄S)n, wherein n=1, 2, 3, 4, 5, 6, 7, 8, 9, or 10 (SEQ ID NO: 477).

In some instances, the VHH is attached to a human Fc region comprising a hinge, CH2, and CH3 region of a human immunoglobulin (Ig). In certain cases, the human Ig is human IgG1, human IgG2, human IgG3, human IgG4, or human IgG4PAA. In some cases, the human Fc region is attached at the N-terminal of the VHH. In other cases, the human Fc region is attached at the C-terminal of the VHH.

In certain instances, the binding molecule further comprises a half-life extension moiety. In some cases, the half-life extension moiety is polyethylene glycol, XTEN, human serum albumin, transferrin, carboxy-terminal peptide, a homo-amino acid polymer, a proline-alanine-serine polymer, an elastin-like peptide, or hyaluronic acid. In certain cases, the half-life extension moiety is attached at the N-terminal of the VHH. In other cases, the half-life extension moiety is attached at the C-terminal of the VHH.

In some instances, the binding molecule is bispecific and also comprises a binding moiety that specifically binds to a different antigen than what the VHH binds (i.e., CD38, BCMA, GPRC5D, or FcRH5). In some cases, the binding moiety is attached at the N-terminal of the VHH. In other cases, the binding moiety is attached at the C-terminal of the VHH. In certain instances, the different antigen is an antigen expressed on a T cell or a NK cell.

In another aspect the disclosure provides a bispecific antibody comprising a VHH that specifically binds any one of human and cynomolgus CD38, BCMA, GPRC5D, or FcRH5, and a binding moiety that binds a different antigen. In some instances, the VHH comprises the CDRs or the amino acid sequence of any one VHH described herein. In some instances, the binding moiety binds an antigen on a T cell or NK cell. In some cases, the binding moiety is attached at the N-terminal of the VHH. In other cases, the binding moiety is attached at the C-terminal of the VHH. In certain cases, the binding moiety is a scFv, Fab, or a second VHH.

In a further aspect the disclosure provides a bispecific antibody comprising a means for specifically binding any one of human and cynomolgus CD38, BCMA, GPRC5D, or FcRH5, and a binding moiety that specifically binds to a different antigen than CD38, BCMA, GPRC5D, or FcRH5. In some cases, the different antigen is an antigen expressed on a T cell or a NK cell. In certain cases, the binding moiety is a scFv, Fab, or a second VHH.

In another aspect, the disclosure features a chimeric antigen receptor (CAR) comprising a binding molecule described herein, a hinge region, a transmembrane domain and at least one intracellular signaling domain, wherein the hinge region is between the binding molecule and the transmembrane domain, and the at least one intracellular signaling domain is attached to the C-terminus of the transmembrane domain.

In another aspect, the disclosure relates to a CAR comprising a means for specifically binding any one of human and cynomolgus CD38, BCMA, GPRC5D, or FcRH5, a hinge region, a transmembrane domain and at least one intracellular signaling domain, wherein the hinge region is between the binding molecule and the transmembrane domain, and the at least one intracellular signaling domain is attached to the C-terminus of the transmembrane domain.

In some instances, the hinge is from CD28 or CD8alpha. In certain instances, the transmembrane domain is the CD28 transmembrane domain. In some cases, the at least one intracellular signaling domain is from any one of CD27, CD28, CD134, CD137, 4-1BB, or CD3%.

In another aspect, the disclosure features a T cell expressing a CAR described herein.

In a different aspect, the disclosure relates to a NK cell expressing a CAR described herein.

In one aspect, the disclosure features a pharmaceutical composition comprising a means for specifically binding any one of human and cynomolgus CD38, BCMA, GPRC5D, or FcRH5 and a pharmaceutically acceptable carrier.

In a different aspect, the disclosure provides a pharmaceutical composition comprising a bispecific antibody comprising a means for specifically binding any one of human and cynomolgus CD38, BCMA, GPRC5D, or FcRH5, and a binding moiety that specifically binds to a different antigen than CD38, BCMA, GPRC5D, or FcRH5. In some instances, the different antigen is an antigen expressed on a T cell or a NK cell. In some cases, the binding moiety is a scFv, Fab, or a second VHH.

In another aspect, the disclosure relates to a pharmaceutical composition comprising a binding molecule or a bispecific antibody described herein, and a pharmaceutically acceptable carrier.

In a different aspect, the disclosure features nucleic acid encoding a binding molecule or a bispecific antibody, or a CAR described herein. In some instances, provided are a vector or vectors comprising the nucleic acid(s). Also provided are host cells comprising the nucleic acid(s) or vector or vectors. In some cases, the host cell is a CHO cell, a COS cell, a 293 cell, a NIH3T3 cell, or a HeLa cell.

In one aspect, the disclosure provides a method of making a binding molecule or bispecific antibody described herein. The method comprises culturing a host cell described herein under conditions that facilitate the expression of the binding molecule or bispecific antibody, and isolating the binding molecule or bispecific antibody. In some cases, the method further comprises formulating the binding molecule or bispecific antibody as a pharmaceutical composition.

In another aspect, the disclosure relates to a method of treating a cancer in a human subject in need thereof. The method comprises administering to the human subject a therapeutically effective amount of a binding molecule, a bispecific antibody, or a T or NK cell described herein. In some cases, the cancer is multiple myeloma (MM). In certain cases, the MM is Stage I, Stage II, Stage III MM, MGUS, or SMM.

In a different aspect, the disclosure features a polynucleotide comprising an mRNA comprising: (i) a 5′ UTR; (ii) an open reading frame (ORF) encoding a binding molecule of or a bispecific antibody described herein; (iii) a stop codon; and (iv) a 3′ UTR.

In some instances, the mRNA comprises a microRNA (miR) binding site. In certain cases, the microRNA is expressed in an immune cell of hematopoietic lineage or a cell that expresses TLR7 and/or TLR8 and secretes pro-inflammatory cytokines and/or chemokines. In some cases, the microRNA binding site is for a microRNA selected from miR-126, miR-142, miR-144, miR-146, miR-150, miR-155, miR-16, miR-21, miR-223, miR-24, miR-27, miR-26a, or any combination thereof. In other cases, the microRNA binding site is for a microRNA selected from miR126-3p, miR-142-3p, miR-142-5p, miR-155, or any combination thereof. In certain cases, the microRNA binding site is located in the 3′ UTR of the mRNA.

In some instances, the mRNA comprises a 5′ terminal cap. In certain cases, the 5′ terminal cap comprises a Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In some instances, the mRNA comprises a poly-A region. In certain cases, the poly-A region is at least about 10, at least about 20, at least about 30, at least about 40, at least about 50, at least about 60, at least about 70, at least about 80, at least about 90 nucleotides in length, or at least about 100 nucleotides in length. In other cases, the poly-A region is about 10 to about 200, about 20 to about 180, about 50 to about 160, about 70 to about 140, or about 80 to about 120 nucleotides in length.

In some instances, the mRNA comprises at least one chemically modified nucleobase, sugar, backbone, or any combination thereof. In some cases, the at least one chemically modified nucleobase is selected from the group consisting of pseudouracil (ψ), N1-methylpseudouracil (m1ψ), 1-ethylpseudouracil, 2-thiouracil (s2U), 4′-thiouracil, 5-methylcytosine, 5-methyluracil, 5-methoxyuracil, and any combination thereof. In certain cases, at least about 25%, at least about 30%, at least about 40%, at least about 50%, at least about 60%, at least about 70%, at least about 80%, at least about 90%, at least about 95%, at least about 99%, or 100% of the uracils are N1-methylpseudouracils.

In some instances, the open reading frame consists of nucleosides selected from the group consisting of (i) uridine or a modified uridine, (ii) cytidine or a modified cytidine, (iii) adenosine or a modified adenosine, and (iv) guanosine or a modified guanosine. In certain cases, the modified uridine is 1-methylpseudouridine.

In some instances, the mRNA comprises a 5′terminal cap comprising Cap1 and a poly-A region 100 nucleotides in length (SEQ ID NO: 493).

In some instances, all uracils of the polynucleotide are N1-methylpseudouracils.

In another aspect, the disclosure relates to a pharmaceutical composition comprising a polynucleotide described herein, and a delivery agent.

In some instances, the delivery agent comprises a lipid nanoparticle. In some cases, the lipid nanoparticle has a mean particle size of from 80 nm to 160 nm. In certain cases, the lipid nanoparticle has a polydispersity index (PDI) of from 0.02 to 0.2 and/or a lipid: nucleic acid ratio of from 10 to 20. In some cases, the lipid nanoparticle comprises a neutral lipid, an ionizable amino lipid, a polyethyleneglycol (PEG) lipid, and/or a sterol. In certain cases, the lipid nanoparticle comprises a neutral lipid that is 1,2-distearoyl-sn-glycero-3-phosphocholine. In some cases, the lipid nanoparticle comprises an ionizable amino lipid. In certain instances, the lipid nanoparticle comprises a PEG lipid that is PEG 2000 dimyristoyl glycerol or 134-hydroxy-3,6,9,12,15,18,21,24,27,30,33,36,39,42,45,48,51,54,57,60,63,66,69,72,75,78,81,84,87, 90,93,96,99,102,105,108,111,114,117,120,123,126,129,132-tetratetracontaoxatetratriacontahectyl stearate or a salt thereof. In certain cases, the lipid nanoparticle comprises a sterol that is cholesterol, adosterol, agosterol A, atheronals, avenasterol, azacosterol, blazein, cerevisterol, colestolone, cycloartenol, daucosterol, 7-dehydrocholesterol, 5-dehydroepisterol, 7-dehydrositosterol, 20a,22R-dihydroxycholesterol, dinosterol, epibrassicasterol, episterol, ergosterol, ergosterol, fecosterol, fucosterol, fungisterol, ganoderenic acid, ganoderic acid, ganoderiol, ganodermadiol, 7-hydroxycholesterol, 22R-hydroxycholesterol, 27-hydroxycholesterol, inotodiol, lanosterol, lathosterol, lichesterol, lucidadiol, lumisterol, oxycholesterol, oxysterol, parkeol, saringosterol, spinasterol, sterol ester, trametenolic acid, zhankuic acid, or zymosterol. In one case, the sterol is cholesterol.

In another aspect, the disclosure provides a kit comprising (i) a binding molecule; a bispecific antibody; a T or NK cell; a pharmaceutical composition; or a polynucleotide described herein, and (ii) a package insert instructing a user of the kit to administer the binding molecule, bispecific antibody, T cell or NK cell, pharmaceutical composition, or polynucleotide to a human subject in need thereof.

In yet another aspect, the disclosure relates to a method of treating a cancer in a human subject in need thereof. The method comprises administering to the human subject a therapeutically effective amount of a polynucleotide or a pharmaceutical composition described herein. In some instances, the cancer is multiple myeloma (MM). In some cases, the MM is Stage I, Stage II, Stage III MM, MGUS, or SMM.

In another aspect the disclosure features a lipid nanoparticle comprising an mRNA that encodes a binding molecule comprising a VHH that specifically binds (i) CD38 and comprises a VHH CDR1, VHH CDR2, and VHH CDR3 of any one of the VHH sequences of SEQ ID NOs.: 1 to 12; (ii) BCMA and comprises a VHH CDR1, VHH CDR2, and VHH CDR3 of any one of the VHH sequences of SEQ ID NOs.: 52 to 102; (iii) GPRC5D and comprises a VHH CDR1, VHH CDR2, and VHH CDR3 of any one of the VHH sequences of SEQ ID NOs.: 259 to 264; or (iv) FcRH5 and comprises a VHH CDR1, VHH CDR2, and VHH CDR3 of any one of the VHH sequences of SEQ ID NOs.: 321-325. In some cases, the VHH specifically binds (i) CD38 and comprises the VHH CDR1, VHH CDR2, and VHH CDR3 of any one of the VHHs of Table 5 or A; (ii) BCMA and comprises the VHH CDR1, VHH CDR2, and VHH CDR3 of any one of the VHHs of Table 9 or B; (iii) GPRC5D and comprises the VHH CDR1, VHH CDR2, and VHH CDR3 of any one of the VHHs of Table 12 or C; or (iv) FcRH5 and comprises the VHH CDR1, VHH CDR2, and VHH CDR3 of any one of the VHHs of Table 16 or D. In certain cases, the VHH specifically binds to: (i) CD38 and comprises an amino acid sequence that is at least 85%, at least 90%, at least 95%, at least 96%, at least 97%, at least 98%, or at least 99% identical to any one VHH of Table 4; (ii) BCMA and comprises an amino acid sequence that is at least 85%, at least 90%, at least 95%, at least 96%, at least 97%, at least 98%, or at least 99% identical to any one VHH of Table 8; (iii) GPRC5D and comprises an amino acid sequence that is at least 85%, at least 90%, at least 95%, at least 96%, at least 97%, at least 98%, or at least 99% identical to any one VHH of Table 11; (iv) FcRH5 and comprises an amino acid sequence that is at least 85%, at least 90%, at least 95%, at least 96%, at least 97%, at least 98%, or at least 99% identical to any one VHH of Table 15; (v) CD38 and comprises an amino acid sequence that is at least 95%, at least 96%, at least 97%, at least 98%, at least 99% or 100% identical to any one VHH of Table 7; (vi) BCMA and comprises an amino acid sequence that is at least 95%, at least 96%, at least 97%, at least 98%, at least 99% or 100% identical to any one VHH of Table 10; (vii) GPRC5D and comprises an amino acid sequence that is at least 95%, at least 96%, at least 97%, at least 98%, at least 99% or 100% identical to any one VHH of Table 14; or (viii) FcRH5 and comprises an amino acid sequence that is at least 95%, at least 96%, at least 97%, at least 98%, at least 99% or 100% identical to any one VHH of Table 18. In certain cases, the VHH binds to cell surface expressed CD38, BCMA, GPRC5D, or FcRH5, respectively, with an EC50 of 0.1 nM to 10 nM. In some cases, the binding molecule further comprises an agent selected from the group consisting of a linker (e.g., glycine serine linker), a VHH, an Fab, a scFv, a multimerization module, a moiety that facilitates the polypeptide crossing the blood brain barrier, and a half-life extension moiety. In some cases, the agent is attached or linked to the N-terminus of the polypeptide. In other cases, the agent is attached or linked to the C-terminus of the polypeptide.

In certain instances, the lipid nanoparticle comprises an ionizable amino lipid, a PEG-lipid, a structural lipid, and a phospholipid. In certain cases the lipid nanoparticle comprises: about 47.5 mol % of ionizable amino lipid; about 39 mol % of cholesterol; about 10.5 mol % of DSPC; and about 3 mol % of PEG-lipid. In certain cases, the lipid nanoparticle comprises: 47.5 mol % of an ionizable amino lipid; 39 mol % of cholesterol; 10.5 mol % of DSPC; and 3 mol % of a PEG-lipid. In some cases, the ionizable amino acid lipid is heptadecan-9-yl 8-((2-hydroxyethyl) (8-(nonyloxy)-8-oxooctyl)amino) octanoate which has the formula

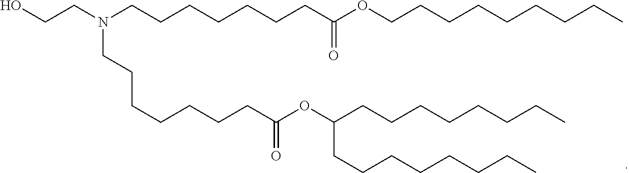 [SMILES: CCCCCCCCCOC(=O)CCCCCCCN(CCO)CCCCCCCC(=O)OC(CCCCCCCC)CCCCCCCC]

or a salt thereof. In other cases, the ionizable amino acid lipid is heptadecan-9-yl 8-((2-hydroxyethyl) (6-oxo-6-(undecyloxy) hexyl)amino) octanoate or a salt thereof. In some cases, the PEG lipid is 134-hydroxy-3,6,9,12,15,18,21,24,27,30,33,36,39,42,45,48,51,54,57,60,63,66,69,72,75,78,81,8 4,87,90,93,96,99,102,105,108,111,114,117,120,123,126,129,132-tetratetracontaoxatetratriacontahectyl stearate which has the formula

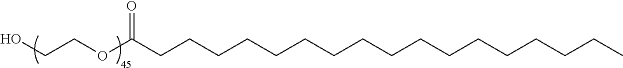

or a salt thereof. In other cases, the PEG-lipid is PEG-DMG or a salt thereof. In certain cases, the mRNA further comprises a 5′ terminal cap, a 5′UTR, a 3′UTR, and a poly A tail. In some cases, all uridines of the mRNA are 1-methylpseudouridine. In some cases, the mRNA comprises a 5′terminal cap (e.g., Cap1), a 5′UTR, a 3′UTR, and a poly-A region 100 nucleotides in length (SEQ ID NO: 493), and all uracils of the polynucleotide are N1-methylpseudouracils.

In yet another aspect, the disclosure relates to a method of treating a cancer in a human subject in need thereof. The method comprises administering to the human subject a therapeutically effective amount of an LNP described herein. In some instances, the cancer is multiple myeloma (MM). In some cases, the MM is Stage I, Stage II, Stage III MM, MGUS, or SMM. In some cases, the administering is performed subcutaneously. In other cases, the administering is performed intravenously. In yet other cases, the administering is performed intramuscularly.

In yet another aspect, the disclosure relates to a kit comprising LNP described above and a package insert instructing a user of the kit to administer the LNP to a human subject in need thereof. In some instances, the human subject has a cancer. In some instances, the cancer is multiple myeloma (MM). In some cases, the MM is Stage I, Stage II, Stage III MM, MGUS, or SMM.

The following drawings and detailed description are exemplary and explanatory, but it is not intended to be limiting.

### BRIEF DESCRIPTION OF THE DRAWINGS

FIG. 1 shows FACS binding curves for select VHH-His formatted anti-CD38 VHH-His antibodies to determine EC50 values against H929 cells.

FIG. 2 shows FACS binding curves for select VHH-His formatted anti-CD38 VHH-His antibodies to determine EC50 values against RPMI8226 cells expressing CD38.

FIG. 3 shows FACS binding curves for select VHH-His formatted anti-GPRC5D to determine EC50 values against CHO-HuGPRC5D which express full length GPRC5D.

FIG. 4 shows FACS binding curves for select VHH-His formatted anti-GPRC5D to determine EC50 values against H929-GFP-Luc expressing GPRC5D.

FIG. 5 shows FACS binding curves for select VHH-His formatted anti-FcRH5 to determine EC50 values against CyFcRH5-RPMI8226, expressing cyno FCRH5.

FIG. 6 shows FACS binding curves for select VHH-His formatted anti-FcRH5 to determine EC50 values against HuFcRH5-RPMI8226, expressing full length human FCRH5.

FIG. 7 shows FACS binding curves for Fc formatted anti-BCMA to determine EC50 values against CHO-HuBCMA and H929 cells.

FIG. 8 shows FACS binding curves for Fc formatted anti-BCMA to determine EC50 values against RPMI8226 cells.

### DETAILED DESCRIPTION

The compositions and methods of the disclosure feature CD38, BCMA, GPRC5D, and/or FcRH5 heavy chain variable domains (VHH) (anti-CD38, anti-BCMA, anti-GPRC5D, and/or anti-FcRH5 VHH domains) and complementarity determining regions (CDRs) thereof, as well as antibodies and other related binding proteins that comprise the disclosed VHH domains or CDRs. The disclosure also provides nucleic acids encoding the disclosed proteins, and methods of using such antibodies, binding proteins, and nucleic acids. In some embodiments, the antibodies are single domain antibodies (e.g., a VHH). In some embodiments, the antibodies are single-domain antibodies (sdAbs) or single chain Fv (scFv) molecules. In some embodiments, the antibodies are bispecific antibodies (i.e., engagers) that bind to CD38, BCMA, GPRC5D, or FcRH5 and another antigenic target.

The compositions and methods of the disclosure exhibit a series of beneficial biochemical properties. For example, VHH domains described herein and antibodies are capable of binding CD38, BCMA, GPRC5D, and/or FcRH5 with high affinity.

### Definitions

As used herein, the term “about” refers to a stated numerical term and a value that is no more than 10% above or below the value being described. For example, the term “about 5 nM” indicates disclosure of both the stated value of 5 nM and a range of from 4.5 nM to 5.5 nM.

As used herein, the term “CD38 antibody” or “CD38-antibody” refers to an antibody or fragment thereof that specifically binds to, or is immunologically reactive with, CD38. Similarly, a “CD38 binding protein” or “anti-CD38 binding protein” refers to any protein comprising at least one domain (such as a VHH domain disclosed herein) that specifically binds to or is immunologically reactive with CD38. Accordingly, a “CD38 binding protein” or “anti-CD38 binding protein” includes, for example, anti-CD38 antibodies (both monospecific and bispecific), and other constructs that bind to CD38.

As used herein, the term “BCMA antibody” or “BCMA-antibody” refers to an antibody or fragment thereof that specifically binds to, or is immunologically reactive with, BCMA. Similarly, a “BCMA binding protein” or “anti-BCMA binding protein” refers to any protein comprising at least one domain (such as a VHH domain disclosed herein) that specifically binds to or is immunologically reactive with BCMA. Accordingly, a “BCMA binding protein” or “anti-BCMA binding protein” includes, for example, anti-BCMA antibodies (both monospecific and bispecific), and other constructs that bind to BCMA.

As used herein, the term “GPRC5D antibody” or “GPRC5D-antibody” refers to an antibody or fragment thereof that specifically binds to, or is immunologically reactive with, GPRC5D. Similarly, a “GPRC5D binding protein” or “anti-GPRC5D binding protein” refers to any protein comprising at least one domain (such as a VHH domain disclosed herein) that specifically binds to or is immunologically reactive with GPRC5D. Accordingly, a “GPRC5D binding protein” or “anti-GPRC5D binding protein” includes, for example, anti-GPRC5D antibodies (both monospecific and bispecific), and other constructs that bind to GPRC5D.

As used herein, the term “FcRH5 antibody” or “FcRH5-antibody” refers to an antibody or fragment thereof that specifically binds to, or is immunologically reactive with, FcRH5. Similarly, a “FcRH5 binding protein” or “anti-FcRH5 binding protein” refers to any protein comprising at least one domain (such as a VHH domain disclosed herein) that specifically binds to or is immunologically reactive with FcRH5. Accordingly, a “FcRH5 binding protein” or “anti-FcRH5 binding protein” includes, for example, anti-FcRH5 antibodies (both monospecific and bispecific), and other constructs that bind to FcRH5.

As used herein, the term “antibody” (Ab) refers to an immunoglobulin molecule, or a molecule having an immunoglobulin-like scaffold, that specifically binds to, or is immunologically reactive with, a particular antigen. The term “antibody” includes polyclonal, monoclonal, genetically engineered, and otherwise modified forms of antibodies, including, but not limited to, chimeric antibodies, humanized antibodies, and heteroconjugate antibodies (e.g., bi-tri-, quad-, and multispecific antibodies, diabodies, triabodies, and tetrabodies).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, e.g., a single-domain antibody (sdAb), Fab, F(ab′)₂, Fab Fv, VHH, scFv, SMIP, diabody, a triabody, an affibody, an aptamer, or recombinant fragments thereof. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, e.g., Bird et al., Science 242:423-426 (1988), and Huston et al., Proc. Natl. Acad. Sci. USA 85:5879-5883 (1988)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, term “biologic” refers to a medicinal preparation that is created by biological processes rather than chemical synthesis. Exemplary biologics include certain vaccines, antibodies, cell preparations, tissue preparations, recombinant proteins, nucleic acids, cytokines, growth factors, enzymes, peptides, proteins, carbohydrates, or combinations thereof. Biologics also include biosimilar molecules (or biosimilars), which are molecular entities that are structurally similar to and have no clinically meaningful differences in terms of safety, purity, and potency from known biologics.

As used herein, the term “bispecific antibodies” refers to monoclonal, often human or humanized antibodies that have binding specificities for at least two different antigens. Bispecific CD38, BCMA, GPRC5D, or FcRH5 antibodies of the invention may have binding specificities that are directed towards CD38, BCMA, GPRC5D, or FcRH5 and any other antigen, e.g., for a cell-surface protein, receptor, receptor subunit, or tissue-specific antigen. Bispecific CD38, BCMA, GPRC5D, or FcRH5 antibodies of the invention may have binding specificities that are directed towards a first antigen selected from CD38, BCMA, GPRC5D, and FcRH5 and a second antigen selected from CD38, BCMA, GPRC5D, and FcRH5. A bispecific antibody may also be an antibody or antigen-binding fragment thereof that includes two separate antigen-binding domains (e.g., two scFvs joined by a linker). The scFvs may bind the same antigen or different antigens. For the purposes of the present disclosure, the term “engager” may be used interchangeably with “bispecific antibody.”

As used herein, the term “multispecific antibodies” refers to monoclonal, often human or humanized antibodies that have binding specificities for at least two different antigens, e.g., bispecific antibodies. Multispecific CD16 antibodies of the invention may have binding specificities that are directed towards CD16 and any other antigen(s), e.g., for a cell-surface protein, receptor, receptor subunit, or tissue-specific antigen. A multispecific antibody may also be an antibody or antigen-binding fragment thereof that includes multiple separate antigen-binding domains (e.g., two scFvs joined by a linker). The scFvs may bind the same antigen or different antigens. Such structures can include, but not limited to, IgG-Fv, IgG-(scFv)₂, DVD-Ig, (scFv)₂-(scFv)₂-Fc and (scFv)₂-Fc-(scFv)₂. In case of IgG-(scFv)₂, the scFv can be attached to either the N-terminal or the C-terminal end of either the heavy chain or the light chain. Exemplary multi-specific molecules that include Fc regions and into which CD16 antibodies or antigen-binding fragments thereof are disclosed in Kontermann, 2012, mAbs 4 (2): 182-197, Yazaki et al., Protein Engineering, Design & Selection 26 (3): 187-193 (2013), and Grote et al., in Proetzel & Ebersbach (eds.), Antibody Methods and Protocols, Methods in Molecular Biology vol. 901, chapter 16:247-263 (2012); incorporated herein by reference. In some embodiments, antibody fragments can be components of multi-specific molecules without Fc regions, based on fragments of IgG or DVD or scFv. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, 2003, Nature Medicine 9:129-134; incorporated herein by reference. Multi-specific antibodies also include tetravalent bispecific antibody.

As used herein, the term “chimeric” antibody refers to an antibody having portions of its sequence derived from at least two different sources, such as variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, bison, llama, camel, or shark among others). Methods for producing chimeric antibodies are known in the art. See, e.g., Morrison, Science 229 (4719): 1202-7 (1985); Oi et al., BioTechniques. 4:214-221 (1986); Gillies et al., J. Immunol. Methods 125:191-202 (1985); U.S. Pat. Nos. 5,807,715; 4,816,567; and 4,816,397; incorporated herein by reference.

As used herein, the term “complementarity determining region” or “CDR” refers to a hypervariable region found in the light chain and/or the heavy chain variable domains of an antibody. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The invention includes antibodies comprising modifications in these hybrid hypervariable positions. The variable domains of native heavy and light chains each comprise four framework regions that primarily adopt a β-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see, Kabat et al., Sequences of Proteins of Immunological Interest (National Institute of Health, Bethesda, Md. (1987); incorporated herein by reference). As used herein, numbering of immunoglobulin amino acid residues is performed according to the immunoglobulin amino acid residue numbering system of Kabat et al., unless otherwise indicated.

As used herein, the terms “conservative mutation,” “conservative substitution,” “conservative amino acid substitution,” and the like refer to a substitution of one or more amino acids for one or more different amino acids that exhibit similar physicochemical properties, such as polarity, electrostatic charge, and/or steric volume. These properties are summarized for each of the twenty naturally-occurring amino acids in Table 1 below.

TABLE 1
Representative physicochemical properties of naturally-occurring amino acids

| | | | | Electrostatic | |
|---|---|---|---|---|---|
| | 3 | 1 | Side- | character at | |
| | Letter | Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| | | | | Both neutral and | |
| Histidine | His | H | polar | cationic forms in | large |
| | | | | equilibrium at pH | |
| | | | | 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “conjugate” refers to a compound formed by the chemical bonding of a reactive functional group of one molecule with an appropriately reactive functional group of another molecule. Conjugates may additionally be produced, e.g., as two polypeptide domains covalently bound to one another as part of a single polypeptide chain that is synthesized by the translation of a single RNA transcript encoding both polypeptides in frame with one another.

As used herein in the context of a CD38-, BCMA-, GPRC5D-, or FcRH5-binding protein, the term “construct” refers to a fusion protein containing a first polypeptide domain bound to a second polypeptide domain. The polypeptide domains may each independently be anti-CD38, anti-BCMA, anti-GPRC5D, or anti-FcRH5 single chain polypeptides, for instance, as described herein. The first polypeptide domain may be covalently bound to the second polypeptide domain, for instance, by way of a linker, such as a peptide linker or a disulfide bridge, among others. Exemplary linkers that may be used to join the polypeptide domains of a CD38, BCMA, GPRC5D, or FcRH5 construct include, without limitation, those that are described in Leriche et al., Bioorg. Med. Chem., 20:571-582 (2012), the disclosure of which is incorporated herein by reference in its entirety.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, and/or linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies comprising two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structure, peptides configured in this way typically trimerize so as to position the V_{H} and V_{L} domains of neighboring peptide chains spatially proximal to one another to permit proper folding (see, Holliger et al., Proc. Natl. Acad. Sci. USA 90:6444-48 (1993); incorporated herein by reference).

As used herein, a “tetravalent bispecific antibody” or “tetravalent bsAB” refers to an antibody comprising two peptide chains. Each of the peptide chains contains two sdAB sequences. For example, each peptide chain may include an anti-CD38, anti-BCMA, anti-GPRC5D, or anti-FcRH5 first sdAB and a second sdAB that binds to a different target than the first sdAB (e.g., the second dsAB does not bind CD38, BCMA, GPRC5D, or FcRH5, respectively). The peptide chains can dimerize at the hinge region of their respective Fc domains, thus resulting in a tetravalent construct.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582 (2012)).

As used herein, the term “heterospecific antibodies” refers to monoclonal (e.g., human or humanized) antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537 (1983)). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986); incorporated herein by reference. Heterospecific antibodies can include Fc mutations that enforce correct chain association in multi-specific antibodies, as described by Klein et al., mAbs 4 (6): 653-663 (2012); incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Human antibodies can be made by a variety of methods known in the art including phage display methods using antibody libraries derived from human immunoglobulin sequences. See, U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, or immunoglobulin chains or fragments thereof (such as Fv, Fab, Fab′, F(ab′)₂ or other target-binding subdomains of antibodies), which contain minimal sequences derived from non-human immunoglobulin. In general, a humanized antibody will contain substantially all of at least one, and typically two, variable domains, in which all or substantially all of the CDRs correspond to those of a non-human immunoglobulin. All or substantially all of the FRs may also be those of a human immunoglobulin sequence. The humanized antibody may also contain at least a portion of an immunoglobulin constant region (Fc), typically that of a human immunoglobulin consensus sequence. Methods of antibody humanization are known in the art. See, e.g., Riechmann et al., Nature 332:323-7 (1988); U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; the disclosure of each of which is incorporated herein by reference.

As used herein, the term “lipid nanoparticle” refers to a transfer vehicle including one or more lipids (e.g., cationic lipids, non-cationic lipids, and PEG-modified lipids). Exemplary lipid nanoparticles are formulated to deliver one or more mRNA to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Lipid nanoparticles may contain a cationic lipid, or a lipid species with a net positive charge at a selected pH (e.g., physiological pH), to encapsulate and/or enhance the delivery of mRNA into the target cells.

As used herein, the terms “messenger RNA” or “mRNA” refer to any polynucleotide which encodes a polypeptide of interest and which is capable of being translated to produce the encoded polypeptide of interest in vitro, in vivo, in situ, or ex vivo. Traditionally, the basic components of an mRNA molecule include a coding region, a 5′UTR, a 3′UTR, a 5′ cap, and a poly-A tail.

As used herein, the terms “modified messenger RNA” or “modified mRNA” refer to mRNA polynucleotides that include naturally occurring and/or non-naturally occurring modifications, for example, of a sugar, a nucleobase, or an internucleoside linkage (e.g., to a linking phosphate, to a phosphodiester linkage, or to the phosphodiester backbone). Non-natural modified nucleotides may be introduced during synthesis of post-synthesis of the polynucleotides to achieve desired functions or properties. The modifications may be present on an internucleoside linkage, purine or pyrimidine base, or sugar. The modification may be introduced with chemical synthesis or with a polymerase enzyme at the terminal of a chain or anywhere else in the chain. Any of the regions of a polynucleotide may be chemically modified.

As used herein, the term “monoclonal antibody” refers to an antibody that is derived from a single clone, including any eukaryotic, prokaryotic, or phage clone, and not the method by which it is produced.

As used herein, the term “nucleic acid” includes any compound containing a continuous segment of nucleosides joined by way of one or more internucleoside linkages (e.g., polymers of nucleosides linked by way of phosphodiester bonds). Exemplary nucleic acids include ribonucleic acids (RNA, in particular mRNA), deoxyribonucleic acids (DNA), threose nucleic acids (TNA), glycol nucleic acids (GNA), peptide nucleic acids (PNA), locked nucleic acids (LNA), or hybrids thereof. Nucleic acids also include RNAi inducers, RNAi agents, siRNAs, shRNAs, miRNAs, antisense RNAs, ribozymes, catalytic DNAs, tRNAs, RNAs that induce triple spiral formation, aptamers, vectors, and the like. In a preferred embodiment, the nucleic acid is one or more modified messenger RNAs (modified mRNAs).

As used herein, the terms “percent (%) sequence identity,” “percent (%) identity,” and the like, with respect to a reference polynucleotide or polypeptide sequence, is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as: 100 multiplied by (the fraction X/Y) where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “primatized antibody” refers to an antibody comprising framework regions from primate-derived antibodies and other regions, such as CDRs and/or constant regions, from antibodies of a non-primate source. Methods for producing primatized antibodies are known in the art. See, e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “operatively linked” in the context of a polynucleotide fragment is intended to mean that the two polynucleotide fragments are joined such that the amino acid sequences encoded by the two polynucleotide fragments remain in-frame.

As used herein, the term “pharmacokinetic profile” refers to the absorption, distribution, metabolism, and clearance of a therapeutic agent (e.g., a polypeptide, such as an anti-CD38, anti-BCMA, anti-GPRC5D, or anti-FcRH5 antibody, antigen-binding fragment thereof, single-chain polypeptide, or construct of the disclosure) over time following administration of the drug to a patient.

As used herein, the term “regulatory sequence” includes promoters, enhancers, and other expression control elements (e.g., polyadenylation signals) that control the transcription or translation, e.g., of antibody chain genes. Such regulatory sequences are described, for example, in Goeddel, Gene Expression Technology: Methods in Enzymology 185 (Academic Press, San Diego, CA, 1990); incorporated herein by reference.

As used herein, the term “scFv” refers to a single-chain Fv antibody in which the variable domains of the heavy chain and the light chain from an antibody have been joined to form one chain. ScFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). ScFv molecules are known in the art and are described, e.g., in U.S. Pat. No. 5,892,019, Flo et al., Gene 77:51 (1989); Bird et al., Science 242:423 (1988); Pantoliano et al., Biochemistry 30:10117 (1991); Milenic et al., Cancer Research 51:6363 (1991); and Takkinen et al., Protein Engineering 4:837 (1991). The V_{L} and V_{H} domains of a scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. ScFv fragments are described, for example, in WO 2011/084714; incorporated herein by reference.

As used herein, the terms “single-domain antibody,” “sdAb,” “nanobody,” and “VHH antibody” are used interchangeably to refer to a single-chain antibody fragment that contains only a single heavy-chain variable domain. Unlike a traditional, full-length antibody, which includes heavy chains and light chains, each containing a corresponding variable domain (i.e., a heavy chain variable domain, V_{H}, and a light chain variable domain, V_{L}) having three CDRs, a single-domain antibody only includes one heavy-chain variable domain having a total of three CDRs (referred to herein as CDR-H1, CDR-H2, and CDR-H3).

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a K_{D} of less than 100 nM. For example, an antibody or antigen-binding fragment thereof that specifically binds to an antigen will bind to the antigen via the antigen binding domain with a K_{D} of up to 100 nM (e.g., between 1 μM and 100 nM). An antibody or antigen-binding fragment thereof that does not exhibit specific binding to a particular antigen or epitope thereof will exhibit a K_{D} of greater than 100 nM (e.g., greater than 500 nm, 1 μM, 100 μM, 500 μM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “subject” and “patient” refer to an organism that receives treatment (e.g., by administration of an CD38, BCMA, GPRC5D, and/or FcRH5 polypeptide, such as an antibody, antigen-binding fragment thereof, single-chain polypeptide, or construct described herein) for a particular disease or condition. Examples of subjects and patients include mammals, such as humans, primates, pigs, goats, rabbits, hamsters, cats, dogs, guinea pigs, members of the bovidae family (such as cattle, bison, buffalo, and yaks, among others), sheep, and horses, among others. A patient that may be treated using the compositions and methods described herein may have an established disease, in which case the patient has been diagnosed as having the disease and has shown symptoms of the disease for a prolonged period of time (e.g., over the course of days, weeks, months, or years). Alternatively, a patient may be symptomatic for a particular disease, but has yet to be diagnosed with the disease by a physician. Other patients that may be treated using the compositions and methods described herein include those that have been diagnosed as having a disease or disorder, and may or may not be showing symptoms of the disease as of yet.

As used herein, the term “transfection” refers to any of a wide variety of techniques commonly used for the introduction of a nucleic acid molecule, e.g., exogenous DNA or RNA into a prokaryotic or eukaryotic host cell, e.g., electroporation, lipofection, calcium-phosphate precipitation, DEAE-dextran transfection and the like.

As used herein, the terms “treat” or “treatment” refer to therapeutic treatment, in which the object is to inhibit or slow down (lessen) an undesired physiological change or disorder. Beneficial or desired clinical results of treatment include, without limitation, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already having the condition or disorder, as well as those prone to have the condition or disorder or those in which the condition or disorder is to be inhibited.

As used herein the term “variable region CDR” includes amino acids in a CDR or complementarity determining region as identified using sequence or structure-based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616 (1977) and Kabat, et al., Sequences of Proteins of Immunological Interest, Fifth Edition, U.S. Department of Health and Human Services, NIH Publication No. 91-3242 (1991); by Chothia et al., J. Mol. Biol. 196:901-917 (1987), and by MacCallum et al., J. Mol. Biol. 262:732-745 (1996) where the definitions include overlapping or subsets of amino acid residues when compared against each other. In certain embodiments, the term “CDR” is a CDR as defined by Kabat based on sequence comparisons.

As used herein, the term “vector” includes a nucleic acid vector, e.g., a DNA vector, such as a plasmid, an RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of antibodies, antibody fragments, and/or binding proteins described herein include plasmids that contain regulatory sequences, such as promoter and enhancer regions, which direct gene transcription. Other useful vectors for expression of antibodies, antibody fragments, and/or binding proteins contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “V_{H}” refers to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (V_{H}) followed by a number of constant domains. Each light chain of a native antibody has a variable domain at the amino terminus (V_{L}) and a constant domain at the carboxy terminus.

### Structural Characteristics of Exemplary Anti-CD38, Anti-BCMA, Anti-GPRC5D, or Anti-FcRH5 Antibodies

Among the molecular features of anti-CD38, anti-BCMA, anti-GPRC5D, and anti-FcRH5 antibodies and binding proteins comprising anti-CD38, anti-BCMA, anti-GPRC5D, and anti-FcRH5 VHH domains described herein, it will be appreciated by one of skill in the art that the CDRs are those regions that predominantly dictate the CD38-, BCMA-, GPRC5D-, and FcRH5-binding properties of the molecule. This section provides amino acid sequence information for the CDRs of anti-CD38, anti-BCMA, anti-GPRC5D, and anti-FcRH5 VHH domains and antibodies comprising such anti-CD38, anti-BCMA, anti-GPRC5D, and anti-FcRH5 VHH domains of the disclosure.

In some embodiments, the disclosure provides an antibody or binding protein having one, two, or three of the CDRs described in Tables 5, 9, 13, and, 17, or Tables A, B, C, or D, below. In one instance, the disclosure features an antibody or binding protein comprising the three CDRs of any one VHH disclosed herein. The CDRs can be based on any CDR definition of the art. In some cases, the CDRs are based on Kabat, Chothia, enhanced Chothia, Aho, AbM, Contact, or IMGT definitions. In specific cases, the three CDRS are the three CDRS of any one clone disclosed in Tables 5, 9, 13, and, 17, or Tables A, B, C, or D, below

For example, in some embodiments, an anti-CD38, anti-BCMA, anti-GPRC5D, and anti-FcRH5 VHH antibody or binding protein of the disclosure is an antibody (e.g., a VHH or bispecific antibody) or antigen-binding fragment thereof (e.g., a scFv) having one or more of the CDRs disclosed herein.

Examples of CDRs for anti-CD38 antibodies, antigen-binding fragments, or binding proteins include:
(a) a CDR1 having the amino acid sequence selected from the group consisting of GFILDTYS (SEQ ID NO: 13); GFIFSDKV (SEQ ID NO: 16); RSIFEINTMT (SEQ ID NO: 19); GFSLDYYH (SEQ ID NO: 22); GAIVSAES (SEQ ID NO: 25); GTFSSINL (SEQ ID NO: 28); GSISGLNT (SEQ ID NO: 31); GSSVSMNS (SEQ ID NO: 34); GFIYSIST (SEQ ID NO: 37); GRYFRINA (SEQ ID NO: 40); GTFSSIAL (SEQ ID NO: 43); and GIIFRIFS (SEQ ID NO: 46); or an amino acid sequence having up to two amino acid substitutions (e.g., conservative amino acid substitutions) relative to SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, or SEQ ID NO: 46; and
(b) a CDR2 having the amino acid sequence selected from the group consisting of: ISSRDGNT (SEQ ID NO: 14); ITPGGTAT (SEQ ID NO: 17); SRGATT (SEQ ID NO: 20); ISSSDGYT (SEQ ID NO: 23); IISGSKS (SEQ ID NO: 26); DYTEGTT (SEQ ID NO: 29); IISGTMT (SEQ ID NO: 32); ITPGDRI (SEQ ID NO: 35); ITSGGNT (SEQ ID NO: 38); ISNDGST (SEQ ID NO: 41); VSIGGVT (SEQ ID NO: 44); and ITSGGNT (SEQ ID NO: 47); or an amino acid sequence having up to two amino acid substitutions (e.g., conservative amino acid substitutions) relative to SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, or SEQ ID NO: 47; and
(c) a CDR3 having the amino acid sequence selected from the group consisting of: AAGAQAHCTIFTSYFNSDYYRRYNY (SEQ ID NO: 15); RIGGPGGRYDN (SEQ ID NO: 18); SADRYGFGYGDNDY (SEQ ID NO: 21); AASPRRLACAGSLYPPLSADFSS (SEQ ID NO: 24); KRTERIWINNPQVY (SEQ ID NO: 27); WLMVRAGDVY (SEQ ID NO: 30); TFKEITRDSRSY (SEQ ID NO: 33); NIGATRPPFGA (SEQ ID NO: 36); NTAWRETIVSRV (SEQ ID NO: 39); NVKALPFLSSNELSY (SEQ ID NO: 42); WRMEGAGDVY (SEQ ID NO: 45); and NVAIPSGIVDRSA (SEQ ID NO: 48); or an amino acid sequence having up to two amino acid substitutions (e.g., conservative amino acid substitutions) relative to SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, or SEQ ID NO: 45.

Examples of CDRs for anti-BCMA antibodies, antigen-binding fragments, or binding proteins include:
(a) a CDR1 having the amino acid sequence selected from the group consisting of GRTLSPYT (SEQ ID NO: 103); GRTLNNYV (SEQ ID NO: 106); GSIFAYHV (SEQ ID NO: 109); GRTFSDYT (SEQ ID NO: 113); GRPLRMYN (SEQ ID NO: 116); GSTFSRYA (SEQ ID NO: 119); GFTLSSYW (SEQ ID NO: 122); GFTYSSYW (SEQ ID NO: 125); GGTLEYYA (SEQ ID NO: 128); GFTFSSYW (SEQ ID NO: 131); GRIDSGYT (SEQ ID NO: 134); GFTFGSYW (SEQ ID NO: 137); GHTLNSYA (SEQ ID NO: 140); GFTFSTYS (SEQ ID NO: 143); GHTFSNSA (SEQ ID NO: 146); GRTFSGYT (SEQ ID NO: 149); GRTFSSYA (SEQ ID NO: 152); GRTIGSFV (SEQ ID NO: 155); GFTFSNYA (SEQ ID NO: 158); VRYFSTYA (SEQ ID NO: 161); GFTFDDYA (SEQ ID NO: 163); GFNLTSDA (SEQ ID NO: 166); GRTFASFA (SEQ ID NO: 169); GFTLGYYA (SEQ ID NO: 174); GFTFEDYA (SEQ ID NO: 177); GFTFEDYA (SEQ ID NO: 177); GNFLRFNA (SEQ ID NO: 181); GSFSSIDT (SEQ ID NO: 184); EQNFSADY (SEQ ID NO: 187); EQNFSTDD (SEQ ID NO: 190); RSIFSINA (SEQ ID NO: 193); GSIFSINP (SEQ ID NO: 196); GRPIDTYA (SEQ ID NO: 200); GRTISTYA (SEQ ID NO: 203); GRPLECYA (SEQ ID NO: 206); GRRLEGYT (SEQ ID NO: 209); GPRLEGYT (SEQ ID NO: 212); GRTFNLYA (SEQ ID NO: 215); GVTFSNYV (SEQ ID NO: 217); GRTVSNYA (SEQ ID NO: 221); GRTFSNYA (SEQ ID NO: 224); and GRTFSRYA (SEQ ID NO: 227); or an amino acid sequence having up to two amino acid substitutions (e.g., conservative amino acid substitutions) relative to SEQ ID NO: 103, SEQ ID NO: 106, SEQ ID NO: 109, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 161, SEQ ID NO: 163, SEQ ID NO: 166, SEQ ID NO: 169, SEQ ID NO: 174, SEQ ID NO: 177, SEQ ID NO: 177, SEQ ID NO: 181, SEQ ID NO: 184, SEQ ID NO: 187, SEQ ID NO: 190, SEQ ID NO: 193, SEQ ID NO: 196, SEQ ID NO: 200, SEQ ID NO: 203, SEQ ID NO: 206, SEQ ID NO: 209, SEQ ID NO: 212, SEQ ID NO: 215, SEQ ID NO: 217, SEQ ID NO: 221, SEQ ID NO: 224, or SEQ ID NO: 227; and
(b) a CDR2 having the amino acid sequence selected from the group consisting of: ITARGDWT (SEQ ID NO: 104); MWWSGGSP (SEQ ID NO: 107); ITSGGST (SEQ ID NO: 110); MWWSGGSP (SEQ ID NO: 107); SVWTDGKP (SEQ ID NO: 114); ISWSGKTT (SEQ ID NO: 117); ISWSGGST (SEQ ID NO: 120); IKPESGIT (SEQ ID NO: 123); ISTGGDTTDT (SEQ ID NO: 126); ITWSGGST (SEQ ID NO: 129); INTDGDST (SEQ ID NO: 132); VVGSDGRD (SEQ ID NO: 135); IDTSGGHV (SEQ ID NO: 138); ISRSGEKT (SEQ ID NO: 141); IDARGVNT (SEQ ID NO: 144); ISLNGGNT (SEQ ID NO: 147); AVGSDGST (SEQ ID NO: 150); VSWTGDNT (SEQ ID NO: 153); VNWRGSST (SEQ ID NO: 156); INANSDTT (SEQ ID NO: 159); ISWNGGST (SEQ ID NO: 164); ISRT (SEQ ID NO: 167); ISWSGGFT (SEQ ID NO: 170); IASSDGST (SEQ ID NO: 172); ISGRDGST (SEQ ID NO: 175); TSKNDRMP (SEQ ID NO: 178); ISSGGRT (SEQ ID NO: 182); INRGGDT (SEQ ID NO: 185); ISNSGRT (SEQ ID NO: 188); ITNSGTT (SEQ ID NO: 191); ITNGGTT (SEQ ID NO: 194); FTSGGTT (SEQ ID NO: 197); VSWAGVYT (SEQ ID NO: 201); VSWAGSYT (SEQ ID NO: 204); ISGNGVNT (SEQ ID NO: 207); ITRNRVNT (SEQ ID NO: 213); ISRNRVNT (SEQ ID NO: 210); INRSGTP (SEQ ID NO: 222); and ITRSGTST (SEQ ID NO: 228); or an amino acid sequence having up to two amino acid substitutions (e.g., conservative amino acid substitutions) relative to SEQ ID NO: 104, SEQ ID NO: 107, SEQ ID NO: 110, SEQ ID NO: 107, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, SEQ ID NO: 156, SEQ ID NO: 159, SEQ ID NO: 164, SEQ ID NO: 167, SEQ ID NO: 170, SEQ ID NO: 172, SEQ ID NO: 175, SEQ ID NO: 178, SEQ ID NO: 182, SEQ ID NO: 185, SEQ ID NO: 188, SEQ ID NO: 191, SEQ ID NO: 194, SEQ ID NO: 197, SEQ ID NO: 201, SEQ ID NO: 204, SEQ ID NO: 207, SEQ ID NO: 213, SEQ ID NO: 210, SEQ ID NO: 222, or SEQ ID NO: 228; and
(c) a CDR3 having the amino acid sequence selected from the group consisting of: VRDLLGRDDY (SEQ ID NO: 105); AATWVGTSEYRH (SEQ ID NO: 108); AIGYYSGSYYWERSGDY (SEQ ID NO: 111); AATWVGTSEYQH (SEQ ID NO: 112); QALTGGSWILDY (SEQ ID NO: 115); FMNVWADTSDSSADAY (SEQ ID NO: 118); FGDIIRSGERSDYEY (SEQ ID NO: 121); VREDYDSAYVGDY (SEQ ID NO: 124); VALNLWGTDLEHDY (SEQ ID NO: 127); AAQFVEVEILVRSYEY (SEQ ID NO: 130); AAIVTRSDGHQYDY (SEQ ID NO: 133); AATNYYSDYLDHLSRGY (SEQ ID NO: 136); ARVENTWESIY (SEQ ID NO: 139); GAWNFVKNDAY (SEQ ID NO: 142); GGWEFAT (SEQ ID NO: 145); ATLGFAS (SEQ ID NO: 148); VKLDSGAWSLAE (SEQ ID NO: 151); AAWNWGHHEYTY (SEQ ID NO: 154); ARWSWDIGADFGS (SEQ ID NO: 157); VSDSYIGGLYATYVY (SEQ ID NO: 160); AAPYGSSQNLEYDY (SEQ ID NO: 162); VPVSHSDSVCGSPYMDY (SEQ ID NO: 165); AADNAADRCSLSIYNYNL (SEQ ID NO: 168); GEWGGFSL (SEQ ID NO: 171); VAPCFWFDTVIAGTDPRYDY (SEQ ID NO: 173); AAVRGPIVSMDPDLCRPVEFDY (SEQ ID NO: 176); AATNGPAITLFPCHINYWLYDN (SEQ ID NO: 179); ASADWRSPTPFPCGVSRSLYDH (SEQ ID NO: 180); WSAPDY (SEQ ID NO: 183); AGSFTLATGDDFGS (SEQ ID NO: 186); VAEPFSFRRRA (SEQ ID NO: 189); GESTTGWAECDFGC (SEQ ID NO: 192); NANSRYGVGWYNY (SEQ ID NO: 195); NVRGGHCDPRYWREY (SEQ ID NO: 198); NVRGGHYDPRYWREY (SEQ ID NO: 199); AATKLPWNTIVMVQRSYCDY (SEQ ID NO: 202); AATKLPWNTSVMVKRSVYDY (SEQ ID NO: 205); AATTVPVINLEISHMTY (SEQ ID NO: 208); AATNLPGITLLMSHMNYCDY (SEQ ID NO: 211); AATTVPVINLQVSHINY (SEQ ID NO: 214); AAKVFPMATLDDDVYDY (SEQ ID NO: 216); AAESHRRNTIVIVTTPDEYDY (SEQ ID NO: 218); AADPSSGYNLFARTVVAFARYDY (SEQ ID NO: 219); AAHPAGAHGGLIYKN (SEQ ID NO: 220); AADPTGAHVGAIYKN (SEQ ID NO: 223); AADLRGSSWYFDGVDY (SEQ ID NO: 225); AADLRGSSWYFDGMDY (SEQ ID NO: 226); and AAHEAQYSSRWSGTEKGYDY (SEQ ID NO: 229); or an amino acid sequence having up to two amino acid substitutions (e.g., conservative amino acid substitutions) relative to SEQ ID NO: 105, SEQ ID NO: 108, SEQ ID NO: 111, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157, SEQ ID NO: 160, SEQ ID NO: 162, SEQ ID NO: 165, SEQ ID NO: 168, SEQ ID NO: 171, SEQ ID NO: 173, SEQ ID NO: 176, SEQ ID NO: 179, SEQ ID NO: 180, SEQ ID NO: 183, SEQ ID NO: 186, SEQ ID NO: 189, SEQ ID NO: 192, SEQ ID NO: 195, SEQ ID NO: 198, SEQ ID NO: 199, SEQ ID NO: 202, SEQ ID NO: 205, SEQ ID NO: 208, SEQ ID NO: 211, SEQ ID NO: 214, SEQ ID NO: 216, SEQ ID NO: 218, SEQ ID NO: 219, SEQ ID NO: 220, SEQ ID NO: 223, SEQ ID NO: 225, SEQ ID NO: 226, or SEQ ID NO: 229.

Example of CDRs for anti-GPRc5D antibodies, antigen-binding fragments, or binding proteins include:
(a) a CDR1 having the amino acid sequence selected from the group consisting of GRTVSSYA (SEQ ID NO: 265); GRTASAYV (SEQ ID NO: 268); GIIFSASN (SEQ ID NO: 271); GGFGMMYS (SEQ ID NO: 274); RIRFSINV (SEQ ID NO: 277); and SERTFRSYT (SEQ ID NO: 280); or an amino acid sequence having up to two amino acid substitutions (e.g., conservative amino acid substitutions) relative to SEQ ID NO: 268, SEQ ID NO: 271, SEQ ID NO: 274, SEQ ID NO: 277, or SEQ ID NO: 280; and
(b) a CDR2 having the amino acid sequence selected from the group consisting of: ISWSGRST (SEQ ID NO: 266); ISGGA (SEQ ID NO: 269); VTGGGSI (SEQ ID NO: 272); RTIDGST (SEQ ID NO: 275); IAAGGTT (SEQ ID NO: 278); and ISWSHSST (SEQ ID NO: 281); or an amino acid sequence having up to two amino acid substitutions (e.g., conservative amino acid substitutions) relative to SEQ ID NO: 266, SEQ ID NO: 269, SEQ ID NO: 272, SEQ ID NO: 275, SEQ ID NO: 278, or SEQ ID NO: 281; and
(c) a CDR3 having the amino acid sequence selected from the group consisting of: ATSRAVIPGRDWNYYEY (SEQ ID NO:267); AAERGMRRLTESYQYDY (SEQ ID NO: 270); NARRSYSH (SEQ ID NO: 273); NAKPLNGRLTQY (SEQ ID NO: 276); NAVLSTLVLPSTY (SEQ ID NO: 279); and AADLRLLPEEYDY (SEQ ID NO: 282); or an amino acid sequence having up to two amino acid substitutions (e.g., conservative amino acid substitutions) relative to SEQ ID NO: 267, SEQ ID NO: 270, SEQ ID NO: 273, SEQ ID NO: 276, SEQ ID NO: 279, or SEQ ID NO: 282.

Examples of CDRs for anti-FcRH5 antibodies, antigen-binding fragments, or binding proteins include:
(a) a CDR1 having the amino acid sequence selected from the group consisting of GITVSRND (SEQ ID NO: 326); VHIISHYS (SEQ ID NO: 329); GHTLSTYA (SEQ ID NO: 332); GRTFSTYA (SEQ ID NO: 335); and GSHFSIAT (SEQ ID NO: 338); or an amino acid sequence having up to two amino acid substitutions (e.g., conservative amino acid substitutions) relative to SEQ ID NO: 326, SEQ ID NO: 329, SEQ ID NO: 332, SEQ ID NO: 335, or SEQ ID NO: 338; and
(b) a CDR2 having the amino acid sequence selected from the group consisting of: IMNRVGST (SEQ ID NO: 327); IPVSGRVP (SEQ ID NO: 330); IARDGGAT (SEQ ID NO: 333); IDTTGSAS (SEQ ID NO: 336); and LSSSGRP (SEQ ID NO: 339); or an amino acid sequence having up to two amino acid substitutions (e.g., conservative amino acid substitutions) relative to SEQ ID NO: 327, SEQ ID NO: 330, SEQ ID NO: 333, SEQ ID NO: 336, or SEQ ID NO: 339; and
(c) a CDR3 having the amino acid sequence selected from the group consisting of: NALNTVITWP (SEQ ID NO: 328); AAYPRKGLEGNEYEY (SEQ ID NO: 331); AASSMFSTAKRDYSY (SEQ ID NO: 334); AAARRYSTAPGDYDY (SEQ ID NO: 337); and WGQGTQVIVSS (SEQ ID NO: 478); or an amino acid sequence having up to two amino acid substitutions (e.g., conservative amino acid substitutions) relative to SEQ ID NO: 328, SEQ ID NO: 331, SEQ ID NO: 334, SEQ ID NO: 337, or SEQ ID NO: 478.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH1, shown

(CD38-VHH1, SEQ ID NO: 1)
QVQLVESGGGLVQPGDSLRLSCAASGFILDTYSVAWFRQAPGKEREGVSC
ISSRDGNTFYSDSVKGRFTISRDNAKNTVYLQMNSLKSEDTAVYTCAAGA
QAHCTIFTSYFNSDYYRRYNYWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 1. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 1. In some embodiments, the antibody or binding protein contains a VHH domain having the amino acid sequence of SEQ ID NO: 1.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH2, shown below (CDR sequences shown in bold):

(CD38-VHH2, SEQ ID NO: 2)
QVQLVESGGGLVQPGGSLRLSCAASGFIFSDKVMSWVRQAPGKGREWVST
ITPGGTATSYTESVKGRFTISRDNAKNTLYLQMNNLKPDDTALYYCRIGG
PGGRYDNWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 2. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 2. In some embodiments, the antibody or NO: 2.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH3, shown below (CDR sequences shown in bold):

(CD38-VHH3, SEQ ID NO: 3)
QVQLVESGGGLVQAGGSLTLSCAASRSIFEINTMTMGWYRQAPGKQRE
LIAASRGATTNYADSVKGRFTISRDNAKNTVYLQMNSLKPEDTAVYYC
SADRYGFGYGDNDYWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 3. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 3. In some embodiments, the antibody or binding protein contains a VHH domain having the amino acid sequence of SEQ ID NO: 3.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH4, shown below (CDR sequences shown in bold):

(CD38-VHH4, SEQ ID NO: 4)
QVQLVESGGGLVQPGGSLRLSCAASGFSLDYYHIGWFRQAPGKEREGI
SCISSSDGYTLYADSVKGRFTISRDNAKNTVYLQMNSLKPEDTAVYYC
AASPRRLACAGSLYPPLSADFSSWGQGTQVTVSS.

In some embodiments, the anti-CD38 antibody or binding protein contains a the amino acid sequence of SEQ ID NO: 4. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 4. In some embodiments, the antibody or binding protein contains a VHH domain having the amino acid sequence of SEQ ID NO: 4.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH5, shown below (CDR sequences shown in bold):

(CD38-VHH5, SEQ ID NO: 5)
QVQLVESGGGLVQTGGSLRLSCVASGAIVSAESMGWYRQAPGNQRELV
AEIISGSKSNYGESVKGRFTISRDNAENTVYLQMNSVKPEDTAVYYCK
RTERIWTNNPQVYWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 5. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 5. In some embodiments, the antibody or binding protein contains a VHH domain having the amino acid sequence of SEQ ID NO: 5.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH6, shown

(CD38-VHH6, SEQ ID NO: 6)
QVQLVESGGGLVQAGGSLRLSCAASGTFSSINLMGYYRQAPGKQREFV
ARDYTEGTTDYADSVKGRFTISRDNTKKTVFLQMNNLKPEDTAIYYCW
LMVRAGDVYWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 6. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 6. In some embodiments, the antibody or binding protein contains a VHH domain having the amino acid sequence of SEQ ID NO: 6.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH7, shown below (CDRs are underlined):

(CD38-VHH7, SEQ ID NO: 7)
QVQLVESGGGLVHTGGSLRLSCAASGSISGLNTMGWYRQAPGKQRELV
ATIISGTMTRYADSVKGRFTISKDNAKNTVYLQMNSLKPEDTAVYYCT
FKEITRDSRSYWGQGTQVTVSS .

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 7. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 7. In some embodiments, the antibody or NO: 7.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH8, shown below (CDR sequences shown in bold):

(CD38-VHH8, SEQ ID NO: 8)
QVQLVESGGGLVQAGGSLRLSCAASGSSVSMNSMAWYRQAPGKQRELV
ALITPGDRINYADFVKGRFTISRDNAKNTVNLQMNNLKPEDTAVYYCN
IGATRPPFGAWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 8. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 8. In some embodiments, the antibody or binding protein contains a VHH domain having the amino acid sequence of SEQ ID NO: 8.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH9, shown below (CDR sequences shown in bold):

(CD38-VHH9, SEQ ID NO: 9)
QVQLVESGGGLVQAGGSLRLSCVASGFIYSISTMGWYRQAPGKQRELV
ATITSGGNTNYADSVKGRFTISRDNAQNTVYLQMNNLKPEDTAIYYCN
TAWRETIVSRVWGQGTQVTVSS.

In some embodiments, the anti-CD38 antibody or binding protein contains a the amino acid sequence of SEQ ID NO: 9. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 9. In some embodiments, the antibody or binding protein contains a VHH domain having the amino acid sequence of SEQ ID NO: 9.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH10, shown below (CDR sequences shown in bold):

(CD38-VHH10, SEQ ID NO: 10)
QVQLVESGGGLVQAGGSLKLACVVSGRYFRINAMGWYRQAPGKQRDMV
ASISNDGSTNYGDVVKGRFTISRDNNKNTVYLQMNSLKPEDTAVYSCN
VKALPFLSSNELSYWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 10. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 10. In some embodiments, the antibody or binding protein contains a VHH domain having the amino acid sequence of SEQ ID NO: 10.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH11, shown

(CD38-VHH11, SEQ ID NO: 11)
QVQLVESGGGLVQAGGSLKLSCAASGTFSSIALMGYYRQGPGKQREFV
ARVSIGGVTEYADSVKGRFTISGDNAKKTVDLQMNNLKPDDTGVYYCW
RMEGAGDVYWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 11. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 11. In some embodiments, the antibody or binding protein contains a VHH domain having the amino acid sequence of SEQ ID NO: 11.

In some embodiments, the anti-CD38 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH12, shown below (CDR sequences shown in bold):

(CD38-VHH12, SEQ ID NO: 12)
QVQLVESGGGLVQAGGSLRLSCVASGIIFRIFSMGWYRQAPGKQRELV
ATITSGGNTNYAHSVKGRFTISRDDAKNTVNLQMNSLKPEDTAVYYCN
VAIPSGIVDRSAWGQGTQVTVSS.

In some embodiments, the anti-CD38 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 12. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 12. In some embodiments, the antibody or NO: 12.

In some embodiments, the anti-CD38 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH1-H9, shown below (CDR sequences shown in bold):

(CD38-VHH1-H9, SEQ ID NO: 49)
EVQLVESGGGLVQPGGSLRLSCAASGFILDTYSVAWFRQAPGKEREGV
SCISSRDGNTFYSDSVKGRFTISRDNAKNTVYLQMNSLRAEDTAVYTC
AAGAQAHCTIFTSYFNSDYYRRYNYWGQGTLVTVSS.

In some embodiments, the anti-CD38 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 49. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 49. In some embodiments, the antibody or binding protein contains a humanized VHH domain having the amino acid sequence of SEQ ID NO: 49.

In some embodiments, the anti-CD38 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH2-H10, shown below (CDR sequences shown in bold):

(CD38-VHH2-H10, SEQ ID NO: 50)
EVQLVESGGGLVQPGGSLRLSCAASGFIFSDKVMSWVRQAPGKGREWV
STITPGGTATSYTESVKGRFTISRDNSKNTLYLQMNSLKADDTAVYYC
RIGGPGGRYDNWGQGTLVTVSS.

In some embodiments, the anti-CD38 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 50. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 50. In some embodiments, the antibody or binding protein contains a humanized VHH domain having the amino acid sequence of SEQ ID NO: 50.

In some embodiments, the anti-CD38 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, CD38-VHH3-H9, shown below (CDR sequences shown in bold):

(CD38-VHH3-H9, SEQ ID NO: 51)
EVQLVESGGGLVQPGGSLRLSCAASRSIFEINTMTMGWYRQAPGKQRE
LISASRGATTNYADSVKGRFTISRDNAKNTVYLQMNSLKAEDTAVYYC
SADRYGFGYGDNDYWGQGTQVTVSS.

In some embodiments, the anti-CD38 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 51. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 51. In some embodiments, the antibody or binding protein contains a humanized VHH domain having the amino acid sequence of SEQ ID NO: 51.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH1, shown

(BCMA-VHH1, SEQ ID NO: 52)
AVQLVDSGGGLVTAGDSLTLSCVASGRTLSPYTAGWFRQAPGREREFV
ALITARGDWTSYSDSVKGRFTISRDNAKNTIYLQMNSLEPEDTALYYC
VRDLLGRDDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 52. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 52. In some embodiments, the antibody or binding protein contains a VHH domain having the amino acid sequence of SEQ ID NO: 52.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH2, shown below (CDR sequences shown in bold):

(BCMA-VHH2, SEQ ID NO: 53)
HVQLVESGGGLVQTGGSLRLSCAASGRTLNNYVVAWFRQAPGKEREFV
ATMWWSGGSPWYSDNVKGRFTISRDNAKNTLYLQMNSLRPEDTAVYYC
AATWVGTSEYRHWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 53. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 53. In some embodiments, the antibody or NO: 53.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3, shown below (CDR sequences shown in bold):

(BCMA-VHH3, SEQ ID NO: 54)
QVQLVESGGGLVQAGGSLRLSCAASGSIFAYHVMGWYRQAPGKQRELV
ALITSGGSTNYTDSVKGRFTISRDNAKNTVYLQMNSLKPEDTAVYYCA
IGYYSGSYYWERSGDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 54. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 54. In some embodiments, the antibody or binding protein contains a VHH domain having the amino acid sequence of SEQ ID NO: 54.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH4, shown below (CDR sequences shown in bold):

(BCMA-VHH4, SEQ ID NO: 55)
QVKLEESGGGLVQTGGSLRLSCAASGRTLNNYVVAWFRQAPGKEREFV
ATMWWSGGSPWYSDNVKGRFTISRDNAKNTLYLQMNSLRPEDTAVYYC
AATWVGTSEYQHWGQGTQVTVSS.

In some embodiments, the anti-BCMA antibody or binding protein contains a the amino acid sequence of SEQ ID NO: 55. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 55. In some embodiments, the antibody or binding protein contains a VHH domain having the amino acid sequence of SEQ ID NO: 55.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH5, shown below (CDR sequences shown in bold):

(BCMA-VHH5, SEQ ID NO: 56)
QVKLEESGGGLVQAGGSLRLSCAASGRTFSDYTVAWFRQAPGSEREFV
AASVWTDGKPYYVDSVRGRFTISRDNAKNTVWLQMDSLQPDDTAVYYC
QALTGGSWILDYWGQGTPVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 56. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 56. In some embodiments, the antibody or binding protein contains a VHH domain having the amino acid sequence of SEQ ID NO: 56.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH6, shown

(BCMA-VHH6, SEQ ID NO: 57)
QVKLEESGGGLVQAGDSLTLSCAASGRPLRMYNMGWFRQAPGKEREFV
AFISWSGKTTYYSDAVKGRFVISRDNAKRTVYLQMDSLKPDDTGDYYC
FMNVWADTSDSSADAYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 57. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 57. In some embodiments, the antibody or binding protein contains a VHH domain having the amino acid sequence of SEQ ID NO: 57.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH7, shown below (CDR sequences shown in bold):

(BCMA-VHH7, SEQ ID NO: 58)
EVQLVDSGGGLVQAGGSLRLSCAASGSTFSRYAMGWIRQAPGKEREFV
AAISWSGGSTYYADSVKGRFTISRDVAKNTVSLQMNSLKPEDAGVYFC
FGDIIRSGERSDYEYWGQGTRVTVTS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 58. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 58. In some embodiments, the antibody or NO: 58.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH8, shown below (CDR sequences shown in bold):

(BCMA-VHH8, SEQ ID NO: 59)
QVQLVQSGGGLVLPGGSLRLSCAASGFTLSSYWMHWLRQAPGKGLEWV
SAIKPESGITYYAESMKGRFTISRDNAKNTLYLQMNSLKSEDSALYYC
VREDYDSAYVGDYWGKGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 59. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 59. In some embodiments, the antibody or binding protein contains a VHH domain having the amino acid sequence of SEQ ID NO: 59.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH9, shown below (CDR sequences shown in bold):

(BCMA-VHH9, SEQ ID NO: 60)
QVKLEESGGGFVQPGGSLRLSCAASGFTYSSYWMHWVRQAPGKGLEWV
SGISTGGDTTDTYYADSVKGRFTISRDNAKNTLYLQMNSLKPEDTAVY
YCVALNLWGTDLEHDYWGRGTQVTVSS.

In some embodiments, the anti-BCMA antibody or binding protein contains a the amino acid sequence of SEQ ID NO: 60. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 60. In some embodiments, the antibody or binding protein contains a VHH domain having the amino acid sequence of SEQ ID NO: 60.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH10, shown below (CDR sequences shown in bold):

(BCMA-VHH10, SEQ ID NO: 61)
HVQLVDSGGGLVRSGGSLRLSCVMSGGTLEYYAMAWFRQAPGKERETV
AAITWSGGSTYYVDSVKGRFTISRDNAKNTLYLQMNSLKPEDTAVYYC
AAQFVEVEILVRSYEYWGQGTQVTVST.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 61. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 61. In some embodiments, the antibody or binding protein contains a VHH domain having the amino acid sequence of SEQ ID NO: 61.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH11, shown

(BCMA-VHH11, SEQ ID NO: 62)
QVQLVDSGGGLVQPGGSLRLSCAASGFTFSSYWMYWVRQAPGKGLEWV
SAINTDGDSTYYADSVKGRFTVSRDVAGNMIYLQMTNLKPEDTAVYTC
AAIVTRSDGHQYDYWGRGTQVTVSV.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 62. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 62. In some embodiments, the antibody or binding protein contains a VHH domain having the amino acid sequence of SEQ ID NO: 62.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH12, shown below (CDR sequences shown in bold):

(BCMA-VHH12, SEQ ID NO: 63)
QVQLVESGGGSVQAGGSLRLSCAASGRIDSGYTMAWFRQAPGSEREFV
AAVVGSDGRDYYIDSVRGRFTVSRDSAKNTVYLEMTGLKPEDTAVYYC
AATNYYSDYLDHLSRGYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 63. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 63. In some embodiments, the antibody or NO: 63.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH13, shown below (CDR sequences shown in bold):

(BCMA-VHH13, SEQ ID NO: 64)
QVQLVDSGGRLVQAGDSLRLSCSASGFTFGSYWMFWVRQAPGKGLEWV
SAIDTSGGHVYYGDSVNGRFTISRDNAKNTLYLQMNSLKPEDTALYYC
ARVENTWESIYWGQGTTVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 64. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 64. In some embodiments, the antibody or binding protein contains a VHH domain having the amino acid sequence of SEQ ID NO: 64.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH14, shown below (CDR sequences shown in bold):

(BCMA-VHH14, SEQ ID NO: 65)
QVQLVESGGGLAQSGGSLELSCAASGHTLNSYAMGWFRQAPGKEREFV
AAISRSGEKTYYADSVKGRFTISGDNAKNTAALQMGRLKPEDTAVYYC
GAWNFVKNDAYWGQGTQVTVSA.

In some embodiments, the anti-BCMA antibody or binding protein contains a the amino acid sequence of SEQ ID NO: 65. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 65. In some embodiments, the antibody or binding protein contains a VHH domain having the amino acid sequence of SEQ ID NO: 65.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH15, shown below (CDR sequences shown in bold):

(BCMA-VHH15, SEQ ID NO: 66)
QVQLVESGGGLVQPGGSLRLSCAASGFTFSTYSMYWVRQAPGKGLEWV
SAIDARGVNTYYADSVRGRFTVSRDNAKNTLYLQMNNLKLEDTALYYC
GGWEFATWGRGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 66. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 66. In some embodiments, the antibody or binding protein contains a VHH domain having the amino acid sequence of SEQ ID NO: 66.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH16, shown

(BCMA-VHH16, SEQ ID NO: 67)
QVKLEESGGGLVQAGGSLRLSCAASGHTFSNSAMGWIRQAPGKEREFV
AAISLNGGNTHYAESVKGRFAISRDNAKNTMYLQMNSLKPEDTAVYYC
ATLGFASWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 67. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 67. In some embodiments, the antibody or binding protein contains a VHH domain having the amino acid sequence of SEQ ID NO: 67.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH17, shown below (CDR sequences shown in bold):

(BCMA-VHH17, SEQ ID NO: 68)
QVQLVESGGGLVQAGGSLRLSCAASGRTFSGYTMAWFRQAPGSEREFV
AAAVGSDGSTYYVDSVKGRFTMSRDSAKNTVYLMMTSLQPVDSAVYFC
VKLDSGAWSLAEWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 68. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 68. In some embodiments, the antibody or NO: 68.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH18, shown below (CDR sequences shown in bold):

(BCMA-VHH18, SEQ ID NO: 69)
PVQLVESGGGLVQAGGSLRLSCAASGRTFSSYAMGWFRQAPGKERE
FVAGVSWTGDNTGYADSVKGRFTISRDNFKSTVYLEMNSLKPEDTA
VYFCAAWNWGHHEYTYWGRGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 69. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 69. In some embodiments, the antibody or binding protein contains a VHH domain having the amino acid sequence of SEQ ID NO: 69.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH19, shown below (CDR sequences shown in bold):

(BCMA-VHH19, SEQ ID NO: 70)
HVQLVESGGGLVQPGGSLRLSCSVSGRTIGSFVMGWFRQAPGKERE
FVAAVNWRGSSTYYADSVRGRFTISRGNAATTMSLQMNSLKPEDAA
LYYCARWSWDIGADFGSWGQGTQVTVSS.

In some embodiments, the anti-BCMA antibody or binding protein contains a the amino acid sequence of SEQ ID NO: 70. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 70. In some embodiments, the antibody or binding protein contains a VHH domain having the amino acid sequence of SEQ ID NO: 70.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH20, shown below (CDR sequences shown in bold):

(BCMA-VHH20, SEQ ID NO: 71)
QVQLVESGGGLVQPGGSLRLSCAASGFTFSNYAMIWVRQAPGKGLE
WVSSINANSDTTAYSDSVKGRFAISRDNARNTLYLQMNSLKPEDTA
VYFCVSDSYIGGLYATYVYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 71. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 71. In some embodiments, the antibody or binding protein contains a VHH domain having the amino acid sequence of SEQ ID NO: 71.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH21, shown

(BCMA-VHH21, SEQ ID NO: 72)
QVKLEESGGGLVQAGGSLSLSCKASVRYFSTYAMGWFRQAPGKERE
FVAAISWSGGSTYYADSVKGRFTISRDNAKNTVYLEMNSLKPEDTA
VYYCAAPYGSSQNLEYDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 72. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 72. In some embodiments, the antibody or binding protein contains a VHH domain having the amino acid sequence of SEQ ID NO: 72.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH22, shown below (CDR sequences shown in bold):

(BCMA-VHH22, SEQ ID NO: 73)
HVQLVDSGGGLVQAGGSLRLSCAASGFTFDDYAMSWVRQAPGKGLE
WVSAISWNGGSTYYAESMKGRFTISRDNTKNTLYLQMNSLKPEDSA
VYYCVPVSHSDSVCGSPYMDYWGKGTLVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 73. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 73. In some embodiments, the antibody or NO: 73.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH23, shown below (CDR sequences shown in bold):

(BCMA-VHH23, SEQ ID NO: 74)
QVKLEESGGGLVHPGGSLTLSCVASGFNLTSDAVGWFSQAAPGKDL
EPISCISRTESDTVQGRFAISRNSAKNTVYLQMNSLQPGDTAVYRC
AADNAADRCSLSIYNYNLWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 74. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 74. In some embodiments, the antibody or binding protein contains a VHH domain having the amino acid sequence of SEQ ID NO: 74.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH24, shown below (CDR sequences shown in bold):

(BCMA-VHH24, SEQ ID NO: 75)
QVKLEESGGGLVQAGGSLRLSCQVSGRTFASFAMAWFRQAPGKERE
FVAGISWSGGFTSYADSVKGRFTISRDNAKHTVYLQMNSLKTEDTA
MYYCGEWGGFSLLGQGTQVTVSS.

In some embodiments, the anti-BCMA antibody or binding protein contains a the amino acid sequence of SEQ ID NO: 75. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 75. In some embodiments, the antibody or binding protein contains a VHH domain having the amino acid sequence of SEQ ID NO: 75.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH25, shown below (CDR sequences shown in bold):

(BCMA-VHH25, SEQ ID NO: 76)
QVQLVESGGGLVQAGGSLRLSCAASGFTFDDYAIGWFRQAPGKERE
GVSCIASSDGSTFYADSVKGRFTISSDNANNTVYLQMNNLNREDTA
VYYCVAPCFWFDTVIAGTDPRYDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 76. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 76. In some embodiments, the antibody or binding protein contains a VHH domain having the amino acid sequence of SEQ ID NO: 76.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH26, shown

(BCMA-VHH26, SEQ ID NO: 77)
QVQLVESGGGLVQAGGSLRLSCAASGFTLGYYAIGWFRQAPGKERE
GISCISGRDGSTYYAGSVKGRFTISSDNAKNTVYLQMNSLKPVDTA
VYYCAAVRGPIVSMDPDLCRPVEFDYRGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 77. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 77. In some embodiments, the antibody or binding protein contains a VHH domain having the amino acid sequence of SEQ ID NO: 77.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH27, shown below (CDR sequences shown in bold):

(BCMA-VHH27, SEQ ID NO: 78)
QVQLVESGGGLVQAGGSLRLSCATSGFTFEDYAIAWFRQAPGKERE
AVSCTSKNDRMPYYAPSVKDRFTISTDNGKNTVYLQMNSLKPEDTA
VYYCAATNGPAITLFPCHINYWLYDNWGPGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 78. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 78. In some embodiments, the antibody or NO: 78.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH28, shown below (CDR sequences shown in bold):

(BCMA-VHH28, SEQ ID NO: 79)
QVQLVESGGGLVQAGGSLRLSCATSGFTFEDYAIAWFRQAPGKERE
AVSCTSKNDRMPYYAPSVKGRFTISTDNGKNTAYLQMNSLKPEDTA
VYYCASADWRSPTPFPCGVSRSLYDHWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 79. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 79. In some embodiments, the antibody or binding protein contains a VHH domain having the amino acid sequence of SEQ ID NO: 79.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH29, shown below (CDR sequences shown in bold):

(BCMA-VHH29, SEQ ID NO: 80)
QVQLVESGGGLVQAGGSLRLSCAASGNFLRFNAMGWFRQAPGKQRE
LVAMISSGGRTNYVDSVKGRFTVSRDNAKNTVTLQMNSLKPEDTAV
YYCWSAPDYWGQGTQVTVSS.

In some embodiments, the anti-BCMA antibody or binding protein contains a the amino acid sequence of SEQ ID NO: 80. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 80. In some embodiments, the antibody or binding protein contains a VHH domain having the amino acid sequence of SEQ ID NO: 80.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH30, shown below (CDR sequences shown in bold):

(BCMA-VHH30, SEQ ID NO: 81)
QVQLVESGGGLVQAGGSLRLSCVVSGSFSSIDTVDWYRQAPGKQRE
LVATINRGGDTDYKDSVKGRFTISKDNAKNSVHLQMNNLKPEDTAV
YTCAGSFTLATGDDFGSWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 81. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 81. In some embodiments, the antibody or binding protein contains a VHH domain having the amino acid sequence of SEQ ID NO: 81.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH31, shown

(BCMA-VHH31, SEQ ID NO: 82)
QVQLVESGGGLVQAGGSLRLTCTASEQNFSADYMAWYRQAPGKERE
LIATISNSGRTHYVDSMAGRFTISRDNAKNTVYLQLDSLKPGDTAV
YYCVAEPFSFRRRAWGRGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 82. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 82. In some embodiments, the antibody or binding protein contains a VHH domain having the amino acid sequence of SEQ ID NO: 82.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH32, shown below (CDR sequences shown in bold):

(BCMA-VHH32, SEQ ID NO: 83)
QVQLVESGGGLVQAGGSLRLTCTASEQNFSTDDMAWYRQAPGKERG
LIATITNSGTTHYVDSMAGRFTISRDNAENTVYLQMNSLIPEDTAV
YYCGESTTGWAECDFGCWGRRTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 83. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 83. In some embodiments, the antibody or NO: 83.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH33, shown below (CDR sequences shown in bold):

(BCMA-VHH33, SEQ ID NO: 84)
QVQLVESGGGLVQPGGSLRLSCAASRSIFSINAMGWYRQAPGKQRE
LVATITNGGTTNYADSVKGRFTISRDNAENTVYLQMNSLKPEDTAV
YYCNANSRYGVGWYNYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 84. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 84. In some embodiments, the antibody or binding protein contains a VHH domain having the amino acid sequence of SEQ ID NO: 84.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH34, shown below (CDR sequences shown in bold):

(BCMA-VHH34, SEQ ID NO: 85)
QVQLVESGGGLVQPGGSLRLSCAASGSIFSINPMGWYRQAPGKQRE
LVAAFTSGGTTNYADSVKGRFTISRDNAKNTVYLEMNSLKPEDTAV
YYCNVRGGHCDPRYWREYWGQGTQVTVSS.

In some embodiments, the anti-BCMA antibody or binding protein contains a the amino acid sequence of SEQ ID NO: 85. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 85. In some embodiments, the antibody or binding protein contains a VHH domain having the amino acid sequence of SEQ ID NO: 85.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH35, shown below (CDR sequences shown in bold):

(BCMA-VHH35, SEQ ID NO: 86)
QVQLVESGGGLVQPGGSLRLSCAASGSIFSINPMGWYRQAPGKQRE
LVAAFTSGGTTNYADSVKGRFTISRDNAKNTVYLQMNSLKPEDTAA
YYCNVRGGHYDPRYWREYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 86. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 86. In some embodiments, the antibody or binding protein contains a VHH domain having the amino acid sequence of SEQ ID NO: 86.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH36, shown

(BCMA-VHH36, SEQ ID NO: 87)
QVQLVESGGGLVQAGGSLRLSCAASGRPIDTYAMGWFRQAPGKERE
FVAVVSWAGVYTYYADSVKGRFTISRDNAKNTVVLQVNRLKPEDTA
VYYCAATKLPWNTIVMVQRSYCDYWSLGTHVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 87. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 87. In some embodiments, the antibody or binding protein contains a VHH domain having the amino acid sequence of SEQ ID NO: 87.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH37, shown below (CDR sequences shown in bold):

(BCMA-VHH37, SEQ ID NO: 88)
QVQLVESGGGLVQAGGSRRLSCAASGRTISTYAMGWFRQAPGKERE
FVAVVSWAGSYTYYADSVKGRFTISRDNAKNTVVLQVNSLKPEDTA
VYYCAATKLPWNTSVMVKRSVYDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 88. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 88. In some embodiments, the antibody or NO: 88.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH38, shown below (CDR sequences shown in bold):

(BCMA-VHH38, SEQ ID NO: 89)
QVQLVESGGGLVQAGDSLRLSCATSGRPLECYAMGWFRQAPGKERE
FVAGISGNGVNTYYADSLKGRFTISRDNAKNTMNLQMNRLKPEDTA
VYFCAATTVPVINLEISHMTYCGKGTLGTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 89. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 89. In some embodiments, the antibody or binding protein contains a VHH domain having the amino acid sequence of SEQ ID NO: 89.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH39, shown below (CDR sequences shown in bold):

(BCMA-VHH39, SEQ ID NO: 90)
QVQLVESGGGLVQAGDSLSLSCTSSGRRLEGYTMGWFRQPPGKERE
FVAAISRNRVNTYYADSLKGRFTISRDNAKNTINLQMDRLKPEDTA
VYYCAATNLPGITLLMSHMNYCDYWTLGTQVTVSS.

In some embodiments, the anti-BCMA antibody or binding protein contains a the amino acid sequence of SEQ ID NO: 90. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 90. In some embodiments, the antibody or binding protein contains a VHH domain having the amino acid sequence of SEQ ID NO: 90.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH40, shown below (CDR sequences shown in bold):

(BCMA-VHH40, SEQ ID NO: 91)
QVQLVESGGGLVQVGDSLNLSCTTSGPRLEGYTMGWFRQPPGKERE
FVAVITRNRVNTYYANSLKGQFTFSKNNAKNTIILQMDRLKPEDTA
VYFCAATTVPVINLQVSHINYWSKGTLVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 91. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 91. In some embodiments, the antibody or binding protein contains a VHH domain having the amino acid sequence of SEQ ID NO: 91.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH41, shown

(BCMA-VHH41, SEQ ID NO: 92)
QVQLVESGGGLVQAGDSLSLSCTSSGRRLEGYTMGWFRQPPGKERE
FVAAISRNRVNTYYADSLKGRFTISRDNAKNTINLQMDRLKPEDTA
VYFCAATTVPVINLQVSHINYWGKGTLVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 92. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 92. In some embodiments, the antibody or binding protein contains a VHH domain having the amino acid sequence of SEQ ID NO: 92.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH42, shown below (CDR sequences shown in bold):

(BCMA-VHH42, SEQ ID NO: 93)
QVQLVESGGGLVQAGDSLSLSCTSSGRRLEGYTMGWFRQPPGKERE
FVAAISRNRVNTYYADSLKGRFTIPRDNAKNTINLQMDRLKPEDTA
VYFCAATTVPVINLQVSHINYWGKGTLVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 93. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 93. In some embodiments, the antibody or NO: 93.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH43, shown below (CDR sequences shown in bold):

(BCMA-VHH43, SEQ ID NO: 94)
QVQLVESGGGLVQVGGSLRLSCTASGRTFNLYAMGWFRQAPGEERE
FVGVITWSGGSTGYADSVKGRFAISRDNAKNTVYLQMNNLKPEDTA
LYYCAAKVFPMATLDDDVYDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 94. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 94. In some embodiments, the antibody or binding protein contains a VHH domain having the amino acid sequence of SEQ ID NO: 94.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH44, shown below (CDR sequences shown in bold):

(BCMA-VHH44, SEQ ID NO: 95)
QVQLVESGGGLVQAGATLRLSCAASGVTFSNYVMGWFRQAPGKERE
FVATISWSGGSTYYADSVKGRFTISRDNAKNAVYLQMNSLKPEDTA
VYYCAAESHRRNTIVIVTTPDEYDYWGQGTQVTVSS.

In some embodiments, the anti-BCMA antibody or binding protein contains a the amino acid sequence of SEQ ID NO: 95. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 95. In some embodiments, the antibody or binding protein contains a VHH domain having the amino acid sequence of SEQ ID NO: 95.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH45, shown below (CDR sequences shown in bold):

(BCMA-VHH45, SEQ ID NO: 96)
QVQLVESGGGLVQAGGSLRLSCAASGRTFSSYAMGWFRQAPGKERE
FVAAISWSGGSTYYADSVKGRFTISRDNAKNTVYLQMNSLKPEDTA
VYYCAADPSSGYNLFARTVVAFARYDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 96. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 96. In some embodiments, the antibody or binding protein contains a VHH domain having the amino acid sequence of SEQ ID NO: 96.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH46, shown

(BCMA-VHH46, SEQ ID NO: 97)
QVQLVESGGGLVQAGGSLRLSCAASGRTFSSYAMGWFRQAPGKERE
FVAAISWSGGSTYYADSVKGRFTIPRDSAKNAVYLQMNSLKPEDTA
TYCCAAHPAGAHGGLIYKNWGRGTHVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 97. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 97. In some embodiments, the antibody or binding protein contains a VHH domain having the amino acid sequence of SEQ ID NO: 97.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH47, shown below (CDR sequences shown in bold):

(BCMA-VHH47, SEQ ID NO: 98)
QVQLVESGGGLVQAGGSLRLSCVAFGRTVSNYAVAWFRQAPGKERR
FVAAINRSGTPYYGDSVKDRFTISRDSAKNAVYLQMNSLKPEDTAT
YSCAADPTGAHVGAIYKNWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 98. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 98. In some embodiments, the antibody or NO: 98.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH48, shown below (CDR sequences shown in bold):

(BCMA-VHH48, SEQ ID NO: 99)
QVQLVESGGGLVQAGGSLRLSCVAFGRTFSNYAVAWFRQAPGKERRFV
AAINRSGTPYYGDSVKGRFTISRDNAENTVYLQMNSLKPEDTAVYYCA
ADLRGSSWYFDGVDYWGKGTLVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 99. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 99. In some embodiments, the antibody or binding protein contains a VHH domain having the amino acid sequence of SEQ ID NO: 99.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH49, shown below (CDR sequences shown in bold):

(BCMA-VHH49, SEQ ID NO: 100)
QVQLVESGGGLVQAGGSLRLSCAASGRTFSSYAMGWFRQAPGKEREFV
AAISWSGGSTYYADSVKGRFTISRDNAKNTVYLQMNSLKPEDTAVYYC
AADLRGSSWYFDGMDYWGKGTLVTVSS.

In some embodiments, the anti-BCMA antibody or binding protein contains a the amino acid sequence of SEQ ID NO: 100. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 100. In some embodiments, the antibody or binding protein contains a VHH domain having the amino acid sequence of SEQ ID NO: 100.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH50, shown below (CDR sequences shown in bold):

(BCMA-VHH50, SEQ ID NO: 101)
QVQLVESGGGLVQSGGSLRLSCAASGRTFSRYAMGWFRQAPGKEREFV
AAITRSGTSTYYADSVKGRFTISRDNAKNTMYLQMNSLKPEDTAVYYC
AAHEAQYSSRWSGTEKGYDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 101. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 101. In some embodiments, the antibody or binding protein contains a VHH domain having the amino acid sequence of SEQ ID NO: 101.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH51, shown

(BCMA-VHH51, SEQ ID NO: 102)
QVQLVESGGGLVQAGGSLRLSCAASGRTFSSYAMGWFRQAPGKEREFV
AAISWSGGSTYYADSVKGRFTTSRDNAKSTVYLQMNSLKPEDTAVYYC
AADPSSGYNLFARTVVAFARYDYWGQGTQVTVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 102. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 102. In some embodiments, the antibody or binding protein contains a VHH domain having the amino acid sequence of SEQ ID NO: 102.

In some embodiments, the anti-BCMA antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH52, shown below (CDR sequences shown in bold):

(BCMA-VHH52, SEQ ID NO: 473)
QVQLVESGGGLVQAGGSLRLSCATSGRPLESYAMGWFRQAPGKEREFV
AAVSWNRGYTYYADSLKGRFTISRDNAKNTMVLQMNRLKPEDTAVYYC
AATDFPWNTPVMGKRSLYEYWRWGTRTNVSS.

In some embodiments, the anti-BCMA antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 473. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 473. In some embodiments, the antibody or binding protein contains a VHH domain having the amino acid sequence of SEQ ID NO: 473.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H1, shown below (CDR sequences shown in bold):

(BCMA-VHH3-H1, SEQ ID NO: 230)
QVQLVESGGGLVQPGGSLRLSCAASGSIFAYHVMGWYRQAPGKQRELV
ALITSGGSTNYTDSVKGRFTISRDNSKNTVYLQMNSLRAEDTAVYYCA
IGYYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 230. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 230. In some embodiments, the antibody or binding protein contains a humanized VHH domain having the amino acid sequence of SEQ ID NO: 230.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H2, shown below (CDR sequences shown in bold):

(BCMA-VHH3-H2, SEQ ID NO: 231)
EVQLVESGGGLVQPGGSLRLSCAASGSIFAYHVMGWYRQAPGKQLELV
SLITSGGSTNYTDSVKGRFTISRDNSKNTLYLQMNSLRAEDTAVYYCA
IGYYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 231. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 231. In some embodiments, the antibody or binding protein contains a humanized VHH domain having the amino acid sequence of SEQ ID NO: 231.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H3, shown below (CDR sequences shown in bold):

(BCMA-VHH3-H3, SEQ ID NO: 232)
QVQLVESGGGVVQPGGSLRLSCAASGSIFAYHVMGWYRQAPGKQRELV
SLITSGGSTNYTDSVKGRFTISRDNAKNTLYLQMNSLRAEDTAVYYCA
IGYYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 232. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 232. In some embodiments, the antibody or binding protein contains a humanized VHH domain having the amino acid sequence of SEQ ID NO: 232.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H4, shown below (CDR sequences shown in bold):

(BCMA-VHH3-H4, SEQ ID NO: 233)
EVQLVESGGGLVQPGGSLRLSCAASGSIFAYHVMGWYRQAPGKQRELV
ALITSGGSTNYTDSVKGRFTISRDNAKNTLYLQMNSLRAEDTAVYYCA
IGYYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 233. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 233. In some embodiments, the antibody or binding protein contains a humanized VHH domain having the amino acid sequence of SEQ ID NO: 233.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H5, shown below (CDR sequences shown in bold):

(BCMA-VHH3-H5, SEQ ID NO: 234)
QVQLVESGGGVVQPGGSLRLSCAASGSIFAYHVMGWYRQAPGKQLELV
ALITSGGSTNYTDSVKGRFTISRDNAKNTLYLQMNSLRAEDTAVYYCA
IGYYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 234. In some embodiments, the 100% identical) to the amino acid sequence of SEQ ID NO: 234. In some embodiments, the antibody or binding protein contains a humanized VHH domain having the amino acid sequence of SEQ ID NO: 234.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H6, shown below (CDR sequences shown in bold):

(BCMA-VHH3-H6, SEQ ID NO: 235)
QVQLVESGGGLVQPGGSLRLSCAASGSIFAYHVMGWYRQAPGKQRELV
ALITSGGSTNYTDSVKGRFTISRDNAKNTVYLQMNSLRAEDTAVYYCA
IGYYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 235. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 235. In some embodiments, the antibody or binding protein contains a humanized VHH domain having the amino acid sequence of SEQ ID NO: 235.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H7, shown below (CDR sequences shown in bold):

(BCMA-VH3-H7, SEQ ID NO: 236)
QVQLVESGGGVVQPGGSLRLSCAASGSIFAYHVMGWYRQAPGKQRELV
SLITSGGSTNYTDSVKGRFTISRDNAKNTVYLQMNSLRAEDTAVYYCA
IGYYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 236. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 236. In some embodiments, the antibody or binding protein contains a humanized VHH domain having the amino acid sequence of SEQ ID NO: 236.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H8, shown below (CDR sequences shown in bold):

(BCMA-VHH3-H8, SEQ ID NO: 237)
QVQLVESGGGLVQPGGSLRLSCAASGSIFAYHVMGWYRQAPGKQRELV
ALITSGGSTNYTDSVKGRFTISRDNAKNTVYLQMNSLRAEDTAVYYCA
IGYYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 237. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 237. In some embodiments, the antibody or binding protein contains a humanized VHH domain having the amino acid sequence of SEQ ID NO: 237.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H9, shown below (CDR sequences shown in bold):

(BCMA-VHH3-H9, SEQ ID NO: 238)
QVQLVESGGGVVQPGGSLRLSCAASGSIFAYHVMGWYRQAPGKQRELV
ALITSGGSTNYTDSVKGRFTISRDNSKNTVYLQMNSLRAEDTAVYYCA
IGYYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 238. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 238. In some embodiments, the antibody or binding protein contains a humanized VHH domain having the amino acid sequence of SEQ ID NO: 238.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H10, shown below (CDR sequences shown in bold):

(BCMA-VHH3-H10, SEQ ID NO: 239)
QVQLVESGGGVVQPGGSLRLSCAASGSIFAYHVMGWYRQAPGKQRELV
ALITSGGSTNYTDSVKGRFTISRDNAKNTLYLQMNSLRAEDTAVYYCA
IGYYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 239. In some embodiments, the 100% identical) to the amino acid sequence of SEQ ID NO: 239. In some embodiments, the antibody or binding protein contains a humanized VHH domain having the amino acid sequence of SEQ ID NO: 239.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H11, shown below (CDR sequences shown in bold):

(BCMA-VHH3-H11, SEQ ID NO: 240)
EVQLVESGGGLVQPGGSLRLSCAASGSIFAYHVMGWYRQAPGKQRELVA
LITSGGSTNYTDSVKGRFTISRDNSKNTLYLQMNSLRAEDTAVYYCAIG
YYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 240. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 240. In some embodiments, the antibody or binding protein contains a humanized VHH domain having the amino acid sequence of SEQ ID NO: 240.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H12, shown below (CDR sequences shown in bold):

(BCMA-VHH3-H12, SEQ ID NO: 241)
EVQLVESGGGLVQPGGSLRLSCAASGSIFAYHVMGWYRQAPGKQLELVS
LITSGGSTNYTDSVKGRFTISRDNSKNTVYLQMNSLRAEDTAVYYCAIG
YYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 241. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 241. In some embodiments, the antibody or binding protein contains a humanized VHH domain having the amino acid sequence of SEQ ID NO: 241.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H13, shown below (CDR sequences shown in bold):

(BCMA-VHH3-H13, SEQ ID NO: 242)
QVQLVESGGGLVQPGGSLRLSCAASGSIFAYHVMGWYRQAPGKQLELVA
LITSGGSTNYTDSVKGRFTISRDNSKNTVYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 242. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 242. In some embodiments, the antibody or binding protein contains a humanized VHH domain having the amino acid sequence of SEQ ID NO: 242.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H14, shown below (CDR sequences shown in bold):

(BCMA-VHH3-H14, SEQ ID NO: 243)
EVQLVESGGGLVQPGGSLRLSCAASGSIFAYHVMGWYRQAPGKQRELVA
LITSGGSTNYTDSVKGRFTISRDNSKNTVYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 243. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 243. In some embodiments, the antibody or binding protein contains a humanized VHH domain having the amino acid sequence of SEQ ID NO: 243.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H15, shown below (CDR sequences shown in bold):

(BCMA-VHH3-H15, SEQ ID NO: 244)
QVQLVESGGGLVQPGGSLRLSCAASGSIFAYHVMGWYRQAPGKQRELVA
LITSGGSTNYTDSVKGRFTISRDNSKNTVYLQMNSLRAEDTAVYYCAIG
YYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 244. In some embodiments, the 100% identical) to the amino acid sequence of SEQ ID NO: 244. In some embodiments, the antibody or binding protein contains a humanized VHH domain having the amino acid sequence of SEQ ID NO: 244.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H16, shown below (CDR sequences shown in bold):

(BCMA-VHH3-H16, SEQ ID NO: 245)
QVQLVESGGGVVQPGGSLRLSCAASGSIFAYHVMGWYRQAPGKQRELVS
LITSGGSTNYTDSVKGRFTISRDNSKNTLYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 245. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 245. In some embodiments, the antibody or binding protein contains a humanized VHH domain having the amino acid sequence of SEQ ID NO: 245.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H17, shown below (CDR sequences shown in bold):

(BCMA-VHH3-H17, SEQ ID NO: 246)
QVQLVESGGGVVQPGGSLRLSCAASGSIFAYHVMGWYRQAPGKQLELVA
LITSGGSTNYTDSVKGRFTISRDNSKNTVYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 246. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 246. In some embodiments, the antibody or binding protein contains a humanized VHH domain having the amino acid sequence of SEQ ID NO: 246.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H18, shown below (CDR sequences shown in bold):

(BCMA-VHH3-H18, SEQ ID NO: 247)
EVQLVESGGGLVQPGGSLRLSCAASGSIFAYHVMGWYRQAPGKQRELVS
LITSGGSTNYTDSVKGRFTISRDNSKNTVYLQMNSLRAEDTAVYYCAIG
YYSGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 247. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 247. In some embodiments, the antibody or binding protein contains a humanized VHH domain having the amino acid sequence of SEQ ID NO: 247.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H19, shown below (CDR sequences shown in bold):

(BCMA-VHH3-H19, SEQ ID NO: 248)
EVQLVESGGGVVQPGGSLRLSCAASGSIFAYHVMGWYRQAPGKQRELVA
LITSGGSTNYTDSVKGRFTISRDNSKNTLYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 248. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 248. In some embodiments, the antibody or binding protein contains a humanized VHH domain having the amino acid sequence of SEQ ID NO: 248.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H20, shown below (CDR sequences shown in bold):

(BCMA-VHH3-H20, SEQ ID NO: 249)
EVQLVESGGGVVQPGGSLRLSCAASGSIFAYHVMGWYRQAPGKQRELVA
LITSGGSTNYTDSVKGRFTISRDNSKNTVYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 249. In some embodiments, the 100% identical) to the amino acid sequence of SEQ ID NO: 249. In some embodiments, the antibody or binding protein contains a humanized VHH domain having the amino acid sequence of SEQ ID NO: 249.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H21, shown below (CDR sequences shown in bold):

(BCMA-VHH3-H21, SEQ ID NO: 250)
QVQLVESGGGLVQPGGSLRLSCAASGSIFAYHVMGWYRQAPGKQRELVA
LITSGGSTNYTDSVKGRFTISRDNSKNTLYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 250. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 250. In some embodiments, the antibody or binding protein contains a humanized VHH domain having the amino acid sequence of SEQ ID NO: 250.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H22, shown below (CDR sequences shown in bold):

(BCMA-VHH3-H22, SEQ ID NO: 251)
QVQLVESGGGLVQPGGSLRLSCAASGSIFAYHVMGWYRQAPGKQLELVS
LITSGGSTNYTDSVKGRFTISRDNSKNTLYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 251. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 251. In some embodiments, the antibody or binding protein contains a humanized VHH domain having the amino acid sequence of SEQ ID NO: 251.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H23, shown below (CDR sequences shown in bold):

(BCMA-VHH3-H23, SEQ ID NO: 252)
EVQLVESGGGVVQPGGSLRLSCAASGSIFAYHVMGWYRQAPGKQRELVS
LITSGGSTNYTDSVKGRFTISRDNSKNTVYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 252. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 252. In some embodiments, the antibody or binding protein contains a humanized VHH domain having the amino acid sequence of SEQ ID NO: 252.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H24, shown below (CDR sequences shown in bold):

(BCMA-VHH3-H24, SEQ ID NO: 253)
EVQLVESGGGLVQPGGSLRLSCAASGSIFAYHVMGWYRQAPGKQRELVS
LITSGGSTNYTDSVKGRFTISRDNSKNTLYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 253. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 253. In some embodiments, the antibody or binding protein contains a humanized VHH domain having the amino acid sequence of SEQ ID NO: 253.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H25, shown below (CDR sequences shown in bold):

(BCMA-VHH3-H25, SEQ ID NO: 254)
QVQLVESGGGLVQPGGSLRLSCAASGSIFAYHVMGWYRQAPGKQRELVS
LITSGGSTNYTDSVKGRFTISRDNAKNTLYLQMNSLRAEDTAVYYCAIG
YYSGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 254. In some embodiments, the 100% identical) to the amino acid sequence of SEQ ID NO: 254. In some embodiments, the antibody or binding protein contains a humanized VHH domain having the amino acid sequence of SEQ ID NO: 254.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H26, shown below (CDR sequences shown in bold):

(BCMA-VHH3-H26, SEQ ID NO: 255)
QVQLVESGGGLVQPGGSLRLSCAASGSIFAYHVMGWYRQAPGKQLELVAL
ITSGGSTNYTDSVKGRFTISRDNSKNTVYLQMNSLRAEDTAVYYCAIGYY
SGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 255. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 255. In some embodiments, the antibody or binding protein contains a humanized VHH domain having the amino acid sequence of SEQ ID NO: 255.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H27, shown below (CDR sequences shown in bold):

(BCMA-VHH3-H27, SEQ ID NO: 256)
QVQLVESGGGVVQPGGSLRLSCAASGSIFAYHVMGWYRQAPGKQRELVSL
ITSGGSTNYTDSVKGRFTISRDNAKNTVYLQMNSLRAEDTAVYYCAIGYY
SGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 256. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 256. In some embodiments, the antibody or binding protein contains a humanized VHH domain having the amino acid sequence of SEQ ID NO: 256.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H28, shown below (CDR sequences shown in bold):

(BCMA-VHH3-H28, SEQ ID NO: 257)
QVQLVESGGGLVQPGGSLRLSCAASGSIFAYHVMGWYRQAPGKQRELVSL
ITSGGSTNYTDSVKGRFTISRDNSKNTVYLQMNSLRAEDTAVYYCAIGYY
SGSYYWERSGDYWGQGTLVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 257. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 257. In some embodiments, the antibody or binding protein contains a humanized VHH domain having the amino acid sequence of SEQ ID NO: 257.

In some embodiments, the anti-BCMA antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, BCMA-VHH3-H29, shown below (CDR sequences shown in bold):

(BCMA-VHH3-H29, SEQ ID NO: 258)
QVQLVESGGGLVQPGGSLRLSCAASGSIFAYHVMGWYRQAPGKQRELVSL
ITSGGSTNYTDSVKGRFTISRDNSKNTVYLQMNSLRAEDTAVYYCAIGYY
SGSYYWERSGDYWGQGTQVTVSS.

In some embodiments, the anti-BCMA antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 258. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 258. In some embodiments, the antibody or binding protein contains a humanized VHH domain having the amino acid sequence of SEQ ID NO: 258.

In some embodiments, the anti-GPRC5D antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH1, shown below (CDR sequences shown in bold):

(GPRC5D-VHH1, SEQ ID NO: 259)
SVQLVESEGGLVQAGGSLRLSCVASGRTVSSYAMGWFRQAPGKEREFVAA
ISWSGRSTYYADSMKGRFTISRDNAKNTVYLQMNNLRPEDTAVYYCATSR
AVIPGRDWNYYEYSGQGTQVTVSS.

In some embodiments, the anti-GPRC5D antibody or binding protein contains a VHH domain having an amino acid sequence that is at least 90% identical (e.g., at the amino acid sequence of SEQ ID NO: 259. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 259. In some embodiments, the antibody or binding protein contains a VHH domain having the amino acid sequence of SEQ ID NO: 259.

In some embodiments, the anti-GPRC5D antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH2, shown below (CDR sequences shown in bold):

(GPRC5D-VHH2, SEQ ID NO: 260)
QVQLVESGGGLVQAGGSLRLSCAASGRTASAYVMGWFRQAPGKERDFVAG
ISGGAYYADSVKGRFTIARDNAKNTVYLQMNSLKPEDTAVYYCAAERGMR
RLTESYQYDYWGQGTQVTVSS.

In some embodiments, the anti-GPRC5D antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 260. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 260. In some embodiments, the antibody or binding protein contains a VHH domain having the amino acid sequence of SEQ ID NO: 260.

In some embodiments, the anti-GPRC5D antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3, shown below (CDR sequences shown in bold):

(GPRC5D-VHH3, SEQ ID NO: 261)
QVKLVESGGGLVQPGGSLRLSCVGSGIIFSASNLAWYRQALGKQRELVAG
VTGGGSINYADSVKGRFTISRDNAKNTIYLQMNSLKPEDTAVYYCNARRS
YSHWGQGTRVTVSS.

In some embodiments, the anti-GPRC5D antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 261. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 261. In some embodiments, the antibody or binding protein contains a VHH domain having the amino acid sequence of SEQ ID NO: 261.

In some embodiments, the anti-GPRC5D antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4, shown below (CDR sequences shown in bold):

(GPRC5D-VHH4, SEQ ID NO: 262)
QVQLVESGGGLVQSGGSLRLSCTASGGFGMMYSMGWYRQAPGKQRELVAA
RTIDGSTNYADSVKDRFTVFRENNRNTVALEMDSLKPEDTAVYYCNAKPL
NGRLTQYWGQGTQVTVSS.

In some embodiments, the anti-GPRC5D antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 262. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 262. In some embodiments, the antibody or binding protein contains a VHH domain having the amino acid sequence of SEQ ID NO: 262.

a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH5, shown below (CDR sequences shown in bold):

(GPRC5D-VHH5, SEQ ID NO: 263)
QVHLVESGGGLVQAGGSLRLSCAASRIRFSINVMGWYRQAPGKQRELVAT
IAAGGTTNYADSVKGRFTISRDNAKNTVYLQMNSLKPDDTAVYYCNAVLS
TLVLPSTYWGQGTQVTVSS.

In some embodiments, the anti-GPRC5D antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 263. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 263. In some embodiments, the antibody or binding protein contains a VHH domain having the amino acid sequence of SEQ ID NO: 263.

In some embodiments, the anti-GPRC5D antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH6, shown below (CDR sequences shown in bold):

(GPRC5D-VHH6, SEQ ID NO: 264)
QVHLVESGGGLVQPGGSLRLSCIASSERTFRSYTMGWFRQAPGKEREFVA
AISWSHSSTYYADSVKGRFTISRDNAKNTVYLQMNNLKPEDTAVYYCAAD
LRLLPEEYDYYGQGTQVTVSS.

In some embodiments, the anti-GPRC5D antibody or binding protein contains a VHH domain having an amino acid sequence that is at least 90% identical (e.g., at the amino acid sequence of SEQ ID NO: 264. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 264. In some embodiments, the antibody or binding protein contains a VHH domain having the amino acid sequence of SEQ ID NO: 264.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH1-H1, shown below (CDR sequences shown in bold):

(GPRC5D-VHH1-H1, SEQ ID NO: 283)
QVQLVESGGGLVQPGGSLRLSCVASGRTVSSYAMGWFRQAPGKEREFVAA
ISWSGRSTYYADSMKGRFTISRDNAKNTVYLQMNSLRAEDTAVYYCATSR
AVIPGRDWNYYEYSGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 283. In some embodiments, the anti-GPRC5D antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 283. In some embodiments, the antibody or binding protein contains a humanized VHH domain having the amino acid sequence of SEQ ID NO: 283.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, GPRC5D-VHH1-H2, shown below (CDR sequences shown in bold):

(GPRC5D-VHH1-H2, SEQ ID NO: 284)
QVQLVESGGGLVQPGGSLRLSCVASGRTVSSYAMGWFRQAPGKEREFVAA
ISWSGRSTYYADSMKGRFTISRDNAKNTVYLQMNSLRAEDTAVYYCATSR
AVIPGRDWNYYEYSGQGTLVTVSS.

In some embodiments, the anti-GPRC5Dantibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 284. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 284. In some embodiments, the antibody or binding protein contains a humanized VHH domain having the amino acid sequence of SEQ ID NO: 284.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH1-H3, shown below (CDR sequences shown in bold):

(GPRC5D-VHH1-H3, SEQ ID NO: 285)
QVQLVESGGGLVQPGGSLRLSCVASGRTVSSYAMGWFRQAPGKEREFVAA
ISWSGRSTYYADSMKGRFTISRDNAKNTVYLQMNSLRAEDTAVYYCATSR
AVIPGRDWNYYEYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 285. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 285. In some embodiments, the antibody or binding protein contains a humanized VHH domain having the amino acid sequence of SEQ ID NO: 285 identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH1-H4, shown below (CDR sequences shown in bold):

(GPRC5D-VHH1-H4, SEQ ID NO: 286)
QVQLVESGGGLVQPGGSLRLSCAASGRTVSSYAMGWFRQAPGKEREFVAA
ISWSGRSTYYADSMKGRFTISRDNAKNTVYLQMNSLRAEDTAVYYCATSR
AVIPGRDWNYYEYSGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 286. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 286. In some embodiments, the antibody or binding protein contains a humanized VHH domain having the amino acid sequence of SEQ ID NO: 286.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH1-H5, shown below (CDR sequences shown in bold):

(GPRC5D-VHH1-H5, SEQ ID NO: 287)
QVQLVESGGGLVQPGGSLRLSCAASGRTVSSYAMGWFRQAPGKEREFVAA
ISWSGRSTYYADSMKGRFTISRDNAKNTVYLQMNSLRAEDTAVYYCATSR
AVIPGRDWNYYEYSGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 287. In some 97%, 98%, 99%, or 100% identical) to the amino acid sequence of SEQ ID NO: 287. In some embodiments, the antibody or binding protein contains a humanized VHH domain having the amino acid sequence of SEQ ID NO: 287.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH1-H6, shown below (CDR sequences shown in bold):

(GPRC5D-VHH1-H6, SEQ ID NO: 288)
QVQLVESGGGLVQPGGSLRLSCAASGRTVSSYAMGWFRQAPGKEREFVSA
ISWSGRSTYYADSMKGRFTISRDNAKNTLYLQMNSLRAEDTAVYYCATSR
AVIPGRDWNYYEYSGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 288. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 288. In some embodiments, the antibody or binding protein contains a humanized VHH domain having the amino acid sequence of SEQ ID NO: 288.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, GPRC5D-VHH1-H7, shown below (CDR sequences shown in bold):

(GPRC5D-VHH1-H7, SEQ ID NO: 289)
QVQLVESGGGLVQPGGSLRLSCAASGRTVSSYAMGWFRQAPGKEREFVSA
ISWSGRSTYYADSMKGRFTISRDNAKNTLYLQMNSLRAEDTAVYYCATSR
AVIPGRDWNYYEYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 289. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 289. In some embodiments, the antibody or binding protein contains a humanized VHH domain having the amino acid sequence of SEQ ID NO: 289.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH1-H8, shown below (CDR sequences shown in bold):

(GPRC5D-VHH1-H8, SEQ ID NO: 290)
QVQLVESGGGLVQPGGSLRLSCVASGRTVSSYAMGWFRQAPGKEREFVSA
ISWSGRSTYYADSMKGRFTISRDNAKNTLYLQMNSLRAEDTAVYYCATSR
AVIPGRDWNYYEYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 290. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 290. In some embodiments, the antibody or binding protein contains a humanized VHH domain having the amino acid sequence of SEQ ID NO: 290.

identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH1-H9, shown below (CDR sequences shown in bold):

(GPRC5D-VHH1-H9, SEQ ID NO: 291)
QVQLVESGGGLVQPGGSLRLSCAASGRTVSSYAMGWFRQAPGKEREFVSA
ISWSGRSTYYADSMKGRFTISRDNAKNTVYLQMNSLRAEDTAVYYCATSR
AVIPGRDWNYYEYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 291. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 291. In some embodiments, the antibody or binding protein contains a humanized VHH domain having the amino acid sequence of SEQ ID NO: 291.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H1, shown below (CDR sequences shown in bold):

(GPRC5D-VHH3-H1, SEQ ID NO: 292)
QVKLVESGGGLVQPGGSLRLSCVGSGIIFSASNLAWYRQAPGKQRELVAG
VTGGGSINYADSVKGRFTISRDNAKNTIYLQMNSLRPEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 292. In some embodiments, the anti-GPRC5D antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 292. In some embodiments, the antibody or binding protein contains a humanized VHH domain having the amino acid sequence of SEQ ID NO: 292.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H2, shown below (CDR sequences shown in bold):

(GPRC5D-VHH3-H2, SEQ ID NO: 293)
QVKLVESGGGLVQPGGSLRLSCVGSGIIFSASNLAWYRQAPGKQRELVAG
VTGGGSINYADSVKGRFTISRDNAKNTIYLQMNSLRAEDTAVYYCNARRS
YSHWGQGTRVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 293. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 293. In some embodiments, the antibody or binding protein contains a humanized VHH domain having the amino acid sequence of SEQ ID NO: 293.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, GPRC5D-VHH3-H3, shown below (CDR sequences shown in bold):

(GPRC5D-VHH3-H3, SEQ ID NO: 294)
QVKLVESGGGLVQPGGSLRLSCVGSGIIFSASNLAWYRQAPGKQRELVAG
VTGGGSINYADSVKGRFTISRDNAKNTIYLQMNSLRAEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 294. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 294. In some embodiments, the antibody or binding protein contains a humanized VHH domain having the amino acid sequence of SEQ ID NO: 294.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H4, shown below (CDR sequences shown in bold):

(GPRC5D-VHH3-H4, SEQ ID NO: 295)
QVKLVESGGGLVQPGGSLRLSCVGSGIIFSASNLAWYRQAPGKQRELVAG
VTGGGSINYADSVKGRFTISRDNSKNTIYLQMNSLRAEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 295. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 295. In some embodiments, the antibody or binding protein contains a humanized VHH domain having the amino acid sequence of SEQ ID NO: 295.

identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H5, shown below (CDR sequences shown in bold):

(GPRC5D-VHH3-H5, SEQ ID NO: 296)
QVQLVESGGGLVQPGGSLRLSCVGSGIIFSASNLAWYRQAPGKQRELVAG
VTGGGSINYADSVKGRFTISRDNAKNTIYLQMNSLRAEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 296. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 296. In some embodiments, the antibody or binding protein contains a humanized VHH domain having the amino acid sequence of SEQ ID NO: 296.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H6, shown below (CDR sequences shown in bold):

(GPRC5D-VHH3-H6, SEQ ID NO: 297)
QVKLVESGGGLVQPGGSLRLSCAASGIIFSASNLAWYRQAPGKQRELVAG
VTGGGSINYADSVKGRFTISRDNAKNTIYLQMNSLRAEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 297. In some 97%, 98%, 99%, or 100% identical) to the amino acid sequence of SEQ ID NO: 297. In some embodiments, the antibody or binding protein contains a humanized VHH domain having the amino acid sequence of SEQ ID NO: 297.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H7, shown below (CDR sequences shown in bold):

(GPRC5D-VHH3-H7, SEQ ID NO: 298)
QVQLVESGGGLVQPGGSLRLSCAASGIIFSASNLAWYRQAPGKQRELVSG
VTGGGSINYADSVKGRFTISRDNSKNTIYLQMNSLRAEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 298. In some embodiments, the anti-GPRC5D antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 298. In some embodiments, the antibody or binding protein contains a humanized VHH domain having the amino acid sequence of SEQ ID NO: 298.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, GPRC5D-VH3-H8, shown below (CDR sequences shown in bold):

(GPRC5D-VH3-H8, SEQ ID NO: 299)
QVQLVESGGGLVQPGGSLRLSCAGSGIIFSASNLAWYRQAPGKQRELVAG
VTGGGSINYADSVKGRFTISRDNAKNTIYLQMNSLRAEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 299. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 299. In some embodiments, the antibody or binding protein contains a humanized VHH domain having the amino acid sequence of SEQ ID NO: 299.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H9, shown below (CDR sequences shown in bold):

(GPRC5D-VHH3-H9, SEQ ID NO: 300)
QVQLVESGGGLVQPGGSLRLSCAASGIIFSASNLAWYRQAPGKQRELVAG
VTGGGSINYADSVKGRFTISRDNAKNTIYLQMNSLRAEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 300. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 300

In some embodiments, the antibody or binding protein contains a humanized VHH domain having the amino acid sequence of SEQ ID NO: 300, identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H10, shown below (CDR sequences shown in bold):

(GPRC5D-VHH3-H10, SEQ ID NO: 301)
QVQLVESGGGLVQPGGSLRLSCVASGIIFSASNLAWYRQAPGKQRELVAG
VTGGGSINYADSVKGRFTISRDNAKNTIYLQMNSLRAEDTAVYYCNARRS
YSHWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 301. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 301. In some embodiments, the antibody or binding protein contains a humanized VHH domain having the amino acid sequence of SEQ ID NO: 301.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH3-H11, shown below (CDR sequences shown in bold):

(GPRC5D-VHH3-H11, SEQ ID NO: 302)
QVQLVESGGGLVQPGGSLRLSCVGSGIIFSASNLAWYRQAPGKQRELVAG
VTGGGSINYADSVKGRFTISRDNAKNTIYLQMNSLRAEDTAVYYCNARRS
YSHWGQGTRVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 302. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 302. In some embodiments, the antibody or binding protein contains a humanized VHH domain having the amino acid sequence of SEQ ID NO: 302.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH2-H1, shown below (CDR sequences shown in bold):

(GPRC5D-VHH2-H1, SEQ ID NO: 303)
QVQLVESGGGLVQPGGSLRLSCAASGRTASAYVMGWFRQAPGKERD
FVAGISGGAYYADSVKGRFTIARDNAKNTVYLQMNSLRAEDTAVYY
CAAERGMRRLTESYQYDYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 303. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 303. In some embodiments, the antibody or binding protein contains a humanized VHH domain having the amino acid sequence of SEQ ID NO: 303.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH2-H2, shown below (CDR sequences shown in bold):

(GPRC5D-VHH2-H2, SEQ ID NO: 304)
QVQLVESGGGLVQPGGSLRLSCAASGRTASAYVMGWFRQAPGKERD
FVAGISGGAYYADSVKGRFTIARDNAKNTVYLQMNSLRAEDTAVYY
CAAERGMRRLTESYQYDYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 304. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 304. In some embodiments, the antibody or binding protein contains a humanized VHH domain having the amino acid sequence of SEQ ID NO: 304.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH2-H3, shown below (CDR sequences shown in bold):

(GPRC5D-VHH2-H3, SEQ ID NO: 305)
QVQLVESGGGLVQPGGSLRLSCAASGRTASAYVMGWFRQAPGKERE
FVAGISGGAYYADSVKGRFTIARDNAKNTVYLQMNSLRAEDTAVYY
CAAERGMRRLTESYQYDYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 305. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 305. In some embodiments, the antibody or binding protein contains a humanized VHH domain having the amino acid sequence of SEQ ID NO: 305.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH2-H4, shown below (CDR sequences shown in bold):

(GPRC5D-VHH2-H4, SEQ ID NO: 306)
QVQLVESGGGLVQPGGSLRLSCAASGRTASAYVMGWFRQAPGKERD
FVAGISGGAYYADSVKGRFTISRDNAKNTVYLQMNSLRAEDTAVYY
CAAERGMRRLTESYQYDYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 306. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 306. In some embodiments, the antibody or binding protein contains a humanized VHH domain having the amino acid sequence of SEQ ID NO: 306.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH2-H5, shown below (CDR sequences shown in bold):

(GPRC5D-VHH2-H5, SEQ ID NO: 307)
QVQLVESGGGLVQPGGSLRLSCAASGRTASAYVMGWFRQAPGKERE
FVAGISGGAYYADSVKGRFTISRDNSKNTVYLQMNSLRAEDTAVYY
CAAERGMRRLTESYQYDYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 307. In some embodiments, the anti-GPRC5D antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 307. In some embodiments, the antibody or binding protein contains a humanized VHH domain having the amino acid sequence of SEQ ID NO: 307.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H1, shown below (CDR sequences shown in bold):

(GPRC5D-VHH4-H1, SEQ ID NO: 308)
QVQLVESGGGLVQPGGSLRLSCAASGGFGMMYSMGWYRQAPGKQRE
LVAARTIDGSTNYADSVKDRFTISRENNRNTLYLQNDSLRAEDTAV
YYCNAKPLNGRLTQYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 308. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 308. In some embodiments, the antibody or binding protein contains a humanized VHH domain having the amino acid sequence of SEQ ID NO: 308.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H2, shown below (CDR sequences shown in bold):

(GPRC5D-VHH4-H2, SEQ ID NO: 309)
QVQLVESGGGLVQPGGSLRLSCAASGGFGMMYSMGWYRQAPGKQRE
LVAARTIDGSTNYADSVKDRFTISRENNRNTLYLQMDSLRAEDTAV
YYCNAKPLNGRLTQYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 309. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 309. In some embodiments, the antibody or binding protein contains a humanized VHH domain having the amino acid sequence of SEQ ID NO: 309.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H3, shown below (CDR sequences shown in bold):

(GPRC5D-VHH4-H3, SEQ ID NO: 310)
QVQLVESGGGLVQPGGSLRLSCAASGGFGMMYSMGWYRQAPGKQRE
LVAARTIDGSTNYADSVKDRFTVSRENNRNTLALQNDSLRAEDTAV
YYCNAKPLNGRLTQYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 310. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 310. In some embodiments, the antibody or binding protein contains a humanized VHH domain having the amino acid sequence of SEQ ID NO: 310.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H4, shown below (CDR sequences shown in bold):

(GPRC5D-VHH4-H4, SEQ ID NO: 311)
QVQLVESGGGLVQPGGSLRLSCTASGGFGMMYSMGWYRQAPGKQRE
LVAARTIDGSTNYADSVKDRFTIFRENNRNTLYLQMDSLRAEDTAV
YYCNAKPLNGRLTQYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 311. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 311. In some embodiments, the antibody or binding protein contains a humanized VHH domain having the amino acid sequence of SEQ ID NO: 311.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H5, shown below (CDR sequences shown in bold):

(GPRC5D-VHH4-H5, SEQ ID NO: 312)
QVQLVESGGGLVQPGGSLRLSCAASGGFGMMYSMGWYRQAPGKQRE
LVAARTIDGSTNYADSVKDRFTISRENNRNTVYLQNDSLRAEDTAV
YYCNAKPLNGRLTQYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 312. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 312. In some embodiments, the antibody or binding protein contains a humanized VHH domain having the amino acid sequence of SEQ ID NO: 312.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H6, shown below (CDR sequences shown in bold):

(GPRC5D-VHH4-H6, SEQ ID NO: 313)
QVQLVESGGGLVQPGGSLRLSCTASGGFGMMYSMGWYRQAPGKQRE
LVAARTIDGSTNYADSVKDRFTVFRENNRNTVYLQNDSLRAEDTAV
YYCNAKPLNGRLTQYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 313. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 313. In some embodiments, the antibody or binding protein contains a humanized VHH domain having the amino acid sequence of SEQ ID NO: 313.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H7, shown below (CDR sequences shown in bold):

(GPRC5D-VHH4-H7, SEQ ID NO: 314)
QVQLVESGGGLVQPGGSLRLSCTASGGFGMMYSMGWYRQAPGKQRE
LVAARTIDGSTNYADSVKDRFTVFRENNRNTVYLQMDSLRAEDTAV
YYCNAKPLNGRLTQYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 314. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 314. In some embodiments, the antibody or binding protein contains a humanized VHH domain having the amino acid sequence of SEQ ID NO: 314.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H8, shown below (CDR sequences shown in bold):

(GPRC5D-VHH4-H8, SEQ ID NO: 315)
QVQLVESGGGLVQPGGSLRLSCTASGGFGMMYSMGWYRQAPGKQRE
LVAARTIDGSTNYADSVKDRFTVFRENNRNTVALQNDSLRAEDTAV
YYCNAKPLNGRLTQYWGQGTQVTVSS.

In some embodiments, the anti-GPRC5Dantibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 315. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 315. In some embodiments, the antibody or binding protein contains a humanized VHH domain having the amino acid sequence of SEQ ID NO: 315.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H9, shown below (CDR sequences shown in bold):

(GPRC5D-VH4-H9, SEQ ID NO: 316)
QVQLVESGGGLVQPGGSLRLSCAASGGFGMMYSMGWYRQAPGKQRE
LVAARTIDGSTNYADSVKDRFTIFRENNRNTVALQNDSLRAEDTAV
YYCNAKPLNGRLTQYWGQGTQVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 316. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 316. In some embodiments, the antibody or binding protein contains a humanized VHH domain having the amino acid sequence of SEQ ID NO: 316.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H10, shown below (CDR sequences shown in bold):

(GPRC5D-VHH4-H10, SEQ ID NO: 317)
QVQLVESGGGLVQPGGSLRLSCTASGGFGMMYSMGWYRQAPGKQRE
LVAARTIDGSTNYADSVKDRFTVFRENNRNTVYLQMDSLRAEDTAV
YYCNAKPLNGRLTQYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 317. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 317. In some embodiments, the antibody or binding protein contains a humanized VHH domain having the amino acid sequence of SEQ ID NO: 317.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H11, shown below (CDR sequences shown in bold):

(GPRC5D-VHH4-H11, SEQ ID NO: 318)
QVQLVESGGGLVQPGGSLRLSCAASGGFGMMYSMGWYRQAPGKQRELVA
ARTIDGSTNYADSVKDRFTVFRENNRNTVYLQMDSLRAEDTAVYYCNAK
PLNGRLTQYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 318. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 318. In some embodiments, the antibody or binding protein contains a humanized VHH domain having the amino acid sequence of SEQ ID NO: 318.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H12, shown below (CDR sequences shown in bold):

(GPRC5D-VHH4-H12, SEQ ID NO: 319)
QVQLVESGGGLVQPGGSLRLSCAASGGFGMMYSMGWYRQAPGKQRELVA
ARTIDGSTNYADSVKDRFTVSRENNRNTVALQNDSLRAEDTAVYYCNAK
PLNGRLTQYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 319. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 319. In some embodiments, the antibody or binding protein contains a humanized VHH domain having the amino acid sequence of SEQ ID NO: 319.

In some embodiments, the anti-GPRC5D antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, GPRC5D-VHH4-H13, shown below (CDR sequences shown in bold):

(GPRC5D-VHH4-H13, SEQ ID NO: 320)
QVQLVESGGGLVQPGGSLRLSCTASGGFGMMYSMGWYRQAPGKQRELVA
ARTIDGSTNYADSVKDRFTVSRENNRNTVYLQMDSLRAEDTAVYYCNAK
PLNGRLTQYWGQGTLVTVSS.

In some embodiments, the anti-GPRC5D antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 320. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 320. In some embodiments, the antibody or binding protein contains a humanized VHH domain having the amino acid sequence of SEQ ID NO: 320.

In some embodiments, the anti-FcRH5 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH1, shown below (CDR sequences shown in bold):

(FcRH5-VHH1, SEQ ID NO: 321)
QVQFVESGGGLVQAGGSLRLSCAASGITVSRNDMGWYRQAPGKQRELVA
IMNRVGSTDTADSVKDRFTISRDNTKNTLYLQMNNLKPEDTAVYYCNAL
NTVITWPWGQGTQVTVSS.

In some embodiments, the anti-FcRH5 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 321. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 321. In some embodiments, the antibody or binding protein contains a VHH domain having the amino acid sequence of SEQ ID NO: 321.

In some embodiments, the anti-FcRH5 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH2, shown below (CDR sequences shown in bold):

(FcRH5-VHH2, SEQ ID NO: 322)
QVQLVESGGGLVQAGGSLRLSCTASVHIISHYSMGWFRQAPGKERELVA
AIPVSGRVPYYLESVKGRFTISRDNAKNRLYLQMNNLKAEDTAVYYCAA
YPRKGLEGNEYEYWGQGTQVTVSS.

In some embodiments, the anti-FcRH5 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 322. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 322. In some embodiments, the antibody or binding protein contains a VHH domain having the amino acid sequence of SEQ ID NO: 322.

In some embodiments, the anti-FcRH5 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH3, shown below (CDR sequences shown in bold):

(FcRH5-VHH3, SEQ ID NO: 323)
QVQLVESGGGSVQAGGSLRLSCVDSGHTLSTYAMGWFRQAPGKEREFVA
AIARDGGATYLSGSAQGRATISRDNAKSTVYLQMNNLKPDDTAVYYCAA
SSMFSTAKRDYSYWGQGTQVTVSS.

In some embodiments, the anti-FcRH5 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 323. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 323. In some embodiments, the antibody or binding protein contains a VHH domain having the amino acid sequence of SEQ ID NO: 323.

In some embodiments, the anti-FcRH5 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH4, shown

(FcRH5-VHH4, SEQ ID NO: 324)
QVHLVESGGTLVQAGGSLRLSCAASGRTFSTYAMGWFRQGPGKEREFVA
VIDTTGSASAYAGSVRGRFTVSRDNTENTVYLQMNTLKPEDTAVYYCAA
ARRYSTAPGDYDYWGQGTQVTVSS.

In some embodiments, the anti-FcRH5 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 324. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 324. In some embodiments, the antibody or binding protein contains a VHH domain having the amino acid sequence of SEQ ID NO: 324.

In some embodiments, the anti-FcRH5 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH5, shown below (CDR sequences shown in bold):

(FcRH5-VHH5, SEQ ID NO: 325)
QVQLVESGGGLVQAGGSLRLSCAASGSHFSIATMGWYRQAPGMERELVA
VLSSSGRPTYADSVKGRFAIYRNNSMNIVELQMNGLKPEDTAVYVCKAN
LKRFFIEERYRDYWGQGTQVIVSS.

In some embodiments, the anti-FcRH5 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 325. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 325. In some embodiments, the antibody or binding protein contains a VHH domain having the amino acid sequence of SEQ ID NO: 325.

In some embodiments, the anti-FcRH5 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH1-H1, shown below (CDR sequences shown in bold):

(FcRH5-VHH1-H1, SEQ ID NO: 341)
QVQLVESGGGLIQPGGSLRLSCAASGITVSRNDMGWYRQAPGKQRELVA
IMNRVGSTDTADSVKGRFTISRDNTKNSLYLQMNSLRAEDTAVYYCNAL
NTVITWPWGQGTQVTVSS.

In some embodiments, the anti-FcRH5 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 341. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 341. In some embodiments, the antibody or binding protein contains a humanized VHH domain having the amino acid sequence of SEQ ID NO: 341.

In some embodiments, the anti-FcRH5 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH1-H2, shown below (CDR sequences shown in bold):

(FcRH5-VHH1-H2, SEQ ID NO: 342)
QVQFVESGGGLIQPGGSLRLSCAASGITVSRNDMGWYRQAPGKQRELVA
IMNRVGSTDTADSVKGRFTISRDNTKNSLYLQMNSLRAEDTAVYYCNAL
NTVITWPWGQGTQVTVSS.

In some embodiments, the anti-FcRH5 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 342. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 342. In some embodiments, the antibody or binding protein contains a humanized VHH domain having the amino acid sequence of SEQ ID NO: 342.

In some embodiments, the anti-FcRH5 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH1-H3, shown below (CDR sequences shown in bold):

(FcRH5-VHH1-H3, SEQ ID NO: 343)
QVQLVESGGGLVQPGGSLRLSCAASGITVSRNDMGWYRQAPGKQRELVA
IMNRVGSTDTADSVKGRFTISRDNTKNSLYLQMNSLRAEDTAVYYCNAL
NTVITWPWGQGTQVTVSS.

In some embodiments, the anti-FcRH5 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 343. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 343. In some embodiments, the antibody or binding protein contains a humanized VHH domain having the amino acid sequence of SEQ ID NO: 343.

In some embodiments, the anti-FcRH5 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH1-H4, shown below (CDR sequences shown in bold):

(FcRH5-VHH1-H4, SEQ ID NO: 344)
QVQLVESGGGLIQPGGSLRLSCAASGITVSRNDMGWYRQAPGKQRELVA
IMNRVGSTDTADSVKGRFTISRDNTKNTLYLQMNSLRAEDTAVYYCNAL
NTVITWPWGQGTQVTVSS.

In some embodiments, the anti-FcRH5 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 344. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 344. In some embodiments, the antibody or binding protein contains a humanized VHH domain having the amino acid sequence of SEQ ID NO: 344.

In some embodiments, the anti-FcRH5 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, FcRH5-VHH1-H5, shown below (CDR sequences shown in bold):

(FcRH5-VHH1-H5, SEQ ID NO: 345)
QVQFVESGGGLVQPGGSLRLSCAASGITVSRNDMGWYRQAPGKQRELVA
IMNRVGSTDTADSVKGRFTISRDNTKNTLYLQMNSLRAEDTAVYYCNAL
NTVITWPWGQGTQVTVSS.

In some embodiments, the anti-FcRH5 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 345. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 345. In some embodiments, the antibody or binding protein contains a humanized VHH domain having the amino acid sequence of SEQ ID NO: 345.

In some embodiments, an antibody or antigen-binding fragment is a murine-specific antibody or antigen-binding fragment, e.g., the antibody or binding protein specifically binds the murine antigen. In some embodiments, an antibody or antigen-binding fragment is a rat-specific antibody or antigen-binding fragment, e.g., the antibody or binding protein specifically binds the rat antigen. In some embodiments, an antibody or antigen-binding fragment is a llama-specific antibody or antigen-binding fragment, e.g., the antibody or binding protein specifically binds the llama antigen. In some embodiments, an antibody or antigen-binding fragment is a human-specific antibody or antigen-binding fragment, e.g., the antibody or binding protein specifically binds the human antigen. In some embodiments, an antibody or antigen-binding fragment is human-specific even if the antibody or binding protein is not human or humanized.

### Multispecific Antibodies

In another aspect, the present disclosure provides multispecific antibodies, for example, bispecific antibodies (BsAbs), that may have binding specificities that are directed towards CD38, BCMA, GPRC5D, and/or FaRH5 and any other antigen, e.g., for a cell-surface protein, receptor, receptor subunit, or tissue-specific antigen, or other non-CD38, BCMA, GPRC5D, or FcRH5 antigens. Multispecific antibodies typically comprise at least two different variable domains, wherein each variable domain is capable of specifically binding to a separate antigen (i.e. CD38, BCMA. GPRC5D, or FcRH5 and any other antigen). Each antigen-binding domain of a bispecific antibody can comprise a heavy chain variable domain (VH), a light chain variable domain (VL), or a VHH and a VL. In the context of a bispecific antigen-binding fragment comprising a first and a second antigen-binding domain (e.g., a bispecific antibody), each antigen binding domain comprises at least one CDR that alone, or in combination with one or more additional CDRs and/or framework regions, specifically binds to a particular antigen (i.e., CD38, BCMA, GPRC5D, or FCRH5, any other antigen).

The first antigen-binding domain and the second antigen-binding domain may be directly or indirectly connected to one another to form a bispecific antigen-binding fragment (i.e., bispecific ScFv) further bound to an Fc domain. Alternatively, the first antigen-binding domain and the second antigen-binding domain may each be connected to a separate Fc domain. Bispecific antigen-binding fragments of the present disclosure may comprise two Fe domains that are each individually part of a separate antibody heavy chain. The first and second Fe domains may be of the same sequence, or the Fc domains may have a mutation in the CH3 domain intended for the facilitation or ease of purification of heterodimeric (i.e., bispecific) molecules.

A multispecific antibody may also be an antibody or antigen-binding fragment thereof that includes at least two separate antigen-binding domains (e.g., two scFvs joined by a linker). The scFvs may bind the same antigen or different antigens.

A bispecific antibody can also comprise multiple chains. A bispecific antibody may be an antibody or antigen-binding fragment thereof that includes a F(ab) with binding specificity directed towards a first antigen and a VHH domain with binding specificity directed towards a second antigen joined by a linker. In some embodiments, a bispecific antibody may be a tetravalent bispecific antibody.

In some embodiments, multispecific antibodies of the present disclosure are secreted (e.g., released from a cell, for example, into the extracellular milieu).

Multispecific antibodies of the present disclosure can include any anti-CD38, anti-BCMA, anti-GPRC5D, and/or anti-FCRH5 CDRs, or VHH domains described herein.

Multispecific antibodies of the present disclosure can comprise binding specificities that are directed towards CD38, BCMA, GPRC5D, and/or FCRH5 and any other antigen. Any other antigen may be or comprise, for example, a cancer cell antigen/marker, an immune cell antigen (e.g, a T cell activation marker), a pathogenic antigen, or any other non-CD38, BCMA, GPRC5D, or FcRH5 antigen.

The disclosed multispecific antibodies may be produced by any means known in the art for producing multispecific antibodies, so long as the resulting multispecific antibody retains the functional characteristic of being able to specifically bind CD38, BCMA, GPRC5D, and/or FCRH5 and at least one other antigen. In some embodiments, the BsAbs may be created using the methods described in Labrijin et al., Proc. Natl. Acad. Sci. USA, 110(13):5145-50 (2013). Briefly, the two parental Abs, each containing single matched point mutations in the CH3 domains, are separately expressed and then mixed under reducing conditions in vitro. This separates the Abs into half-molecules, followed by reassembly, to form bispecific antibodies, and is compatible with large-scale manufacturing of bispecific antibodies. However, this is simply one exemplary method for making a multispecific antibody. Those of skill in the art will be aware that other methods of producing multispecific antibodies are available, and the present disclosure is not intended to be limited solely to the methods of making and type of multispecific antibodies disclosed herein.

Other multispecific antibody formats or technologies may be used to make the multispecific antigen-binding molecules of the present disclosure. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, scFv-based or diabody bispecific formats, IgG-scFv fusions, dual variable domain (DVD)—Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab2 bispecific formats (see, e.g., Klein et al. 2012, mAbs 4:6, 1-1.1, and references cited therein, for a review of the foregoing formats).

The disclosed multispecific antibodies can be made from or incorporate the CDRs or variable regions from polyclonal, monoclonal, chimeric, human, partially or fully humanized, and/or recombinant antibodies. Thus, the “parent” antibodies for the disclosed multispecific antibodies are not particularly limited; however, they are preferably fully human or humanized. In some embodiments, the parent antibody can be a polyclonal antibody. In some embodiments, the parent antibody can be a monoclonal. In some embodiments, the parent antibody can be a human antibody.

### Affinity of Antibodies, Antigen-Binding Fragments, or Binding Proteins of the Disclosure

### Thermodynamic Properties of Anti-CD38, Anti-BCMA, Anti-GPRC5D, and Anti-FcRH5 Antibodies, Antigen-Binding Fragments, or Binding Proteins

Antibodies, antigen-binding fragments, or binding proteins of the disclosure may have an affinity for CD38, BCMA, GPRC5D, and/or FcRH5 of, for example, from 1 nM to 100 nM (e.g., from 10 nM to 90 nM, from 20 nM to 80 nM, from 30 nM to 70 nM, from 40 nM to 60 nM, or about 50 nM). In some embodiments, antibodies, antigen-binding fragments, or binding proteins of the disclosure have an affinity for CD38, BCMA, GPRC5D, and/or FcRH5 of from about 1 nM to about 100 nM. In some embodiments, antibodies, antigen-binding fragments, or binding proteins of the disclosure have an affinity for CD38, BCMA, GPRC5D, and/or FcRH5 of from about 1 nM to about 90 nM. In some embodiments, antibodies, antigen-binding fragments, or binding proteins of the disclosure have an affinity CD38, BCMA, GPRC5D, and/or FcRH5 of from about 1 nM to about 80 nM. In some embodiments, antibodies, antigen-binding fragments, or binding proteins of the disclosure have an affinity for CD38, BCMA, GPRC5D, and/or FcRH5 of from about 1 nM to 60 nM. In some embodiments, antibodies, antigen-binding fragments, or binding proteins of the disclosure have an affinity for CD38, BCMA, GPRC5D, and/or FcRH5 of from about 1 nM to 40 nM. In some embodiments, antibodies, antigen-binding fragments, or binding proteins of the disclosure have an affinity for CD38, BCMA, GPRC5D, and/or FcRH5 of from about 1 nM to 20 nM.

In some embodiments, antibodies, antigen-binding fragments, or binding proteins of the disclosure have an affinity for CD38, BCMA, GPRC5D, and/or FcRH5 of about 100 nM. In some embodiments, antibodies, antigen-binding fragments, or binding proteins of the disclosure have an affinity for CD38, BCMA. GPRC5D, and/or FcRH5 of about 95 nM. In some embodiments, antibodies, antigen-binding fragments, or binding proteins of the disclosure have an affinity for CD38, BCMA, GPRC5D, and/or FcRH5 of about 90 nM. In some embodiments, antibodies, antigen-binding fragments, or binding proteins of the disclosure have an affinity for CD38, BCMA, GPRC5D, and/or FcRH5 of about 85 nM. In some embodiments, antibodies, antigen-binding fragments, or binding proteins of the disclosure have an affinity for CD38, BCMA, GPRC5D, and/or FcRH5 of about 80 nM. In some embodiments, antibodies, antigen-binding fragments, or binding proteins of the disclosure have an affinity for CD38, BCMA, GPRC5D, and/or FcRH5 of about 75 nM. In some embodiments, antibodies, antigen-binding fragments, or binding proteins of the disclosure have an affinity for CD38, BCMA, GPRC5D, and/or FcRH5 of about 70 nM. In some embodiments, antibodies, antigen-binding fragments, or binding proteins of the disclosure have an affinity for CD38, BCMA, GPRC5D, and/or FcRH5 of about 65 nM. In some embodiments, antibodies, antigen-binding fragments, or binding proteins of the disclosure have an affinity for CD38, BCMA. GPRC5D, and/or FcRH5 of about 60 nM. In some embodiments, antibodies, antigen-binding fragments, or binding proteins of the disclosure have an affinity for CD38, BCMA, GPRC5D, and/or FcRH5 of about 55 nM. In some embodiments, antibodies, antigen-binding fragments, or binding proteins of the disclosure have an affinity for CD38, BCMA, GPRC5D, and/or FcRH5 of about 50 nM. In some embodiments, antibodies, antigen-binding fragments, or binding proteins of the disclosure have an affinity for CD38, BCMA, GPRC5D, and/or FcRH5 of about 45 nM. In some embodiments, antibodies, antigen-binding fragments, or binding proteins of the disclosure have an affinity for CD38, BCMA, GPRC5D, and/or FcRH5 of about 40 nM. In some embodiments, antibodies, antigen-binding fragments, or binding proteins of the disclosure have an affinity for CD38, BCMA, GPRC5D, and/or FcRH5 of about 35 nM. In some embodiments, antibodies, antigen-binding fragments, or binding proteins of the disclosure have an affinity for CD38, BCMA, GPRC5D, and/or FcRH5 of about 30 nM. In some embodiments, antibodies, antigen-binding fragments, or binding proteins of the disclosure have an affinity for CD38, BCMA, GPRC5D, and/or FcRH5 of about 25 nM. In some embodiments, antibodies, antigen-binding fragments, or binding proteins of the disclosure have an affinity for CD38, BCMA, GPRC5D, and/or FcRH5 of about 20 nM. In some embodiments, antibodies, antigen-binding fragments, or binding proteins of the disclosure have an affinity for CD38, BCMA, GPRC5D, and/or FcRH5 of about 15 nM. In some embodiments, antibodies, antigen-binding fragments, or binding proteins of the disclosure have an affinity for CD38, BCMA. GPRC5D, and/or FcRH5 of about 10 nM. In some embodiments, antibodies, antigen-binding fragments, or binding proteins of the disclosure have an affinity for CD38, BCMA, GPRC5D, and/or FcRH5 of about 5 nM. In some embodiments, antibodies, antigen-binding fragments, or binding proteins of the disclosure have an affinity for CD38, BCMA. GPRC5D, and/or FcRH5 of about 1 nM.

The specific binding of an antibody, antigen-binding fragments, or binding proteins described herein to CD38. BCMA, GPRC5D, and/or FcRH5 can be determined by any of a variety of established methods. The affinity can be represented quantitatively by various measurements, including the concentration of antibody or binding protein needed to achieve the equilibrium constant (K_{D}) of the antibody-, antigen-binding fragment-, or binding proteins—antigen complex dissociation. The equilibrium constant. K_{D}, which describes the interaction of CD38, BCMA, GPRC5D, and/or FcRH5 with an antibody, antigen-binding fragment, or binding proteins described herein is the chemical equilibrium constant for the dissociation reaction of an antigen-antibody, -antigen-binding fragment, or -binding protein complex into solvent-separated antigen and antibody, antigen-binding fragment, or binding proteins that do not interact with one another.

Antibodies, antigen-binding fragments, or binding proteins described herein include those that specifically bind to CD38. BCMA, GPRC5D, and/or FcRH5 with a K_{D} value of less than 100 nM (e.g., less than 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 4 nM, 3 nM, 2 nM, or 1 nM). In some embodiments, the antibodies, antigen-binding fragments, or binding proteins described herein specifically bind to CD38, BCMA, GPRC5D, and/or FCRH5 with a K_{D} value of less than 10 nM (e.g., less than 9 nM, 8 nM, 7 nM, 6 nM, 5 nM, 4 nM, 3 nM, 2 nM, or 1 nM).

Antibodies, antigen-binding fragments, or binding proteins described herein can also be characterized by a variety of in vitro binding assays. Examples of experiments that can be used to determine the K_{D} or EC₅₀ of an anti-CD38, anti-BCMA, anti-GPRC5D, and/or anti-FcRH5 antibody or binding protein include, e.g., surface plasmon resonance, isothermal titration calorimetry, fluorescence anisotropy, FACS-based assays, ELISA-based assays, gene expression assays, and protein expression assays, among others. ELISA represents a particularly useful method for analyzing antibody or binding protein activity, as such assays typically require minimal concentrations of binding domains (e.g., antibodies, antigen-binding fragments, binding proteins). A common signal that is analyzed in a typical ELISA assay is luminescence, which is typically the result of the activity of a peroxidase conjugated to a secondary antibody that specifically binds a primary antibody (e.g., an anti-CD38, anti-BCMA, anti-GPRC5D, and/or anti-FcRH5 antibody, antigen-binding fragment described herein). Antibodies, antigen-binding fragments, or binding proteins described herein may bind CD38. BCMA, GPRC5D. and/or FcRH5 and fragments thereof. Antibodies, antigen-binding fragments, or binding proteins described herein may additionally bind isolated peptides derived from CD38, BCMA, GPRC5D, and/or FcRH5 that structurally pre-organize various residues in a manner that simulates the conformation of the above fragments in the native protein. In a direct ELISA experiment, this binding can be quantified, e.g., by analyzing the luminescence that occurs upon incubation of an HRP substrate (e.g., 2,2′-azino-di-3-ethylbenzthiazoline sulfonate) with an antigen-antibody, antigen-antigen-binding fragment, or antigen-binding protein complex bound to a HRP-conjugated secondary antibody.

### Kinetic Properties of Anti-CD38, Anti-BCMA, Anti-GPRC5D, and Anti-FcRH5 Antibodies, Antigen-Binding Fragments, or Binding Proteins

In addition to the thermodynamic parameters of a CD38, BCMA, GPRC5D, and FcRH5-antibody, -antigen-binding fragment, or -binding protein interaction, it is also possible to quantitatively characterize the kinetic association and dissociation of an antibody, antigen-binding fragment, or binding proteins described herein with CD38, BCMA, GPRC5D, and/or FcRH5. This can be done, e.g., by monitoring the rate of antibody-, antigen-binding fragment-, or binding protein-antigen complex formation according to established procedures. For example, one can use surface plasmon resonance (SPR) to determine the rate constants for the formation (kₒₙ) and dissociation (k_{off}) of an antibody-, antigen-binding fragment-, or binding protein-CD38, -BCMA, -GPRC5D, or -FcRH5 complex. These data also enable calculation of the equilibrium constant of (K_{D}) of antibody-, antigen-binding fragment-, or binding protein-CD38, -BCMA, -GPRC5D, and -FcRH5 complex dissociation, since the equilibrium constant of this unimolecular dissociation can be expressed as the ratio of the k_{off} to kₒₙ values. SPR is a technique that is particularly advantageous for determining kinetic and thermodynamic parameters of antigen-antibody, -antigen-binding fragment, or -binding protein interactions since the experiment does not require that one component be modified by attachment of a chemical label. Rather, the antigen is typically immobilized on a solid metallic surface which is treated in pulses with solutions of increasing concentrations of antibody, antigen-binding fragment, or binding proteins. Antibody-, antigen-binding fragment-, or binding protein-antigen binding induces distortion in the angle of reflection of incident light at the metallic surface, and this change in refractive index over time as antibody or binding protein is introduced to the system can be fit to established regression models in order to calculate the association and dissociation rate constants of an antibody- or antigen-binding-fragment- or binding protein-antigen interaction.

Antibodies, antigen-binding fragments, or binding proteins described herein may exhibit high kₒₙ and low k_{off} values upon interaction with CD38. BCMA, GPRC5D, and/or FcRH5. For example, antibodies, antigen-binding fragments, or binding proteins described herein may exhibit kₒₙ values in the presence of CD38, BCMA, GPRC5D, and/or FcRH5 of greater than 10⁴ M⁻¹s⁻¹ (e.g., 1.0×10⁴ M⁻¹s⁻¹, 1.5 ×10⁴ M⁻¹s⁻¹, 2.0×10⁴ M⁻¹s⁻¹, 2.5×10⁴ M⁻¹s⁻¹. 3.0×10⁴ M⁻¹s⁻¹, 3.5×10⁴ M⁻¹s⁻¹. 4.0×10⁴ M⁻¹s⁻¹, 4.5×10⁴ M⁻¹s⁻¹, 5.0×10⁴ M⁻¹s⁻¹, 5.5×10⁴ M⁻¹s⁻¹, 6.0×10⁴ M⁻¹s⁻¹, 6.5×10⁴ M⁻¹s⁻¹, 7.0×10⁴ M⁻¹s⁻¹, 7.5×10⁴ M⁻¹s⁻¹, 8.0×10⁴ M⁻¹s⁻¹, 8.5×10⁴ M⁻¹s⁻¹, 9.0×10⁴ M⁻¹s⁻¹, 9.5×10⁴ M⁻¹s⁻¹, 1.0×10⁵ M⁻¹s⁻¹, 1.5×10⁵ M⁻¹s⁻¹, 2.0×10⁵ M⁻¹ s⁻¹, 2.5×10⁵ M⁻¹s⁻¹, 3.0×10⁵ M⁻¹s⁻¹, 3.5×10⁵ M⁻¹s⁻¹, 4.0×10⁵ M⁻¹s⁻¹, 4.5×10⁵ M⁻¹s⁻¹, 5.0×10⁵ M⁻¹s⁻¹, 5.5×10⁵ M⁻¹s⁻¹, 6.0×10⁵ M⁻¹s⁻¹, 6.5×10⁵ M⁻¹s⁻¹, 7.0×10⁵ M⁻¹s⁻¹, 7.5×10⁵ M⁻¹s⁻¹, 8.0×10⁵ M⁻¹s⁻¹, 8.5×10⁵ M⁻¹s⁻¹. 9.0×10⁵ M⁻¹s⁻¹, 9.5×10⁵ M⁻¹s⁻¹, or 1.0×10⁶ M⁻¹s⁻¹). Antibodies, antigen-binding fragments, or binding proteins described herein may exhibit low k_{off} values when bound to CD38, BCMA, GPRC5D, and/or FcRH5. For instance, antibodies, antigen-binding fragments, or binding proteins described herein may exhibit k_{off} values of less than 10⁻³ s⁻¹ when complexed to CD38, BCMA, GPRC5D, and/or FCRH5 (e.g., 1.0×10⁻³ s⁻¹, 9.5×10⁻⁴ s⁻¹, 9.0×10⁻⁴ s⁻¹, 8.5×10⁻⁴ s⁻¹, 8.0×10⁻⁴ s⁻¹, 7.5×10⁻⁴ s⁻¹, 7.0×10⁻⁴ s⁻¹, 6.5×10⁻⁴ s⁻¹, 6.0×10⁻⁴ s⁻¹, 5.5×10⁻⁴ s⁻¹,5.0×10⁻⁴ s⁻¹4.5×10⁻⁴ s⁻¹4.0×10⁻⁴ s⁻¹3.5×10⁻⁴ s⁻¹ 3.0×10⁻⁴ s⁻¹ 2.5 ×10⁻⁴ s⁻¹, 2.0×10⁻⁴ s⁻¹, 1.5×10⁻⁴ s⁻¹, 1.0×10⁻⁴ s⁻¹9.5×10⁻⁵ s⁻¹, 9.0×10⁻⁵ s⁻¹, 8.5×10⁻⁵ 1 8.0×10⁵ s⁻¹, 7.5×10⁻⁵s⁻¹, 7.0×10⁵ s⁻¹, 6.5×10⁵ s⁻¹, 6.0×10⁻⁵ s⁻¹, 5.5×10⁻⁵ s⁻¹, 5.0×10⁻⁵ s⁻¹, 4.5×10⁻⁵ s⁻¹, 4.0×10⁻⁵ s⁻¹, 3.5×10⁻⁵ s⁻¹, 3.0×10⁻⁵ s⁻¹. 2.5×10⁻⁵ s⁻¹, 2.0×10⁻⁵ s⁻¹, 1.5×10⁻⁵ s⁻¹, or 1.0×10⁻⁵ s⁻¹).

### Methods for Humanization

Antibodies, antigen-binding fragments, or binding proteins described herein can include fully human, humanized, primatized, and chimeric antibodies that contain one or more of the CDR sequences shown in Tables 5, 9, 13, 17 below. As an example, one strategy that can be used to design humanized antibodies, antigen-binding fragments, or binding proteins described herein is to align the sequences of the V_{H} and/or V_{L} of an antibody or binding protein (e.g., of the present disclosure) with the V_{H} and/or V_{L} of a consensus human antibody. Consensus human antibody heavy chain and light chain sequences are known in the art (see, e.g., the “VBASE” human germline sequence database; see also Kabat, et al., Sequences of Proteins of Immunological Interest, Fifth Edition, U.S. Department of Health and Human Services, NIH Publication No. 91-3242 (1991); Tomlinson et al., J. Mol. Biol. 227:776-98 (1992); and Cox et al., Eur. J. Immunol. 24:827-836 (1994); the disclosure of which is incorporated herein by reference). In this way, the variable domain framework residues and CDRs can be identified by sequence alignment (see, Kabat, supra). One can then substitute, for example, one or more of the CDRs of the consensus human antibody with the corresponding CDR(s) of an antibody or antigen-binding fragment or binding protein of the disclosure, thereby producing a humanized antibody, antigen-binding fragment, or binding protein.

Similarly, this strategy can also be used to produce primatized anti-CD38, anti-BCMA, anti-GPRC5D, and anti-FcRH5 antibodies, antigen-binding fragments, or binding proteins, as one can substitute, for example, one or more, or all, of the CDRs of a primate antibody consensus sequence with, for example, one or more, or all, of the CDRs of an antibody or binding protein of the disclosure. Consensus primate antibody sequences known in the art (see, e.g., U.S. Pat. Nos. 5,658,570; 5.681,722; and 5.693,780; the disclosures of each of which are incorporated herein by reference).

In some embodiments, it may be desirable to import particular framework residues in addition to CDR sequences from an anti-CD38, -BCMA, -GPRC5D, and/or -FcRH5 antibody or binding protein into the V_{H} and/or V_{L} of a human antibody. For instance, U.S. Pat. No. 6,054,297 identifies several instances when it may be advantageous to retain certain framework residues from a particular antibody heavy chain or light chain variable region in the resulting humanized antibody, antigen-binding fragment, or binding proteins. In some embodiments, framework residues may engage in non-covalent interactions with the antigen and thus contribute to the affinity of the antibody, antigen-binding fragment, or binding proteins for the target antigen. In some embodiments, individual framework residues may modulate the conformation of a CDR, and thus indirectly influence the interaction of the antibody, antigen-binding domain, or binding proteins with the antigen. Certain framework residues may form the interface between V_{H} and V_{L} domains, and may therefore contribute to the global antibody, antigen-binding domain, or binding protein structure. In some cases, framework residues may constitute functional glycosylation sites (e.g., Asn-X-Ser/Thr) which may dictate antibody, antigen-binding domain, or binding protein structure and antigen affinity upon attachment to carbohydrate moieties. In cases such as those described above, it may be beneficial to retain certain framework residues of an anti-CD38, anti-BCMA, anti-GPRC5D, and anti-FcRH5 antibody or binding protein in, e.g., a humanized or primatized antibody or antigen-binding fragment or binding protein thereof, as various framework residues may promote high epitope affinity and improved biochemical activity of the antibody or antigen-binding fragment or binding protein thereof.

Examples of the humanized variant sequences of the antibodies, antigen-binding fragments, or binding proteins described herein can be found in Tables 7, 11, 15, and 19 below. Antibodies described herein also include antibody fragments, Fab domains, F(ab′) molecules, F(ab′)₂ molecules, single-chain variable fragments (scFvs), tandem scFv fragments, diabodies, triabodies, dual variable domain immunoglobulins, multi-specific antibodies, bispecific antibodies, VHH, and heterospecific antibodies that contain one or more of the CDRs in Table 8, below, or a CDR having at least 85% sequence identity thereto (e.g., at least 86%, 87%, 88%, 89%, 90%, 91%, 92%, 93%, 94%, 95%, 96%, 97%, 98%, 99%, or 100% sequence identity thereto). These molecules can be expressed recombinantly, e.g., by incorporating polynucleotides encoding these proteins into expression vectors for transfection in a eukaryotic or prokaryotic cell using techniques described herein or known in the art, or synthesized chemically, e.g., by solid phase peptide synthesis methods described herein or known in the art.

### Nucleic Acids and Expression Systems

Anti-CD38, anti-BCMA, anti-GPRC5D, and anti-FcRH5 antibodies, antigen-binding fragments, or binding proteins described herein can be prepared by any of a variety of established techniques. For instance, an anti-CD38, anti-BCMA, anti-GPRC5D, or anti-FcRH5 antibody or antigen-binding fragment described herein can be prepared by recombinant expression of immunoglobulin light and heavy chain genes in a host cell. To express an antibody or antigen-binding fragment or binding protein recombinantly, a host cell can be transfected with one or more recombinant expression vectors carrying DNA fragments encoding the desired antibody chain(s), antigen-binding fragments, and/or additional binding protein domains (e.g., transmembrane domains, hinge domains). For example, the light and/or heavy chains of an antibody or an antigen-binding fragment can be expressed in the host cell and, optionally, secreted into the medium in which the host cells are cultured, from which medium the antibodies can be recovered. Standard recombinant DNA methodologies are used to obtain antibody heavy chain genes, light chain genes, and binding protein domains and to incorporate these genes into recombinant expression vectors and introduce the vectors into host cells, such as those described in Molecular Cloning; A Laboratory Manual, Second Edition (Sambrook. Fritsch and Maniatis (eds), Cold Spring Harbor, N. Y., 1989), Current Protocols in Molecular Biology (Ausubel et al., eds., Greene Publishing Associates (1989), and in U.S. Pat. No. 4,816,397; the disclosures of each of which are incorporated herein by reference.

Suitable vectors include, but are not limited to, viral vectors and non-viral vectors. Viral vectors can include retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., Measles and Sendai), positive strand RNA viruses, such as picomavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments or binding proteins described herein include Norwalk virus, togavirus, flavivirus, reoviruses, papovavirus, hepadnavirus, and hepatitis virus, for example. Non-viral vectors can include plasmids.

In addition to viral vectors, a variety of additional methods have been developed for the incorporation of genes, e.g., those encoding antibody light and heavy chains, single-domain antibodies, single-chain variable fragments (scFvs), tandem scFvs, Fab domains, F(ab′)₂ domains, diabodies, and triabodies, among others, into the genomes of target cells for antibody, antigen-binding fragment, and/or binding protein expression. One such method that can be used for incorporating polynucleotides encoding anti-CD38, anti-BCMA, anti-GPRC5D, and/or anti-FcRH5 antibodies, antigen-binding fragments, or binding proteins into prokaryotic or eukaryotic cells includes the use of transposons.

Another useful method for the integration of nucleic acid molecules encoding anti-CD38, anti-BCMA, anti-GPRC5D, and anti-FcRH5 antibodies, antigen-binding fragments, or binding proteins into the genome of a prokaryotic or eukarvotic cell is the clustered regularly interspaced short palindromic repeats (CRISPR)/Cas system, which is a system that originally evolved as an adaptive defense mechanism in bacteria and archaea against infection by viruses.

Alternative methods for site-specifically cleaving genomic DNA prior to the incorporation of a polynucleotide encoding an anti-CD38, anti-BCMA, anti-GPRC5D, and anti-FcRH5 antibody or binding protein described herein include the use of zinc finger nucleases and transcription activator-like effector nucleases (TALENs). Additional genome editing techniques that can be used to incorporate polynucleotides encoding antibodies, antigen-binding fragments, or binding proteins described herein into the genome of a prokaryotic or eukaryotic cell include the use of ARCUS™ meganucleases that can be rationally designed so as to site-specifically cleave genomic DNA.

To express anti-CD38, anti-BCMA, anti-GPRC5D, and/or anti-FcRH5 antibodies, antigen-binding fragments, or binding proteins described herein, polynucleotides encoding partial or full-length light and heavy chains, e.g., polynucleotides that encode a one or more of the CDR sequences of an antibody or binding protein described herein, can be inserted into expression vectors such that the genes are operatively linked to transcriptional and translational control sequences. The expression vector and expression control sequences are chosen to be compatible with the expression host cell used. Polynucleotides encoding the light chain gene and the heavy chain of an anti-CD38, anti-BCMA, anti-GPRC5D, and/or anti-FcRH5 antibody or binding protein can be inserted into separate vectors, or, optionally, both polvnucleotides can be incorporated into the same expression vector using established techniques described herein or known in the art.

In addition to polynucleotides encoding the heavy and light chains of an antibody, or a polynucleotide encoding a single-chain antibody, an antibody fragment, such as a scFv molecule, or a construct described herein, or a binding protein, the recombinant expression vectors described herein may carry regulatory sequences that control the expression of the antibody chain genes or binding protein domains in a host cell. The design of the expression vector, including the selection of regulatory sequences, may depend on such factors as the choice of the host cell to be transformed or the level of expression of protein desired. For instance, suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Viral regulatory elements, and sequences thereof, are described in detail, for instance, in U.S. Pat. Nos. 5,168,062, 4,510,245, and 4,968,615, the disclosures of each of which are incorporated herein by reference.

In addition to, for example, the antibody chain genes and regulatory sequences, the recombinant expression vectors described herein can carry additional sequences, such as sequences that regulate replication of the vector in host cells (e.g., origins of replication) and selectable marker genes. A selectable marker gene facilitates selection of host cells into which the vector has been introduced (see, e.g., U.S. Pat. Nos. 4,399,216, 4,634,665 and 5,179,017). For example, typically the selectable marker gene confers resistance to cytotoxic drugs, such as G418, puromycin, blasticidin, hygromycin or methotrexate, to a host cell into which the vector has been introduced. Suitable selectable marker genes include the dihydrofolate reductase (DHFR) gene (for use in DHFR” host cells with methotrexate selection/amplification) and the neo gene (for G418 selection). In order to express the light and heavy chains of an anti-CD38, anti-BCMA, anti-GPRC5D, and/or anti-FcRH5 antibody, antibody fragment, or binding protein, the expression vector(s) containing polynucleotides encoding the heavy and light chains can be transfected into a host cell by standard techniques.

### Host Cells for Expression of Anti-CD38, Anti-BCMA, Anti-GPRC5D, and Anti-FcRH5 Antibodies, Antigen-Binding Fragments, or Binding Proteins

It is possible to express the antibodies, antigen-binding fragments, or binding proteins described herein in either prokaryotic or eukaryotic host cells. In some embodiments, expression of antibodies, antigen-binding fragments, or binding proteins is performed in eukaryotic cells, e.g., mammalian host cells, for high secretion of a properly folded and immunologically active antibody or antigen-binding fragments. Exemplary mammalian host cells for expressing the recombinant antibodies, antigen-binding fragments, or binding proteins described herein include Chinese Hamster Ovary (CHO cells) (including DHFR CHO cells, described in Urlaub and Chasin (1980, Proc. Natl. Acad. Sci. USA 77:4216-4220), used with a DHFR selectable marker, e.g., as described in Kaufman and Sharp (1982. Mol. Biol. 159:601-621), NSO myeloma cells, COS cells, 293 cells (e.g., expi293), and SP2/0 cells. Additional cell types that may be useful for the expression of antibodies, antigen-binding fragments, or binding proteins include bacterial cells, such as BL-21(DE3) E. Coli cells, which can be transformed with vectors containing foreign DNA according to established protocols. Additional eukaryotic cells that may be useful for expression of antibodies, antigen-binding fragments, or binding proteins include yeast cells, such as auxotrophic strains of S. cerevisiae, which can be transformed and selectively grown in incomplete media according to established procedures known in the art. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown.

### Half-Life Extension of Anti-CD38, Anti-BCMA, Anti-GPRC5D, and Anti-FcRH5 Antibodies

In some embodiments, an anti-CD38, anti-BCMA, anti-GPRC5D, and anti-FcRH5 antibody or antigen-binding fragment of the disclosure is conjugated to a second molecule. e.g., to extend the half-life of the anti-CD38, anti-BCMA, anti-GPRC5D, and anti-FcRH5 antibody or antigen-binding fragment in vivo. Such molecules that can extend half-life of the anti-CD38, anti-BCMA, anti-GPRC5D. and anti-FcRH5 antibody or antigen-binding fragment are described below, and include polyethylene glycol (PEG), among others. Anti-CD38, anti-BCMA, anti-GPRC5D, and anti-FcRH5 antibodies and fragments thereof can be conjugated to these half-life extending molecules at, e.g., the N-terminus or C-terminus of a light and/or heavy chain of the antibody using any one of a variety of conjugation strategies known in the art. Examples of pairs of reactive functional groups that can be used to covalently tether an anti-CD38, anti-BCMA, anti-GPRC5D, and/or anti-FcRH5 antibody or fragment thereof to a half-life extending or other molecule include, without limitation, thiol pairs, carboxylic acids and amino groups, ketones and amino groups, aldehydes and amino groups, thiols and alpha,beta-unsaturated moieties (such as maleimides or dehydroalanine), thiols and alpha-halo amides, carboxylic acids and hydrazides, aldehydes and hydrazides, and ketones and hydrazides.

Anti-CD38, anti-BCMA, anti-GPRC5D, and/or anti-FcRH5 antibodies can be conjugated to various molecules for the purpose of improving the half-life, solubility, and stability of the protein in aqueous solution. Examples of such molecules include polyethylene glycol (PEG), murine serum albumin (MSA), bovine serum albumin (BSA), and human serum albumin (HSA), among others. For instance, one can conjugate an anti-CD38, anti-BCMA, anti-GPRC5D, and/or anti-FcRH5 antibody or antigen-binding fragment to carbohydrate moieties in order to evade detection of the antibody antigen-binding fragment by the immune system of the patient receiving treatment. This process of hyperglycosylation reduces the immunogenicity of therapeutic proteins by sterically inhibiting the interaction of the protein with B cell receptors in circulation. Additionally, anti-CD38, anti-BCMA, anti-GPRC5D, and anti-FcRH5 antibodies, antigen-binding fragments, or binding proteins can be conjugated to molecules that prevent clearance from human serum and improve the pharmacokinetic profile of the antibodies, antigen-binding fragments, or binding proteins.

Anti-CD38, anti-BCMA, anti-GPRC5D, and anti-FcRH5 antibodies, antigen-binding fragments, or binding proteins can be covalently appended directly to a half-life extending or other molecule by chemical conjugation as described. Alternatively, fusion proteins containing anti-CD38, anti-BCMA, anti-GPRC5D, and/or anti-FcRH5 antibodies, antigen-binding fragments, or binding proteins can be expressed recombinantly from a cell (e.g., a eukaryotic cell or prokaryotic cell). This can be accomplished, for example, by incorporating a polynucleotide encoding the fusion protein into the genome of a cell (e.g., using techniques described herein or known in the art). Optionally, antibodies and fragments thereof described herein can be joined to a half-life extending molecule by forming a covalent bond between the antibody and a linker. This linker can then be subsequently conjugated to another molecule, or the linker can be conjugated to another molecule prior to ligation to the anti-CD38, anti-BCMA, anti-GPRC5D, and/or anti-FcRH5 antibody or antigen-binding fragment. Examples of linkers that can be used for the formation of a conjugate include polypeptide linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Fusion proteins containing polypeptide linkers can be made using chemical synthesis techniques, such as those described herein, or through recombinant expression of a polynucleotide encoding the fusion protein in a cell (e.g., a prokaryotic or eukaryotic cell). Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582 (2012)).

### Nucleic Acids Encoding Anti-CD38, Anti-BCMA, Anti-GPRC5D, and Anti-FcRH5 Antibodies or Binding Proteins

This section provides exemplary nucleic acids that may be used to encode antibodies or antigen-binding fragments of the disclosure. The nucleic acid molecules of the disclosure may include one or more alterations. Herein, in a nucleotide, nucleoside, or polynucleotide (such as the nucleic acids of the invention (e.g., an mRNA or an oligonucleotide)), the terms “alteration” or, as appropriate, “alternative” refer to alteration with respect to A, G, U or C ribonucleotides.

The alterations may be various distinct alterations. In some embodiments, where the nucleic acid is an mRNA, the coding region, the flanking regions, and/or the terminal regions may contain one, two, or more (optionally different) nucleoside or nucleotide alterations. In some embodiments, an alternative polynucleotide introduced to a cell may exhibit reduced degradation in the cell, as compared to an unaltered polynucleotide.

The polynucleotides can include any useful alteration, such as to the sugar, the nucleobase, or the internucleoside linkage (e.g., to a linking phosphate, to a phosphodiester linkage, or to the phosphodiester backbone). In certain embodiments, alterations (e.g., one or more alterations) are present in each of the sugar and the internucleoside linkage. Alterations according to the present invention may be alterations of ribonucleic acids (RNAs) to deoxyribonucleic acids (DNAs) (e.g., the substitution of the 2′OH of the ribofuranosyl ring to 2′H), threose nucleic acids (TNAs), glycol nucleic acids (GNAs), peptide nucleic acids (PNAs), locked nucleic acids (LNAs) or hybrids thereof. Additional alterations are described herein.

In certain embodiments, it may be desirable for a nucleic acid molecule introduced into the cell to be degraded intracellularly. For example, degradation of a nucleic acid molecule may be preferable if precise timing of protein production is desired. Thus, in some embodiments, the invention provides an alternative nucleic acid molecule containing a degradation domain, which is capable of being acted on in a directed manner within a cell.

The polynucleotides can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, vectors, etc.). In some embodiments, the polynucleotides may include one or more messenger RNAs (mRNAs) having one or more alternative nucleoside or nucleotides (i.e., mRNA molecules). In some embodiments, the polynucleotides may include one or more oligonucleotides having one or more alternative nucleoside or nucleotides. In some embodiments, a composition of the invention includes an mRNA and/or one or more oligonucleotides having one or more alternative nucleoside or nucleotides.

### Modified Nucleic Acids

According to Aduri et al., (Aduri, R. et al., Journal of Chemical Theory and Computation. 3(4):1464-75(2006)), there are 107 naturally occurring nucleosides, including 1-methyladenosine, 2-methylthio-N6-hydroxynorvalyl carbamoyladenosine, 2-methyladenosine, 2-O-ribosylphosphate adenosine, N6-methyl-N6-threonylcarbamoyladenosine, N6-acetyladenosine, N6-glycinylcarbamoyladenosine, N6-isopentenyladenosine, N6-methyladenosine, N6-threonylcarbamoyladenosine, N6,N6-dimethyladenosine, N6-(cis-hydroxyisopentenyl)adenosine. N6-hydroxynorvalylcarbamoyladenosine, 1,2-O-dimethyladenosine, N6,2-O-dimethyladenosine, 2-O-methyladenosine, N6,N6,O-2-trimethyladenosine, 2-methylthio-N6-(cis-hydroxyisopentenyl) adenosine, 2-methylthio-N6-methyladenosine, 2-methylthio-N6-isopentenyladenosine. 2-methylthio-N6-threonyl carbamoyladenosine, 2-thiocytidine, 3-methylcytidine, N4-acetylcytidine, 5-formylcytidine, N4-methylcytidine, 5-methylcytidine, 5-hydroxymethylcytidine, lysidine, N4-acetyl-2-O-methylcytidine, 5-formyl-2-O-methylcytidine, 5,2-O-dimethylcytidine, 2-O-methylcytidine. N4,2-O-dimethylcytidine, N4,N4,2-O-trimethylcytidine, 1-methylguanosine, N2,7-dimethylguanosine, N2-methylguanosine, 2-O-ribosylphosphate guanosine, 7-methylguanosine, under modified hydroxywybutosine, 7-aminomethyl-7-deazaguanosine, 7-cyano-7-deazaguanosine, N2,N2-dimethylguanosine, 4-demethylwyosine, epoxyqueuosine, hydroxywybutosine, isowyosine. N2,7,2-O-trimethylguanosine. N2,2-O-dimethylguanosine, 1,2-O-dimethylguanosine, 2-O-methylguanosine, N2,N2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine. 3-(3-amino-3-carboxypropyl)uridine, 3-methyluridine, 4-thiouridine. 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine. 5-(isopentenylaminomethyl)uridine. 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine. 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine. 5-methylaminomethyl-2-selenouridine, 5-methoxycarbonylmethyl-2-thiouridine. 5-taurinomethyl-2-thiouridine, pseudouridine, 1-methyl-3-(3-amino-3-carboxypropyl)pseudouridine, 1-methylpseudouridine, 3-methylpseudouridine, 2-O-methylpseudouridine, inosine, 1-methylinosine, 1,2-O-dimethylinosine, and 2-O-methylinosine. Each of these may be components of nucleic acids of the present invention.

### Nucleosides Containing Modified Sugars

The alternative nucleosides and nucleotides (e.g., building block molecules), which may be incorporated into a polynucleotide (e.g., RNA or mRNA, as described herein), can be altered on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone): multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

### Alterations on the Nucleobase

The present disclosure provides for alternative nucleosides and nucleotides. As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

Exemplary non-limiting alterations include an amino group, a thiol group, an alkyl group, a halo group, or any described herein. The alternative nucleotides may by synthesized by any useful method, as described herein (e.g., chemically, enzymatically, or recombinantly to include one or more alternative or alternative nucleosides).

In some embodiments, a nucleic acid of the invention (e.g., an mRNA or an oligonucleotide) includes one or more 2′-OMe nucleotides, 2′-methoxyethyl nucleotides (2′-MOE nucleotides). 2′-F nucleotide, 2′-NH2 nucleotide. 2′fluoroarabino nucleotides (FANA nucleotides), locked nucleic acid nucleotides (LNA nucleotides), or 4′-S nucleotides.

The alternative nucleotide base pairing encompasses not only the standard adenosine-thymine, adenosine-uracil, and guanosine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

The alternative nucleosides and nucleotides can include an alternative nucleobase. Examples of nucleobases found in RNA include, but are not limited to, adenine, guanine, cytosine, and uracil. Examples of nucleobase found in DNA include, but are not limited to, adenine, guanine, cytosine, and thymine. These nucleobases can be altered or wholly replaced to provide polynucleotide molecules having enhanced properties (e.g., resistance to nucleases and stability), and these properties may manifest through disruption of the binding of a major groove binding partner.

In some embodiments, the alternative nucleobase is an alternative uracil. Exemplary nucleobases and nucleosides having an alternative uracil include pseudouridine (yr), pyridin-4-one ribonucleoside, 5-aza-uridine. 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-melhoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaninomethyl-2-seleno-uridine (mnm⁵se²U), 5-carbamoylmethyl-uridine (ncm⁵U), 5-carboxymethylaminomethyl-uridine (cnnm⁵U), 5-carboxymethylaninomethyl-2-thio-uridine (cnnm⁵s²U), 5-propynyl-uridine, 1-propynyl-pseudouridine, 5-taurinomethyl-uridine (τm⁵U). 1-taurinomethvl-pseudouridine, 5-taurinomethyl-2-thio-uridine(τm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine. 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine. 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythynidine, 2′-F-ara-uridine, 2′-F-uridine, 2′-OH-ara-uridine, 5-(2-carbomethoxyvinyl) uridine, and 5-[3-(1-E-propenylamino)uridine.

In preferred embodiments, the nucleic acid is modified to contain 1-methylpseudouridine (m¹ψ) in lieu of uridine at each instance.

In some embodiments, the alternative nucleobase is an alternative cytosine. Exemplary nucleobases and nucleosides having an alternative cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C). 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine. 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴2Cm), 1-thio-cytidine, 2′-F-ara-cytidine, 2′-F-cytidine, and 2′-OH-ara-cytidine.

In some embodiments, the alternative nucleobase is an alternative adenine. Exemplary nucleobases and nucleosides having an alternative adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine. 7-deaza-8-aza-2-amino-purine. 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A). 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methvl-N6-threonylcarbamovl-adenosine (m⁶t⁶A), 2-methylthio-N6-threonylcarbamoyl-adenosine (ms²g⁶A). N6,N6-dimethyl-adenosine (m⁶2A), N6-hydroxynorvalylcarbamoyl-adenosine (hn⁶A), 2-methylthio-N6-hydroxynorvalylcarbamoyl-adenosine (ms² hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶2Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the alternative nucleobase is an alternative guanine. Exemplary nucleobases and nucleosides having an alternative guanine include inosine (I), 1-methvl-inosine (m¹I), wyosine (imG), methvlwyosine (mimG), 4-demethyl-wyosine (imG-14), isowvosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine. 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²2Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im). 2′-O-ribosylguanosine (phosphate) (Gr(p)), 1-thio-guanosine, O6-methyl-guanosine, 2′-F-ara-guanosine, and 2′-F-guanosine.

The nucleobase of the nucleotide can be independently selected from a purine, a pyrimidine, a purine, or pyrimidine analog. For example, the nucleobase can each be independently selected from adenine, cytosine, guanine, uracil, or hypoxanthine. In some embodiments, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl, and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil). 4-thiouracil, 8-halo (e.g., 8-bromo), 8-amino, 8-thiol, 8-thioalkyl. 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. When the nucleotides are depicted using the shorthand A, G, C, T or U, each letter refers to the representative base and/or derivatives thereof (e.g., A includes adenine or adenine analogs (e.g., 7-deaza adenine)).

In some embodiments, the polynucleotides of the invention contain 5-methoxy-uracil, uracil, 5-methyl-cytosine, and cytosine as the only uracils and cytosines. In some embodiments, the polynucleotides of the invention contain 5-methoxy-uracil, uracil, 5-trifluoromethyl-cytosine, and cytosine as the only uracils and cytosines. In some embodiments, the polynucleotides of the invention contain 5-methoxy-uracil, uracil, 5-hydroxymethyl-cytosine, and cytosine as the only uracils and cytosines. In some embodiments, the polynucleotides of the invention contain 5-methoxy-uracil, uracil, 5-bromo-cytosine, and cytosine as the only uracils and cytosines. In some embodiments, the polynucleotides of the invention contain 5-methoxy-uracil, uracil, 5-iodo-cytosine, and cytosine as the only uracils and cytosines. In some embodiments, the polynucleotides of the invention contain 5-methoxy-uracil, uracil, 5-methoxy-cytosine, and cytosine as the only uracils and cytosines. In some embodiments, the polynucleotides of the invention contain 5-methoxy-uracil, uracil, 5-ethyl-cytosine, and cytosine as the only uracils and cytosines. In some embodiments, the polynucleotides of the invention contain 5-methoxy-uracil, uracil, 5-phenyl-cytosine, and cytosine as the only uracils and cytosines. In some embodiments, the polynucleotides of the invention contain 5-methoxy-uracil, uracil, 5-ethnyl-cytosine, and cytosine as the only uracils and cytosines. In some embodiments, the polynucleotides of the invention contain 5-methoxy-uracil, uracil, N4-methyl-cytosine, and cytosine as the only uracils and cytosines. In some embodiments, the polynucleotides of the invention contain 5-methoxy-uracil, uracil, 5-fluoro-cytosine, and cytosine as the only uracils and cytosines. In some embodiments, the polynucleotides of the invention contain 5-methoxy-uracil, uracil, N4-acetyl-cytosine, and cytosine as the only uracils and cytosines. In some embodiments, the polynucleotides of the invention contain 5-methoxy-uracil, uracil, pseudoisocytosine, and cytosine as the only uracils and cytosines. In some embodiments, the polynucleotides of the invention contain 5-methoxy-uracil, uracil, 5-formyl-cytosine, and cytosine as the only uracils and cytosines. In some embodiments, the polynucleotides of the invention contain 5-methoxy-uracil, uracil, 5-aminoallyl-cytosine, and cytosine as the only uracils and cytosines. In some embodiments, the polynucleotides of the invention contain 5-methoxy-uracil, uracil, 5-carboxy-cytosine, and cytosine as the only uracils and cytosines.

In some embodiments, the polynucleotides of the invention contain 1-methyl-pseudouracil, uracil, 5-methyl-cytosine, and cytosine as the only uracils and cytosines. In some embodiments, the polynucleotides of the invention contain 1-methyl-pseudouracil, uracil, 5-trifluoromethyl-cytosine, and cytosine as the only uracils and cytosines. In some embodiments, the polynucleotides of the invention contain 1-methyl-pseudouracil, uracil, 5-hydroxymethyl-cytosine, and cytosine as the only uracils and cytosines. In some embodiments, the polynucleotides of the invention contain 1-methyl-pseudouracil, uracil, 5-bromo-cytosine, and cytosine as the only uracils and cytosines. In some embodiments, the polynucleotides of the invention contain 1-methyl-pseudouracil, uracil, 5-iodo-cytosine, and cytosine as the only uracils and cytosines. In some embodiments, the polynucleotides of the invention contain 1-methyl-pseudouracil, uracil, 5-methoxy-cytosine, and cytosine as the only uracils and cytosines. In some embodiments, the polynucleotides of the invention contain 1-methyl-pseudouracil, uracil, 5-ethyl-cytosine, and cytosine as the only uracils and cytosines. In some embodiments, the polynucleotides of the invention contain 1-methyl-pseudouracil, uracil, 5-phenyl-cytosine, and cytosine as the only uracils and cytosines. In some embodiments, the polynucleotides of the invention contain 1-methyl-pseudouracil, uracil, 5-ethnyl-cytosine, and cytosine as the only uracils and cytosines. In some embodiments, the polynucleotides of the invention contain 1-methyl-pseudouracil, uracil, N4-methyl-cytosine, and cytosine as the only uracils and cytosines. In some embodiments, the polynucleotides of the invention contain 1-methyl-pseudouracil, uracil, 5-fluoro-cytosine, and cytosine as the only uracils and cytosines. In some embodiments, the polynucleotides of the invention contain 1-methyl-pseudouracil, uracil, N4-acetyl-cytosine, and cytosine as the only uracils and cytosines. In some embodiments, the polynucleotides of the invention contain 1-methyl-pseudouracil, uracil, pseudoisocytosine, and cytosine as the only uracils and cytosines. In some embodiments, the polynucleotides of the invention contain 1-methyl-pseudouracil, uracil, 5-formyl-cytosine, and cytosine as the only uracils and cytosines. In some embodiments, the polynucleotides of the invention contain 1-methyl-pseudouracil, uracil, 5-aminoallyl-cytosine, and cytosine as the only uracils and cytosines. In some embodiments, the polynucleotides of the invention contain 1-methyl-pseudouracil, uracil, 5-carboxy-cytosine, and cytosine as the only uracils and cytosines.

In some embodiments, the polynucleotides of the invention contain 5-methoxy-uridine, uridine, 5-methyl-cytidine, and cytidine as the only uridines and cytidines. In some embodiments, the polynucleotides of the invention contain 5-methoxy-uridine, uridine, 5-trifluoromethyl-cytidine, and cytidine as the only uridines and cytidines. In some embodiments, the polynucleotides of the invention contain 5-methoxy-uridine, uridine, 5-hydroxymethyl-cytidine, and cytidine as the only uridines and cytidines. In some embodiments, the polynucleotides of the invention contain 5-methoxy-uridine, uridine, 5-bromo-cytidine, and cytidine as the only uridines and cytidines. In some embodiments, the polynucleotides of the invention contain 5-methoxy-uridine, uridine, 5-iodo-cytidine, and cytidine as the only uridines and cytidines. In some embodiments, the polynucleotides of the invention contain 5-methoxy-uridine, uridine, 5-methoxy-cytidine, and cytidine as the only uridines and cytidines. In some embodiments, the polynucleotides of the invention contain 5-methoxy-uridine, uridine, 5-ethyl-cytidine, and cytidine as the only uridines and cytidines. In some embodiments, the polynucleotides of the invention contain 5-methoxy-uridine, uridine, 5-phenyl-cytidine, and cytidine as the only uridines and cytidines. In some embodiments, the polynucleotides of the invention contain 5-methoxy-uridine, uridine, 5-ethnyl-cytidine, and cytidine as the only uridines and cytidines. In some embodiments, the polynucleotides of the invention contain 5-methoxy-uridine, uridine, N4-methyl-cytidine, and cytidine as the only uridines and cytidines. In some embodiments, the polynucleotides of the invention contain 5-methoxy-uridine, uridine, 5-fluoro-cytidine, and cytidine as the only uridines and cytidines. In some embodiments, the polynucleotides of the invention contain 5-methoxy-uridine, uridine, N4-acetyl-cytidine, and cytidine as the only uridines and cytidines. In some embodiments, the polynucleotides of the invention contain 5-methoxy-uridine, uridine, pseudoisocytidine, and cytidine as the only uridines and cytidines. In some embodiments, the polynucleotides of the invention contain 5-methoxy-uridine, uridine, 5-formyl-cytidine, and cytidine as the only uridines and cytidines. In some embodiments, the polynucleotides of the invention contain 5-methoxy-uridine, uridine, 5-aminoallyl-cytidine, and cytidine as the only uridines and cytidines. In some embodiments, the polynucleotides of the invention contain 5-methoxy-uridine, uridine, 5-carboxy-cytidine, and cytidine as the only uridines and cytidines.

In some embodiments, the polynucleotides of the invention contain 1-methyl-pseudouridine, uridine. 5-methyl-cytidine, and cytidine as the only uridines and cytidines. In some embodiments, the polynucleotides of the invention contain 1-methvl-pseudouridine, uridine. 5-trifluoromethyl-cytidine, and cytidine as the only uridines and cytidines. In some embodiments, the polynucleotides of the invention contain 1-methyl-pseudouridine, uridine, 5-hydroxymethyl-cytidine, and cytidine as the only uridines and cytidines. In some embodiments, the polynucleotides of the invention contain 1-methyl-pseudouridine, uridine, 5-bromo-cytidine, and cytidine as the only uridines and cytidines. In some embodiments, the polynucleotides of the invention contain 1-methyl-pseudouridine, uridine, 5-iodo-cytidine, and cytidine as the only uridines and cytidines. In some embodiments, the polynucleotides of the invention contain 1-methyl-pseudouridine, uridine, 5-methoxy-cytidine, and cytidine as the only uridines and cytidines. In some embodiments, the polynucleotides of the invention contain 1-methyl-pseudouridine, uridine, 5-ethyl-cytidine, and cytidine as the only uridines and cytidines. In some embodiments, the polynucleotides of the invention contain 1-methyl-pseudouridine, uridine, 5-phenyl-cytidine, and cytidine as the only uridines and cytidines. In some embodiments, the polynucleotides of the invention contain 1-methyl-pseudouridine, uridine, 5-ethnyl-cytidine, and cytidine as the only uridines and cytidines. In some embodiments, the polynucleotides of the invention contain 1-methyl-pseudouridine, uridine, N4-methyl-cytidine, and cytidine as the only uridines and cytidines. In some embodiments, the polynucleotides of the invention contain 1-methyl-pseudouridine, uridine, 5-fluoro-cytidine, and cytidine as the only uridines and cytidines. In some embodiments, the polynucleotides of the invention contain 1-methyl-pseudouridine, uridine, N4-acetyl-cytidine, and cytidine as the only uridines and cytidines. In some embodiments, the polynucleotides of the invention contain 1-methyl-pseudouridine, uridine, pseudoisocytidine, and cytidine as the only uridines and cytidines. In some embodiments, the polynucleotides of the invention contain 1-methyl-pseudouridine, uridine, 5-formyl-cytidine, and cytidine as the only uridines and cytidines. In some embodiments, the polynucleotides of the invention contain 1-methyl-pseudouridine, uridine, 5-aminoallyl-cytidine, and cytidine as the only uridines and cytidines. In some embodiments, the polynucleotides of the invention contain 1-methyl-pseudouridine, uridine, 5-carboxy-cytidine, and cytidine as the only uridines and cytidines.

In some embodiments, the polynucleotides of the invention contain the uracil of one of the nucleosides of Table 2 and uracil as the only uracils. In other embodiments, the polynucleotides of the invention contain a uridine of Table 2 and uridine as the only uridines.

TABLE 2
Exemplary uracil-containing nucleosides
Nucleoside Name

5-methoxy-uridine
1-Methyl-pseudo-uridine
Pseudouridine
5-methyl-uridine
5-bromo-uridine
2-thio-uridine
4-thiouridine
2′-O-methyluridine
5-methyl-2-thiouridine
5,2′-O-dimethyluridine
5-aminomethyl-2-thiouridine
5-(1-Propynyl)ara-uridine
2′-O-Methyl-5-(1-propynyl)uridine
5-Vinylarauridine
(Z)-5-(2-Bromo-vinyl)ara-uridine
(E)-5-(2-Bromo-vinyl)ara-uridine
(Z)-5-(2-Bromo-vinyl)uridine
(E)-5-(2-Bromo-vinyl)uridine
5-Cyanouridine
5-Formyluridine
5-Dimethylaminouridine
5-Trideuteromethyl-6-deuterouridine
5-(2-Furanyl)uridine
5-Phenylethynyluridine
4′-Carbocyclic uridine
4′-Ethynyluridine
4′-Azidouridine
2′-Deoxy-2′,2′-difluorouridine
2′-Deoxy-2′-b-fluorouridine
2′-Deoxy-2′-b-chlorouridine
2′-Deoxy-2′-b-bromouridine
2′-Deoxy-2′-b-iodouridine
5′-Homo-uridine
2′-Deoxy-2′-a-mercaptouridine
2′-Deoxy-2′-a-thiomethoxyuridine
2′-Deoxy-2′-a-azidouridine
2′-Deoxy-2′-a-aminouridine
2′-Deoxy-2′-b-mercaptouridine
2′-Deoxy-2′-b-thiomethoxyuridine
2′-Deoxy-2′-b-azidouridine
2′-Deoxy-2′-b-aminouridine
2′-b-Trifluoromethyluridine
2′-a-Trifluoromethyluridine
2′-b-Ethynyluridine
2′-a-Ethynyluridine
1-ethyl-pseudo-uridine
1-propyl-pseudo-uridine
1-iso-propyl-pseudo-uridine
1-(2,2,2-trifluoroethyl)-pseudo-uridine
1-cyclopropyl-pseudo-uridine
1-cyclopropylmethyl-pseudo-uridine
1-phenyl-pseudo-uridine
1-benzyl-pseudo-uridine
1-aminomethyl-pseudo-uridine
pseudo-uridine-1-2-ethanoic acid
1-(3-amino-3-carboxypropyl)pseudo-uridine
1-methyl-3-(3-amino-3-carboxypropyl)pseudo-uridine
6-methyl-pseudo-uridine
6-trifluoromethyl-pseudo-uridine
6-methoxy-pseudo-uridine
6-phenyl-pseudo-uridine
6-iodo-pseudo-uridine
6-bromo-pseudo-uridine
6-chloro-pseudo-uridine
6-fluoro-pseudo-uridine
4-Thio-pseudo-uridine
2-Thio-pseudo-uridine
Alpha-thio-pseudo-uridine
1-Me-alpha-thio-pseudo-uridine
1-butyl-pseudo-uridine
1-tert-butyl-pseudo-uridine
1-pentyl-pseudo-uridine
1-hexyl-pseudo-uridine
1-trifluoromethyl-pseudo-uridine
1-cyclobutyl-pseudo-uridine
1-cyclopentyl-pseudo-uridine
1-cyclohexyl-pseudo-uridine
1-cycloheptyl-pseudo-uridine
1-cyclooctyl-pseudo-uridine
1-cyclobutylmethyl-pseudo-uridine
1-cyclopentylmethyl-pseudo-uridine
1-cyclohexylmethyl-pseudo-uridine
1-cycloheptylmethyl-pseudo-uridine
1-cyclooctylmethyl-pseudo-uridine
1-p-tolyl-pseudo-uridine
1-(2,4,6-trimethyl-phenyl)pseudo-uridine
1-(4-methoxy-phenyl)pseudo-uridine
1-(4-amino-phenyl)pseudo-uridine
1(4-nitro-phenyl)pseudo-uridine
pseudo-uridine-N1-p-benzoic acid
1-(4-methyl-benzyl)pseudo-uridine
1-(2,4,6-trimethyl-benzyl)pseudo-uridine
1-(4-methoxy-benzyl)pseudo-uridine
1-(4-amino-benzyl)pseudo-uridine
1-(4-nitro-benzyl)pseudo-uridine
pseudo-uridine-N1-methyl-p-benzoic acid
1-(2-amino-ethyl)pseudo-uridine
1-(3-amino-propyl)pseudo-uridine
1-(4-amino-butyl)pseudo-uridine
1-(5-amino-pentyl)pseudo-uridine
1-(6-amino-hexyl)pseudo-uridine
pseudo-uridine-N1-3-propionic acid
pseudo-uridine-N1-4-butanoic acid
pseudo-uridine-N1-5-pentanoic acid
pseudo-uridine-N1-6-hexanoic acid
pseudo-uridine-N1-7-heptanoic acid
1-(2-amino-2-carboxyethyl)pseudo-uridine
1-(4-amino-4-carboxybutyl)pseudo-uridine
3-alkyl-pseudo-uridine
6-ethyl-pseudo-uridine
6-propyl-pseudo-uridine
6-iso-propyl-pseudo-uridine
6-butyl-pseudo-uridine
6-tert-butyl-pseudo-uridine
6-(2,2,2-trifluoroethyl)-pseudo-uridine
6-ethoxy-pseudo-uridine
6-trifluoromethoxy-pseudo-uridine
6-phenyl-pseudo-uridine
6-(substituted-phenyl)-pseudo-uridine
6-cyano-pseudo-uridine
6-azido-pseudo-uridine
6-amino-pseudo-uridine
6-ethylcarboxylate-pseudo-uridine
6-hydroxy-pseudo-uridine
6-methylamino-pseudo-uridine
6-dimethylamino-pseudo-uridine
6-hydroxyamino-pseudo-uridine
6-formyl-pseudo-uridine
6-(4-morpholino)-pseudo-uridine
6-(4-thiomorpholino)-pseudo-uridine
1-me-4-thio-pseudo-uridine
1-me-2-thio-pseudo-uridine
1,6-dimethyl-pseudo-uridine
1-methyl-6-trifluoromethyl-pseudo-uridine
1-methyl-6-ethyl-pseudo-uridine
1-methyl-6-propyl-pseudo-uridine
1-methyl-6-iso-propyl-pseudo-uridine
1-methyl-6-butyl-pseudo-uridine
1-methyl-6-tert-butyl-pseudo-uridine
1-methyl-6-(2,2,2-trifluoroethyl)pseudo-uridine
1-methyl-6-iodo-pseudo-uridine
1-methyl-6-bromo-pseudo-uridine
1-methyl-6-chloro-pseudo-uridine
1-methyl-6-fluoro-pseudo-uridine
1-methyl-6-methoxy-pseudo-uridine
1-methyl-6-ethoxy-pseudo-uridine
1-methyl-6-trifluoromethoxy-pseudo-uridine
1-methyl-6-phenyl-pseudo-uridine
1-methyl-6-(substituted phenyl)pseudo-uridine
1-methyl-6-cyano-pseudo-uridine
1-methyl-6-azido-pseudo-uridine
1-methyl-6-amino-pseudo-uridine
1-methyl-6-ethylcarboxylate-pseudo-uridine
1-methyl-6-hydroxy-pseudo-uridine
1-methyl-6-methylamino-pseudo-uridine
1-methyl-6-dimethylamino-pseudo-uridine
1-methyl-6-hydroxyamino-pseudo-uridine
1-methyl-6-formyl-pseudo-uridine
1-methyl-6-(4-morpholino)-pseudo-uridine
1-methyl-6-(4-thiomorpholino)-pseudo-uridine
1-alkyl-6-vinyl-pseudo-uridine
1-alkyl-6-allyl-pseudo-uridine
1-alkyl-6-homoallyl-pseudo-uridine
1-alkyl-6-ethynyl-pseudo-uridine
1-alkyl-6-(2-propynyl)-pseudo-uridine
1-alkyl-6-(1-propynyl)-pseudo-uridine
1-Hydroxymethylpseudouridine
1-(2-Hydroxyethyl)pseudouridine
1-Methoxymethylpseudouridine
1-(2-Methoxyethyl)pseudouridine
1-(2,2-Diethoxyethyl)pseudouridine
(±)1-(2-Hydroxypropyl)pseudouridine
(2R)-1-(2-Hydroxypropyl)pseudouridine
(2S)-1-(2-Hydroxypropyl)pseudouridine
1-Cyanomethylpseudouridine
1-Morpholinomethylpseudouridine
1-Thiomorpholinomethylpseudouridine
1-Benzyloxymethylpseudouridine
1-(2,2,3,3,3-Pentafluoropropyl)pseudouridine
1-Thiomethoxymethylpseudouridine
1-Methanesulfonylmethylpseudouridine
1-Vinylpseudouridine
1-Allylpseudouridine
1-Homoallylpseudouridine
1-Propargylpseudouridine
1-(4-Fluorobenzyl)pseudouridine
1-(4-Chlorobenzyl)pseudouridine
1-(4-Bromobenzyl)pseudouridine
1-(4-Iodobenzyl)pseudouridine
1-(4-Methylbenzyl)pseudouridine
1-(4-Trifluoromethylbenzyl)pseudouridine
1-(4-Methoxybenzyl)pseudouridine
1-(4-Trifluoromethoxybenzyl)pseudouridine
1-(4-Thiomethoxybenzyl)pseudouridine
1-(4-Methanesulfonylbenzyl)pseudouridine
Pseudouridine 1-(4-methylbenzoic acid)
Pseudouridine 1-(4-methylbenzenesulfonic acid)
1-(2,4,6-Trimethylbenzyl)pseudouridine
1-(4-Nitrobenzyl)pseudouridine
1-(4-Azidobenzyl)pseudouridine
1-(3,4-Dimethoxybenzyl)pseudouridine
1-(3,4-Bis-trifluoromethoxybenzyl)pseudouridine
1-Acetylpseudouridine
1-Trifluoroacetylpseudouridine
1-Benzoylpseudouridine
1-Pivaloylpseudouridine
1-(3-Cyclopropyl-prop-2-ynyl)pseudouridine
Pseudouridine 1-methylphosphonic acid diethyl ester
Pseudouridine 1-methylphosphonic acid
Pseudouridine 1-[3-(2-ethoxy)]propionic acid
Pseudouridine 1-[3-{2-(2-ethoxy)-ethoxy}] propionic acid
Pseudouridine TP 1-[3-{2-(2-[2-ethoxy]-ethoxy)-ethoxy}]propionic acid
Pseudouridine 1-[3-{2-(2-[2-(2-ethoxy)-ethoxy]-ethoxy)-ethoxy}]propionic
acid
Pseudouridine 1-[3-{2-(2-[2-{2(2-ethoxy)-ethoxy}-ethoxy]-ethoxy)-
ethoxy}]propionic acid
1-{3-[2-(2-Aminoethoxy)-ethoxy]-propionyl} pseudouridine
1-[3-(2-{2-[2-(2-Aminoethoxy)-ethoxy]-ethoxy}-ethoxy)-
propionyl]pseudouridine
1-Biotinylpseudouridine
1-Biotinyl-PEG2-pseudouridine
5-Oxyacetic acid-methyl ester-uridine
3-Methyl-pseudo-uridine
5-trifluoromethyl-uridine
5-methyl-amino-methyl-uridine
5-carboxy-methyl-amino-methyl-uridine
5-carboxymethylaminomethyl-2′-OMe-uridine
5-carboxymethylaminomethyl-2-thio-uridine
5-methylaminomethyl-2-thio-uridine
5-methoxy-carbonyl-methyl-uridine
5-methoxy-carbonyl-methyl-2′-OMe-uridine
5-oxyacetic acid-uridine
3-(3-amino-3-carboxypropyl)-uridine
5-(carboxyhydroxymethyl)uridine methyl ester
5-(carboxyhydroxymethyl)uridine
2′-OMe-pseudo-uridine
2′-Azido-2′-deoxy-uridine
2′-Amino-2′-deoxy-uridine
2′-F-5-Methyl-2′-deoxy-uridine
5-iodo-2′-fluoro-deoxyuridine
2′-bromo-deoxyuridine
2,2′-anhydro-uridine
2′-Azido-deoxyuridine
5-Methoxycarbonylmethyl-2-thiouridine
5-Methylaminomethyl-2-thiouridine
5-Carbamoylmethyluridine
5-Carbamoylmethyl-2′-O-methyluridine
1-Methyl-3-(3-amino-3-carboxypropyl) pseudouridine
5-Methylaminomethyl-2-selenouridine
5-Carboxymethyluridine
5-Methyldihydrouridine
5-Taurinomethyluridine
5-Taurinomethyl-2-thiouridine
5-(iso-Pentenylaminomethyl)uridine
5-(iso-Pentenylaminomethyl)-2-thiouridine
5-(iso-Pentenylaminomethyl)-2′-O-methyluridine
2′-O-Methylpseudouridine
2-Thio-2′-O-methyluridine
3,2′-O-Dimethyluridine
5-Methoxy-carbonylmethyl-uridine
5-hydroxy-uridine
5-Isopentenyl-aminomethyl-uridine

In some embodiments, the polynucleotides ofthe invention contain the cytosine ofone ofthe nucleosides ofTable 3 and cytosine as the only cytosines. In other embodiments, the polynucleotides ofthe invention contain a cytidine ofTable 3 and cytidine as the only cytidines.

TABLE 3
Exemplary cytosine containing nucleosides
Nucleoside Name

α-thio-cytidine
Pseudoisocytidine
pyrrolo-cytidine
5-methyl-cytidine
N4-acetyl-cytidine
5-Bromo-cytidine
5-Trifluoromethyl-cytidine
5-Hydroxymethyl-cytidine
5-Iodo-cytidine
5-Ethyl-cytidine
5-Methoxy-cytidine
5-Ethynyl-cytidine
5-Fluoro-cytidine
5-Phenyl-cytidine
N4-Bz-cytidine
N4-Methyl-cytidine
5-Pseudo-iso-cytidine
5-Formyl-cytidine
5-Aminoallyl-cytidine
2′-O-methylcytidine
2′-O-Methyl-5-(1-propynyl)cytidine
5-(1-Propynyl)ara-cytidine
5-Ethynylara-cytidine
5-Ethynylcytidine
5-Cyanocytidine
5-(2-Chloro-phenyl)-2-thiocytidine
5-(4-Amino-phenyl)-2-thiocytidine
N4,2′-O-Dimethylcytidine
3′-Ethynylcytidine
4′-Carbocyclic cytidine
4′-Ethynylcytidine
4′-Azidocytidine
2′-Deoxy-2′,2′-difluorocytidine
2′-Deoxy-2′-b-fluorocytidine
2′-Deoxy-2′-b-chlorocytidine
2′-Deoxy-2′-b-bromocytidine
2′-Deoxy-2′-b-iodocytidine
5′-Homo-cytidine
2′-Deoxy-2′-a-mercaptocytidine
2′-Deoxy-2′-a-thiomethoxycytidine
2′-Deoxy-2′-a-azidocytidine
2′-Deoxy-2′-a-aminocytidine
2′-Deoxy-2′-b-mercaptocytidine
2′-Deoxy-2′-b-thiomethoxycytidine
2′-Deoxy-2′-b-azidocytidine
2′-Deoxy-2′-b-aminocytidine
2′-b-Trifluoromethylcytidine
2′-a-Trifluoromethylcytidine
2′-b-Ethynylcytidine
2′-a-Ethynylcytidine
(E)-5-(2-Bromo-vinyl)cytidine
2′-Azido-2′-deoxy-cytidine
2′-Amino-2′-deoxy-cytidine
5-aminoallyl-cytidine
2,2′-anhydro-cytidine
N4-amino-cytidine
2′-O-Methyl-N4-acetyl-cytidine
2′-fluoro-N4-acetyl-cytidine
2′-fluor-N4-Bz-cytidine
2′-O-methyl-N4-Bz-cytidine
N4,2′-O-Dimethylcytidine
5-Formyl-2′-O-methylcytidine

### Alterations on the Internucleoside Linkage

The alternative nucleotides, which may be incorporated into a polynucleotide molecule, can be altered on the internucleoside linkage (e.g., phosphate backbone). Herein, in the context of the polynucleotide backbone, the phrases “phosphate” and “phosphodiester” are used interchangeably. Backbone phosphate groups can be altered by replacing one or more of the oxygen atoms with a different substituent.

The alternative nucleosides and nucleotides can include the wholesale replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl and/or methoxy. As a non-limiting example, two non-bridging oxygens at the same position (e.g., the alpha (α), beta (β) or gamma (γ) position) can be replaced with a sulfur (thio) and a methoxy.

The replacement of one or more of the oxygen atoms at the a position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment. While not wishing to be bound by theory, phosphorothioate linked polynucleotide molecules are expected to also reduce the innate immune response through weaker binding/activation of cellular innate immune molecules.

In specific embodiments, an alternative nucleoside includes an alpha-thio-nucleoside (e.g., 5′-O-(1-thiophosphate)-adenosine, 5′-O-(1-thiophosphate)-cytidine (α-thio-cytidine). 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein below.

### Combinations of Alternative Sugars, Nucleobases, and Internucleoside Linkages

The polynucleotides of the invention can include a combination of alterations to the sugar, the nucleobase, and/or the internucleoside linkage. These combinations can include any one or more alterations described herein.

### Synthesis of Polynucleotides

The polynucleotide molecules for use in accordance with the invention may be prepared according to any useful technique, as described herein. The alternative nucleosides and nucleotides used in the synthesis of polynucleotide molecules disclosed herein can be prepared from readily available starting materials using the following general methods and procedures. Where typical or preferred process conditions (e.g., reaction temperatures, times, mole ratios of reactants, solvents, pressures, etc.) are provided, a skilled artisan would be able to optimize and develop additional process conditions. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography (e.g., high performance liquid chromatography (HPLC) or thin layer chromatography).

Preparation of polynucleotide molecules of the present invention can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, (1991), which is incorporated herein by reference in its entirety.

The reactions of the processes described herein can be carried out in suitable solvents, which can be readily selected by one of skill in the art of organic synthesis. Suitable solvents can be substantially nonreactive with the starting materials (reactants), the intermediates, or products at the temperatures at which the reactions are carried out (i.e., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected.

Resolution of racemic mixtures of alternative polynucleotides or nucleic acids (e.g., polynucleotides or mRNA molecules) can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

Alternative nucleosides and nucleotides (e.g., building block molecules) can be prepared according to the synthetic methods described in Ogata et al., J. Org. Chem. 74:2585-2588 (2009); Purmal et al., Nucl. Acids Res. 22(1): 72-78 (1994); Fukuhara et al., Biochemistry, 1(4): 563-568 (1962); and Xu et al., Tetrahedron, 48(9): 1729-1740 (1992), each of which are incorporated by reference in their entirety.

If the polynucleotide includes one or more alternative nucleosides or nucleotides, the polynucleotides of the invention may or may not be uniformly altered along the entire length of the molecule. For example, one or more or all types of nucleotide (e.g., purine or pyrimidine, or any one or more or all of A. G, U. C) may or may not be uniformly altered in a polynucleotide of the invention, or in a given predetermined sequence region thereof. In some embodiments, all nucleotides X in a polynucleotide of the invention (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations, nucleotide alterations, and/or internucleoside linkages (e.g., backbone structures) may exist at various positions in the polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′ or 3′ terminal alteration. The polynucleotide may contain from 1% to 100% alternative nucleosides, nucleotides, or internucleoside linkages (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100. In some embodiments, the remaining percentage is accounted for by the presence of A. G, U. or C.

When referring to percentage incorporation by an alternative nucleoside, nucleotide, or internucleoside linkage, in some embodiments the remaining percentage necessary to total 100% is accounted for by the corresponding natural nucleoside, nucleotide, or internucleoside linkage. In other embodiments, the remaining percentage necessary to total 100% is accounted for by a second alternative nucleoside, nucleotide, or internucleoside linkage.

### Messenger RNA

The present invention features compositions including one or more mRNAs, where each mRNA encodes a polypeptide (e.g., an anti-CD38, anti-BCMA, anti-GPRC5D, anti-FcRH5 antibody or antigen-binding fragment described herein or a chimeric molecule comprising such an antigen binding fragment, e.g., a multispecific molecule comprising a VHH domain of the invention). Exemplary mRNAs of the disclosure each include (i) a 5′-cap structure; (ii) a 5′-UTR; (iii) an open reading frame encoding the polypeptide; (iv) a 3′-untranslated region (3′-UTR); and (v) a poly-A region.

In some embodiments, the mRNA includes from about 30 to about 3,000 (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, or from 2,500 to 3,000) nucleotides.

mRNA: 5-cap

The 5′-cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Alterations to the nucleic acids of the present invention may generate a non-hydrolyzable cap structure preventing decapping and thus increasing mRNA half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with α-thio-guanosine nucleotides according to the manufacturer's instructions to create a phosphorothioate linkage in the 5′-ppp-5′ cap. Additional alternative guanosine nucleotides may be used such as α-methyl-phosphonate and seleno-phosphate nucleotides.

Additional alterations include, but are not limited to. 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

5′-cap structures include those described in International Patent Publication Nos. WO2008/127688, WO2008/016473, and WO2011/015347, each of which is incorporated herein by reference in its entirety.

Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs may be chemically (i.e., non-enzymatically) or enzymatically synthesized and/linked to a nucleic acid molecule.

For example, the Anti-Reverse Cap Analog (ARCA) cap contains two guanosines linked by a 5′-5′-triphosphate group, wherein one guanosine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G)). The 3′-O atom of the other, unaltered, guanosine becomes linked to the 5′-terminal nucleotide of the capped nucleic acid molecule (e.g., an mRNA or mmRNA). The N7- and 3′-O-methlyated guanosine provides the terminal moiety of the capped nucleic acid molecule (e.g., mRNA or mmRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In some embodiments, the cap is a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the contents of which are herein incorporated by reference in its entirety.

In some embodiments, the cap analog is a N7-(4-chlorophenoxyethyl) substituted dicnucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O}G(5′)ppp(5′)G cap analog (see, e.g., the various cap analogs and the methods of synthesizing cap analogs described in Kore et al. Bioorganic & Medicinal Chemistry 21:4570-4574 (2013); the contents of which are herein incorporated by reference in its entirety). In some embodiments, a cap analog of the present invention is a 4-chloro/bromophenoxyethyl analog.

While cap analogs allow for the concomitant capping of a nucleic acid molecule in an in vitro transcription reaction, up to 20% of transcripts remain uncapped. This, as well as the structural differences of a cap analog from endogenous 5′-cap structures of nucleic acids produced by the endogenous, cellular transcription machinery, may lead to reduced translational competency and reduced cellular stability.

Nucleic acids of the invention (e.g., mRNAs of the invention) may also be capped post-transcriptionally, using enzymes. 5′ cap structures produced by enzymatic capping may enhance binding of cap binding proteins, increase half-life, reduce susceptibility to 5′ endonucleases and/or reduce 5′ decapping, as compared to synthetic 5′-cap structures known in the art (or to a wild-type, natural or physiological 5′-cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanosine cap nucleotide wherein the cap guanosine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include 7mG(5′)ppp(5′)N,pN2p (cap 0), 7mG(5′)ppp(5′)NlmpNp (cap 1), 7mG(5′)-ppp(5′)NlmpN2mp (cap 2), and m(7)Gpppm(3)(6,6,2′)Apm(2′)Apm(2′)Cpm(2)(3,2′)Up (cap 4).

According to the present invention, 5′ terminal caps may include endogenous caps or cap analogs. According to the present invention, a 5′ terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the nucleic acids described herein may contain a modified 5′-cap. A modification on the 5′-cap may increase the stability of mRNA, increase the half-life of the mRNA, and could increase the mRNA translational efficiency. The modified 5′-cap may include, but is not limited to, one or more of the following modifications: modification at the 2′ and/or 3′ position of a capped guanosine triphosphate (GTP), a replacement of the sugar ring oxygen (that produced the carbocyclic ring) with a methylene moiety (CH₂), a modification at the triphosphate bridge moiety of the cap structure, or a modification at the nucleobase (G) moiety.

mRNA: Coding Region

Provided are nucleic acids that encode antibodies or antigen-binding fragments of the disclosure. As recognized by those skilled in the art, protein fragments, functional protein domains, and homologous proteins are also considered to be within the scope of this present disclosure. For example, provided herein is any protein fragment of a reference protein (meaning a polypeptide sequence at least one amino acid residue shorter than a reference polypeptide sequence but otherwise identical) 10, 20, 30, 40, 50, 60, 70, 80, 90, 100, or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the present disclosure. In certain embodiments, a protein sequence to be utilized in accordance with the present disclosure includes 2, 3, 4, 5, 6, 7, 8, 9, 10, or more mutations as shown in any of the sequences provided or referenced herein.

mRNA: Poly-A Tail

During RNA processing, a long chain of adenosine nucleotides (poly(A) tail) is normally added to mRNA molecules to increase the stability of the mRNA. Immediately after transcription, the 3′ end of the transcript is cleaved to free a 3′ hydroxyl. Then poly(A) polymerase adds a chain of adenosine nucleotides to the RNA. The process, called polyadenylation, adds a poly-A tail that is between 100 and 250 residues long (SEQ ID NO: 494).

Methods for the stabilization of RNA by incorporation of chain-terminating nucleosides at the 3′-terminus include those described in International Patent Publication No. WO2013/103659, incorporated herein in its entirety.

Poly(A) tail deadenylation by 3′ exonucleases is a key step in cellular mRNA degradation in eukaryotes. By blocking 3′ exonucleases, the functional half-life of mRNA can be increased, resulting in increased protein expression. Chemical and enzymatic ligation strategies to modify the 3′ end of mRNA with reverse chirality adenosine (LA10) and/or inverted deoxythymidine (IdT) are known to those of skill in the art and have been demonstrated to extend mRNA half-life in cellular and in vivo studies. In some embodiments, the poly(A)tail of the mRNA includes a 3′ LA10 or IdT modification. For example, as described in International Patent Publication No. WO2017/049275, the tail modifications of which are incorporated by reference in their entirety.

Additional strategies have been explored to further stabilize mRNA, including: chemical modification of the 3′ nucleotide (e.g., conjugation of a morpholino to the 3′ end of the poly(A)tail); incorporation of stabilizing sequences after the poly(A) tail (e.g., a co-polymer, a stem-loop, or a triple helix); and/or annealing of structured oligos to the 3′ end of an mRNA, as described, for example, in International Patent Publication No. WO2017/049286, the stabilized linkages of which are incorporated by reference in their entirety.

Annealing an oligonucleotide (e.g., an oligonucleotide conjugate) with a complex secondary structure (e.g., a triple-helix structure or a stem-loop structure) at the 3′end may provide nuclease resistance and increase half-life of mRNA.

Unique poly(A) tail lengths may provide certain advantages to the RNAs of the present invention. Generally, the length of a poly(A) tail of the present invention is greater than 30 nucleotides in length. In some embodiments, the poly(A) tail is greater than 35 nucleotides in length. In some embodiments, the length is at least 40 nucleotides. In another embodiment, the length is at least 45 nucleotides. In some embodiments, the length is at least 50 nucleotides. In some embodiments, the length is at least 55 nucleotides. In another embodiment, the length is at least 60 nucleotides. In another embodiment, the length is at least 65 nucleotides. In another embodiment, the length is at least 70 nucleotides. In some embodiments, the length is at least 80 nucleotides. In some embodiments, the length is at least 90 nucleotides. In some embodiments, the length is at least 100 nucleotides. In some embodiments, the length is at least 120 nucleotides. In some embodiments, the length is at least 140 nucleotides. In some embodiments, the length is at least 160 nucleotides. In some embodiments, the length is at least 180 nucleotides. In some embodiments, the length is at least 200 nucleotides. In some embodiments, the length is at least 250 nucleotides. In some embodiments, the length is at least 300 nucleotides. In some embodiments, the length is at least 350 nucleotides. In some embodiments, the length is at least 400 nucleotides. In some embodiments, the length is at least 450 nucleotides. In some embodiments, the length is at least 500 nucleotides. In some embodiments, the length is at least 600 nucleotides. In some embodiments, the length is at least 700 nucleotides. In some embodiments, the length is at least 800 nucleotides. In some embodiments, the length is at least 900 nucleotides. In some embodiments, the length is at least 1000 nucleotides. In some embodiments, the length is at least 1100 nucleotides. In some embodiments, the length is at least 1200 nucleotides. In some embodiments, the length is at least 1300 nucleotides. In some embodiments, the length is at least 1400 nucleotides. In some embodiments, the length is at least 1500 nucleotides. In some embodiments, the length is at least 1600 nucleotides. In some embodiments, the length is at least 1700 nucleotides. In some embodiments, the length is at least 1800 nucleotides. In some embodiments, the length is at least 1900 nucleotides. In some embodiments, the length is at least 2000 nucleotides. In some embodiments, the length is at least 2500 nucleotides. In some embodiments, the length is at least 3000 nucleotides.

In some embodiments, the poly(A) tail may be 80 nucleotides, 120 nucleotides, or 160 nucleotides in length. In some embodiments, the poly(A) tail may be 20, 40, 80, 100, 120, 140 or 160 nucleotides in length.

In some embodiments, the poly(A) tail is designed relative to the length of the mRNA. This design may be based on the length of the coding region of the mRNA, the length of a particular feature or region of the mRNA, or based on the length of the ultimate product expressed from the RNA. When relative to any additional feature of the RNA (e.g., other than the mRNA portion which includes the poly(A) tail), poly(A) tail may be 10, 20, 30, 40, 50, 60, 70, 80, 90 or 100% greater in length than the additional feature. The poly(A) tail may also be designed as a fraction of the mRNA to which it belongs. In this context, the poly(A) tail may be 10, 20, 30, 40, 50, 60, 70, 80, or 90% or more of the total length of the construct or the total length of the construct minus the poly(A) tail.

In some embodiments, engineered binding sites and/or the conjugation of nucleic acids or mRNA for poly(A) binding protein may be used to enhance expression. The engineered binding sites may be sensor sequences which can operate as binding sites for ligands of the local microenvironment of the nucleic acids and/or mRNA. As a non-limiting example, the nucleic acids and/or mRNA may include at least one engineered binding site to alter the binding affinity of poly(A) binding protein (PABP) and analogs thereof. The incorporation of at least one engineered binding site may increase the binding affinity of the PABP and analogs thereof.

Additionally, multiple distinct nucleic acids or mRNA may be linked together to the PABP (poly(A) binding protein) through the 3′-end using nucleotides at the 3′-terminus of the poly(A) tail. Transfection experiments can be conducted in relevant cell lines and protein production can be assayed by ELISA at 12 hr, 24 hr, 48 hr, 72 hr, and day 7 post-transfection. As a non-limiting example, the transfection experiments may be used to evaluate the effect on PABP or analogs thereof binding affinity as a result of the addition of at least one engineered binding site.

In some embodiments, a poly(A) tail may be used to modulate translation initiation. While not wishing to be bound by theory, the poly-A tail recruits PABP which in turn can interact with translation initiation complex and thus may be essential for protein synthesis.

In some embodiments, a poly(A) tail may also be used in the present invention to protect against 3′-5′ exonuclease digestion.

In some embodiments, the nucleic acids or mRNA of the present invention are designed to include a poly-A-G Quartet. The G-quartet is a cyclic hydrogen bonded array of four guanosine nucleotides that can be formed by G-rich sequences in both DNA and RNA. In this embodiment, the G-quartet is incorporated at the end of the poly-A tail. The resultant nucleic acid or mRNA may be assayed for stability, protein production and other parameters including half-life at various time points. It has been discovered that the poly-A-G quartet results in protein production equivalent to at least 75% of that seen using a poly-A tail of 120 nucleotides alone (SEQ ID NO: 495).

In some embodiments, the nucleic acids or mRNA of the present invention may include a poly(A) tail and may be stabilized by the addition of a chain terminating nucleoside. The nucleic acids and/or mRNA with a poly(A) tail may further include a 5′cap structure.

In some embodiments, the nucleic acids or mRNA of the present invention may include a poly-A-G Quartet. The nucleic acids and/or mRNA with a poly-A-G Quartet may further include a 5′cap structure.

In some embodiments, the chain terminating nucleoside which may be used to stabilize the nucleic acid or mRNA including a poly(A) tail or poly-A-G Quartet may be, but is not limited to, those described in International Patent Publication No. WO2013103659, incorporated herein by reference in its entirety. In some embodiments, the chain terminating nucleosides which may be used with the present invention includes, but is not limited to, 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or a —O— methylnucleoside.

In some embodiments, the mRNA which includes a poly(A) tail or a poly-A-G Quartet may be stabilized by an alteration to the 3′region of the nucleic acid that can prevent and/or inhibit the addition of oligo(U) (see, e.g., International Patent Publication No. WO2013/103659, incorporated herein by reference in its entirety).

In yet another embodiment, the mRNA, which includes a poly(A) tail or a poly-A-G Quartet, may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

mRNA: Stem-Loops

In some embodiments, the nucleic acids of the present invention (e.g., the mRNA of the present invention) may include a stem-loop such as, but not limited to, a histone stem-loop. The stem-loop may be a nucleotide sequence that is about 25 or about 26 nucleotides in length such as, but not limited to, SEQ ID NOs: 7-17 as described in International Patent Publication No. WO2013/103659, incorporated herein by reference in its entirety. The histone stem-loop may be located 3′ relative to the coding region (e.g., at the 3′ terminus of the coding region). As a non-limiting example, the stem-loop may be located at the 3′ end of a nucleic acid described herein.

In some embodiments, the stem-loop may be located in the second terminal region. As a non-limiting example, the stem-loop may be located within an untranslated region (e.g., 3′-UTR) in the second terminal region.

In some embodiments, the nucleic acid such as, but not limited to mRNA, which includes the histone stem-loop may be stabilized by the addition of at least one chain terminating nucleoside. Not wishing to be bound by theory, the addition of at least one chain terminating nucleoside may slow the degradation of a nucleic acid and thus can increase the half-life of the nucleic acid.

In some embodiments, the chain terminating nucleoside may be, but is not limited to, those described in International Patent Publication No. WO2013/103659, incorporated herein by reference in its entirety. In some embodiments, the chain terminating nucleosides which may be used with the present invention includes, but is not limited to, 3′-deoxyadenosine (cordycepin), 3′-deoxyuridine, 3′-deoxycytosine, 3′-deoxyguanosine, 3′-deoxythymine. 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2-deoxynucleoside, or a —O— methylnucleoside.

In some embodiments, the nucleic acid such as, but not limited to mRNA, which includes the histone stem-loop may be stabilized by an alteration to the 3′region of the nucleic acid that can prevent and/or inhibit the addition of oligo(U) (see, e.g., International Patent Publication No. WO2013/103659, incorporated herein by reference in its entirety).

In yet another embodiment, the nucleic acid such as, but not limited to, mRNA, which includes the histone stem-loop may be stabilized by the addition of an oligonucleotide that terminates in a 3′-deoxynucleoside. 2′,3′-dideoxynucleoside 3′-O-methylnucleosides, 3′-O-ethylnucleosides, 3′-arabinosides, and other alternative nucleosides known in the art and/or described herein.

In some embodiments, the nucleic acids of the present invention may include a histone stem-loop, a poly(A) tail sequence, and/or a 5′-cap structure. The histone stem-loop may be before and/or after the poly-A tail sequence. The nucleic acids including the histone stem-loop and a poly(A) tail sequence may include a chain terminating nucleoside described herein.

In some embodiments, the nucleic acids of the present invention may include a histone stem-loop and a 5′-cap structure. The 5′-cap structure may include, but is not limited to, those described herein and/or known in the art.

In some embodiments, the nucleic acids described herein may include at least one histone stem-loop and a poly(A) sequence or polyadenylation signal. Non-limiting examples of nucleic acid sequences encoding for at least one histone stem-loop and a poly-A sequence or a polyadenylation signal are described in International Patent Publication Nos. WO2013/120497, WO2013/120629, WO2013/120500, WO2013/120627, WO2013/120498. WO2013/120626, WO2013/120499 and WO2013/120628, the contents of each of which are incorporated herein by reference in their entirety. In some embodiments, the nucleic acid encoding for a histone stem-loop and a poly(A) sequence or a polyadenylation signal may code for a pathogen antigen or fragment thereof such as the nucleic acid sequences described in International Patent Publication Nos. WO2013/120499 and WO2013/120628, the contents of both of which are incorporated herein by reference in their entirety. In some embodiments, the nucleic acid encoding for a histone stem-loop and a poly(A) sequence or a polyadenylation signal may code for a therapeutic protein such as the nucleic acid sequences described in International Patent Publication Nos. WO2013/120497 and WO2013/120629, the contents of both of which are incorporated herein by reference in their entirety. In some embodiments, the nucleic acid encoding for a histone stem-loop and a poly(A) sequence or a polyadenylation signal may code for a tumor antigen or fragment thereof such as the nucleic acid sequences described in International Patent Publication Nos. WO2013/120500 and WO2013/120627, the contents of both of which are incorporated herein by reference in their entirety. In some embodiments, the nucleic acid encoding for a histone stem-loop and a poly(A) sequence or a polyadenylation signal may code for an autoimmune self-antigen such as the nucleic acid sequences described in International Patent Publication Nos. WO2013/120498 and WO2013/120626, the contents of both of which are incorporated herein by reference in their entirety.

mRNA: Triple Helices

In some embodiments, nucleic acids of the present invention (e.g., the mRNA of the present invention) may include a triple helix on the 3′ end of the nucleic acid. The 3′ end of the nucleic acids of the present invention may include a triple helix alone or in combination with a poly(A) tail.

In some embodiments, the nucleic acid of the present invention may include at least a first and a second U-rich region, a conserved stem-loop region between the first and second region and an A-rich region. The first and second U-rich region and the A-rich region may associate to form a triple helix on the 3′ end of the nucleic acid. This triple helix may stabilize the nucleic acid, enhance the translational efficiency of the nucleic acid and/or protect the 3′ end from degradation. Triple helices include, but are not limited to, the triple helix sequence of metastasis-associated lung adenocarcinoma transcript 1 (MALAT1), MEN-β and polyadenylated nuclear (PAN) RNA (see, Wilusz et al., Genes & Development 26:2392-2407 (2012); herein incorporated by reference in its entirety).

In some embodiments, the triple helix may be formed from the cleavage of a MALAT1 sequence prior to the cloverleaf structure. While not meaning to be bound by theory, MALAT1 is a long non-coding RNA which, when cleaved, forms a triple helix and a tRNA-like cloverleaf structure. The MALAT1 transcript then localizes to nuclear speckles and the tRNA-like cloverleaf localizes to the cytoplasm (Wilusz et al., Cell. 135(5): 919-932 (2008); incorporated herein by reference in its entirety).

As a non-limiting example, the terminal end of the nucleic acid of the present invention including the MALAT1 sequence can then form a triple helix structure, after RNaseP cleavage from the cloverleaf structure, which stabilizes the nucleic acid (Peart et al., WIREs RNA. 4(5):491-506 (2013): incorporated herein by reference in its entirety).

In some embodiments, the nucleic acids or mRNA described herein include a MALAT1 sequence. In some embodiments, the nucleic acids or mRNA may be polyadenylated. In yet another embodiment, the nucleic acids or mRNA is not polyadenylated but has an increased resistance to degradation compared to unaltered nucleic acids or mRNA.

In some embodiments, the nucleic acids of the present invention may include a MALAT1 sequence in the second flanking region (e.g., the 3′-UTR). As a non-limiting example, the MALAT1 sequence may be human or mouse.

mRNA: Translation Enhancer Elements (TEEs)

The term “translational enhancer element” or “translation enhancer element” (herein collectively referred to as “TEE”) refers to sequences that increase the amount of polypeptide or protein produced from an mRNA. TEEs are conserved elements in the UTR which can promote translational activity of a nucleic acid such as, but not limited to, cap-dependent or cap-independent translation. The conservation of these sequences has been previously shown by Pinek et al., Nucleic Acids Research. 41(16): 7625-7634 (2013); incorporated herein by reference in its entirety) across 14 species including humans.

In some embodiments, the 5′-UTR of the mRNA includes at least one TEE. The TEE may be located between the transcription promoter and the start codon. The mRNA with at least one TEE in the 5′-UTR may include a cap at the 5′-UTR. Further, at least one TEE may be located in the 5′-UTR of mRNA undergoing cap-dependent or cap-independent translation.

The TEEs known may be in the 5′-leader of the Gx homeodomain protein (Chappell et al., Proc. Natl. Acad. Sci. USA 101:9590-9594 (2004), incorporated herein by reference in their entirety).

In another non-limiting example, TEEs are disclosed as SEQ ID NOs: 1-35 in US Patent Publication No. US20090226470. SEQ ID NOs: 1-35 in US Patent Publication No. US20130177581, SEQ ID NOs: 1-35 in International Patent Publication No. WO2009075886, SEQ ID NOs: 1-5, and 7-645 in International Patent Publication No. WO2012009644, SEQ ID NO: 1 in International Patent Publication No. WO1999024595, SEQ ID NO: 1 in U.S. Pat. No. 6,310,197, and SEQ ID NO: 1 in U.S. Pat. No. 6,849,405, each of which is incorporated herein by reference in its entirety.

The TEE may be an internal ribosome entry site (IRES), HCV-IRES or an IRES element such as, but not limited to, those described in U.S. Pat. No. 7,468,275, US Patent Publication Nos. US20070048776 and US20110124100 and International Patent Publication Nos. WO2007025008 and WO2001055369, each of which is incorporated herein by reference in its entirety. The IRES elements may include, but are not limited to, the Gtx sequences (e.g., Gtx9-nt, Gtx8-nt, Gtx7-nt) described by Chappell et al. (Proc. Natl. Acad. Sci. USA 101:9590-9594 (2004)) and Zhou et al. (PNAS 102:6273-6278 (2005)) and in US Patent Publication Nos. US20070048776 and US20110124100 and International Patent Publication No. WO2007025008, each of which is incorporated herein by reference in its entirety.

Additional exemplary TEEs are disclosed in U.S. Pat. Nos. 6,310,197, 6,849,405, 7,456,273, 7,183,395: US Patent Publication Nos. US20090226470, US20070048776, US20110124100, US20090093049, US20130177581; International Patent Publication Nos. WO2009075886, WO2007025008, WO2012009644, WO2001055371 WO1999024595; and European Patent Publications Nos. EP2610341A1 and EP2610340A1; each of which is incorporated herein by reference in its entirety.

In some embodiments, the polynucleotides, primary constructs, alternative nucleic acids and/or mRNA may include at least one TEE that is described in International Patent Publication Nos. WO1999024595, WO2012009644, WO2009075886, WO2007025008, WO1999024595, European Patent Publication Nos. EP2610341A1 and EP2610340A1, U.S. Pat. Nos. 6,310,197, 6,849,405, 7,456,273, 7,183,395, US Patent Publication No. US20090226470. US20110124100, US20070048776, US20090093049, and US20130177581 each of which is incorporated herein by reference in its entirety. The TEE may be located in the 5′-UTR of the mRNA.

In some embodiments, the polynucleotides, primary constructs, alternative nucleic acids and/or mmRNA may include at least one TEE that has at least 50%, at least 55%, at least 60%, at least 65%, at least 70%, at least 75%, at least 80%, at least 85%, at least 90%, at least 95% or at least 99% identity with the TEEs described in US Patent Publication Nos. US20090226470, US20070048776, US20130177581 and US20110124100, International Patent Publication Nos. WO1999024595, WO2012009644, WO2009075886 and WO2007025008, European Patent Publication No. EP2610341A1 and EP2610340A1, and U.S. Pat. Nos. 6,310,197, 6,849,405, 7,456,273, and 7,183,395, each of which is incorporated herein by reference in its entirety.

Multiple copies of a specific TEE can be present in mRNA. The TEEs in the translational enhancer polynucleotides can be organized in one or more sequence segments. A sequence segment can harbor one or more of the specific TEEs exemplified herein, with each TEE being present in one or more copies. When multiple sequence segments are present in a translational enhancer polynucleotide, they can be homogenous or heterogeneous. Thus, the multiple sequence segments in a translational enhancer polynucleotide can harbor identical or different types of the specific TEEs exemplified herein, identical or different number of copies of each of the specific TEEs, and/or identical or different organization of the TEEs within each sequence segment.

In some embodiments, the 5′-UTR of the mRNA may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 5′-UTR of mRNA of the present invention may be the same or different TEE sequences. The TEE sequences may be in a pattern such as ABABAB or AABBAABBAABB or ABCABCABC or variants thereof repeated once, twice, or more than three times. In these patterns, each letter, A, B, or C represent a different TEE sequence at the nucleotide level.

In some embodiments, the 5′-UTR may include a spacer to separate two TEE sequences. As a non-limiting example, the spacer may be a 15 nucleotide spacer and/or other spacers known in the art. As another non-limiting example, the 5′-UTR may include a TEE sequence-spacer module repeated at least once, at least twice, at least 3 times, at least 4 times, at least 5 times, at least 6 times, at least 7 times, at least 8 times and at least 9 times, or more than 9 times in the 5′-UTR.

In some embodiments, the TEE in the 5′-UTR of the mRNA of the present invention may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in US Patent Publication Nos. US20090226470, US20070048776, US20130177581 and US20110124100, International Patent Publication Nos. WO1999024595. WO2012009644, WO2009075886 and WO2007025008, European Patent Publication Nos. EP2610341A1 and EP2610340A1, and U.S. Pat. Nos. 6,310,197, 6,849,405, 7,456,273, and 7,183,395 each of which is incorporated herein by reference in its entirety. In some embodiments, the TEE in the 5′-UTR of the mRNA of the present invention may include a 5-30 nucleotide fragment, a 5-25 nucleotide fragment, a 5-20 nucleotide fragment, a 5-15 nucleotide fragment, a 5-10 nucleotide fragment of the TEE sequences disclosed in US Patent Publication Nos. US20090226470, US20070048776, US20130177581, and US20110124100, International Patent Publication No. WO1999024595, WO2012009644, WO2009075886, and WO2007025008, European Patent Publication No. EP2610341A1 and EP2610340A1, and U.S. Pat. No. 6,310,197. U.S. Pat. Nos. 6,849,405, 7,456,273, and 7,183,395; each of which is incorporated herein by reference in its entirety.

In some embodiments, the TEE in the 5′-UTR of the mRNA of the present invention may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in Chappell et al. (Proc. Natl. Acad. Sci. USA 101:9590-9594 (2004)) and Zhou et al. (PNAS 102:6273-6278 (2005)), in Supplemental Table 1 and in Supplemental Table 2 disclosed by Wellensiek et al. (Nature Methods. 10(8):747-750 (2013)); each of which is herein incorporated by reference in its entirety. In some embodiments, the TEE in the 5′-UTR of the polynucleotides, primary constructs, alternative nucleic acids and/or mmRNA of the present invention may include a 5-30 nucleotide fragment, a 5-25 nucleotide fragment, a 5-20 nucleotide fragment, a 5-15 nucleotide fragment, a 5-10 nucleotide fragment of the TEE sequences disclosed in Chappell et al. (Proc. Natl. Acad. Sci. USA 101:9590-9594 (2004)) and Zhou et al. (PNAS 102:6273-6278 (2005)), in Supplemental Table 1 and in Supplemental Table 2 disclosed by Wellensiek et al. (Nature Methods. 10(8):747-750 (2013)); each of which is incorporated herein by reference in its entirety.

In some embodiments, the TEE used in the 5′-UTR of the mRNA of the present invention is an IRES sequence such as, but not limited to, those described in U.S. Pat. No. 7,468,275 and International Patent Publication No. WO2001055369, each of which is incorporated herein by reference in its entirety.

In some embodiments, the TEEs used in the 5′-UTR of the mRNA of the present invention may be identified by the methods described in US Patent Publication Nos. US20070048776 and US20110124100 and International Patent Publication Nos. WO2007025008 and WO2012009644, each of which is incorporated herein by reference in its entirety.

In some embodiments, the TEEs used in the 5′-UTR of the mRNA of the present invention may be a transcription regulatory element described in U.S. Pat. Nos. 7,456,273 and 7,183,395, US Patent Publication No. US20090093049, and International Publication No. WO2001055371, each of which is incorporated herein by reference in its entirety. The transcription regulatory elements may be identified by methods known in the art, such as, but not limited to, the methods described in U.S. Pat. Nos. 7,456,273 and 7,183,395, US Patent Publication No. US20090093049, and International Publication No. WO2001055371, each of which is incorporated herein by reference in its entirety.

In yet another embodiment, the TEE used in the 5′-UTR of the mRNA of the present invention is an oligonucleotide or portion thereof as described in U.S. Pat. Nos. 7,456,273 and 7,183,395, US Patent Publication No. US20090093049, and International Publication No. WO2001055371, each of which is incorporated herein by reference in its entirety.

The 5′-UTR including at least one TEE described herein may be incorporated in a monocistronic sequence such as, but not limited to, a vector system or a nucleic acid vector. As a non-limiting example, the vector systems and nucleic acid vectors may include those described in U.S. Pat. Nos. 7,456,273 and 7,183,395, US Patent Publication Nos. US20070048776. US20090093049, and US20110124100 and International Patent Publication Nos. WO2007025008 and WO2001055371, each of which is incorporated herein by reference in its entirety.

In some embodiments, the TEEs described herein may be located in the 5′-UTR and/or the 3′-UTR of the mRNA. The TEEs located in the 3′-UTR may be the same and/or different than the TEEs located in and/or described for incorporation in the 5′-UTR.

In some embodiments, the 3′-UTR of the mRNA may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 3′-UTR of the polynucleotides, primary constructs, alternative nucleic acids and/or mmRNA of the present invention may be the same or different TEE sequences. The TEE sequences may be in a pattern such as ABABAB or AABBAABBAABB or ABCABCABC or variants thereof repeated once, twice, or more than three times. In these patterns, each letter, A, B, or C represent a different TEE sequence at the nucleotide level.

In some embodiments, the 3′-UTR may include a spacer to separate two TEE sequences. As a non-limiting example, the spacer may be a 15-nucleotide spacer and/or other spacers known in the art. As another non-limiting example, the 3′-UTR may include a TEE sequence-spacer module repeated at least once, at least twice, at least 3 times, at least 4 times, at least 5 times, at least 6 times, at least 7 times, at least 8 times and at least 9 times or more than 9 times in the 3′-UTR.

mRNA: Heterologous 5′-UTRs

5′-UTRs of an mRNA of the invention may be homologous or heterologous to the coding region found in the mRNA. Multiple 5′ UTRs may be included in mRNA and may be the same or of different sequences. Any portion of the mRNA, including none, may be codon optimized and any may independently contain one or more different structural or chemical alterations, before and/or after codon optimization.

Shown in Lengthy Table 21 in International Patent Publication No. WO 2014/081507, and in Lengthy Table 21 and in Table 22 in International Patent Publication No. WO 2014/081507, the contents of each of which are incorporated herein by reference in their entirety, is a listing of the start and stop site of mRNAs. In Table 21 each 5′-UTR (5′-UTR-005 to 5′-UTR 68511) is identified by its start and stop site relative to its native or wild type (homologous) transcript (ENST; the identifier used in the ENSEMBL database).

To alter one or more properties of the mRNA of the invention, 5′-UTRs which are heterologous to the coding region of the mRNA are engineered into the mRNA. The mRNA (e.g., an mRNA in a composition described herein) is administered to cells, tissue, or organisms, and outcomes such as protein level, localization, and/or half-life are measured to evaluate the beneficial effects the heterologous 5′-UTR may have on mRNA. Variants of the 5′ UTRs may be utilized wherein one or more nucleotides are added or removed to the termini, including A, T, C or G. 5′-UTRs may also be codon-optimized or altered in any manner described herein.

mRNA: RNA Motifs for RNA Binding Proteins

RNA binding proteins (RBPs) can regulate numerous aspects of co- and post-transcription gene expression, such as, but not limited to, RNA splicing, localization, translation, turnover, polyadenylation, capping, alteration, export, and localization. RNA-binding domains (RBDs), such as, but not limited to, RNA recognition motif (RR) and hnRNP K-homology (KH) domains, typically regulate the sequence association between RBPs and their RNA targets (Ray et al., Nature. 499:172-177 (2013); incorporated herein by reference in its entirety). In some embodiments, the canonical RBDs can bind short RNA sequences. In some embodiments, the canonical RBDs can recognize structure RNAs.

In some embodiments, to increase the stability of the mRNA of interest, an mRNA encoding HuR is co-transfected or co-injected along with the mRNA of interest into the cells or into the tissue. These proteins can also be tethered to the mRNA of interest in vitro and then administered to the cells together. Poly A tail binding protein, PABP interacts with eukaryotic translation initiation factor eIF4G to stimulate translational initiation. Co-administration of mRNAs encoding these RBPs along with the mRNA drug and/or tethering these proteins to the mRNA drug in vitro and administering the protein-bound mRNA into the cells can increase the translational efficiency of the mRNA. The same concept can be extended to co-administration of mRNA along with mRNAs encoding various translation factors and facilitators as well as with the proteins themselves to influence RNA stability and/or translational efficiency.

In some embodiments, the nucleic acids and/or mRNA may include at least one RNA-binding motif such as, but not limited to an RNA-binding domain (RBD).

In some embodiments, the RBD may be any of the RBDs, fragments or variants thereof descried by Ray et al., (Nature. 499:172-177 (2013); incorporated herein by reference in its entirety).

In some embodiments, the nucleic acids or mRNA of the present invention may include a sequence for at least one RNA-binding domain (RBDs). When the nucleic acids or mRNA of the present invention include more than one RBD, the RBDs do not need to be from the same species or even the same structural class.

In some embodiments, at least one flanking region (e.g., the 5′-UTR and/or the 3′-UTR) may include at least one RBD. In some embodiments, the first flanking region and the second flanking region may both include at least one RBD. The RBD may be the same or each of the RBDs may have at least 60% (e.g., at least 70%, 80%, or 90%) sequence identity to the other RBD. As a non-limiting example, at least on RBD may be located before, after and/or within the 3′-UTR of the nucleic acid or mRNA of the present invention. As another non-limiting example, at least one RBD may be located before or within the first 300 nucleosides of the 3′-UTR.

In some embodiments, the nucleic acids and/or mRNA of the present invention may include at least one RBD in the first region of linked nucleosides. The RBD may be located before, after, or within a coding region (e.g., the ORF).

In another embodiment, the first region of linked nucleosides and/or at least one flanking region may include at least on RBD. As a non-limiting example, the first region of linked nucleosides may include a RBD related to splicing factors and at least one flanking region may include a RBD for stability and/or translation factors.

In some embodiments, the nucleic acids and/or mRNA of the present invention may include at least one RBD located in a coding and/or non-coding region of the nucleic acids and/or mRNA.

In some embodiments, at least one RBD may be incorporated into at least one flanking region to increase the stability of the nucleic acid and/or mRNA of the present invention.

In some embodiments, an antisense locked nucleic acid (LNA) oligonucleotides and exon-junction complexes (EJCs) may be used in the RNA binding protein motif The LNA and EJCs may be used around a start codon (-4 to +37 where the A of the AUG codons is +1) in order to decrease the accessibility to the first start codon (AUG).

Nucleic Acids as Agents for Delivering Anti-CD38, Anti-BCMA, Anti-GPRC5D, and/or Anti-FcRH5 Antibodies or Binding Proteins

The compositions of the disclosure can be administered not only as antibodies or antigen-binding fragments, but also in the form of nucleic acids. The exemplary nucleic acids described herein may be used to deliver antibodies or antigen-binding fragments to a subject. These nucleic acids (e.g., RNAs, such as mRNAs) may be used as therapeutic agents to express antibodies or antigen-binding fragments of the disclosure as a therapy to treat a target disease.

### Pharmaceutical Compositions

Pharmaceutical compositions containing an anti-CD38, anti-BCMA, anti-GPRC5D, anti-FcRH5 antibody, antigen-binding fragment, binding protein, or nucleic acid encoding the same, described herein can be prepared using methods known in the art. Pharmaceutical compositions described herein may contain an anti-CD38, anti-BCMA, anti-GPRC5D, anti-FcRH5 antibody, antigen-binding fragment, binding protein, or a nucleic acid encoding the same, described herein in combination with one or more pharmaceutically acceptable excipients. For instance, pharmaceutical compositions described herein can be prepared using physiologically acceptable carriers, excipients, or stabilizers (Remington's Pharmaceutical Sciences (19th ed., 1995), incorporated herein by reference), and in a desired form, e.g., in the form of lyophilized formulations or aqueous solutions. The compositions can also be prepared so as to contain the active agent (e.g., an anti-CD38, anti-BCMA, anti-GPRC5D, anti-FcRH5 antibody, antigen-binding fragment, binding protein, or a nucleic acid encoding the same) at a desired concentration. For example, a pharmaceutical composition described herein may contain at least 10% (e.g., 10%, 20%, 30%, 40%, 50%, 60%. 70%, 80%. 90%, 95%, 97%, 98%, 99%, 99.5%. 99.9%, or 100%) active agent by weight (w/w).

Additionally, an active agent that can be incorporated into a pharmaceutical formulation can itself have a desired level of purity. For example, a polypeptide or nucleic acid described herein may be characterized by a certain degree of purity after isolating the antibody or binding protein from cell culture media or after chemical synthesis. An antibody, antigen-biding fragment, binding protein, or nucleic acid described herein may be at least 10% pure prior to incorporating the antibody, antigen-biding fragment, binding protein or nucleic acid into a pharmaceutical composition (e.g., 10%, 20%, 30%, 40%, 50%, 60%, 70%, 80%, 90%, 95%, 97%, 98%, 99%, 99.5%, 99.9%, 99.99%, or 100% pure).

Pharmaceutical compositions can be prepared for storage as lyophilized formulations or aqueous solutions by mixing the active agent having the desired degree of purity with optional pharmaceutically acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences (19th ed., 1995), incorporated herein by reference). Such additives must be nontoxic to the recipients at the dosages and concentrations employed.

### Buffering Agents

Buffering agents help to maintain the pH in the range which approximates physiological conditions. Suitable buffering agents for use with the pharmaceutical compositions of the disclosure include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.), and acetate buffers (e.g., acetic acid-sodium acetate mixture, acetic acid-sodium hydroxide mixture, etc.). Additionally, phosphate buffers, histidine buffers, and trimethylamine salts such as Tris can be used.

### Preservatives

Preservatives can be added to a composition described herein, for example, to inhibit microbial growth. Suitable preservatives for use with the pharmaceutical compositions of the disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonifiers, also known as “stabilizers,” can be added to ensure isotonicity of liquid compositions described herein and include polhydric sugar alcohols, for example trihydric or higher sugar alcohols, such as glycerin, arabitol, xylitol, sorbitol, and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols: amino acids such as arginine, lysine, glycine, glutamine, asparagine, histidine, alanine, omithine, L-leucine. 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate: low molecular weight polypeptides (e.g., peptides of 10 residues or fewer); proteins such as HSA, BSA, MSA, gelatin, or immunoglobulins; hydrophilic polymers, such as polyvinylpyrrolidone monosaccharides, such as xylose, mannose, fructose, glucose; disaccharides such as lactose, maltose, sucrose and trisaccharides such as raffinose; and polysaccharides such as dextran.

### Detergents

In some embodiments, non-ionic surfactants or detergents (also known as “wetting agents”) are added to the pharmaceutical composition, for example, to help solubilize the therapeutic agent as well as to protect the therapeutic agent against agitation-induced aggregation, which also permits the formulation to be exposed to shear surface stressed without causing denaturation of the protein. Suitable non-ionic surfactants include, for example and without limitation, polysorbates (20, 80, etc.), polyoxamers (184, 188 etc.), Pluronic polyols, polyoxyethylene sorbitan monoethers (TWEEN®-20, TWEEN®-80, etc.).

### Other Pharmaceutical Carriers

Alternative pharmaceutically acceptable carriers that can be incorporated into a pharmaceutical composition described herein may include dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils, but not limited to. A pharmaceutical composition described herein may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

### Lipid Nanoparticle (LNP) Compositions

The present disclosure provides LNP compositions with advantageous properties. The lipid nanoparticle compositions described herein may be used for the delivery of therapeutic and/or prophylactic agents, e.g., mRNAs, to mammalian cells or organs. For example, the lipid nanoparticles described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In some embodiments, the present application provides pharmaceutical compositions comprising: (a) a delivery agent comprising a lipid nanoparticle; and (b) a polynucleotide encoding an antibody or antigen-binding fragment of the disclosure.

### Lipid Nanoparticles

In some embodiments, polynucleotides of the present disclosure (e.g., mRNA) are included in a lipid nanoparticle (LNP). Lipid nanoparticles according to the present disclosure may comprise: (i) an ionizable lipid (e.g., an ionizable amino lipid; (ii) a sterol or other structural lipid; (iii) a non-cationic helper lipid or phospholipid; and (iv) a PEG-modified lipid. In some embodiments, lipid nanoparticles according to the present disclosure further comprise one or more polynucleotides of the present disclosure (e.g., mRNA).

The lipid nanoparticles according to the present disclosure can be generated using components, compositions, and methods as are generally known in the art, see, for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492: PCT/US2016/059575 and PCT/US2016/069491 all of which are incorporated by reference herein in their entirety.

In some embodiments, the lipid nanoparticle comprises: (i) 20 to 60 mol. % ionizable cationic lipid (e.g., ionizable amino lipid), (ii) 25 to 55 mol. % sterol or other structural lipid, (iii) 5 to 25 mol. % non-cationic lipid (e.g., phospholipid), and (iv) 0.5 to 15 mol. % PEG-modified lipid.

In some embodiments, the lipid nanoparticle comprises: (i) 40 to 50 mol. % ionizable cationic lipid (e.g., ionizable amino lipid), (ii) 30 to 45 mol. % sterol or other structural lipid. (iii) 5 to 15 mol. % non-cationic lipid (e.g., phospholipid), and (iv) 1 to 5 mol. % PEG-modified lipid.

In some embodiments, the lipid nanoparticle comprises: (i) 45 to 50 mol. % ionizable cationic lipid (e.g., ionizable amino lipid), (ii) 35 to 45 mol. % sterol or other structural lipid, (iii) 8 to 12 mol. % non-cationic lipid (e.g., phospholipid), and (iv) 1.5 to 3.5 mol. % PEG-modified lipid.

In the following sections. “Compounds” numbered with an “I-” prefix (e.g., “Compound I-1,” “Compound 1-2,” “Compound I-3,” “Compound I-VI,” etc., indicate specific ionizable lipid compounds. Likewise, compounds numbered with a “P-” prefix (e.g., “Compound P-I,” etc.) indicate a specific PEG-modified lipid compound.

### Ionizable Amino Lipids

In some embodiments, the lipid nanoparticle of the present disclosure comprises an ionizable cationic lipid (e.g., an ionizable amino lipid) that is a compound of Formula (I):

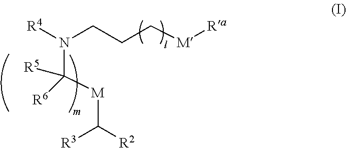

or its N-oxide, or a salt or isomer thereof, wherein R′^{a} is R′^{branched}; wherein
R′^{branched} is:

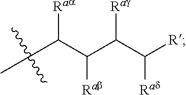

wherein

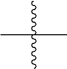

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

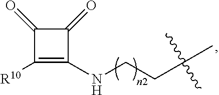

wherein

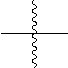

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and
—OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, in Formula (I), R′^{a} is R′^{branched}; R′^{branched} is

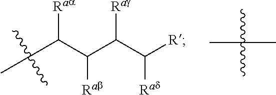

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, in Formula (I), R′^{a} is R′^{branched}; R′^{branched} is

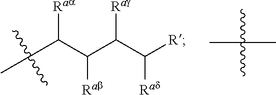

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (I). R′^{a} is R′^{branched};
R′^{branched} is

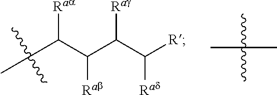

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

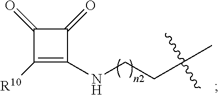

R¹⁰ is NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}:
R′^{branched} is

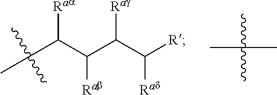

denotes a point of attachment; R^{aα}, R^{aβ}, and R^{aδ} are each H; R^{aδ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (I) is selected from:

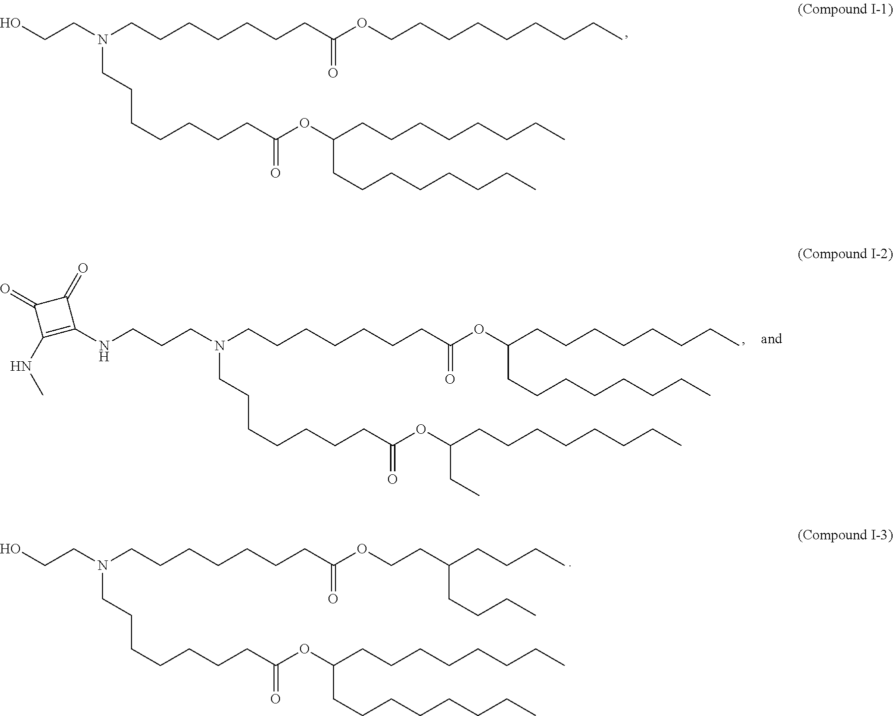 [SMILES: CCCCCCCCCOC(=O)CCCCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the compound of Formula (I) is:

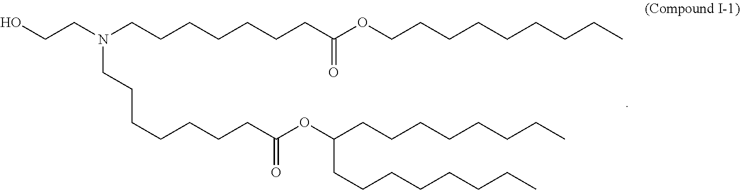 [SMILES: CCCCCCCCCOC(=O)CCCCCCCN(CCO)CCCCCCCC(=O)OC(CCCCCCCC)CCCCCCCC]

In some embodiments, the compound of Formula (I) is:

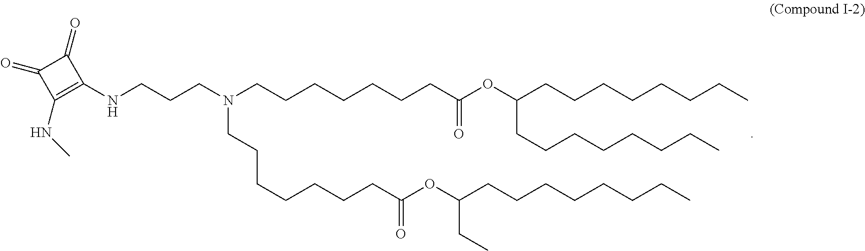 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is:

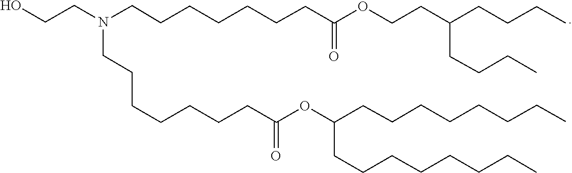 [SMILES: CCCCCCCCC(CCCCCCCC)OC(=O)CCCCCCCN(CCO)CCCCCCCC(=O)OCCC(CCCC)CCCC]

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g. —delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the present disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present disclosure is an analog or variant of DSPC.

### Alternative Lipids

In certain embodiments, a phospholipid useful or potentially useful in the present disclosure comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the present disclosure is oleic acid.

In certain embodiments, the alternative lipid is one of the following:

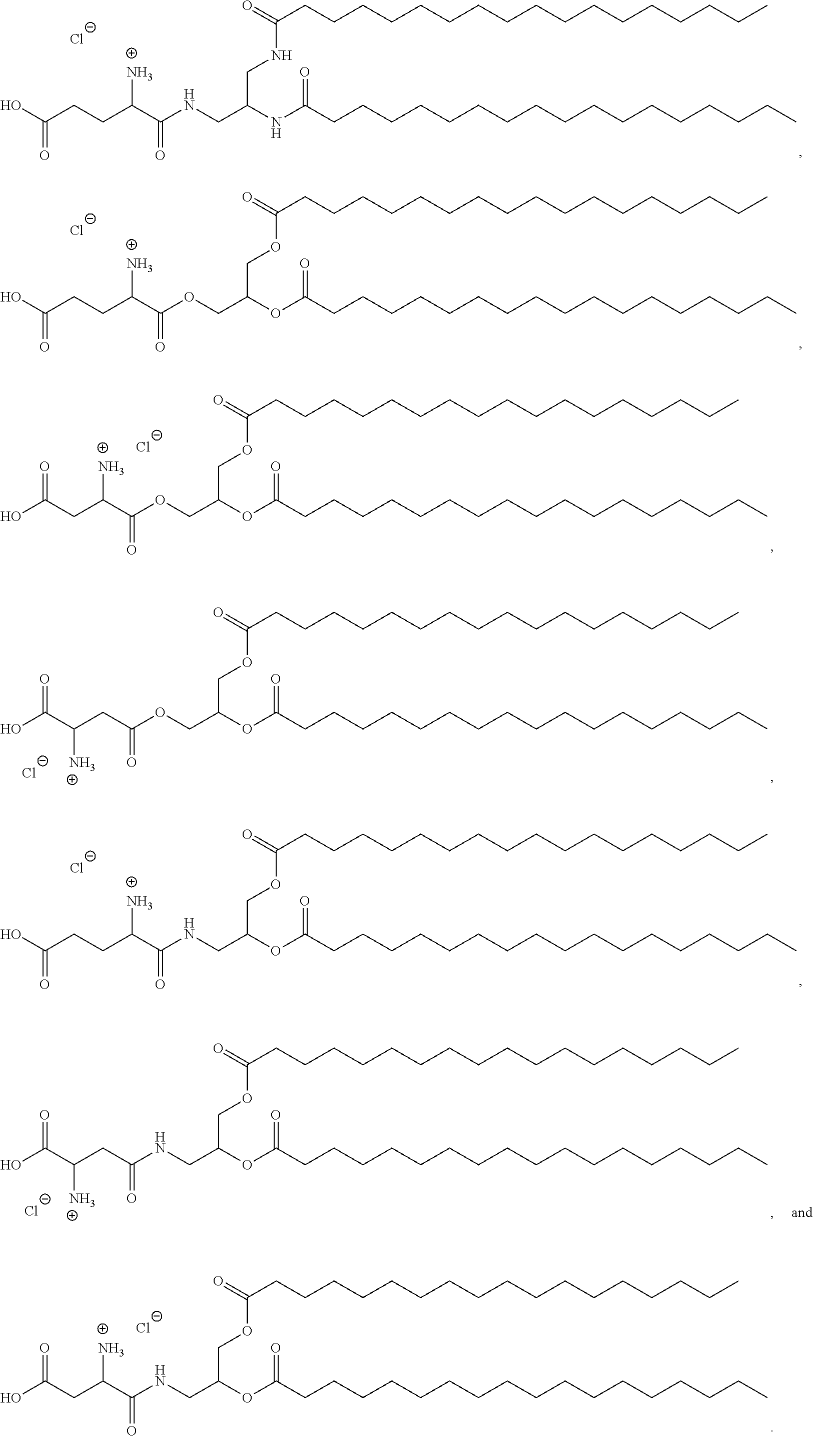

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol.

In certain embodiments, the structural lipid is an analog of cholesterol. In certain embodiments, the structural lipid is alpha-tocopherol.

In some embodiments, the structural lipids may be one or more of the structural lipids described in U.S. Application No. 62/520,530.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1.2-diacyloxypropan-3-anines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipahnitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆.

In some embodiments, a PEG moiety, for example an mPEG-NH2, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various Formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, PEG lipids useful in the present disclosure can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present disclosure.

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the present disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkvlglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP of the present disclosure comprises an ionizable cationic lipid of Formula I, a phospholipid comprising DSPC, a structural lipid, and a PEG lipid comprising PEG-DMG.

In some embodiments, a LNP of the present disclosure comprises an ionizable cationic lipid of Formula I, a phospholipid comprising DSPC, a structural lipid, and a PEG lipid.

In some embodiments, a LNP of the present disclosure comprises an ionizable cationic lipid of

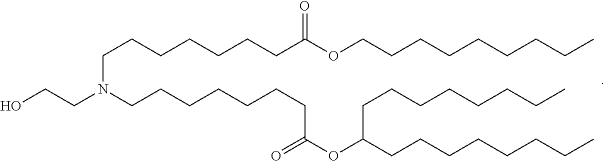 [SMILES: CCCCCCCCCOC(=O)CCCCCCCN(CCO)CCCCCCCC(=O)OC(CCCCCCCC)CCCCCCCC]

In some embodiments, a LNP of the present disclosure comprises an ionizable cationic lipid of

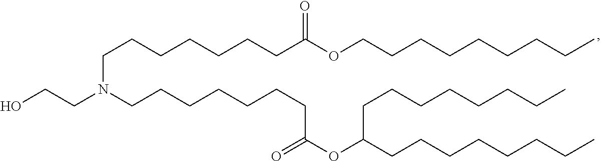 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP of the present disclosure comprises an ionizable cationic lipid of

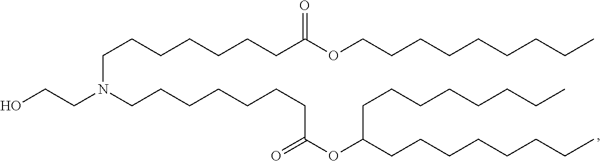 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid.

In some embodiments, a LNP of the present disclosure comprises an ionizable cationic lipid of

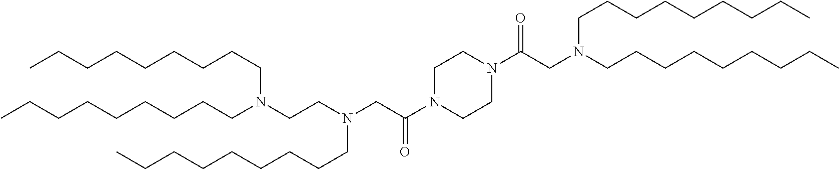 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid.

In some embodiments, a LNP of the present disclosure comprises an ionizable cationic lipid of

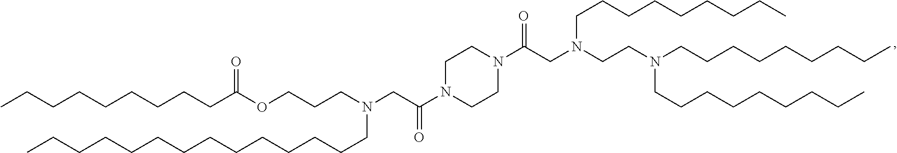 [SMILES: CCCCCCCCCCCCCCN(CCCOC(=O)CCCCCCCCC)CC(=O)N1CCN(C(=O)CN(CCCCCCCCC)CCN(CCCCCCCCC)CCCCCCCCC)CC1]

a phospholipid comprising DOPE, a structural lipid comprising cholesterol, and a PEG lipid.

In some embodiments, a LNP of the present disclosure comprises an N:P ratio of from about 2:1 to about 30:1.

In some embodiments, a LNP of the present disclosure comprises an N:P ratio of about 6:1.

In some embodiments, a LNP of the present disclosure comprises an N:P ratio of about 3:1.

In some embodiments, a LNP of the present disclosure comprises a wt/wt ratio of the ionizable cationic lipid component to the RNA of from about 10:1 to about 100:1.

In some embodiments, a LNP of the present disclosure comprises a wt/wt ratio of the ionizable cationic lipid component to the RNA of about 20:1.

In some embodiments, a LNP of the present disclosure comprises a wt/wt ratio of the ionizable cationic lipid component to the RNA of about 10:1.

In some embodiments, a LNP of the present disclosure has a mean diameter from about 50 nm to about 150 nm.

In some embodiments, a LNP of the present disclosure has a mean diameter from about 70 nm to about 120 nm.

### Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1.48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In some embodiments, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In some embodiments, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

### Nanoparticle Compositions

In some embodiments, the pharmaceutical compositions disclosed herein are Formulated as lipid nanoparticles (LNP). Accordingly, the present disclosure also provides nanoparticle compositions comprising (i) a lipid composition comprising a delivery agent such as compound as described herein, and (ii) a polynucleotide encoding a polypeptide. In such nanoparticle composition, the lipid composition disclosed herein can encapsulate the polynucleotide encoding a polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, a lipid nanoparticle comprises an ionizable amino lipid, a structural lipid, a phospholipid, and mRNA. In some embodiments, the LNP comprises an ionizable amino lipid, a PEG-modified lipid, a sterol and a structural lipid. In some embodiments, the LNP has a molar ratio of about 40-50% ionizable amino lipid; about 5-15% structural lipid; about 30-45% sterol; and about 1-5% PEG-modified lipid.

In some embodiments, the lipid nanoparticle comprises 47-49 mol. % ionizable cationic lipid (e.g. ionizable amino lipid, e.g., Compound I-1, Compound I-2, or Compound I-3), 10-12 mol. % non-cationic lipid (e.g., phospholipid, e.g., DSPC), 38-40 mol. % sterol (e.g., cholesterol) or other structural lipid, and 1-3 mol. % PEG-modified lipid (e.g., PEG-DMG or Compound P-I).

For instance, in some embodiments, the lipid nanoparticle (“LNP-1”) may comprise the following components at the following molar ratios:
(i) 45-50 mol. % Compound I-1
(ii) 35-45 mol. % sterol (e.g., cholesterol);
(iii) 8-12 mol. % phospholipid (e.g., DSPC or DOPE); and
(iv) 1.5-3.5 mol. % PEG-lipid.

For instance, in some embodiments, the lipid nanoparticle (“LNP-1A”) may comprise the following components at the following molar ratios:
(i) 45-50 mol. % Compound I-1
(ii) 35-45 mol. % Cholesterol;
(iii) 8-12 mol. % DSPC; and
(iv) 1.5-3.5 mol. % PEG-lipid.

For instance, in some embodiments, the lipid nanoparticle (“LNP-1B”) may comprise the following components at the following molar ratios:
(i) 45-50 mol. % Compound I-1
(ii) 35-45 mol. % Cholesterol;
(iii) 8-12 mol. % DSPC; and
(iv) 1.5-3.5 mol. % PEG-lipid.

In some embodiments, the lipid nanoparticle (“LNP-2”) may comprise the following:
(i) 45-50 mol. % Compound I-2;
(ii) 35-45 mol. % sterol (e.g., Cholesterol);
(iii) 8-12 mol. % phospholipid (e.g., DSPC or DOPE); and
(iv) 1.5-3.5 mol. % PEG-lipid.

In some embodiments, the lipid nanoparticle (“LNP-2A”) may comprise the following:
(i) 45-50 mol. % Compound I-2;
(ii) 35-45 mol. % Cholesterol;
(iii) 8-12 mol. % DSPC; and
(iv) 1.5-3.5 mol. % PEG-lipid.

For instance, in some embodiments, the lipid nanoparticle (“LNP-2B”) may comprise the following components at the following molar ratios:
(i) 45-50 mol. % Compound I-2;
(ii) 35-45 mol. % Cholesterol;
(iii) 8-12 mol. % DSPC; and
(iv) 1.5-3.5 mol. % PEG-lipid.

In some embodiments, the lipid nanoparticle (“LNP-3”) may comprise the following:
(i) 45-50 mol. % Compound I-3;
(ii) 35-45 mol. % sterol (e.g., Cholesterol);
(iii) 8-12 mol. % phospholipid (e.g., DSPC or DOPE); and
(iv) 1.5-3.5 mol. % PEG-lipid.

In some embodiments, the lipid nanoparticle (“LNP-3A”) may comprise the following:
(i) 45-50 mol. % Compound I-3;
(ii) 35-45 mol. % Cholesterol;
(iii) 8-12 mol. % DSPC; and
(iv) 1.5-3.5 mol. % PEG-lipid.

In some embodiments, the lipid nanoparticle (“LNP-3B”) may comprise the following:
(i) 45-50 mol. % Compound I-3;
(ii) 35-45 mol. % Cholesterol;
(iii) 8-12 mol. % DSPC; and
(iv) 1.5-3.5 mol. % PEG-lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable amino lipid. As used herein, the term “ionizable amino lipid” has its ordinary meaning in the art and may refer to a lipid comprising one or more charged moieties. In some embodiments, an ionizable amino lipid may be positively charged or negatively charged. An ionizable amino lipid may be positively charged, in which case it can be referred to as “cationic lipid”. In certain embodiments, an ionizable amino lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively- charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

The ionizable amino lipid is sometimes referred to in the art as an “ionizable cationic lipid”. In some embodiments, the ionizable amino lipid may have a positively charged hydrophilic head and a hydrophobic tail that are connected via a linker structure.

In addition to these, an ionizable amino lipid may also be a lipid including a cyclic amine group.

In some embodiments, the ionizable amino lipid may be selected from, but not limited to, an ionizable amino lipid described in International Publication Nos. WO2013086354 and WO2013116126; the contents of each of which are herein incorporated by reference in their entirety.

In yet another embodiment, the ionizable amino lipid may be selected from, but not limited to, Formula CLI-CLXXXXII of U.S. Pat. No. 7,404,969: each of which is herein incorporated by reference in their entirety.

In some embodiments, the lipid may be a cleavable lipid such as those described in International Publication No. WO2012170889, herein incorporated by reference in its entirety. In some embodiments, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In some embodiments, the polynucleotide encoding a polypeptide are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 un, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the nanoparticles have a diameter from about 10 to 500 nm. In some embodiments, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1.22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In some embodiments, the polynucleotides described herein can be Formulated for controlled release and/or targeted delivery. As used herein, “controlled release” refers to a pharmaceutical composition or compound release profile that conforms to a particular pattern of release to effect a therapeutic outcome. In some embodiments, the polynucleotides can be encapsulated into a delivery agent described herein and/or known in the art for controlled release and/or targeted delivery. As used herein, the term “encapsulate” means to enclose, surround or encase. As it relates to the formulation of the compounds of the present disclosure, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the present disclosure can be enclosed, surrounded or encased within the delivery agent. “Partial encapsulation” or “partially encapsulate” means that less than 10, 10, 20, 30, 40 50 or less of the pharmaceutical composition or compound of the present disclosure can be enclosed, surrounded or encased within the delivery agent.

In some embodiments, the therapeutic nanoparticle polynucleotide can be Formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the polynucleotides described herein can be Formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle polynucleotide can be Formulated to be target specific, such as those described in Intl. Pub. Nos. WO2008121949. WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see, Zhigaltsev et al., Langmuir. 28:3633-40 (2012); Belliveau et al., Molecular Therapy-Nucleic Acids. 1:e37 (2012); Chen et al., J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM) from the Institut fGr Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the polynucleotides described herein can be Formulated in lipid nanoparticles using microfluidic technology (see, Whitesides, George M., Nature 442: 368-373 (2006); and Abraham et al., Science 295: 647-651 (2002); each of which is herein incorporated by reference in its entirety). In some embodiments, the polynucleotides can be Formulated in lipid nanoparticles using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, poly caprolactones, polyamides, polyacetals, polyethers, polyesters, poly(orthoesters-), polycyanoacrylates, polyvinyl alcohols, polyurethanes, polyphosphazenes, polyacrylates, polymethacrylates, polycyanoacrylates, polyureas, polystyrenes, polyamines, polyesters, poly anhydrides, polyethers, polyurethanes, polymethacrylates, polyacrylates, polycyanoacrylates, or combinations thereof.

### Methods of Using Anti-CD38, Anti-BCMA, Anti-GPRC5D, Anti-FCRH5 Antibodies, Antigen-Binding Fragments, and Binding Proteins

In some aspects, antibodies, antigen-binding fragments, and/or binding proteins of the present disclosure are administered to a subject in need thereof. In some aspects, the subject in need thereof is a subject with a disease, disorder, and/or condition that may be treated with technologies described herein. Specifically, a disease or disorder which would benefit from targeting cells expressing anti-CD38, anti-BCMA, anti-GPRC5D, and/or anti FCRH5 for reduction or elimination.

In some aspects, the subject in need thereof is a subject with cancer, an infection, an inflammatory condition, or an autoimmune condition.

In some aspects, the subject in need thereof is a mammal. In some embodiments, a mammal includes, for example and without limitation, a household pet (e.g., a dog, a cat, a rabbit, a ferret, a hamster), a livestock or fanr animal (e.g., a cow, a pig, a sheep, a goat, a pig, a chicken or another poultry), a horse, a monkey, a laboratory animal (e.g., a mouse, a rat, a rabbit) and a human. In a preferred embodiment, the subject in need thereof is a human. Technologies of the present disclosure can be practiced in any subject in need thereof that is likely to benefit from administration of technologies of the present disclosure (e.g., a subject with cancer).

In some embodiments, a subject in need thereof is a human. In some embodiments, the human is male. In some embodiments, the human is female. In some embodiments, the human is an adult (e.g., 18 or more years of age). In some embodiments, the adult is greater than 18 years old, greater than 25 years old, greater than 30 years old, greater than 40 years old, greater than 50 years old, greater than 55 years old, greater than 60 years old, greater than 65 years old, greater than 70 years old, greater than 75 years old, greater than 80 years old, greater than 85 years old, greater than 90 years old, greater than 95 years old, greater than 100 years old, or greater than 105 years old in age. In some embodiments, the human is a child. In some embodiments, the child is greater than 2 years old, greater than 3 years old, greater than 4 years old, greater than 5 years old, greater than 6 years old, greater than 7 years old, greater than 8 years old, greater than 9 years old, greater than 10 years old, greater than 11 years old, greater than 12 years old, greater than 13 years old, greater than 14 years old, greater than 15 years old, or greater than 16 years old in age.

In some aspects, a subject in need thereof is administered an antibody or binding protein of the present disclosure.

### Routes of Administration and Dosing

Anti-CD38, anti-BCMA, anti-GPRC5D, and anti-FcRH5 antibodies or antigen-binding fragments of the disclosure, and nucleic acids encoding the same, can be administered to a subject (e.g., a mammalian subject, such as a human) by a variety of routes. In some embodiments, the antibody or nucleic acid is administered to the subject intravenously, subcutaneously, intramuscularly, parenterally, intrathecally, intracerebroventricularly, transdermally, or orally.

The most suitable route for administration in any given case will depend on the particular therapeutic agent administered, the patient, pharmaceutical formulation methods, and various patient-specific parameters, such as the patient's age, body weight, sex, severity of the diseases being treated, the patient's diet, and the patient's excretion rate.

An appropriate dosage of anti-CD38, anti-BCMA, anti-GPRC5D, and anti-FcRH5 antibodies, antigen-binding fragments, or binding proteins, or nucleic acids encoding the same of the present disclosure will vary with the particular condition, disease and/or disease being treated, various subject-specific parameters (e.g., age, weight, physical condition of the subject), severity of the particular condition, disease, and/or disorder being treated, the nature of current or combination therapy (if any), the specific route of administration and other factors within the knowledge and expertise of a health practitioner. In some embodiments, a maximally tolerated dose of technologies described herein is to be used, e.g., the highest safe dose according to sound medical judgement. In some embodiments, technologies described herein are administered in an effective amount, e.g., a dose sufficient to provide one or more medically desirable results.

A therapeutic regiment for use in accordance with technologies described herein may include administration of such technologies or compositions comprising such technologies once a day, once every two days, once every three days, twice a week, once a week, once every two weeks, once every three weeks, once every month or four weeks, once every six weeks, once every two months or eight weeks, once every three months or twelve weeks. In some certain embodiments, a subject receives a single dose of a technology described herein. In certain embodiments, a subject receives a plurality of doses of a technology described herein (e.g., at least two, at least three, at least four, at least five, at least six, at least eight, at least ten, or more doses). In some embodiments, technologies described herein are administered over a period of time, such as one week, two weeks, three weeks, four weeks, six weeks, two months, three months, four months, five months, six months, one year or more. Appropriate therapeutic regimens are readily understood by medical practitioners and such regimens may be designed by a medical practitioner for a particular patient (e.g., a patient-specific regimen).

### Kits

Also included herein are kits that contain anti-CD38, anti-BCMA, anti-GPRC5D, and/or anti-FcRH5 antibodies, antigen-binding fragments, binding proteins, and/or nucleic acids encoding the same. In some embodiments, the kits provided herein contain one or more cells engineered to express and secrete an anti-CD38, anti-BCMA, anti-GPRC5D, and/or anti-FcRH5 antibody, antigen-binding fragment, of binding protein of the disclosure, such as a cell containing a nucleic acid molecule of the disclosure.

A kit described herein may include reagents that can be used to produce a pharmaceutical composition of the invention. Optionally, kits described herein may include reagents that can induce the expression of anti-CD38, anti-BCMA, anti-GPRC5D, and/or anti-FcRH5 antibodies, antigen-binding fragments, or relate proteins of the present disclosure within cells (e.g., mammalian cells).

Other kits described herein may include tools for engineering a prokaryotic or eukaryotic cell (e.g., a CHO cell or a BL21(DE3) E. Coli cell or an immune cell) so as to express and secrete an anti-CD38, anti-BCMA, anti-GPRC5D, and/or anti-FcRH5 antibody or binding protein described herein. For example, a kit may contain CHO cells stored in an appropriate media and optionally frozen according to methods known in the art. The kit may also contain a nucleic acid encoding the desired antibody or binding protein as well as reagents for expressing the antibody or binding protein in the cell.

A kit described herein may also provide an anti-CD38, anti-BCMA, anti-GPRC5D, and/or anti-FcRH5 antibody, antigen-binding fragment or binding protein of the disclosure, or a nucleic acid encoding the same in combination with a package insert describing how the antibody, antigen-binding fragment, binding protein, or nucleic acid may be administered to a subject, for example, for the treatment of a disease, disorder and/or condition (e.g., cancer).

### Examples

The following examples are put forth so as to provide those of ordinary skill in the art with a description of how the compositions and methods claimed herein can be performed, made, and evaluated, and are intended to be purely exemplary and are not intended to limit the scope of the present disclosure.

Methods: The Following Materials and Methods were Used in the Examples.

Immunization of Llamas: Animals were immunized either with protein or mRNA. For protein immunizations a primary immunization typically contained 200 μg of the appropriate target protein in complete Freund's Adjuvant (CFA), given subcutaneously. Formulated mRNA (200-1000 pg) was administered intramuscularly, using the same schedule as protein. Following the initial immunizations, boost immunizations were given approximately every two weeks. Protein boosts were formulated with incomplete Freund's Adjuvant (IFA). If available the cyno and human forms of the proteins were alternatively injected for a total of 4 injections. Test bleeds were taken on day 52 post immunization, to determine antibody titers against the immunized targets in the serum. A final intravenous immunization was given on day 64. The second bleed was performed on day 70, for a production bleed of 600 ml and was used to isolate and cryopreserve PBMCs. For mRNA immunizations, sequences encoding the desired immunogen(s) were formulated and mRNA was immunized intramuscularly. The immunization schedule was the same for protein immunizations.

Selection and culturing of immune reactive B-cells: Immunoreactive B-cells were selected by Fluorescence Activated Cell Sorting (FACS) using fluorescently labeled rabbit anti-VHH antibodies, and when available, in combination with fluorescently labeled antigen. B-cells were selected for positive staining with fluorescent rabbit anti-VHH antibodies, and fluorescently labeled antigen when available. Appropriately stained cells were distributed into plates.

B-cell culture: B-cells are cultured based on methods available on mammalian B cell culture (see, e.g., Kwakkenbos et al., Nat. Med. 16(1):123-128 (2010); WO2013076139 A1; Carbonetti et al., JIM 448: 66-73 (2017), herein incorporated by reference in their entirety). B-cells were distributed into 96 well plates at varied numbers of cells/well, as indicated below, and co-cultured with species specific irradiated CD40L expressing feeder cells and the appropriate cytokines. The culture plates were incubated for up to 10-12 days in a CO₂ incubator at 37° C. under 5% CO₂. Typically, between days 8-11 proliferating B cell cultures supernatants were taken and screened for antibody binding to the indicated target antigens. Cell pellets for all wells were flash-frozen for potential generation of cDNA. Antibody sequencing was performed on wells exhibiting selected Ab target binding.

Yeast Display: Yeast surface display was performed as outlined in Van Deventer JA, Wittrup K_{D}, Methods Mol Biol., 2014. Any differences and changes are outlined below. For library generation. PBMCs from immunized llama were used to make a VHH display library. Total RNA was extracted with TRIzol (Invitrogen 15596026) from ~10⁹ PBMCs and 5 pg RNA reactions were reverse transcribed into cDNA using the SuperScript III First-Strand Synthesis System (Invitrogen 18080051). Using the appropriate primer sets, a nested PCR approach was used to specifically amplify the VHH region from IgG2 and IgG3. Using homologous recombination, the VHH insert was inserted into a pCTCON2 yeast surface display vector during electroporation. The library size was approximately 10⁸. The library was then enriched for binders using magnetic assisted cell sorting (MACS). Two negative sorts were performed followed by a positive selection with 33 pmol of biotinylated BCMA-huFC fusion protein (bio-BCMA). Another enrichment with MACs was performed as stated above but with 6.7 pmol of bio-BCMA. Enrichment was then performed with fluorescence assisted cell sorting (FACS). Bio-BCMA was added at 100 nM under equilibrium conditions to select for the top binders. The library was then grown and enriched again by FACS with 10 nM of bio-BCMA under equilibrium conditions. The starting library, the library after each enrichment step, and the final library were all analyzed by next-generation sequencing. The top 24 clones were chosen based on their enrichment values and synthesized into proteins to assay for cell binding.

Screening for antibody hits via ELISA or FACS: Typically ELISA Assay plates were prepared by coating each plate with 50 μl/well of 1-3 ug of purified protein and incubating over night at 4° C. The Assay plates were then washed three times with PBS (pH 7.0), 0.05% Tween-20 (wash buffer) prior to coating with diluted supernatants and standards from the dilution plates. Dilution plates were prepared and contained both standards and unknowns. Standards were typically diluted 1:3 starting at 2.5 μg/ml, and a buffer blank was included for a negative control. The sera from the B-cell culture plates were diluted in the range of 1:2-1:5 onto the dilution plate. Typically 20-50 μl of diluted supernatant was transferred from the dilution plates onto the washed assay plates. Following transfer of sera from the dilution plate, assay plates were typically incubated 1 h at room temperature. Assay plates were then washed 3 times with wash buffer and a secondary antibody added and incubated for 1 h (anti-camelid Fc-HRP, diluted at 1:500). Assay plates were again washed 3 times with wash buffer before TMB developer was added, incubated for 5-15 min, and then stopped with 50 μl of 1N H2SO4. Plates were read at 450 nM. Wells that were greater than 3× background were determined to be positive.

Sequencing of Antibodies from B-cells: mRNA was isolated from B cell culture lysates using the Dynabeads mRNAdirect purification kit (Invitrogen, 61012) according to manufacturer's instructions. cDNA synthesis was performed using Superscript IV first strand synthesis system (Invitrogen, 18091050). Two step PCR was performed according to standard procedures, to amplify the variable domain of the heavy chain with historical primers used in Llama antibody discovery (see, e.g., Conrath et al., Antimicro.—Agents Chemother, v45(10):2807-2812 (2001); Els Pardon, Nat. Protoc. 9(3):674-93 (2014), herein incorporated by reference in their entirety). cDNA of the molecules of interest were then sequenced using one of two methods; Sanger sequencing or Next Generation Sequencing (NGS), in both cases, the primary PCR involved using CALL001 forward primer and CALL002 reverse primer that anneal to the leader sequence and 5′ end of CH2 domain of the heavy chain respectively. For Sanger sequencing the resulting PCR sequence could be sequenced directly (Quintarabio, MA). For NGS, the PCR product was used in a nested PCR with forward and reverse primers listed in the above references linked with NGS adaptors, to amplify a 400 bp VH or VHH amplicon. The nested PCR amplicon was used in a final PCR reaction using barcoded primers that bind to the adaptors introduced in the nested PCR. The amplicons were then sequenced by the commercially available MiSeq system (Illumina corp).

VHH expression: Recombinant antibody expression was performed in 2 ml cultures. Small scale transfections of the engineered plasmid constructs in expi293 cells were set up in 24 deep well plates. (See, e.g., Vazquez-Lombardi et al., Nat Protoc 13, 99-11 7 (2018).) The suspension cultures were shaken at 250 rpm and incubated at 8% CO₂ and 85% humidity for protein expression. 30 ml transfections of expi293 cells were cultured for 5 days, supernatants harvested, and the sdAb proteins purified from the supernatants using Ni Sepharose excel resin (Cytiva; cat #17371201). The purified proteins were buffer exchanged with 1×PBS, pH7.4 using 3 kD cut off amicon filters. The purified proteins were analyzed by SDS-PAGE under non-reducing conditions to check for purity and validation of molecular weight. V5 or FLAG-tagged sdAb VHH clones exhibited abundant protein expression. An average of 4-5 mg protein was purified from 30 ml transfected culture supernatants.

Purification of single domain antibodies: Expressed single domain antibodies with a 6×His C-terminal tag (SEQ ID NO: 492) were purified from ExpiCHO media by capture on TALON (TAKRA) cobalt immobilized metal affinity chromatography (IMAC) resin. Resin was prewashed in 50 mM sodium phosphate buffer pH 7.4 (wash buffer) with 15× the volume of resin, then centrifuged for 2 min at 700×g to pellet the resin. The supernatant was discarded and the resin wash repeated. 3 mL of IMAC resin was added to the filtered media containing the expressed protein, then allowed to bind overnight with gentle shaking. The media resin mixture was gravity loaded into a 25 ml column. Columns containing IMAC resin were then washed with 10 column volumes (−30 ml) of wash buffer (PBS pH 7.4 (Life Tech cat #10010-023), 2 mM imidazole), followed by 2 times with 10 ml of wash buffer. Protein was then eluted using a total of 7.5 mL of 150 mM imidazole/PBS pH 7, in three 2.5 mL aliquots. Proteins were then dialyzed exhaustively using Slide-A-Lvzer® 10 or 3K as appropriate, (Dialysis cassette. Pierce) versus 1×PBS (100 mM NaPO₄ pH 6.8, 200 mM NaCl).

Humanization: Antibody humanization was done using the general methods of CDR grafting (Hanf et al., Methods. 65(1):68-76 (2014)). Briefly sequences the complementarity-determining regions (CDRs) of the antibody were annotated using the IMGT numbering scheme. Each Vhh nucleotide sequence is generated and used to identify the nearest human germline VH sequences by searching for similar sequences with the NCBI IgBLAST program. Common J and D gene sequences were attached to the VH as the acceptor. Next the most similar human VH sequences are identified using BLASTp and used to choose the nearest framework sequences into which the antibody CDR sequences are grafted replacing the human CDRs.

Rosetta or Alpha Fold was used to create the structural 3D homology model the of the appropriate CDRs that were grafted into the acceptor framework. The framework residues that were critical for huVH/VL interactions are back mutated to llama sequence canonical llama residues, also potentially structural defects due to mismatches at the graft interface can be fixed by mutating some framework residues to llama, or by mutating some residues on the CDRs' backside to human or to a de novo designed sequence. CDR stabilizing or overall fold stabilizing sequences were then back-mutated to the corresponding llama sequence to maintain the biophysical properties and target binding affinity.

This typically generates about 20 humanized variants, each of the VHH variants was expressed as SASA fusion (anti-BSA VHH, Genscript) in 293 cells and the binding affinity validated, if binding and stability are maintained with several variants, then the most humanized is selected.

Protein characterization: SDS PAGE was run on each sample using gradient gels NuPAGE Bis-Tris 4-12% gradient gels using a MES running buffer (Thermo Fisher Scientific). Samples were prepared with either reducing or nonreducing sample buffer and briefly heated to 95° C. N-ethyl maleimide was added to samples electrophoresed in non-reducing buffer to cap any free thiols and prevent unwanted disulfide scrambling as the samples cooled. Molecular weight standards (Blue Plus protein, Thermofisher) were included on the SDS-PAGE. Non-denaturing protein electrophoresis was performed with 1 g of each purified protein sample; reducing conditions were performed by mixing each purified sample with 10 μl of Sample Reducing Agent (Invitrogen, Carlsbad, CA) and heating at 70° C. for 10 min before electrophoresis on NuPAGE 4-12% Bis-Tris Mini Gels 1.0 mm (Invitrogen, Carlsbad, CA). The bands were visualized by SimplyBlue™ SafeStain (Invitrogen, Carlsbad. CA) staining, and the gel was dried using DryEase Mini-Gel Drying System (Invitrogen, Carlsbad, CA). All procedures were performed according to the manufacturer's instructions.

Analysis of native molecule homogeneity and determination of molecular weight was performed using Size Exclusion Chromatography with Light Scattering (SEC-LS) when indicated. Size exclusion chromatography (SEC) was carried out on a Zenix SEC 300 4.6×300 mm (Sepax Technologies) in 20 mM sodium phosphate pH 7.2, 150 mM NaCl (PBS), 0.05% NaAzide at a flow rate of 0.35 ml/min using an Agilent 1260 UPLC. In addition to UV detection, the eluent was monitored with a refractive index detector (Waters, Milford. MA). Light scattering was monitored using a Wyatt Dawn 18 angle, coupled with an Optrex refractometer. Intact mass of molecules was determined mass spectrometry (Merrigen, Lowell MA).

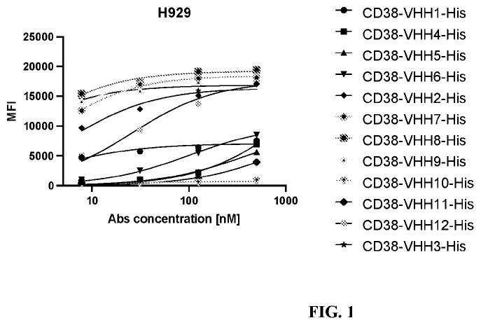

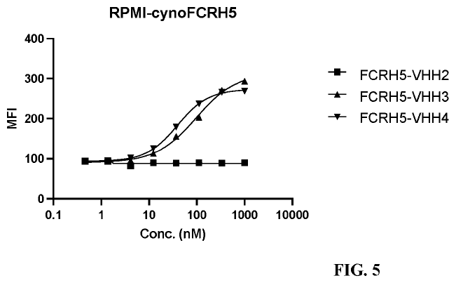

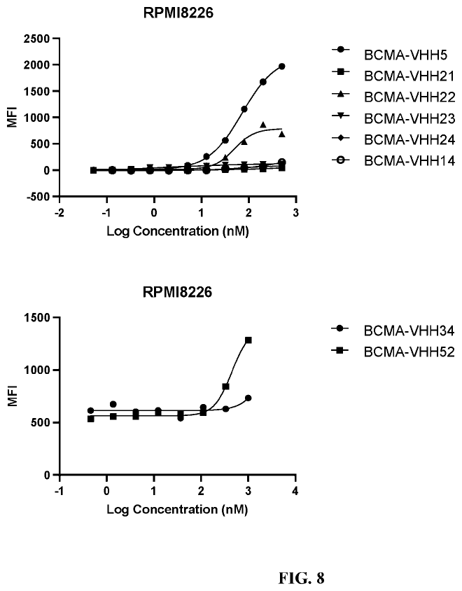

Generation and selection of VHH antibodies to multiple myeloma targets; Protein constructs were analyzed for binding affinity as described in the methods section. Molecules were determined to be homogeneous as described in the methods. Relevant binding values are summarized in Tables 6, 10, 14, and 18, and in FIGS. 1-8. All VHH antibodies affinities were measured with either monovalent or bivalent format as indicated.

### Example 1. Anti-CD38

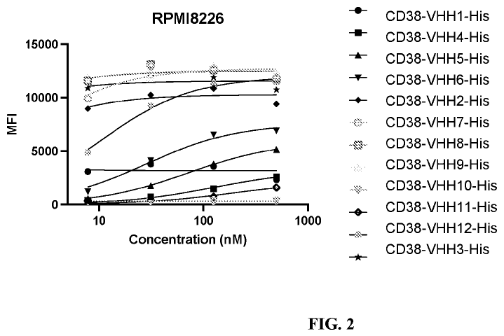

For the discovery of multiple myeloma targeting antibodies against the NK cell antigen CD38, immunizations were carried out as described in the methods above using both human and cynomolgus sequences. Molecules used for immunization were Human and Cyno CD38 Protein purchased from ACRO Biosystems. Antibodies against CD38 were isolated from the PBMC of immunized animals upon appropriate titer levels. Antibodies were isolated using B-cell sorting as previously described with 2 cells plated per well. Binding curves for selected anti-CD38 antibodies against H929 and RPM18226 cell lines (Table 6, FIGS. 1 and 2 respectively) were generated using FACS analysis as described in the methods. Sequences of the 12 clones that show binding to CD38 are shown in Table 4 below. CDRs for the 12 selected clones are shown in Table 5. EC50 antibody-cell surface antigen binding values can be found in Table 6.

TABLE 4
Amino acid sequences of anti-CD38 VHH domains. CDRs are
underlined

| | | SEQ ID |
|---|---|---|
| Clone | Amino acid sequence | NO |
| CD38-VHH1 | QVQLVESGGGLVQPGDSLRLSCAASGFILDTYSVAWFRQAP | 1 |
| | GKEREGVSCISSRDGNTFYSDSVKGRFTISRDNAKNTVYLQM | |
| | NSLKSEDTAVYTCAAGAQAHCTIFTSYFNSDYYRRYNYWG | |
| | QGTQVTVSS | |
| CD38-VHH2 | QVQLVESGGGLVQPGGSLRLSCAASGFIFSDKVMSWVRQAP | 2 |
| | GKGREWVSTITPGGTATSYTESVKGRFTISRDNAKNTLYLQ | |
| | MNNLKPDDTALYYCRIGGPGGRYDNWGQGTQVTVSS | |
| CD38-VHH3 | QVQLVESGGGLVQAGGSLTLSCAASRSIFEINTMTMGWYRQ | 3 |
| | APGKQRELIAASRGATTNYADSVKGRFTISRDNAKNTVYLQ | |
| | MNSLKPEDTAVYYCSADRYGFGYGDNDYWGQGTQVTVSS | |
| CD38-VHH4 | QVQLVESGGGLVQPGGSLRLSCAASGFSLDYYHIGWFRQAP | 4 |
| | GKEREGISCISSSDGYTLYADSVKGRFTISRDNAKNTVYLQM | |
| | NSLKPEDTAVYYCAASPRRLACAGSLYPPLSADFSSWGQGT | |
| | QVTVSS | |
| CD38-VHH5 | QVQLVESGGGLVQTGGSLRLSCVASGAIVSAESMGWYRQAP | 5 |
| | GNQRELVAEIISGSKSNYGESVKGRFTISRDNAENTVYLQMN | |
| | SVKPEDTAVYYCKRTERIWTNNPQVYWGQGTQVTVSS | |
| CD38-VHH6 | QVQLVESGGGLVQAGGSLRLSCAASGTFSSINLMGYYRQAP | 6 |
| | GKQREFVARDYTEGTTDYADSVKGRFTISRDNTKKTVFLQM | |
| | NNLKPEDTAIYYCWLMVRAGDVYWGQGTQVTVSS | |
| CD38-VHH7 | QVQLVESGGGLVHTGGSLRLSCAASGSISGLNTMGWYRQAP | 7 |
| | GKQRELVATIISGTMTRYADSVKGRFTISKDNAKNTVYLQM | |
| | NSLKPEDTAVYYCTFKEITRDSRSYWGQGTQVTVSS | |
| CD38-VHH8 | QVQLVESGGGLVQAGGSLRLSCAASGSSVSMNSMAWYRQA | 8 |
| | PGKQRELVALITPGDRINYADFVKGRFTISRDNAKNTVNLQM | |
| | NNLKPEDTAVYYCNIGATRPPFGAWGQGTQVTVSS | |
| CD38-VHH9 | QVQLVESGGGLVQAGGSLRLSCVASGFIYSISTMGWYRQAP | 9 |
| | GKQRELVATITSGGNTNYADSVKGRFTISRDNAQNTVYLQM | |
| | NNLKPEDTAIYYCNTAWRETIVSRVWGQGTQVTVSS | |
| CD38- | QVQLVESGGGLVQAGGSLKLACVVSGRYFRINAMGWYRQA | 10 |
| VHH10 | PGKQRDMVASISNDGSTNYGDVVKGRFTISRDNNKNTVYLQ | |
| | MNSLKPEDTAVYSCNVKALPFLSSNELSYWGQGTQVTVSS | |
| CD38- | QVQLVESGGGLVQAGGSLKLSCAASGTFSSIALMGYYRQGP | 11 |
| VHH11 | GKQREFVARVSIGGVTEYADSVKGRFTISGDNAKKTVDLQM | |
| | NNLKPDDTGVYYCWRMEGAGDVYWGQGTQVTVSS | |
| CD38- | QVQLVESGGGLVQAGGSLRLSCVASGIIFRIFSMGWYRQAPG | 12 |
| VHH12 | KQRELVATITSGGNTNYAHSVKGRFTISRDDAKNTVNLQMN | |
| | SLKPEDTAVYYCNVAIPSGIVDRSAWGQGTQVTVSS | |

TABLE 5
Amino acid sequences of anti-CD38 VHH domain CDRs

| Antibody | | | |
|---|---|---|---|
| VHH | CDR1 | CDR2 | CDR3 |
| CD38-VHH1 | GFILDTYS | ISSRDGNT | AAGAQAHCTIFTSYFNSDYYRRY |
| | (SEQ ID NO: 13) | (SEQ ID NO: 14) | NY |
| | | | (SEQ ID NO: 15) |
| CD38-VHH2 | GFIFSDKV | ITPGGTAT | RIGGPGGRYDN |
| | (SEQ ID NO: 16) | (SEQ ID NO: 17) | (SEQ ID NO: 18) |
| CD38-VHH3 | RSIFEINTMT | SRGATT | SADRYGFGYGDNDY |
| | (SEQ ID NO: 19) | (SEQ ID NO: 20) | (SEQ ID NO: 21) |
| CD38-VHH4 | GFSLDYYH | ISSSDGYT | AASPRRLACAGSLYPPLSADFSS |
| | (SEQ ID NO: 22) | (SEQ ID NO: 23) | (SEQ ID NO: 24) |
| CD38-VHH5 | GAIVSAES | IISGSKS | KRTERIWTNNPQVY |
| | (SEQ ID NO: 25) | (SEQ ID NO: 26) | (SEQ ID NO: 27) |
| CD38-VHH6 | GTFSSINL | DYTEGTT | WLMVRAGDVY |
| | (SEQ ID NO: 28) | (SEQ ID NO: 29) | (SEQ ID NO: 30) |
| CD38-VHH7 | GSISGLNT | IISGTMT | TFKEITRDSRSY |
| | (SEQ ID NO: 31) | (SEQ ID NO: 32) | (SEQ ID NO: 33) |
| CD38-VHH8 | GSSVSMNS | ITPGDRI | NIGATRPPFGA |
| | (SEQ ID NO: 34) | (SEQ ID NO: 35) | (SEQ ID NO: 36) |
| CD38-VHH9 | GFIYSIST | ITSGGNT | NTAWRETIVSRV |
| | (SEQ ID NO: 37) | (SEQ ID NO: 38) | (SEQ ID NO: 39) |
| CD38- | GRYFRINA | ISNDGST | NVKALPFLSSNELSY |
| VHH10 | (SEQ ID NO: 40) | (SEQ ID NO: 41) | (SEQ ID NO: 42) |
| CD38- | GTFSSIAL | VSIGGVT | WRMEGAGDVY |
| VHH11 | (SEQ ID NO: 43) | (SEQ ID NO: 44) | (SEQ ID NO: 45) |
| CD38- | GIIFRIFS | ITSGGNT | NVAIPSGIVDRSA |
| VHH12 | (SEQ ID NO: 46) | (SEQ ID NO: 47) | (SEQ ID NO: 48) |

TABLE 6
Anti-CD38 VHH domain binding measured by FACS

| Antibody VHH | Cell line | FACS EC50 (nM) |
|---|---|---|
| CD38-VHH1 | RPMI8226 | <1 |
| CD38-VHH2 | RPMI8226 | 1.01 |
| CD38-VHH3 | RPMI8226 | 0.35 |
| CD38-VHH4 | RPMI8226 | N/A |
| CD38-VHH5 | RPMI8226 | 71.3 |
| CD38-VHH6 | RPMI8226 | 28.4 |
| CD38-VHH7 | RPMI8226 | 1.91 |
| CD38-VHH8 | RPMI8226 | 0.45 |
| CD38-VHH9 | RPMI8226 | 0.51 |
| CD38-VHH10 | RPMI8226 | N/A |
| CD38-VHH11 | RPMI8226 | N/A |
| CD38-VHH12 | RPMI8226 | 10.5 |
| CD38-VHH1 | H929 | 4.43 |
| CD38-VHH2 | H929 | 6.14 |
| CD38-VHH3 | H929 | 1.36 |
| CD38-VHH4 | H929 | N/A |
| CD38-VHH5 | H929 | N/A |
| CD38-VHH6 | H929 | 101 |
| CD38-VHH7 | H929 | 3.55 |
| CD38-VHH8 | H929 | 2.06 |
| CD38-VHH9 | H929 | 2.62 |
| CD38-VHH10 | H929 | N/A |
| CD38-VHH11 | H929 | N/A |
| CD38-VHH12 | H929 | 25.7 |

From the 12 clones tested for binding affinity, three clones were selected for humanization. Humanized sequences can be seen in Table 7 below.

TABLE 7
Amino acid sequences of CD38 humanized variants. CDRs are
underlined

| | | SEQ ID |
|---|---|---|
| Variant | Amino acid sequence | NO: |
| CD38-VHH1-H9 | EVQLVESGGGLVQPGGSLRLSCAASGFILDTYSVAWF | 49 |
| | RQAPGKEREGVSCISSRDGNTFYSDSVKGRFTISRDNA | |
| | KNTVYLQMNSLRAEDTAVYTCAAGAQAHCTIFTSYFN | |
| | SDYYRRYNYWGQGTLVTVSS | |
| CD38-VHH2-H10 | EVQLVESGGGLVQPGGSLRLSCAASGFIFSDKVMSWV | 50 |
| | RQAPGKGREWVSTITPGGTATSYTESVKGRFTISRDNS | |
| | KNTLYLQMNSLKADDTAVYYCRIGGPGGRYDNWGQ | |
| | GTLVTVSS | |
| CD38-VHH3-H9 | EVQLVESGGGLVQPGGSLRLSCAASRSIFEINTMTMG | 51 |
| | WYRQAPGKQRELISASRGATTNYADSVKGRFTISRDN | |
| | AKNTVYLQMNSLKAEDTAVYYCSADRYGFGYGDND | |
| | YWGQGTQVTVSS | |

Provided in Table A below are amino acid sequences of the VHH1, VHH2, and VHH3 of CD38-VHH1-H9, CD38-VHH2-H10, and CD38-VHH3-H9 variants based on exemplary CDR definitions.

TABLE A
Exemplary CDR definitions for anti-CD38 VHHs
IMGT CDR Definition

| Clone Name | VHH-CDR1 | VHH-CDR2 | VHH-CDR3 |
|---|---|---|---|
| CD38-VHH1-H9 | GFILDTYS | ISSRDGNT | AAGAQAHCTIFTSYFNSDYYRRYNY |
| | (SEQ ID NO: 13) | (SEQ ID NO: 14) | (SEQ ID NO: 15) |
| CD38-VHH2-H10 | GFIFSDKV | ITPGGTAT | RIGGPGGRYDN |
| | (SEQ ID NO:16) | (SEQ ID NO: 17) | (SEQ ID NO: 18) |
| CD38-VHH3-H9 | RSIFEINTMT | SRGATT | SADRYGFGYGDNDY |
| | (SEQ ID NO: 19) | (SEQ ID NO: 20) | (SEQ ID NO: 21) |

Kabat CDR Definition

| Clone Name | VHH-CDR1 | VHH-CDR2 | VHH-CDR3 |
|---|---|---|---|
| CD38-VHH1-H9 | TYSVA | CISSRDGNTFYSDSVKG | GAQAHCTIFTSYFNSDYYRRYNY |
| | (SEQ ID NO: 346) | (SEQ ID NO:347) | (SEQ ID NO:348) |
| CD38-VHH2-H10 | DKVMS | TITPGGTATSYTESVKG | GGPGGRYDN |
| | (SEQ ID NO: 349) | (SEQ ID NO: 350) | (SEQ ID NO: 351) |
| CD38-VHH3-H9 | INTMTMG | ASRGATTNYADSVKG | DRYGFGYGDNDY |
| | (SEQ ID NO: 352) | (SEQ ID NO: 353) | (SEQ ID NO: 354) |

Enhanced Chothia CDR Definition

| Clone Name | VHH-CDR1 | VHH-CDR2 | VHH-CDR3 |
|---|---|---|---|
| CD38-VHH1-H9 | GFILDTYSVA | CISSRDGNTF | GAQAHCTIFTSYFNSDYYRRYNY |
| | (SEQ ID NO: 355) | (SEQ ID NO:356) | (SEQ ID NO: 357) |
| CD38-VHH2-H10 | GFIFSDKVMS | TITPGGTATS | GGPGGRYDN |
| | (SEQ ID NO: 358) | (SEQ ID NO: 359) | (SEQ ID NO; 360) |
| CD38-VHH3-H9 | RSIFEINTMTMG | ASRGATTN | DRYGFGYGDNDY |
| | (SEQ ID NO: 361) | (SEQ ID NO: 362) | (SEQ ID NO: 363) |

Contact CDR Definition

| Clone Name | VHH-CDR1 | VHH-CDR2 | VHH-CDR3 |
|---|---|---|---|
| CD38-VHH1-H9 | DTYSVA | GVSCISSRDGNTF | AAGAQAHCTIFTSYFNSDYYRRY |
| | (SEQ ID NO: 364) | (SEQ ID NO: 365) | (SEQ ID NO: 366) |
| CD38-VHH2-H10 | SDKVMS | WVSTITPGGTATS | RIGGPGGRYD |
| | (SEQ ID NO: 367) | (SEQ ID NO: 368) | (SEQ ID NO: 369) |
| CD38-VHH3-H9 | EINTMTMG | LISASRGATTN | SADRYGFGYGDND |
| | (SEQ ID NO: 370) | (SEQ ID NO: 371) | (SEQ ID NO: 372) |

Chothia CDR Definition

| Clone Name | VHH-CDR1 | VHH-CDR2 | VHH-CDR3 |
|---|---|---|---|
| CD38-VHH1-H9 | GFILDTY | SSRDGN | GAQAHCTIFTSYFNSDYYRRYNY |
| | (SEQ ID NO: 373) | (SEQ ID NO: 374) | (SEQ ID NO: 375) |
| CD38-VHH2-H10 | GFIFSDK | TPGGTA | GGPGGRYDN |
| | (SEQ ID NO: 376) | (SEQ ID NO: 377) | (SEQ ID NO: 378) |
| CD38-VHH3-H9 | RSIFEIN | RGAT | DRYGFGYGDNDY |
| | (SEQ ID NO: 379) | (SEQ ID NO:380) | (SEQ ID NO: 381) |

### Example 2. Anti-BCMA

For the discovery of multiple myeloma targeting antibodies against the tumor-associated antigen BCMA, immunizations were carried out as described in the methods above using full length Human and Cynomolgus BCMA as the immunogen. Peptide sequence used for immunization were full length Human and Cyno BCMA. Antibodies against BCMA were isolated from the PBMC of immunized animals upon appropriate titer levels. Antibodies were isolated using yeast or B cell sorting as described in the methods. Sequences of the 52 clones that show binding to BCMA are shown in Table 8 below. CDRs for the 52 selected clones are shown in Table 9.

TABLE 8
Amino acid sequences of anti-BCMA VHH domains. CDRs are underlined

| | | SEQ ID |
|---|---|---|
| Clone | Amino acid sequence | NO |
| BCMA-VHH1 | AVQLVDSGGGLVTAGDSLTLSCVASGRTLSPYTAGWFRQ | 52 |
| | APGREREFVALITARGDWTSYSDSVKGRFTISRDNAKNTIY | |
| | LQMNSLEPEDTALYYCVRDLLGRDDYWGQGTQVTVSS | |
| BCMA-VHH2 | HVQLVESGGGLVQTGGSLRLSCAASGRTLNNYVVAWFRQ | 53 |
| | APGKEREFVATMWWSGGSPWYSDNVKGRFTISRDNAKNT | |
| | LYLQMNSLRPEDTAVYYCAATWVGTSEYRHWGQGTQVT | |
| | VSS | |
| BCMA-VHH3 | QVQLVESGGGLVQAGGSLRLSCAASGSIFAYHVMGWYRQ | 54 |
| | APGKQRELVALITSGGSTNYTDSVKGRFTISRDNAKNTVYL | |
| | QMNSLKPEDTAVYYCAIGYYSGSYYWERSGDYWGQGTQ | |
| | VTVSS | |
| BCMA-VHH4 | QVKLEESGGGLVQTGGSLRLSCAASGRTLNNYVVAWFRQ | 55 |
| | APGKEREFVATMWWSGGSPWYSDNVKGRFTISRDNAKNT | |
| | LYLQMNSLRPEDTAVYYCAATWVGTSEYQHWGQGTQVT | |
| | VSS | |
| BCMA-VHH5 | QVKLEESGGGLVQAGGSLRLSCAASGRTFSDYTVAWFRQ | 56 |
| | APGSEREFVAASVWTDGKPYYVDSVRGRFTISRDNAKNTV | |
| | WLQMDSLQPDDTAVYYCQALTGGSWILDYWGQGTPVTV | |
| | SS | |
| BCMA-VHH6 | QVKLEESGGGLVQAGDSLTLSCAASGRPLRMYNMGWFRQ | 57 |
| | APGKEREFVAFISWSGKTTYYSDAVKGRFVISRDNAKRTV | |
| | YLQMDSLKPDDTGDYYCFMNVWADTSDSSADAYWGQGT | |
| | QVTVSS | |
| BCMA-VHH7 | EVQLVDSGGGLVQAGGSLRLSCAASGSTFSRYAMGWIRQ | 58 |
| | APGKEREFVAAISWSGGSTYYADSVKGRFTISRDVAKNTV | |
| | SLQMNSLKPEDAGVYFCFGDIIRSGERSDYEYWGQGTRVT | |
| | VTS | |
| BCMA-VHH8 | QVQLVQSGGGLVLPGGSLRLSCAASGFTLSSYWMHWLRQ | 59 |
| | APGKGLEWVSAIKPESGITYYAESMKGRFTISRDNAKNTLY | |
| | LQMNSLKSEDSALYYCVREDYDSAYVGDYWGKGTQVTV | |
| | SS | |
| BCMA-VHH9 | QVKLEESGGGFVQPGGSLRLSCAASGFTYSSYWMHWVRQ | 60 |
| | APGKGLEWVSGISTGGDTTDTYYADSVKGRFTISRDNAKN | |
| | TLYLQMNSLKPEDTAVYYCVALNLWGTDLEHDYWGRGT | |
| | QVTVSS | |
| BCMA-VHH10 | HVQLVDSGGGLVRSGGSLRLSCVMSGGTLEYYAMAWFR | 61 |
| | QAPGKERETVAAITWSGGSTYYVDSVKGRFTISRDNAKNT | |
| | LYLQMNSLKPEDTAVYYCAAQFVEVEILVRSYEYWGQGT | |
| | QVTVST | |
| BCMA-VHH11 | QVQLVDSGGGLVQPGGSLRLSCAASGFTFSSYWMYWVRQ | 62 |
| | APGKGLEWVSAINTDGDSTYYADSVKGRFTVSRDVAGNM | |
| | IYLQMTNLKPEDTAVYTCAAIVTRSDGHQYDYWGRGTQV | |
| | TVSV | |
| BCMA-VHH12 | QVQLVESGGGSVQAGGSLRLSCAASGRIDSGYTMAWFRQ | 63 |
| | APGSEREFVAAVVGSDGRDYYIDSVRGRFTVSRDSAKNTV | |
| | YLEMTGLKPEDTAVYYCAATNYYSDYLDHLSRGYWGQG | |
| | TQVTVSS | |
| BCMA-VHH13 | QVQLVDSGGRLVQAGDSLRLSCSASGFTFGSYWMFWVRQ | 64 |
| | APGKGLEWVSAIDTSGGHVYYGDSVNGRFTISRDNAKNTL | |
| | YLQMNSLKPEDTALYYCARVENTWESIYWGQGTTVTVSS | |
| BCMA-VHH14 | QVQLVESGGGLAQSGGSLELSCAASGHTLNSYAMGWFRQ | 65 |
| | APGKEREFVAAISRSGEKTYYADSVKGRFTISGDNAKNTA | |
| | ALQMGRLKPEDTAVYYCGAWNFVKNDAYWGQGTQVTV | |
| | SA | |
| BCMA-VHH15 | QVQLVESGGGLVQPGGSLRLSCAASGFTFSTYSMYWVRQ | 66 |
| | APGKGLEWVSAIDARGVNTYYADSVRGRFTVSRDNAKNT | |
| | LYLQMNNLKLEDTALYYCGGWEFATWGRGTQVTVSS | |
| BCMA-VHH16 | QVKLEESGGGLVQAGGSLRLSCAASGHTFSNSAMGWIRQ | 67 |
| | APGKEREFVAAISLNGGNTHYAESVKGRFAISRDNAKNTM | |
| | YLQMNSLKPEDTAVYYCATLGFASWGQGTQVTVSS | |
| BCMA-VHH17 | QVQLVESGGGLVQAGGSLRLSCAASGRTFSGYTMAWFRQ | 68 |
| | APGSEREFVAAAVGSDGSTYYVDSVKGRFTMSRDSAKNT | |
| | VYLMMTSLQPVDSAVYFCVKLDSGAWSLAEWGQGTQVT | |
| | VSS | |
| BCMA-VHH18 | PVQLVESGGGLVQAGGSLRLSCAASGRTFSSYAMGWFRQ | 69 |
| | APGKEREFVAGVSWTGDNTGYADSVKGRFTISRDNFKSTV | |
| | YLEMNSLKPEDTAVYFCAAWNWGHHEYTYWGRGTQVTV | |
| | SS | |
| BCMA-VHH19 | HVQLVESGGGLVQPGGSLRLSCSVSGRTIGSFVMGWFRQA | 70 |
| | PGKEREFVAAVNWRGSSTYYADSVRGRFTISRGNAATTMS | |
| | LQMNSLKPEDAALYYCARWSWDIGADFGSWGQGTQVTV | |
| | SS | |
| BCMA-VHH20 | QVQLVESGGGLVQPGGSLRLSCAASGFTFSNYAMIWVRQ | 71 |
| | APGKGLEWVSSINANSDTTAYSDSVKGRFAISRDNARNTL | |
| | YLQMNSLKPEDTAVYFCVSDSYIGGLYATYVYWGQGTQV | |
| | TVSS | |
| BCMA-VHH21 | QVKLEESGGGLVQAGGSLSLSCKASVRYFSTYAMGWFRQ | 72 |
| | APGKEREFVAAISWSGGSTYYADSVKGRFTISRDNAKNTV | |
| | YLEMNSLKPEDTAVYYCAAPYGSSQNLEYDYWGQGTQVT | |
| | VSS | |
| BCMA-VHH22 | HVQLVDSGGGLVQAGGSLRLSCAASGFTFDDYAMSWVRQ | 73 |
| | APGKGLEWVSAISWNGGSTYYAESMKGRFTISRDNTKNTL | |
| | YLQMNSLKPEDSAVYYCVPVSHSDSVCGSPYMDYWGKGT | |
| | LVTVSS | |
| BCMA-VHH23 | QVKLEESGGGLVHPGGSLTLSCVASGFNLTSDAVGWFSQA | 74 |
| | APGKDLEPISCISRTESDTVQGRFAISRNSAKNTVYLQMNS | |
| | LQPGDTAVYRCAADNAADRCSLSIYNYNLWGQGTQVTVS | |
| | S | |
| BCMA-VHH24 | QVKLEESGGGLVQAGGSLRLSCQVSGRTFASFAMAWFRQ | 75 |
| | APGKEREFVAGISWSGGFTSYADSVKGRFTISRDNAKHTV | |
| | YLQMNSLKTEDTAMYYCGEWGGFSLLGQGTQVTVSS | |
| BCMA-VHH25 | QVQLVESGGGLVQAGGSLRLSCAASGFTFDDYAIGWFRQ | 76 |
| | APGKEREGVSCIASSDGSTFYADSVKGRFTISSDNANNTVY | |
| | LQMNNLNREDTAVYYCVAPCFWFDTVIAGTDPRYDYWG | |
| | QGTQVTVSS | |
| BCMA-VHH26 | QVQLVESGGGLVQAGGSLRLSCAASGFTLGYYAIGWFRQ | 77 |
| | APGKEREGISCISGRDGSTYYAGSVKGRFTISSDNAKNTVY | |
| | LQMNSLKPVDTAVYYCAAVRGPIVSMDPDLCRPVEFDYR | |
| | GQGTQVTVSS | |
| BCMA-VHH27 | QVQLVESGGGLVQAGGSLRLSCATSGFTFEDYAIAWFRQA | 78 |
| | PGKEREAVSCTSKNDRMPYYAPSVKDRFTISTDNGKNTVY | |
| | LQMNSLKPEDTAVYYCAATNGPAITLFPCHINYWLYDNW | |
| | GPGTQVTVSS | |
| BCMA-VHH28 | QVQLVESGGGLVQAGGSLRLSCATSGFTFEDYAIAWFRQA | 79 |
| | PGKEREAVSCTSKNDRMPYYAPSVKGRFTISTDNGKNTAY | |
| | LQMNSLKPEDTAVYYCASADWRSPTPFPCGVSRSLYDHW | |
| | GQGTQVTVSS | |
| BCMA-VHH29 | QVQLVESGGGLVQAGGSLRLSCAASGNFLRFNAMGWFRQ | 80 |
| | APGKQRELVAMISSGGRTNYVDSVKGRFTVSRDNAKNTV | |
| | TLQMNSLKPEDTAVYYCWSAPDYWGQGTQVTVSS | |
| BCMA-VHH30 | QVQLVESGGGLVQAGGSLRLSCVVSGSFSSIDTVDWYRQA | 81 |
| | PGKQRELVATINRGGDTDYKDSVKGRFTISKDNAKNSVHL | |
| | QMNNLKPEDTAVYTCAGSFTLATGDDFGSWGQGTQVTVS | |
| | S | |
| BCMA-VHH31 | QVQLVESGGGLVQAGGSLRLTCTASEQNFSADYMAWYRQ | 82 |
| | APGKERELIATISNSGRTHYVDSMAGRFTISRDNAKNTVYL | |
| | QLDSLKPGDTAVYYCVAEPFSFRRRAWGRGTQVTVSS | |
| BCMA-VHH32 | QVQLVESGGGLVQAGGSLRLTCTASEQNFSTDDMAWYRQ | 83 |
| | APGKERGLIATITNSGTTHYVDSMAGRFTISRDNAENTVYL | |
| | QMNSLIPEDTAVYYCGESTTGWAECDFGCWGRRTQVTVS | |
| | S | |
| BCMA-VHH33 | QVQLVESGGGLVQPGGSLRLSCAASRSIFSINAMGWYRQA | 84 |
| | PGKQRELVATITNGGTTNYADSVKGRFTISRDNAENTVYL | |
| | QMNSLKPEDTAVYYCNANSRYGVGWYNYWGQGTQVTV | |
| | SS | |
| BCMA-VHH34 | QVQLVESGGGLVQPGGSLRLSCAASGSIFSINPMGWYRQA | 85 |
| | PGKQRELVAAFTSGGTTNYADSVKGRFTISRDNAKNTVYL | |
| | EMNSLKPEDTAVYYCNVRGGHCDPRYWREYWGQGTQVT | |
| | VSS | |
| BCMA-VHH35 | QVQLVESGGGLVQPGGSLRLSCAASGSIFSINPMGWYRQA | 86 |
| | PGKQRELVAAFTSGGTTNYADSVKGRFTISRDNAKNTVYL | |
| | QMNSLKPEDTAAYYCNVRGGHYDPRYWREYWGQGTQVT | |
| | VSS | |
| BCMA-VHH36 | QVQLVESGGGLVQAGGSLRLSCAASGRPIDTYAMGWFRQ | 87 |
| | APGKEREFVAVVSWAGVYTYYADSVKGRFTISRDNAKNT | |
| | VVLQVNRLKPEDTAVYYCAATKLPWNTIVMVQRSYCDY | |
| | WSLGTHVTVSS | |
| BCMA-VHH37 | QVQLVESGGGLVQAGGSRRLSCAASGRTISTYAMGWFRQ | 88 |
| | APGKEREFVAVVSWAGSYTYYADSVKGRFTISRDNAKNT | |
| | VVLQVNSLKPEDTAVYYCAATKLPWNTSVMVKRSVYDY | |
| | WGQGTQVTVSS | |
| BCMA-VHH38 | QVQLVESGGGLVQAGDSLRLSCATSGRPLECYAMGWFRQ | 89 |
| | APGKEREFVAGISGNGVNTYYADSLKGRFTISRDNAKNTM | |
| | NLQMNRLKPEDTAVYFCAATTVPVINLEISHMTYCGKGTL | |
| | GTVSS | |
| BCMA-VHH39 | QVQLVESGGGLVQAGDSLSLSCTSSGRRLEGYTMGWFRQ | 90 |
| | PPGKEREFVAAISRNRVNTYYADSLKGRFTISRDNAKNTIN | |
| | LQMDRLKPEDTAVYYCAATNLPGITLLMSHMNYCDYWTL | |
| | GTQVTVSS | |
| BCMA-VHH40 | QVQLVESGGGLVQVGDSLNLSCTTSGPRLEGYTMGWFRQ | 91 |
| | PPGKEREFVAVITRNRVNTYYANSLKGQFTFSKNNAKNTII | |
| | LQMDRLKPEDTAVYFCAATTVPVINLQVSHINYWSKGTLV | |
| | TVSS | |
| BCMA-VHH41 | QVQLVESGGGLVQAGDSLSLSCTSSGRRLEGYTMGWFRQ | 92 |
| | PPGKEREFVAAISRNRVNTYYADSLKGRFTISRDNAKNTIN | |
| | LQMDRLKPEDTAVYFCAATTVPVINLQVSHINYWGKGTL | |
| | VTVSS | |
| BCMA-VHH42 | QVQLVESGGGLVQAGDSLSLSCTSSGRRLEGYTMGWFRQ | 93 |
| | PPGKEREFVAAISRNRVNTYYADSLKGRFTIPRDNAKNTIN | |
| | LQMDRLKPEDTAVYFCAATTVPVINLQVSHINYWGKGTL | |
| | VTVSS | |
| BCMA-VHH43 | QVQLVESGGGLVQVGGSLRLSCTASGRTFNLYAMGWFRQ | 94 |
| | APGEEREFVGVITWSGGSTGYADSVKGRFAISRDNAKNTV | |
| | YLQMNNLKPEDTALYYCAAKVFPMATLDDDVYDYWGQG | |
| | TQVTVSS | |
| BCMA-VHH44 | QVQLVESGGGLVQAGATLRLSCAASGVTFSNYVMGWFRQ | 95 |
| | APGKEREFVATISWSGGSTYYADSVKGRFTISRDNAKNAV | |
| | YLQMNSLKPEDTAVYYCAAESHRRNTIVIVTTPDEYDYWG | |
| | QGTQVTVSS | |
| BCMA-VHH45 | QVQLVESGGGLVQAGGSLRLSCAASGRTFSSYAMGWFRQ | 96 |
| | APGKEREFVAAISWSGGSTYYADSVKGRFTISRDNAKNTV | |
| | YLQMNSLKPEDTAVYYCAADPSSGYNLFARTVVAFARYD | |
| | YWGQGTQVTVSS | |
| BCMA-VHH46 | QVQLVESGGGLVQAGGSLRLSCAASGRTFSSYAMGWFRQ | 97 |
| | APGKEREFVAAISWSGGSTYYADSVKGRFTIPRDSAKNAV | |
| | YLQMNSLKPEDTATYCCAAHPAGAHGGLIYKNWGRGTH | |
| | VTVSS | |
| BCMA-VHH47 | QVQLVESGGGLVQAGGSLRLSCVAFGRTVSNYAVAWFRQ | 98 |
| | APGKERRFVAAINRSGTPYYGDSVKDRFTISRDSAKNAVY | |
| | LQMNSLKPEDTATYSCAADPTGAHVGAIYKNWGQGTQVT | |
| | VSS | |
| BCMA-VHH48 | QVQLVESGGGLVQAGGSLRLSCVAFGRTFSNYAVAWFRQ | 99 |
| | APGKERRFVAAINRSGTPYYGDSVKGRFTISRDNAENTVY | |
| | LQMNSLKPEDTAVYYCAADLRGSSWYFDGVDYWGKGTL | |
| | VTVSS | |
| BCMA-VHH49 | QVQLVESGGGLVQAGGSLRLSCAASGRTFSSYAMGWFRQ | 100 |
| | APGKEREFVAAISWSGGSTYYADSVKGRFTISRDNAKNTV | |
| | YLQMNSLKPEDTAVYYCAADLRGSSWYFDGMDYWGKGT | |
| | LVTVSS | |
| BCMA-VHH50 | QVQLVESGGGLVQSGGSLRLSCAASGRTFSRYAMGWFRQ | 101 |
| | APGKEREFVAAITRSGTSTYYADSVKGRFTISRDNAKNTM | |
| | YLQMNSLKPEDTAVYYCAAHEAQYSSRWSGTEKGYDYW | |
| | GQGTQVTVSS | |
| BCMA-VHH51 | QVQLVESGGGLVQAGGSLRLSCAASGRTFSSYAMGWFRQ | 102 |
| | APGKEREFVAAISWSGGSTYYADSVKGRFTTSRDNAKSTV | |
| | YLQMNSLKPEDTAVYYCAADPSSGYNLFARTVVAFARYD | |
| | YWGQGTQVTVSS | |
| BCMA-VHH52 | QVQLVESGGGLVQAGGSLRLSCATSGRPLESYAMGWFRQ | 473 |
| | APGKEREFVAAVSWNRGYTYYADSLKGRFTISRDNAKNT | |
| | MVLQMNRLKPEDTAVYYCAATDFPWNTPVMGKRSLYEY | |
| | WRWGTRTNVSS | |

TABLE I
Anti-BCMA VHH domain binding measured by FACS

| Antibody VHH | Cell line | FACS EC50 (nM) |
|---|---|---|
| BCMA-VHH2 | CHO-HuBCMA | 0.8 |
| BCMA-VHH4 | CHO-HuBCMA | 0.57 |
| BCMA-VHH8 | CHO-HuBCMA | 170 |
| BCMA-VHH10 | CHO-HuBCMA | 1255 |
| BCMA-VHH15 | CHO-HuBCMA | 1.76 |
| BCMA-VHH18 | CHO-HuBCMA | 1025 |
| BCMA-VHH1 | H929 | 78 |
| BCMA-VHH3 | H929 | 11 |
| BCMA-VHH6 | H929 | N/A |
| BCMA-VHH7 | H929 | 268 |
| BCMA-VHH9 | H929 | 43.4 |
| BCMA-VHH11 | H929 | 45.9 |
| BCMA-VHH12 | H929 | N/A |
| BCMA-VHH13 | H929 | 15.5 |
| BCMA-VHH16 | H929 | 41.8 |
| BCMA-VHH17 | H929 | 1.01 |
| BCMA-VHH20 | H929 | 83.4 |
| BCMA-VHH5 | RPMI8226 | 72.2 |
| BCMA-VHH21 | RPMI8226 | N/A |
| BCMA-VHH22 | RPMI8226 | 49.2 |
| BCMA-VHH23 | RPMI8226 | N/A |
| BCMA-VHH24 | RPMI8226 | N/A |
| BCMA-VHH14 | RPMI8226 | N/A |

TABLE 9
Amino acid sequences of anti-BCMA VHH domain CDRs

| Antibody | | | |
|---|---|---|---|
| VHH | CDR1 | CDR2 | CDR3 |
| BCMA- | GRTLSPYT | ITARGDWT | VRDLLGRDDY |
| VHH1 | (SEQ ID NO: 103) | (SEQ ID NO: 104) | (SEQ ID NO: 105) |
| BCMA- | GRTLNNYV | MWWSGGSP | AATWVGTSEYRH |
| VHH2 | (SEQ ID NO: 106) | (SEQ ID NO: 107) | (SEQ ID NO: 108) |
| BCMA- | GSIFAYHV | ITSGGST | AIGYYSGSYYWERSGDY (SEQ |
| VHH3 | (SEQ ID NO: 109) | (SEQ ID NO: 110) | ID NO: 111) |
| BCMA- | GRTLNNYV | MWWSGGSP | AATWVGTSEYQH |
| VHH4 | (SEQ ID NO: 106) | (SEQ ID NO: 107) | (SEQ ID NO: 112) |
| BCMA- | GRTFSDYT | SVWTDGKP | QALTGGSWILDY |
| VHH5 | (SEQ ID NO: 113) | (SEQ ID NO: 114) | (SEQ ID NO: 115) |
| BCMA- | GRPLRMYN | ISWSGKTT | FMNVWADTSDSSADAY (SEQ |
| VHH6 | (SEQ ID NO: 116) | (SEQ ID NO: 117) | ID NO: 118) |
| BCMA- | GSTFSRYA | ISWSGGST | FGDIIRSGERSDYEY (SEQ ID |
| VHH7 | (SEQ ID NO: 119) | (SEQ ID NO: 120) | NO: 121) |
| BCMA- | GFTLSSYW | IKPESGIT | VREDYDSAYVGDY (SEQ ID |
| VHH8 | (SEQ ID NO: 122) | (SEQ ID NO: 123) | NO: 124) |
| BCMA- | GFTYSSYW | ISTGGDTTDT | VALNLWGTDLEHDY (SEQ ID |
| VHH9 | (SEQ ID NO: 125) | (SEQ ID NO: 126) | NO: 127) |
| BCMA- | GGTLEYYA | ITWSGGST | AAQFVEVEILVRSYEY (SEQ ID |
| VHH10 | (SEQ ID NO: 128) | (SEQ ID NO: 129) | NO: 130) |
| BCMA- | GFTFSSYW | INTDGDST | AAIVTRSDGHQYDY (SEQ ID |
| VHH11 | (SEQ ID NO: 131) | (SEQ ID NO: 132) | NO: 133) |
| BCMA- | GRIDSGYT | VVGSDGRD | AATNYYSDYLDHLSRGY (SEQ |
| VHH12 | (SEQ ID NO: 134) | (SEQ ID NO: 135) | ID NO: 136) |
| BCMA- | GFTFGSYW | IDTSGGHV | ARVENTWESIY |
| VHH13 | (SEQ ID NO: 137) | (SEQ ID NO: 138) | (SEQ ID NO: 139) |
| BCMA- | GHTLNSYA | ISRSGEKT | GAWNFVKNDAY |
| VHH14 | (SEQ ID NO: 140) | (SEQ ID NO: 141) | (SEQ ID NO: 142) |
| BCMA- | GFTFSTYS | IDARGVNT | GGWEFAT |
| VHH15 | (SEQ ID NO: 143) | (SEQ ID NO: 144) | SEQ ID NO: 145) |
| BCMA- | GHTFSNSA | ISLNGGNT | ATLGFAS |
| VHH16 | (SEQ ID NO: 146) | (SEQ ID NO: 147) | (SEQ ID NO: 148) |
| BCMA- | GRTFSGYT | AVGSDGST | VKLDSGAWSLAE |
| VHH17 | (SEQ ID NO: 149) | (SEQ ID NO: 150) | (SEQ ID NO: 151) |
| BCMA- | GRTFSSYA | VSWTGDNT | AAWNWGHHEYTY |
| VHH18 | (SEQ ID NO: 152) | (SEQ ID NO: 153) | (SEQ ID NO: 154) |
| BCMA- | GRTIGSFV | VNWRGSST | ARWSWDIGADFGS |
| VHH19 | (SEQ ID NO: 155) | (SEQ ID NO: 156) | (SEQ ID NO: 157) |
| BCMA- | GFTFSNYA | INANSDTT | VSDSYIGGLYATYVY (SEQ ID |
| VHH20 | (SEQ ID NO: 158) | (SEQ ID NO: 159) | NO: 160) |
| BCMA- | VRYFSTYA | ISWSGGST | AAPYGSSQNLEYDY |
| VHH21 | (SEQ ID NO: 161) | (SEQ ID NO: 120) | (SEQ ID NO: 162) |
| BCMA- | GFTFDDYA | ISWNGGST | VPVSHSDSVCGSPYMDY (SEQ |
| VHH22 | (SEQ ID NO: 163) | (SEQ ID NO: 164) | ID NO: 165) |
| BCMA- | GFNLTSDA | ISRT | AADNAADRCSLSIYNYNL (SEQ |
| VHH23 | (SEQ ID NO: 166) | (SEQ ID NO: 167) | ID NO: 168) |
| BCMA- | GRTFASFA | ISWSGGFT | GEWGGFSL |
| VHH24 | (SEQ ID NO: 169) | (SEQ ID NO: 170) | (SEQ ID NO: 171) |
| BCMA- | GFTFDDYA | IASSDGST | VAPCFWFDTVIAGTDPRYDY |
| VHH25 | (SEQ ID NO: 163) | (SEQ ID NO: 172) | (SEQ ID NO: 173) |
| BCMA- | GFTLGYYA | ISGRDGST | AAVRGPIVSMDPDLCRPVEFDY |
| VHH26 | (SEQ ID NO: 174) | (SEQ ID NO: 175) | (SEQ ID NO: 176) |
| BCMA- | GFTFEDYA | TSKNDRMP | AATNGPAITLFPCHINYWLYDN |
| VHH27 | (SEQ ID NO: 177) | (SEQ ID NO: 178) | (SEQ ID NO: 179) |
| BCMA- | GFTFEDYA | TSKNDRMP | ASADWRSPTPFPCGVSRSLYDH |
| VHH28 | (SEQ ID NO: 177) | (SEQ ID NO: 178) | (SEQ ID NO: 180) |
| BCMA- | GNFLRFNA | ISSGGRT | WSAPDY |
| VHH29 | (SEQ ID NO: 181) | (SEQ ID NO: 182) | (SEQ ID NO: 183) |
| BCMA- | GSFSSIDT | INRGGDT | AGSFTLATGDDFGS |
| VHH30 | (SEQ ID NO: 184) | (SEQ ID NO: 185) | (SEQ ID NO: 186) |
| BCMA- | EQNFSADY | ISNSGRT | VAEPFSFRRRA |
| VHH31 | (SEQ ID NO: 187) | (SEQ ID NO: 188) | (SEQ ID NO: 189) |
| BCMA- | EQNFSTDD | ITNSGTT | GESTTGWAECDFGC |
| VHH32 | (SEQ ID NO: 190) | (SEQ ID NO: 191) | (SEQ ID NO: 192) |
| BCMA- | RSIFSINA | ITNGGTT | NANSRYGVGWYNY |
| VHH33 | (SEQ ID NO: 193) | (SEQ ID NO: 194) | (SEQ ID NO: 195) |
| BCMA- | GSIFSINP | FTSGGTT | NVRGGHCDPRYWREY |
| VHH34 | (SEQ ID NO: 196) | (SEQ ID NO: 197) | (SEQ ID NO: 198) |
| BCMA- | GSIFSINP | FTSGGTT | NVRGGHYDPRYWREY |
| VHH35 | (SEQ ID NO: 196) | (SEQ ID NO: 197) | (SEQ ID NO: 199) |
| BCMA- | GRPIDTYA | VSWAGVYT | AATKLPWNTIVMVQRSYCDY |
| VHH36 | (SEQ ID NO: 200) | (SEQ ID NO: 201) | (SEQ ID NO: 202) |
| BCMA- | GRTISTY A | VSWAGSYT | AATKLPWNTSVMVKRSVYDY |
| VHH37 | (SEQ ID NO: 203) | (SEQ ID NO: 204) | (SEQ ID NO: 205) |
| BCMA- | GRPLECYA | ISGNGVNT | AATTVPVINLEISHMTY |
| VHH38 | (SEQ ID NO: 206) | (SEQ ID NO: 207) | (SEQ ID NO: 208) |
| BCMA- | GRRLEGYT | ISRNRVNT | AATNLPGITLLMSHMNYCDY |
| VHH39 | (SEQ ID NO: 209) | (SEQ ID NO: 210) | (SEQ ID NO: 211) |
| BCMA- | GPRLEGYT | ITRNRVNT | AATTVPVINLQVSHINY |
| VHH40 | (SEQ ID NO: 212) | (SEQ ID NO: 213) | (SEQ ID NO: 214) |
| BCMA- | GRRLEGYT | ISRNRVNT | AATTVPVINLQVSHINY |
| VHH41 | (SEQ ID NO: 209) | (SEQ ID NO: 210) | (SEQ ID NO: 214) |
| BCMA- | GRRLEGYT | ISRNRVNT | AATTVPVINLQVSHINY |
| VHH42 | (SEQ ID NO: 209) | (SEQ ID NO: 210) | (SEQ ID NO: 214) |
| BCMA- | GRTFNLYA | ITWSGGST | AAKVFPMATLDDDVYDY |
| VHH43 | (SEQ ID NO: 215) | (SEQ ID NO: 129) | (SEQ ID NO: 216) |
| BCMA- | GVTFSNYV | ISWSGGST | AAESHRRNTIVIVTTPDEYDY |
| VHH44 | (SEQ ID NO: 217) | (SEQ ID NO: 120) | (SEQ ID NO: 218) |
| BCMA- | GRTFSSYA | ISWSGGST | AADPSSGYNLFARTVVAFARY |
| VHH45 | (SEQ ID NO: 152) | (SEQ ID NO: 120) | DY |
| | | | (SEQ ID NO: 219) |
| BCMA- | GRTFSSYA | ISWSGGST | AAHPAGAHGGLIYKN |
| VHH46 | (SEQ ID NO: 152) | (SEQ ID NO: 120) | (SEQ ID NO: 220) |
| BCMA- | GRTVSNYA | INRSGTP | AADPTGAHVGAIYKN |
| VHH47 | (SEQ ID NO: 221) | (SEQ ID NO: 222) | (SEQ ID NO: 223) |
| BCMA- | GRTFSNYA | INRSGTP | AADLRGSSWYFDGVDY |
| VHH48 | (SEQ ID NO:224) | (SEQ ID NO: 222) | (SEQ ID NO: 225) |
| BCMA- | GRTFSSYA | ISWSGGST | AADLRGSSWYFDGMDY |
| VHH49 | (SEQ ID NO: 152) | (SEQ ID NO: 120) | (SEQ ID NO: 226) |
| BCMA- | GRTFSRYA | ITRSGTST | AAHEAQYSSRWSGTEKGYDY |
| VHH50 | (SEQ ID NO: 227) | (SEQ ID NO: 228) | (SEQ ID NO: 229) |
| BCMA- | GRTFSSYA | ISWSGGST | AADPSSGYNLFARTVVAFARY |
| VHH51 | (SEQ ID NO: 152) | (SEQ ID NO: 120) | DY |
| | | | (SEQ ID NO: 219) |
| BCMA- | GRPLESYA | VSWNRGYT | AATDFPWNTPVMGKRSLYEY |
| VHH52 | (SEQ ID NO: 474 ) | (SEQ ID NO: 475 ) | (SEQ ID NO: 476 ) |

From the 23 clones tested for cell binding EC50, two clones were selected for humanization. Humanized sequences can be seen in Table 10 below.

TABLE 10
Amino acid sequences of BCMA-VH3 humanized variants. CDRs are underlined

| | | SEQ ID |
|---|---|---|
| Variant | Amino acid sequence | NO: |
| BCMA-VHH3-H1 | QVQLVESGGGLVQPGGSLRLSCAASGSIFAYHVMGW | 230 |
| | YRQAPGKQRELVALITSGGSTNYTDSVKGRFTISRDNS | |
| | KNTVYLQMNSLRAEDTAVYYCAIGYYSGSYYWERSG | |
| | DYWGQGTLVTVSS | |
| BCMA-VHH3-H2 | EVQLVESGGGLVQPGGSLRLSCAASGSIFAYHVMGWY | 231 |
| | RQAPGKQLELVSLITSGGSTNYTDSVKGRFTISRDNSK | |
| | NTLYLQMNSLRAEDTAVYYCAIGYYSGSYYWERSGD | |
| | YWGQGTQVTVSS | |
| BCMA-VHH3-H3 | QVQLVESGGGVVQPGGSLRLSCAASGSIFAYHVMGW | 232 |
| | YRQAPGKQRELVSLITSGGSTNYTDSVKGRFTISRDNA | |
| | KNTLYLQMNSLRAEDTAVYYCAIGYYSGSYYWERSG | |
| | DYWGQGTQVTVSS | |
| BCMA-VHH3-H4 | EVQLVESGGGLVQPGGSLRLSCAASGSIFAYHVMGWY | 233 |
| | RQAPGKQRELVALITSGGSTNYTDSVKGRFTISRDNAK | |
| | NTLYLQMNSLRAEDTAVYYCAIGYYSGSYYWERSGD | |
| | YWGQGTLVTVSS | |
| BCMA-VHH3-H5 | QVQLVESGGGVVQPGGSLRLSCAASGSIFAYHVMGW | 234 |
| | YRQAPGKQLELVALITSGGSTNYTDSVKGRFTISRDNA | |
| | KNTLYLQMNSLRAEDTAVYYCAIGYYSGSYYWERSG | |
| | DYWGQGTQVTVSS | |
| BCMA-VHH3-H6 | QVQLVESGGGLVQPGGSLRLSCAASGSIFAYHVMGW | 235 |
| | YRQAPGKQRELVALITSGGSTNYTDSVKGRFTISRDNA | |
| | KNTVYLQMNSLRAEDTAVYYCAIGYYSGSYYWERSG | |
| | DYWGQGTQVTVSS | |
| BCMA-VHH3-H7 | QVQLVESGGGVVQPGGSLRLSCAASGSIFAYHVMGW | 236 |
| | YRQAPGKQRELVSLITSGGSTNYTDSVKGRFTISRDNA | |
| | KNTVYLQMNSLRAEDTAVYYCAIGYYSGSYYWERSG | |
| | DYWGQGTQVTVSS | |
| BCMA-VHH3-H8 | QVQLVESGGGLVQPGGSLRLSCAASGSIFAYHVMGW | 237 |
| | YRQAPGKQRELVALITSGGSTNYTDSVKGRFTISRDNA | |
| | KNTVYLQMNSLRAEDTAVYYCAIGYYSGSYYWERSG | |
| | DYWGQGTLVTVSS | |
| BCMA-VHH3-H9 | QVQLVESGGGVVQPGGSLRLSCAASGSIFAYHVMGW | 238 |
| | YRQAPGKQRELVALITSGGSTNYTDSVKGRFTISRDNS | |
| | KNTVYLQMNSLRAEDTAVYYCAIGYYSGSYYWERSG | |
| | DYWGQGTLVTVSS | |
| BCMA-VHH3- | QVQLVESGGGVVQPGGSLRLSCAASGSIFAYHVMGW | 239 |
| H10 | YRQAPGKQRELVALITSGGSTNYTDSVKGRFTISRDNA | |
| | KNTLYLQMNSLRAEDTAVYYCAIGYYSGSYYWERSG | |
| | DYWGQGTLVTVSS | |
| BCMA-VHH3- | EVQLVESGGGLVQPGGSLRLSCAASGSIFAYHVMGWY | 240 |
| H11 | RQAPGKQRELVALITSGGSTNYTDSVKGRFTISRDNSK | |
| | NTLYLQMNSLRAEDTAVYYCAIGYYSGSYYWERSGD | |
| | YWGQGTQVTVSS | |
| BCMA-VHH3- | EVQLVESGGGLVQPGGSLRLSCAASGSIFAYHVMGWY | 241 |
| H12 | RQAPGKQLELVSLITSGGSTNYTDSVKGRFTISRDNSK | |
| | NTVYLQMNSLRAEDTAVYYCAIGYYSGSYYWERSGD | |
| | YWGQGTQVTVSS | |
| BCMA-VHH3- | QVQLVESGGGLVQPGGSLRLSCAASGSIFAYHVMGW | 242 |
| H13 | YRQAPGKQLELVALITSGGSTNYTDSVKGRFTISRDNS | |
| | KNTVYLQMNSLRAEDTAVYYCAIGYYSGSYYWERSG | |
| | DYWGQGTLVTVSS | |
| BCMA-VHH3- | EVQLVESGGGLVQPGGSLRLSCAASGSIFAYHVMGWY | 243 |
| H14 | RQAPGKQRELVALITSGGSTNYTDSVKGRFTISRDNSK | |
| | NTVYLQMNSLRAEDTAVYYCAIGYYSGSYYWERSGD | |
| | YWGQGTLVTVSS | |
| BCMA-VHH3- | QVQLVESGGGLVQPGGSLRLSCAASGSIFAYHVMGW | 244 |
| H15 | YRQAPGKQRELVALITSGGSTNYTDSVKGRFTISRDNS | |
| | KNTVYLQMNSLRAEDTAVYYCAIGYYSGSYYWERSG | |
| | DYWGQGTQVTVSS | |
| BCMA-VHH3- | QVQLVESGGGVVQPGGSLRLSCAASGSIFAYHVMGW | 245 |
| H16 | YRQAPGKQRELVSLITSGGSTNYTDSVKGRFTISRDNS | |
| | KNTLYLQMNSLRAEDTAVYYCAIGYYSGSYYWERSG | |
| | DYWGQGTLVTVSS | |
| BCMA-VHH3- | QVQLVESGGGVVQPGGSLRLSCAASGSIFAYHVMGW | 246 |
| H17 | YRQAPGKQLELVALITSGGSTNYTDSVKGRFTISRDNS | |
| | KNTVYLQMNSLRAEDTAVYYCAIGYYSGSYYWERSG | |
| | DYWGQGTLVTVSS | |
| BCMA-VHH3- | EVQLVESGGGLVQPGGSLRLSCAASGSIFAYHVMGWY | 247 |
| H18 | RQAPGKQRELVSLITSGGSTNYTDSVKGRFTISRDNSK | |
| | NTVYLQMNSLRAEDTAVYYCAIGYYSGSYYWERSGD | |
| | YWGQGTQVTVSS | |
| BCMA-VHH3- | EVQLVESGGGVVQPGGSLRLSCAASGSIFAYHVMGW | 248 |
| H19 | YRQAPGKQRELVALITSGGSTNYTDSVKGRFTISRDNS | |
| | KNTLYLQMNSLRAEDTAVYYCAIGYYSGSYYWERSG | |
| | DYWGQGTLVTVSS | |
| BCMA-VHH3- | EVQLVESGGGVVQPGGSLRLSCAASGSIFAYHVMGW | 249 |
| H20 | YRQAPGKQRELVALITSGGSTNYTDSVKGRFTISRDNS | |
| | KNTVYLQMNSLRAEDTAVYYCAIGYYSGSYYWERSG | |
| | DYWGQGTLVTVSS | |
| BCMA-VHH3- | QVQLVESGGGLVQPGGSLRLSCAASGSIFAYHVMGW | 250 |
| H21 | YRQAPGKQRELVALITSGGSTNYTDSVKGRFTISRDNS | |
| | KNTLYLQMNSLRAEDTAVYYCAIGYYSGSYYWERSG | |
| | DYWGQGTLVTVSS | |
| BCMA-VHH3- | QVQLVESGGGLVQPGGSLRLSCAASGSIFAYHVMGW | 251 |
| H22 | YRQAPGKQLELVSLITSGGSTNYTDSVKGRFTISRDNS | |
| | KNTLYLQMNSLRAEDTAVYYCAIGYYSGSYYWERSG | |
| | DYWGQGTLVTVSS | |
| BCMA-VHH3- | EVQLVESGGGVVQPGGSLRLSCAASGSIFAYHVMGW | 252 |
| H23 | YRQAPGKQRELVSLITSGGSTNYTDSVKGRFTISRDNS | |
| | KNTVYLQMNSLRAEDTAVYYCAIGYYSGSYYWERSG | |
| | DYWGQGTLVTVSS | |
| BCMA-VHH3- | EVQLVESGGGLVQPGGSLRLSCAASGSIFAYHVMGWY | 253 |
| H24 | RQAPGKQRELVSLITSGGSTNYTDSVKGRFTISRDNSK | |
| | NTLYLQMNSLRAEDTAVYYCAIGYYSGSYYWERSGD | |
| | YWGQGTLVTVSS | |
| BCMA-VHH3- | QVQLVESGGGLVQPGGSLRLSCAASGSIFAYHVMGW | 254 |
| H25 | YRQAPGKQRELVSLITSGGSTNYTDSVKGRFTISRDNA | |
| | KNTLYLQMNSLRAEDTAVYYCAIGYYSGSYYWERSG | |
| | DYWGQGTLVTVSS | |
| BCMA-VHH3- | QVQLVESGGGLVQPGGSLRLSCAASGSIFAYHVMGW | 255 |
| H26 | YRQAPGKQLELVALITSGGSTNYTDSVKGRFTISRDNS | |
| | KNTVYLQMNSLRAEDTAVYYCAIGYYSGSYYWERSG | |
| | DYWGQGTQVTVSS | |
| BCMA-VHH3- | QVQLVESGGGVVQPGGSLRLSCAASGSIFAYHVMGW | 256 |
| H27 | YRQAPGKQRELVSLITSGGSTNYTDSVKGRFTISRDNA | |
| | KNTVYLQMNSLRAEDTAVYYCAIGYYSGSYYWERSG | |
| | DYWGQGTLVTVSS | |
| BCMA-VHH3- | QVQLVESGGGLVQPGGSLRLSCAASGSIFAYHVMGW | 257 |
| H28 | YRQAPGKQRELVSLITSGGSTNYTDSVKGRFTISRDNS | |
| | KNTVYLQMNSLRAEDTAVYYCAIGYYSGSYYWERSG | |
| | DYWGQGTLVTVSS | |
| BCMA-VHH3- | QVQLVESGGGLVQPGGSLRLSCAASGSIFAYHVMGW | 258 |
| H29 | YRQAPGKQRELVSLITSGGSTNYTDSVKGRFTISRDNS | |
| | KNTVYLQMNSLRAEDTAVYYCAIGYYSGSYYWERSG | |
| | DYWGQGTQVTVSS | |
| BCMA-VHH2-H1 | QVQLVESGGGLVQPGGSLRLSCAASGRTLNNYVVAW | 382 |
| | FRQAPGKEREFVATMWWSGGSPWYSDNVKGRFTISR | |
| | DNSKNTLYLQMNSLRAEDTAVYYCAATWVGTSEYRH | |
| | WGQGTLVTVSS | |
| BCMA-VHH2-H2 | QVQLVESGGGLVQPGGSLRLSCAASGRTLNNYVVAW | 383 |
| | FRQAPGKEREFVSTMWWSGGSPWYSDNVKGRFTISR | |
| | DNSKNTLYLQMNSLRAEDTAVYYCAATWVGTSEYRH | |
| | WGQGTQVTVSS | |
| BCMA-VHH2-H3 | QVQLVESGGGLVQTGGSLRLSCAASGRTLNNYVVAW | 384 |
| | FRQAPGKEREFVATMWWSGGSPWYSDNVKGRFTISR | |
| | DNSKNTLYLQMNSLRPEDTAVYYCAATWVGTSEYRH | |
| | WGQGTLVTVSS | |
| BCMA-VHH2-H4 | EVQLVESGGGLVQTGGSLRLSCAASGRTLNNYVVAW | 385 |
| | FRQAPGKEREFVATMWWSGGSPWYSDNVKGRFTISR | |
| | DNSKNTLYLQMNSLRAEDTAVYYCAATWVGTSEYRH | |
| | WGQGTLVTVSS | |
| BCMA-VHH2-H5 | QVQLVESGGGLVQPGGSLRLSCAASGRTLNNYVVAW | 386 |
| | FRQAPGKEREFVATMWWSGGSPWYSDNVKGRFTISR | |
| | DNSKNTLYLQMNSLRAEDTAVYYCAATWVGTSEYRH | |
| | WGQGTQVTVSS | |
| BCMA-VHH2-H6 | QVQLVESGGGLVQPGGSLRLSCAASGRTLNNYVVAW | 387 |
| | FRQAPGKEREFVATMWWSGGSPWYSDNVKGRFTISR | |
| | DNAKNTLYLQMNSLRAEDTAVYYCAATWVGTSEYR | |
| | HWGQGTLVTVSS | |
| BCMA-VHH2-H7 | HVQLVESGGGLVQPGGSLRLSCAASGRTLNNYVVAW | 388 |
| | FRQAPGKEREFVATMWWSGGSPWYSDNVKGRFTISR | |
| | DNSKNTLYLQMNSLRAEDTAVYYCAATWVGTSEYRH | |
| | WGQGTLVTVSS | |
| BCMA-VHH2-H8 | EVQLVESGGGLVQPGGSLRLSCAASGRTLNNYVVAW | 389 |
| | FRQAPGKEREFVSTMWWSGGSPWYSDNVKGRFTISR | |
| | DNSKNTLYLQMNSLRPEDTAVYYCAATWVGTSEYRH | |
| | WGQGTLVTVSS | |
| BCMA-VHH2-H9 | QVQLVESGGGLVQTGGSLRLSCAASGRTLNNYVVAW | 390 |
| | FRQAPGKEREFVSTMWWSGGSPWYSDNVKGRFTISR | |
| | DNSKNTLYLQMNSLRPEDTAVYYCAATWVGTSEYRH | |
| | WGQGTLVTVSS | |
| BCMA-VHH2- | EVQLVESGGGLVQPGGSLRLSCAASGRTLNNYVVAW | 391 |
| H10 | FRQAPGKEREFVATMWWSGGSPWYSDNVKGRFTISR | |
| | DNAKNTLYLQMNSLRPEDTAVYYCAATWVGTSEYRH | |
| | WGQGTQVTVSS | |
| BCMA-VHH2- | EVQLVESGGGLVQPGGSLRLSCAASGRTLNNYVVAW | 392 |
| H11 | FRQAPGKEREFVSTMWWSGGSPWYSDNVKGRFTISR | |
| | DNAKNTLYLQMNSLRAEDTAVYYCAATWVGTSEYR | |
| | HWGQGTLVTVSS | |
| BCMA-VHH2- | EVQLVESGGGLVQPGGSLRLSCAASGRTLNNYVVAW | 393 |
| H12 | FRQAPGKEREFVATMWWSGGSPWYSDNVKGRFTISR | |
| | DNSKNTLYLQMNSLRAEDTAVYYCAATWVGTSEYRH | |
| | WGQGTQVTVSS | |
| BCMA-VHH2- | QVQLVESGGGLVQTGGSLRLSCAASGRTLNNYVVAW | 394 |
| H13 | FRQAPGKEREFVATMWWSGGSPWYSDNVKGRFTISR | |
| | DNAKNTLYLQMNSLRPEDTAVYYCAATWVGTSEYRH | |
| | WGQGTLVTVSS | |
| BCMA-VHH2- | HVQLVESGGGLVQPGGSLRLSCAASGRTLNNYVVAW | 395 |
| H14 | FRQAPGKEREFVSTMWWSGGSPWYSDNVKGRFTISR | |
| | DNSKNTLYLQMNSLRAEDTAVYYCAATWVGTSEYRH | |
| | WGQGTQVTVSS | |
| BCMA-VHH2- | HVQLVESGGGLVQPGGSLRLSCAASGRTLNNYVVAW | 396 |
| H15 | FRQAPGKEREFVATMWWSGGSPWYSDNVKGRFTISR | |
| | DNAKNTLYLQMNSLRAEDTAVYYCAATWVGTSEYR | |
| | HWGQGTLVTVSS | |
| BCMA-VHH2- | EVQLVESGGGLVQPGGSLRLSCAASGRTLNNYVVAW | 397 |
| H16 | FRQAPGKEREFVATMWWSGGSPWYSDNVKGRFTISR | |
| | DNSKNTLYLQMNSLRAEDTAVYYCAATWVGTSEYRH | |
| | WGQGTLVTVSS | |
| BCMA-VHH2- | EVQLVESGGGLVQTGGSLRLSCAASGRTLNNYVVAW | 398 |
| H17 | FRQAPGKEREFVSTMWWSGGSPWYSDNVKGRFTISR | |
| | DNAKNTLYLQMNSLRAEDTAVYYCAATWVGTSEYR | |
| | HWGQGTLVTVSS | |
| BCMA-VHH2- | HVQLVESGGGLVQTGGSLRLSCAASGRTLNNYVVAW | 399 |
| H18 | FRQAPGKEREFVATMWWSGGSPWYSDNVKGRFTISR | |
| | DNSKNTLYLQMNSLRAEDTAVYYCAATWVGTSEYRH | |
| | WGQGTQVTVSS | |
| BCMA-VHH2- | HVQLVESGGGLVQPGGSLRLSCAASGRTLNNYVVAW | 400 |
| H19 | FRQAPGKEREFVATMWWSGGSPWYSDNVKGRFTISR | |
| | DNSKNTLYLQMNSLRPEDTAVYYCAATWVGTSEYRH | |
| | WGQGTLVTVSS | |
| BCMA-VHH2- | EVQLVESGGGLVQPGGSLRLSCAASGRTLNNYVVAW | 401 |
| H20 | FRQAPGKEREFVSTMWWSGGSPWYSDNVKGRFTISR | |
| | DNSKNTLYLQMNSLRAEDTAVYYCAATWVGTSEYRH | |
| | WGQGTLVTVSS | |

Provided in Table B below are amino acid sequences of the VHH1, VHH2, and VHH3 of BCMA-VHH2-H3, and BCMA-VHH3-H9 variants 0 based on exemplary CDR definitions.

TABLE B
Exemplary CDR definitions for anti-BCMA VHHs
IMGT CDR Definition

| Clone Name | VHH-CDR1 | VHH-CDR2 | VHH-CDR3 |
|---|---|---|---|
| BCMA-VHH3-H9 | GSIFAYHV | ITSGGST | AIGYYSGSYYWERSGDY |
| | (SEQ ID NO: 109) | (SEQ ID NO: 110) | (SEQ ID NO: 111) |
| BCMA-VHH2-H3 | GRTLNNYV | MWWSGGSP | AATWVGTSEYRH |
| | (SEQ ID NO: 106) | (SEQ ID NO: 107) | (SEQ ID NO: 108) |

Kabat CDR Definition

| Clone Name | VHH-CDR1 | VHH-CDR2 | VHH-CDR3 |
|---|---|---|---|
| BCMA-VHH3-H9 | YHVMG | LITSGGSTNYTDSVKG | GYYSGSYYWERSGDY |
| | (SEQ ID NO: 402) | (SEQ ID NO: 403) | (SEQ ID NO: 404) |
| BCMA-VHH2-H3 | NYVVA | TMWWSGGSPWYSDNVKG | TWVGTSEYRH |
| | (SEQ ID NO: 405) | (SEQ ID NO: 406) | (SEQ ID NO: 407) |

Enhanced Chothia CDR Definition

| | VHH-CDR1 | VHH-CDR2 | VHH-CDR3 |
|---|---|---|---|
| BCMA-VHH3-H9 | GSIFAYHVMG | LITSGGSTN | GYYSGSYYWERSGDY |
| | (SEQ ID NO: 408) | (SEQ ID NO: 409) | (SEQ ID NO: 410) |
| BCMA-VHH2-H3 | GRTLNNYVVA | TMWWSGGSPW | TWVGTSEYRH |
| | (SEQ ID NO: 411) | (SEQ ID NO: 412) | (SEQ ID NO: 413) |

Contact CDR Definition

| Clone Name | VHH-CDR1 | VHH-CDR2 | VHH-CDR3 |
|---|---|---|---|
| BCMA-VHH3-H9 | AYHVMG | LVALITSGGSTN | AIGYYSGSYYWERSGD |
| | (SEQ ID NO: 414) | (SEQ ID NO: 415) | (SEQ ID NO: 416) |
| BCMA-VHH2-H3 | NNYVVA | FVATMWWSGGSPW | AATWVGTSEYR |
| | (SEQ ID NO: 417) | (SEQ ID NO: 418) | (SEQ ID NO: 419) |

Chothia CDR Definition

| | | | |
|---|---|---|---|
| BCMA-VHH3-H9 | GSIFAYH | TSGGS | GYYSGSYYWERSGDY |
| | (SEQ ID NO: 420) | (SEQ ID NO: 421) | (SEQ ID NO: 422) |
| BCMA-VHH2-H3 | GRTLNNY | WWSGGS | TWVGTSEYRH |
| | (SEQ ID NO: 423) | (SEQ ID NO: 424) | (SEQ ID NO: 425) |

### Example 3. Anti-GPRC5D

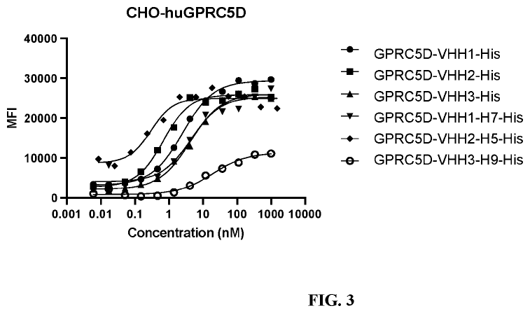

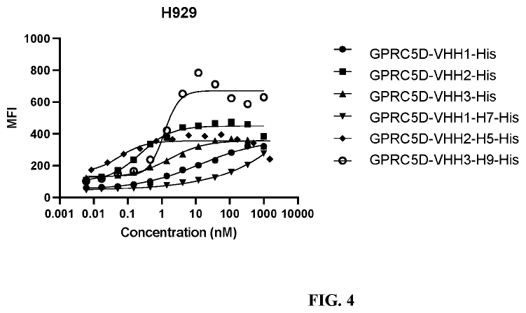

For the discovery of multiple myeloma targeting antibodies against the tumor-associated antigen GPRC5D, immunizations were carried out as described in the methods above using both human and cynomolgus sequences. mRNA molecules used for immunization were full length human GPRC5D and full length cyno GPRC5D. Antibodies against GPRC5D were isolated from the PBMC of immunized animals upon appropriate titer levels. Antibodies were isolated using B-cell sorting as previously described, with 4-100 cells plated per well, and binding curves for selected anti-GPRC5D antibodies against H929 and huGPRC5D CHO cell lines (Table 14, FIGS. 3 and 4 respectively) were generated using FACS analysis as described in the methods. Sequences of the 6 clones that show binding to GPRC5D are shown in Table 11 below. CDRs for the 6 selected clones are shown in Table 12. EC50 antibody-cell surface antigen binding values can be found in Table 13.

TABLE 11
Amino acid sequences of anti-GPRC5D VHH domains. CDRs are underlined

| | | SEQ ID |
|---|---|---|
| Clone | Amino acid sequence | NO |
| GPRC5D-VHH1 | SVQLVESEGGLVQAGGSLRLSCVASGRTVSSYAMGWFRQ | 259 |
| | APGKEREFVAAISWSGRSTYYADSMKGRFTISRDNAKNTV | |
| | YLQMNNLRPEDTAVYYCATSRAVIPGRDWNYYEYSGQGT | |
| | QVTVSS | |
| GPRC5D-VHH2 | QVQLVESGGGLVQAGGSLRLSCAASGRTASAYVMGWFRQ | 260 |
| | APGKERDFVAGISGGAYYADSVKGRFTIARDNAKNTVYLQ | |
| | MNSLKPEDTAVYYCAAERGMRRLTESYQYDYWGQGTQV | |
| | TVSS | |
| GPRC5D-VHH3 | QVKLVESGGGLVQPGGSLRLSCVGSGIIFSASNLAWYRQAL | 261 |
| | GKQRELVAGVTGGGSINYADSVKGRFTISRDNAKNTIYLQ | |
| | MNSLKPEDTAVYYCNARRSYSHWGQGTRVTVSS | |
| GPRC5D-VHH4 | QVQLVESGGGLVQSGGSLRLSCTASGGFGMMYSMGWYR | 262 |
| | QAPGKQRELVAARTIDGSTNYADSVKDRFTVFRENNRNTV | |
| | ALEMDSLKPEDTAVYYCNAKPLNGRLTQYWGQGTQVTVS | |
| | S | |
| GPRC5D-VHH5 | QVHLVESGGGLVQAGGSLRLSCAASRIRFSINVMGWYRQA | 263 |
| | PGKQRELVATIAAGGTTNYADSVKGRFTISRDNAKNTVYL | |
| | QMNSLKPDDTAVYYCNAVLSTLVLPSTYWGQGTQVTVSS | |
| GPRC5D-VHH6 | QVHLVESGGGLVQPGGSLRLSCIASSERTFRSYTMGWFRQ | 264 |
| | APGKEREFVAAISWSHSSTYYADSVKGRFTISRDNAKNTVY | |
| | LQMNNLKPEDTAVYYCAADLRLLPEEYDYYGQGTQVTVS | |
| | S | |

TABLE 12
Amino acid sequences of anti-GPRC5D VHH domain CDRs

| Antibody VHH | CDR1 | CDR2 | CDR3 |
|---|---|---|---|
| GPRC5D-VHH1 | GRTVSSYA | ISWSGRST | ATSRAVIPGRDWNYYEY |
| | (SEQ ID NO: 265) | (SEQ ID NO: 266) | (SEQ ID NO: 267) |
| GPRC5D-VHH2 | GRTASAYV | ISGGA | AAERGMRRLTESYQYDY |
| | (SEQ ID NO: 268) | (SEQ ID NO: 269) | (SEQ ID NO: 270) |
| GPRC5D-VHH3 | GIIFSASN | VTGGGSI | NARRSYSH |
| | (SEQ ID NO: 271) | (SEQ ID NO: 272) | (SEQ ID NO: 273) |
| GPRC5D-VHH4 | GGFGMMYS | RTIDGST | NAKPLNGRLTQY |
| | (SEQ ID NO: 274) | (SEQ ID NO: 275) | (SEQ ID NO: 276) |
| GPRC5D-VHH5 | RIRFSINV | IAAGGTT | NAVLSTLVLPSTY |
| | (SEQ ID NO: 277) | (SEQ ID NO: 278) | (SEQ ID NO: 279) |
| GPRC5D-VHH6 | SERTFRSYT | ISWSHSST | AADLRLLPEEYDY |
| | (SEQ ID NO: 280) | (SEQ ID NO: 281) | (SEQ ID NO: 282) |

TABLE 13
Anti-GPRC5D VHH domain binding measured by FACS

| | | FACS EC50 |
|---|---|---|
| Antibody VHH | Cell line | (nM) |
| GPRC5D-VHH1 | CHO-HuGPRC5D | 2.52 |
| GPRC5D-VHH2 | CHO-HuGPRC5D | 0.68 |
| GPRC5D-VHH3 | CHO-HuGPRC5D | 4.23 |
| GPRC5D-VHH1-H7 | CHO-HuGPRC5D | 4.44 |
| GPRC5D-VHH2-H5 | CHO-HuGPRC5D | 0.3 |
| GPRC5D-VHH3-H9 | CHO-HuGPRC5D | 19.8 |
| GPRC5D-VHH1 | H929 | 13.2 |
| GPRC5D-VHH2 | H929 | 0.23 |
| GPRC5D-VHH3 | H929 | 1.65 |
| GPRC5D-VHH1-H7 | H929 | N/A |
| GPRC5D-VHH2-H5 | H929 | 0.06 |
| GPRC5D-VHH3-H9 | H929 | 1.17 |

From the clones tested for cell binding EC50, four clones were selected for humanization. Humanized sequences are provided in Table 14 below.

TABLE 14
Amino acid sequences of GPRC5D humanized variants. CDRs are underlined

| | | SEQ ID |
|---|---|---|
| Variant | Amino acid sequence | NO: |
| GPRC5D-VHH1-H1 | QVQLVESGGGLVQPGGSLRLSCVASGRTVSSYAMG | 283 |
| | WFRQAPGKEREFVAAISWSGRSTYYADSMKGRFTIS | |
| | RDNAKNTVYLQMNSLRAEDTAVYYCATSRAVIPGR | |
| | DWNYYEYSGQGTQVTVSS | |
| GPRC5D-VHH1-H2 | QVQLVESGGGLVQPGGSLRLSCVASGRTVSSYAMG | 284 |
| | WFRQAPGKEREFVAAISWSGRSTYYADSMKGRFTIS | |
| | RDNAKNTVYLQMNSLRAEDTAVYYCATSRAVIPGR | |
| | DWNYYEYSGQGTLVTVSS | |
| GPRC5D-VHH1-H3 | QVQLVESGGGLVQPGGSLRLSCVASGRTVSSYAMG | 285 |
| | WFRQAPGKEREFVAAISWSGRSTYYADSMKGRFTIS | |
| | RDNAKNTVYLQMNSLRAEDTAVYYCATSRAVIPGR | |
| | DWNYYEYWGQGTQVTVSS | |
| GPRC5D-VHH1-H4 | QVQLVESGGGLVQPGGSLRLSCAASGRTVSSYAMG | 286 |
| | WFRQAPGKEREFVAAISWSGRSTYYADSMKGRFTIS | |
| | RDNAKNTVYLQMNSLRAEDTAVYYCATSRAVIPGR | |
| | DWNYYEYSGQGTQVTVSS | |
| GPRC5D-VHH1-H5 | QVQLVESGGGLVQPGGSLRLSCAASGRTVSSYAMG | 287 |
| | WFRQAPGKEREFVAAISWSGRSTYYADSMKGRFTIS | |
| | RDNAKNTVYLQMNSLRAEDTAVYYCATSRAVIPGR | |
| | DWNYYEYSGQGTLVTVSS | |
| GPRC5D-VHH1-H6 | QVQLVESGGGLVQPGGSLRLSCAASGRTVSSYAMG | 288 |
| | WFRQAPGKEREFVSAISWSGRSTYYADSMKGRFTIS | |
| | RDNAKNTLYLQMNSLRAEDTAVYYCATSRAVIPGR | |
| | DWNYYEYSGQGTLVTVSS | |
| GPRC5D-VHH1-H7 | QVQLVESGGGLVQPGGSLRLSCAASGRTVSSYAMG | 289 |
| | WFRQAPGKEREFVSAISWSGRSTYYDSMKGRFTIS | |
| | RDNAKNTLYLQMNSLRAEDTAVYYCATSRAVIPGR | |
| | DWNYYEYWGQGTLVTVSS | |
| GPRC5D-VHH1-H8 | QVQLVESGGGLVQPGGSLRLSCVASGRTVSSYAMG | 290 |
| | WFRQAPGKEREFVSAISWSGRSTYYADSMKGRFTIS | |
| | RDNAKNTLYLQMNSLRAEDTAVYYCATSRAVIPGR | |
| | DWNYYEYWGQGTLVTVSS | |
| GPRC5D-VHH1-H9 | QVQLVESGGGLVQPGGSLRLSCAASGRTVSSYAMG | 291 |
| | WFRQAPGKEREFVSAISWSGRSTYYADSMKGRFTIS | |
| | RDNAKNTVYLQMNSLRAEDTAVYYCATSRAVIPGR | |
| | DWNYYEYWGQGTLVTVSS | |
| GPRC5D-VHH3-H1 | QVKLVESGGGLVQPGGSLRLSCVGSGIIFSASNLAWY | 292 |
| | RQAPGKQRELVAGVTGGGSINYADSVKGRFTISRDN | |
| | AKNTIYLQMNSLRPEDTAVYYCNARRSYSHWGQGT | |
| | LVTVSS | |
| GPRC5D-VHH3-H2 | QVKLVESGGGLVQPGGSLRLSCVGSGIIFSASNLAWY | 293 |
| | RQAPGKQRELVAGVTGGGSINYADSVKGRFTISRDN | |
| | AKNTIYLQMNSLRAEDTAVYYCNARRSYSHWGQGT | |
| | RVTVSS | |
| GPRC5D-VHH3-H3 | QVKLVESGGGLVQPGGSLRLSCVGSGIIFSASNLAWY | 294 |
| | RQAPGKQRELVAGVTGGGSINYADSVKGRFTISRDN | |
| | AKNTIYLQMNSLRAEDTAVYYCNARRSYSHWGQGT | |
| | LVTVSS | |
| GPRC5D-VHH3-H4 | QVKLVESGGGLVQPGGSLRLSCVGSGIIFSASNLAWY | 295 |
| | RQAPGKQRELVAGVTGGGSINYADSVKGRFTISRDN | |
| | SKNTIYLQMNSLRAEDTAVYYCNARRSYSHWGQGT | |
| | LVTVSS | |
| GPRC5D-VHH3-H5 | QVQLVESGGGLVQPGGSLRLSCVGSGIIFSASNLAWY | 296 |
| | RQAPGKQRELVAGVTGGGSINYADSVKGRFTISRDN | |
| | AKNTIYLQMNSLRAEDTAVYYCNARRSYSHWGQGT | |
| | LVTVSS | |
| GPRC5D-VHH3-H6 | QVKLVESGGGLVQPGGSLRLSCAASGIIFSASNLAWY | 297 |
| | RQAPGKQRELVAGVTGGGSINYADSVKGRFTISRDN | |
| | AKNTIYLQMNSLRAEDTAVYYCNARRSYSHWGQGT | |
| | LVTVSS | |
| GPRC5D-VHH3-H7 | QVQLVESGGGLVQPGGSLRLSCAASGIIFSASNLAWY | 298 |
| | RQAPGKQRELVSGVTGGGSINYADSVKGRFTISRDN | |
| | SKNTIYLQMNSLRAEDTAVYYCNARRSYSHWGQGT | |
| | LVTVSS | |
| GPRC5D-VHH3-H8 | QVQLVESGGGLVQPGGSLRLSCAGSGIIFSASNLAWY | 299 |
| | RQAPGKQRELVAGVTGGGSINYADSVKGRFTISRDN | |
| | AKNTIYLQMNSLRAEDTAVYYCNARRSYSHWGQGT | |
| | LVTVSS | |
| GPRC5D-VHH3-H9 | QVQLVESGGGLVQPGGSLRLSCAASGIIFSASNLAWY | 300 |
| | RQAPGKQRELVAGVTGGGSINYADSVKGRFTISRDN | |
| | AKNTIYLQMNSLRAEDTAVYYCNARRSYSHWGQGT | |
| | LVTVSS | |
| GPRC5D-VHH3- | QVQLVESGGGLVQPGGSLRLSCVASGIIFSASNLAWY | 301 |
| H10 | RQAPGKQRELVAGVTGGGSINYADSVKGRFTISRDN | |
| | AKNTIYLQMNSLRAEDTAVYYCNARRSYSHWGQGT | |
| | LVTVSS | |
| GPRC5D-VHH3- | QVQLVESGGGLVQPGGSLRLSCVGSGIIFSASNLAWY | 302 |
| H11 | RQAPGKQRELVAGVTGGGSINYADSVKGRFTISRDN | |
| | AKNTIYLQMNSLRAEDTAVYYCNARRSYSHWGQGT | |
| | RVTVSS | |
| GPRC5D-VHH2-H1 | QVQLVESGGGLVQPGGSLRLSCAASGRTASAYVMG | 303 |
| | WFRQAPGKERDFVAGISGGAYYADSVKGRFTIARDN | |
| | AKNTVYLQMNSLRAEDTAVYYCAAERGMRRLTESY | |
| | QYDYWGQGTQVTVSS | |
| GPRC5D-VHH2-H2 | QVQLVESGGGLVQPGGSLRLSCAASGRTASAYVMG | 304 |
| | WFRQAPGKERDFVAGISGGAYYADSVKGRFTIARDN | |
| | AKNTVYLQMNSLRAEDTAVYYCAAERGMRRLTESY | |
| | QYDYWGQGTLVTVSS | |
| GPRC5D-VHH2-H3 | QVQLVESGGGLVQPGGSLRLSCAASGRTASAYVMG | 305 |
| | WFRQAPGKEREFVAGISGGAYYADSVKGRFTIARDN | |
| | AKNTVYLQMNSLRAEDTAVYYCAAERGMRRLTESY | |
| | QYDYWGQGTLVTVSS | |
| GPRC5D-VHH2-H4 | QVQLVESGGGLVQPGGSLRLSCAASGRTASAYVMG | 306 |
| | WFRQAPGKERDFVAGISGGAYYADSVKGRFTISRDN | |
| | AKNTVYLQMNSLRAEDTAVYYCAAERGMRRLTESY | |
| | QYDYWGQGTLVTVSS | |
| GPRC5D-VHH2-H5 | QVQLVESGGGLVQPGGSLRLSCAASGRTASAYVMG | 307 |
| | WFRQAPGKEREFVAGISGGAYYADSVKGRFTISRDN | |
| | SKNTVYLQMNSLRAEDTAVYYCAAERGMRRLTESY | |
| | QYDYWGQGTLVTVSS | |
| GPRC5D-VHH4-H1 | QVQLVESGGGLVQPGGSLRLSCAASGGFGMMYSMG | 308 |
| | WYRQAPGKQRELVAARTIDGSTNYADSVKDRFTISR | |
| | ENNRNTLYLQNDSLRAEDTAVYYCNAKPLNGRLTQ | |
| | YWGQGTQVTVSS | |
| GPRC5D-VHH4-H2 | QVQLVESGGGLVQPGGSLRLSCAASGGFGMMYSMG | 309 |
| | WYRQAPGKQRELVAARTIDGSTNYADSVKDRFTISR | |
| | ENNRNTLYLQMDSLRAEDTAVYYCNAKPLNGRLTQ | |
| | YWGQGTQVTVSS | |
| GPRC5D-VHH4-H3 | QVQLVESGGGLVQPGGSLRLSCAASGGFGMMYSMG | 310 |
| | WYRQAPGKQRELVAARTIDGSTNYADSVKDRFTVS | |
| | RENNRNTLALQNDSLRAEDTAVYYCNAKPLNGRLT | |
| | QYWGQGTQVTVSS | |
| GPRC5D-VHH4-H4 | QVQLVESGGGLVQPGGSLRLSCTASGGFGMMYSMG | 311 |
| | WYRQAPGKQRELVAARTIDGSTNYADSVKDRFTIFR | |
| | ENNRNTLYLQMDSLRAEDTAVYYCNAKPLNGRLTQ | |
| | YWGQGTQVTVSS | |
| GPRC5D-VHH4-H5 | QVQLVESGGGLVQPGGSLRLSCAASGGFGMMYSMG | 312 |
| | WYRQAPGKQRELVAARTIDGSTNYADSVKDRFTISR | |
| | ENNRNTVYLQNDSLRAEDTAVYYCNAKPLNGRLTQ | |
| | YWGQGTLVTVSS | |
| GPRC5D-VHH4-H6 | QVQLVESGGGLVQPGGSLRLSCTASGGFGMMYSMG | 313 |
| | WYRQAPGKQRELVAARTIDGSTNYADSVKDRFTVF | |
| | RENNRNTVYLQNDSLRAEDTAVYYCNAKPLNGRLT | |
| | QYWGQGTQVTVSS | |
| GPRC5D-VHH4-H7 | QVQLVESGGGLVQPGGSLRLSCTASGGFGMMYSMG | 314 |
| | WYRQAPGKQRELVAARTIDGSTNYADSVKDRFTVF | |
| | RENNRNTVYLQMDSLRAEDTAVYYCNAKPLNGRLT | |
| | QYWGQGTQVTVSS | |
| GPRC5D-VHH4-H8 | QVQLVESGGGLVQPGGSLRLSCTASGGFGMMYSMG | 315 |
| | WYRQAPGKQRELVAARTIDGSTNYADSVKDRFTVF | |
| | RENNRNTVALQNDSLRAEDTAVYYCNAKPLNGRLT | |
| | QYWGQGTQVTVSS | |
| GPRC5D-VHH4-H9 | QVQLVESGGGLVQPGGSLRLSCAASGGFGMMYSMG | 316 |
| | WYRQAPGKQRELVAARTIDGSTNYADSVKDRFTIFR | |
| | ENNRNTVALQNDSLRAEDTAVYYCNAKPLNGRLTQ | |
| | YWGQGTQVTVSS | |
| GPRC5D-VHH4- | QVQLVESGGGLVQPGGSLRLSCTASGGFGMMYSMG | 317 |
| H10 | WYRQAPGKQRELVAARTIDGSTNYADSVKDRFTVF | |
| | RENNRNTVYLQMDSLRAEDTAVYYCNAKPLNGRLT | |
| | QYWGQGTLVTVSS | |
| GPRC5D-VHH4- | QVQLVESGGGLVQPGGSLRLSCAASGGFGMMYSMG | 318 |
| H11 | WYRQAPGKQRELVAARTIDGSTNYADSVKDRFTVF | |
| | RENNRNTVYLQMDSLRAEDTAVYYCNAKPLNGRLT | |
| | QYWGQGTLVTVSS | |
| GPRC5D-VHH4- | QVQLVESGGGLVQPGGSLRLSCAASGGFGMMYSMG | 319 |
| H12 | WYRQAPGKQRELVAARTIDGSTNYADSVKDRFTVS | |
| | RENNRNTVALQNDSLRAEDTAVYYCNAKPLNGRLT | |
| | QYWGQGTLVTVSS | |
| GPRC5D-VHH4- | QVQLVESGGGLVQPGGSLRLSCTASGGFGMMYSMG | 320 |
| H13 | WYRQAPGKQRELVAARTIDGSTNYADSVKDRFTVS | |
| | RENNRNTVYLQMDSLRAEDTAVYYCNAKPLNGRLT | |
| | QYWGQGTLVTVSS | |

Provided in Table C below are amino acid sequences of the VHH1, VHH2, and VHH3 of GPRC5D-VHH1, GPRC5D-VHH3. GPRC5D-VHH2, and GPRC5D-VHH4 variants based on exemplary CDR definitions.

TABLE C
Exemplary CDR definitions for anti-GPRC5D VHHs
IMGT CDR Definition

| Clone Name | VHH-CDR1 | VHH-CDR2 | VHH-CDR3 |
|---|---|---|---|
| GPRC5D-VHH1-H7 | GRTVSSYA | ISWSGRST | ATSRAVIPGRDWNYYEY |
| | (SEQ ID NO: 265) | (SEQ ID NO: 266) | (SEQ ID NO: 267) |
| GPRC5D-VHH3-H9 | GIIFSASN | VTGGGSI | NARRSYSH |
| | (SEQ ID NO: 271) | (SEQ ID NO: 272) | (SEQ ID NO: 273) |
| GPRC5D-VHH2-H5 | GRTASAYV | ISGGA | AAERGMRRLTESYQYDY |
| | (SEQ ID NO: 268) | (SEQ ID NO: 269) | (SEQ ID NO: 270) |
| GPRC5D-VHH4-H2 | GGFGMMYS | RTIDGST | NAKPLNGRLTQY |
| | (SEQ ID NO: 274) | (SEQ ID NO: 275) | (SEQ ID NO: 276) |

Kabat CDR Definition

| Clone Name | VHH-CDR1 | VHH-CDR2 | VHH-CDR3 |
|---|---|---|---|
| GPRC5D-VHH1-H7 | SYAMG | AISWSGRSTYYADSMKG | SRAVIPGRDWNYYEY |
| | (SEQ ID NO: 426) | (SEQ ID NO: 427) | (SEQ ID NO: 428) |
| GPRC5D-VHH3-H9 | ASNLA | GVTGGGSINYADSVKG | RRSYSH |
| | (SEQ ID NO: 429) | (SEQ ID NO: 430) | (SEQ ID NO: 431) |
| GPRC5D-VHH2-H5 | AYVMG | GISGGAYYADSVKG | ERGMRRLTESYYDY |
| | (SEQ ID NO: 432) | (SEQ ID NO: 433) | (SEQ ID NO: 434) |
| GPRC5D-VHH4-H2 | MYSMG | ARTIDGSTNYADSVKD | KPLNGRLTQY |
| | (SEQ ID NO: 435) | (SEQ ID NO: 436) | (SEQ ID NO: 437) |

Enhanced Chothia CDR Definition

| Clone Name | VHH-CDR1 | VHH-CDR2 | VHH-CDR3 |
|---|---|---|---|
| GPRC5D-VHH1-H7 | GRTVSSYAMG | AISWSGRSTY | SRAVIPGRDWNYYEY |
| | (SEQ ID NO: 438) | (SEQ ID NO: 439) | (SEQ ID NO: 440) |
| GPRC5D-VHH3-H9 | GIIFSASNLA | GVTGGGSIN | RRSYSH |
| | (SEQ ID NO: 441) | (SEQ ID NO: 442) | (SEQ ID NO: 443) |
| GPRC5D-VHH2-H5 | GRTASAYVMG | GISGGAY | ERGMRRLTESYQYDY |
| | (SEQ ID NO: 444) | (SEQ ID NO: 445) | (SEQ ID NO: 446) |
| GPRC5D-VHH4-H2 | GGFGMMYSMG | ARTIDGSTN | KPLNGRLTQY |
| | (SEQ ID NO: 447) | (SEQ ID NO: 448) | (SEQ ID NO: 449) |

Contact CDR Definition

| Clone Name | VHH-CDR1 | VHH-CDR2 | VHH-CDR3 |
|---|---|---|---|
| GPRC5D-VHH1-H7 | SSYAMG | FVSAISWSGRSTY | ATSRAVIPGRDWNYYE |
| | (SEQ ID NO: 450) | (SEQ ID NO: 451) | (SEQ ID NO: 452) |
| \ | SASNLA | LVAGVTGGGSIN | NARRSYS |
| | (SEQ ID NO: 453) | (SEQ ID NO: 454) | (SEQ ID NO: 455) |
| GPRC5D-VHH2-H5 | SAYVMG | FVAGISGGAY | AAERGMRRLTESYQYD |
| | (SEQ ID NO: 456) | (SEQ ID NO: 457) | (SEQ ID NO: 458) |
| GPRC5D-VHH4-H2 | MMYSMG | LVAARTIDGSTN | NAKPLNGRLTQ |
| | (SEQ ID NO: 459) | (SEQ ID NO: 460) | (SEQ ID NO: 461) |

Chothia CDR Definition

| Clone Name | VHH-CDR1 | VHH-CDR2 | VHH-CDR3 |
|---|---|---|---|
| GPRC5D-VHH1-H7 | GRTVSSY | SWSGRS | SRAVIPGRDWNYYEY |
| | (SEQ ID NO: 462) | (SEQ ID NO: 463) | (SEQ ID NO: 464) |
| GPRC5D-VHH3-H9 | GIIFSAS | TGGGS | RRSYSH |
| | (SEQ ID NO: 465) | (SEQ ID NO: 466) | (SEQ ID NO: 491) |
| GPRC5D-VHH2-H5 | GRTASAY | ISGG | ERGMRRLTESYQYDY |
| | (SEQ ID NO: 467) | (SEQ ID NO: 468) | (SEQ ID NO: 469) |
| GPRC5D-VHH4-H2 | GGFGMMY | TIDGS | KPLNGRLTQY |
| | (SEQ ID NO: 470) | (SEQ ID NOL: 471) | (SEQ ID NO: 472) |

### Example 4. Anti-FcRH5

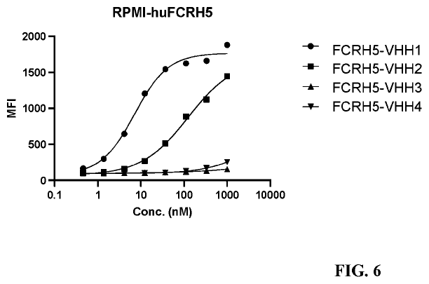

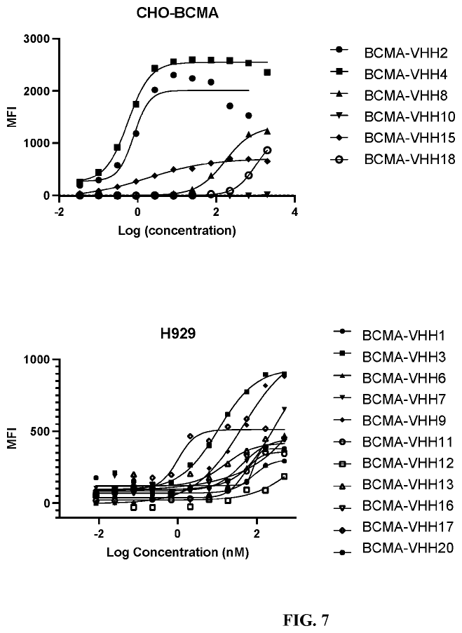

For the discovery of multiple myeloma targeting antibodies against the tumor associated antigen FCRH5, immunizations were carried out as described in the methods above using domain 8 of Human FcRH5 (aa 752-834; Uniprot Ref.; Q96RD9) and Cynomolgus FcRH5 (aa752-834: NCBI Reference Sequence; XP_005541423.1) as the immunogen peptide. Antibodies against FcRH5 were isolated from the PBMC of immunized animals upon appropriate titer levels. Antibodies were isolated using B-cell sorting as previously described, with 2-10 cells plated per well, and binding curves for selected anti-FcRH5 antibodies against Cynomolgus and Human FcRH5 cell lines (Table 18, FIGS. 6 and 7, respectively) were generated using FACS analysis as described in the methods. Sequences of the five clones that show binding to FcRH5 are shown in Table 15 below. CDRs for the five selected clones are shown in Table 16. EC50 antibody-cell surface antigen binding values can be found in Table 17.

TABLE 15
Amino acid sequences of anti-FCRH5 VHH domains. CDRs are underlined

| | | SEQ ID |
|---|---|---|
| Clone | Amino acid sequence | NO |
| FcRH5-VHH1 | QVQFVESGGGLVQAGGSLRLSCAASGITVSRNDMGWYRQA | 321 |
| | PGKQRELVAIMNRVGSTDTADSVKDRFTISRDNTKNTLYLQ | |
| | MNNLKPEDTAVYYCNALNTVITWPWGQGTQVTVSS | |
| FcRH5-VHH2 | QVQLVESGGGLVQAGGSLRLSCTASVHIISHYSMGWFRQAP | 322 |
| | GKERELVAAIPVSGRVPYYLESVKGRFTISRDNAKNRLYLQ | |
| | MNNLKAEDTAVYYCAAYPRKGLEGNEYEYWGQGTQVTVS | |
| | S | |
| FcRH5-VHH3 | QVQLVESGGGSVQAGGSLRLSCVDSGHTLSTYAMGWFRQ | 323 |
| | APGKEREFVAAIARDGGATYLSGSAQGRATISRDNAKSTVY | |
| | LQMNNLKPDDTAVYYCAASSMFSTAKRDYSYWGQGTQVT | |
| | VSS | |
| FcRH5-VHH4 | QVHLVESGGTLVQAGGSLRLSCAASGRTFSTYAMGWFRQG | 324 |
| | PGKEREFVAVIDTTGSASAYAGSVRGRFTVSRDNTENTVYL | |
| | QMNTLKPEDTAVYYCAAARRYSTAPGDYDYWGQGTQVTV | |
| | SS | |
| FcRH5-VHH5 | QVQLVESGGGLVQAGGSLRLSCAASGSHFSIATMGWYRQA | 325 |
| | PGMERELVAVLSSSGRPTYADSVKGRFAIYRNNSMNIVELQ | |
| | MNGLKPEDTAVYVCKANLKRFFIEERYRDYWGQGTQVIVS | |
| | S | |

TABLE 16
Amino acid sequences of anti-FCRH5 VHH domain CDRs

| Antibody VHH | CDR1 | CDR2 | CDR3 |
|---|---|---|---|
| FcRH5-VHH1 | GITVSRND | IMNRVGST | NALNTVITWP |
| | (SEQ ID NO: 326) | (SEQ ID NO: 327) | (SEQ ID NO: 328) |
| FcRH5-VHH2 | VHIISHYS | IPVSGRVP | AAYPRKGLEGNEYEY |
| | (SEQ ID NO: 329) | (SEQ ID NO: 330) | (SEQ ID NO: 331) |
| FcRH5-VHH3 | GHTLSTYA | IARDGGAT | AASSMFSTAKRDYSY |
| | (SEQ ID NO: 332) | (SEQ ID NO: 333) | (SEQ ID NO: 334) |
| FcRH5-VHH4 | GRTFSTYA | IDTTGSAS | AAARRYSTAPGDYDY |
| | (SEQ ID NO: 335) | (SEQ ID NO: 336) | (SEQ ID NO: 337) |
| FcRH5-VHH5 | GSHFSIAT | LSSSGRP | KANLKRFFIEERYRDY |
| | (SEQ ID NO: 338) | (SEQ ID NO: 339) | (SEQ ID NO: 340) |

TABLE 17
Anti-FcRH5 VHH domain binding measured by FACS

| | | FACS EC50 |
|---|---|---|
| Antibody VHH | Cell line | (nM) |
| FcRH5-VHH2 | RPMI8226-huFcRH5 | 125 |
| FcRH5-VHH3 | RPMI8226-huFcRH5 | N/A |
| FcRH5-VHH4 | RPMI8226-huFcRH5 | N/A |
| FcRH5-VHH1 | RPMI8226-huFcRH5 | 7.2 |
| FcRH5-VHH2 | RPMI8226-cyFcRH5 | N/A |
| FcRH5-VHH3 | RPMI8226-cyFcRH5 | 97.2 |
| FcRH5-VHH4 | RPMI8226-cyFcRH5 | 39.5 |

From the clones tested for binding affinity, one clone was selected for humanization. Humanized sequences can be seen in Table 18 below.

TABLE 18
Amino acid sequences of FcRH5 humanized variants. CDRs are underlined

| | | SEQ ID |
|---|---|---|
| Variant | Amino acid sequence | NO: |
| FcRH5-VHH1- | QVQLVESGGGLIQPGGSLRLSCAASGITVSRNDMGWYR | 341 |
| H1 | QAPGKQRELVAIMNRVGSTDTADSVKGRFTISRDNTKN | |
| | SLYLQMNSLRAEDTAVYYCNALNTVITWPWGQGTQVT | |
| | VSS | |
| FcRH5-VHH1- | QVQFVESGGGLIQPGGSLRLSCAASGITVSRNDMGWYR | 342 |
| H2 | QAPGKQRELVAIMNRVGSTDTADSVKGRFTISRDNTKN | |
| | SLYLQMNSLRAEDTAVYYCNALNTVITWPWGQGTQVT | |
| | VSS | |
| FcRH5-VHH1- | QVQLVESGGGLVQPGGSLRLSCAASGITVSRNDMGWYR | 343 |
| H3 | QAPGKQRELVAIMNRVGSTDTADSVKGRFTISRDNTKN | |
| | SLYLQMNSLRAEDTAVYYCNALNTVITWPWGQGTQVT | |
| | VSS | |
| FcRH5-VHH1- | QVQLVESGGGLIQPGGSLRLSCAASGITVSRNDMGWYR | 344 |
| H4 | QAPGKQRELVAIMNRVGSTDTADSVKGRFTISRDNTKN | |
| | TLYLQMNSLRAEDTAVYYCNALNTVITWPWGQGTQVT | |
| | VSS | |
| FcRH5-VHH1- | QVQFVESGGGLVQPGGSLRLSCAASGITVSRNDMGWYR | 345 |
| H5 | QAPGKQRELVAIMNRVGSTDTADSVKGRFTISRDNTKN | |
| | TLYLQMNSLRAEDTAVYYCNALNTVITWPWGQGTQVT | |
| | VSS | |

Provided in Table D below are amino acid sequences of the VHH1, VHH2, and VHH3 of the FcRH5-VHH1-H1 variant based on exemplary CDR definitions.

TABLE D
Exemplary CDR definitions for anti-FcRH5 VHHs
IMGT CDR Definition

| Clone Name | VHH-CDR1 | VHH-CDR2 | VHH-CDR3 |
|---|---|---|---|
| FcRH5-VHH1-H1 | GITVSRND | IMNRVGST | NALNTVITWP |
| | (SEQ ID NO: 326) | (SEQ ID NO: 327) | (SEQ ID NO: 328) |

Kabat CDR Definition

| Clone Name | VHH-CDR1 | VHH-CDR2 | VHH-CDR3 |
|---|---|---|---|
| FcRH5-VHH1-H1 | RNDMG | IMNRVGSTDTADSVKG | LNTVITWP |
| | (SEQ ID NO: 479) | (SEQ ID NO: 480) | (SEQ ID NO: 481) |

Enhanced Chothia CDR Definition

| | | | |
|---|---|---|---|
| FcRH5-VHH1-H1 | GITVSRNDMG | IMNRVGSTD | LNTVITWP |
| | (SEQ ID NO: 482) | (SEQ ID NO: 483) | (SEQ ID NO: 484) |

Contact CDR Definition

| Clone Name | VHH-CDR1 | VHH-CDR2 | VHH-CDR3 |
|---|---|---|---|
| FcRH5-VHH1-H1 | SRNDMG | LVAIMNRVGSTD | NALNTVITW |
| | (SEQ ID NO: 485) | (SEQ ID NO: 486) | (SEQ ID NO: 487) |

Chothia CDR Definition

| Clone Name | VHH-CDR1 | VHH-CDR2 | VHH-CDR3 |
|---|---|---|---|
| FcRH5-VHH1-H1 | GITVSRN | NRVGS | LNTVITWP |
| | (SEQ ID NO: 488) | (SEQ ID NO: 489) | (SEQ ID NO: 490) |

What is claimed is:

1. A binding molecule comprising a VHH that specifically binds
(i) CD38 and comprises a VHH CDR1, VHH CDR2, and VHH CDR3 of any one of the VHH sequences of SEQ ID NOs.: 1 to 12;
(ii) BCMA and comprises a VHH CDR1, VHH CDR2, and VHH CDR3 of any one of the VHH sequences of SEQ ID NOs.: 52 to 102;
(iii) GPRC5D and comprises a VHH CDR1, VHH CDR2, and VHH CDR3 of any one of the VHH sequences of SEQ ID NOs.: 259 to 264: or
(iv) FcRH5 and comprises a VHH CDR1, VHH CDR2, and VHH CDR3 of any one of the VHH sequences of SEQ ID NOs.: 321-325.

2. The binding molecule of claim 1, comprising a VHH that specifically binds
(i) CD38 and comprises the VHH CDRI, VHH CDR2, and VHH CDR3 of any one of the VHHs of Table 5 or A;
(ii) BCMA and comprises the VHH CDR1, VHH CDR2, and VHH CDR3 of any one of the VHHs of Table 9 or B;
(iii) GPRC5D and comprises the VHH CDR1, VHH CDR2, and VHH CDR3 of any one of the VHHs of Table 12 or C; or
(iv) FcRH5 and comprises the VHH CDR1, VHH CDR2, and VHH CDR3 of any one of the VHHs of Table 16 or D.

3. The binding molecule of claim 1 or 2, wherein the VHH is humanized, optionally wherein 1 to 60, 1 to 50, 1 to 40, 1 to 30, 1 to 20, 1 to 15, 1 to 10, 1 to 9, 1 to 8, 1 to 7, 1 to 6, 1 to 5, 1 to 4, 8, 7, 6, 5, 4, 3, 2, or 1 amino acids of the polypeptide of SEQ ID NO:1 are humanize.

4. The binding molecule of claim 3, wherein:
(i) one or more of positions 42, 49, 50, and 52 (numbering based on IMGT numbering) of the llama polypeptide are humanized;
(ii) one or more of positions 42, 49, 50, and 52 (numbering based on IMGT numbering) of the llama polypeptide are not humanized, optionally wherein all of these positions are not humanized;
(iii) positions 42 and/or 52 (numbering based on IMGT numbering) of the llama polypeptide are not humanized; or
(iv) positions 49 and/or 50 (numbering based on IMGT numbering) of the llama polypeptide are humanized, optionally wherein:
position 42 of the VHH is F, Y, V, I, or L;
position 49 of the VHH is E, G, Q, or D;
position 50 of the VHH is R or L; and/or
position 52 of the VHH is G, F, W, L, T, P, A, or M,
wherein the positions are based on IMGT numbering.

5. The binding molecule of any one of claims 1 to 4, comprising a VHH that specifically binds to:
(i) CD38 and comprises an amino acid sequence that is at least 85%, at least 90%, at least 95%, at least 96%, at least 97%, at least 98%, or at least 99% identical to any one VHH of Table 4;
(ii) BCMA and comprises an amino acid sequence that is at least 85%, at least 90%, at least 95%, at least 96%, at least 97%, at least 98%, or at least 99% identical to any one VHH of Table 8;
(iii) GPRC5D and comprises an amino acid sequence that is at least 85%, at least 90%, at least 95%, at least 96%, at least 97%, at least 98%, or at least 99% identical to any one VHH of Table 11;
(iv) FcRH5 and comprises an amino acid sequence that is at least 85%, at least 90%, at least 95%, at least 96%, at least 97%, at least 98%, or at least 99% identical to any one VHH of Table 15;
(v) CD38 and comprises an amino acid sequence that is at least 95%, at least 96%, at least 97%, at least 98%, at least 99% or 100% identical to any one VHH of Table 7;
(vi) BCMA and comprises an amino acid sequence that is at least 95%, at least 96%, at least 97%, at least 98%, at least 99% or 100% identical to any one VHH of Table 10;
(vii) GPRC5D and comprises an amino acid sequence that is at least 95%, at least 96%, at least 97%, at least 98%, at least 99% or 100% identical to any one VHH of Table 14; or
(viii) FcRH5 and comprises an amino acid sequence that is at least 95%, at least 96%, at least 97%, at least 98%, at least 99% or 100% identical to any one VHH of Table 18, optionally wherein the VHH binds to cell surface expressed CD38, BCMA, GPRC5D, or FcRH5 with an EC50 of 0.1 nM to 10 nM.

6. The binding molecule of any one of claims 1 to 5, wherein the VHH is attached to:
(i) a peptide linker or a chemical linker, optionally wherein the linker is a peptide linker that is a glycine linker, a serine linker, or a glycine-serine linker, further optionally wherein the linker is a peptide linker that is (G4S)n, wherein n=1, 2, 3, 4, 5, 6, 7, 8, 9, or 10 (SEQ ID NO: 477);
(ii) a human Fc region comprising a hinge, CH2, and CH₃ region of a human immunoglobulin (Ig); optionally, wherein the human Ig is human IgG1, human IgG2, human IgG3, human IgG4, or human IgG4PAA, wherein the human Fc region is attached at the N- or C-terminal of the VHH; or
(iii) a half-life extension moiety, optionally wherein the half-life extension moiety is polyethylene glycol, XTEN, human serum albumin, transferrin, carboxy-terminal peptide, a homo-amino acid polymer, a proline-alanine-serine polymer, an elastin-like peptide, or hyaluronic acid, wherein the half-life extension moiety is attached at the N- or C-terminal of the VHH.

7. The binding molecule of any one of claims 1 to 6, wherein the binding molecule is bispecific and comprises a binding moiety that specifically binds to a different antigen than the antigen the VHH binds to, optionally wherein the different antigen is CD38, BCMA, GPRC5D, FcRH5, or an antigen expressed on a T cell or a NK cell, and further optionally, wherein the binding moiety is attached at the N- or C-terminal of the VHH, and optionally wherein the binding moiety is a VHH, a scFv, or a Fab.

8. A chimeric antigen receptor (CAR) comprising the binding molecule of any one of claims 1 to 7, a hinge region, a transmembrane domain and at least one intracellular signaling domain, wherein the hinge region is between the binding molecule and the transmembrane domain, and the at least one intracellular signaling domain is attached to the C-terminus of the transmembrane domain, optionally wherein the hinge is from CD28 or CD8alpha; the transmembrane domain is the CD28 transmembrane domain; and the at least one intracellular signaling domain is one from any one of CD27, CD28, CD134, CD137, 4-1BB, or CD3ζ.

9. A T or NK cell expressing the CAR of claim 8.

10. A pharmaceutical composition comprising the binding molecule of any one of claims 1 to 6, or the bispecific antibody of claim 7, and a pharmaceutically acceptable carrier.

11. Nucleic acid encoding the binding molecule of any one of claims 1 to 7, or the bispecific antibody of claim 7, or the CAR of claim 8.

12. A vector or vectors, comprising the nucleic acid of claim 11.

13. A host cell comprising the nucleic acid of claim 11, or the vector or vectors of claim 12, optionally wherein the host cell is a CHO cell, a COS cell, a 293 cell, a NIH3T3 cell, or a HeLa cell.

14. A method of making a binding molecule or bispecific antibody, the method comprising culturing the host cell of claim 13 under conditions that facilitate the expression of the binding molecule or bispecific antibody, and isolating the binding molecule or bispecific antibody, optionally further formulating the binding molecule or bispecific antibody as a pharmaceutical composition.

15. A method of treating a cancer in a human subject in need thereof, the method comprising administering to the human subject a therapeutically effective amount of the binding molecule of any one of claims 1 to 6, the bispecific antibody of claim 7, or a T or NK cell of claim 9, optionally wherein the cancer is multiple myeloma (MM), further optionally wherein the MM is Stage I, Stage II, Stage III MM, MGUS, or SMM.

16. A messenger RNA (mRNA) or mRNAs comprising an open reading frame (ORF) encoding the binding molecule of any one of claims 1 to 6, the bispecific antibody of claim 7, or the CAR of claim 8.

17. A polynucleotide comprising an mRNA comprising: (i) a 5′ UTR; (ii) an ORF encoding a binding molecule of any one of claims 1 to 6, the bispecific antibody of claim 7, or the CAR of claim 8; (iii) a stop codon; and (iv) a 3′ UTR, optionally wherein the mRNA comprises a microRNA (miR) binding site; optionally wherein the miR is expressed in an immune cell of hematopoietic lineage or a cell that expresses TLR7 and/or TLR8 and secretes pro-inflammatory cytokines and/or chemokines; and further optionally wherein the miR binding site is for a miR selected from miR-126, miR126-3p, miR-142, miR-142-3p, miR-142-5p, miR-144, miR-146, miR-150, miR-155, miR-16, miR-21, miR-223, miR-24, miR-27, miR-26a, or any combination thereof; and further optionally wherein the microRNA binding site is located in the 3′ UTR of the mRNA.

18. The polynucleotide of claim 17, wherein the mRNA comprises a 5′ terminal cap, optionally wherein the 5′ terminal cap comprises a CapO, Cap1, ARCA, inosine, N1-methvl-guanosine, 2′-fluoro-guanosine, 7-deaza-guanosine, 8-oxo-guanosin. 2-amino-guanosine, LNA-guanosine, 2-azidoguanosine, Cap2, Cap4, 5′ methylG cap, or an analog thereof.

19. The polynucleotide of claim 17 or 18, wherein the mRNA comprises a poly-A region, optionally wherein the poly-A region is at least about 10, at least about 20, at least about 30, at least about 40, at least about 50, at least about 60, at least about 70, at least about 80, at least about 90 nucleotides in length, or at least about 100 nucleotides in length, or optionally wherein the poly-A region is about 10 to about 200, about 20 to about 180, about 50 to about 160, about 70 to about 140, or about 80 to about 120 nucleotides in length.

20. The polynucleotide of any one of claims 17 to 19, wherein the mRNA comprises at least one chemically modified nucleobase, sugar, backbone, or any combination thereof.

21. The polynucleotide of claim 20, wherein the mRNA comprises at least one chemically modified nucleobase and the at least one chemically modified nucleobase is selected from the group consisting of pseudouracil (ψ), N1-methylpseudouracil (m1ψ), 1-ethylpseudouracil, 2-thiouracil (s²U), 4′-thiouracil, 5-methylcytosine, 5-methyluracil, 5-methoxyuracil, and any combination thereof, optionally wherein at least about 25%, at least about 30%, at least about 40%, at least about 50%, at least about 60%, at least about 70%, at least about 80%, at least about 90%, at least about 95%, at least about 99%, or 100% of the uracils are N1-methylpseudouracils.

22. The polynucleotide of any one of claims 17 to 21, wherein the mRNA comprises a 5′terminal cap comprising Cap1 and a poly-A region 100 nucleotides in length (SEQ ID NO: 493), and wherein all uracils of the polynucleotide are N1-methylpseudouracils.

23. A pharmaceutical composition comprising the mRNA or mRNAs of claim 16 or the polvnucleotide of any one of claims 17 to 22 and a pharmaceutically acceptable excipient.

24. A lipid nanoparticle comprising the mRNA or mRNAs of claim 16 or the polvnucleotide of any one of claims 17 to 22.

25. The lipid nanoparticle of claim 24, wherein the lipid nanoparticle comprises an ionizable amino lipid, a structural lipid, a phospholipid, and a polyethylene glycol (PEG)-modified lipid, optionally wherein (i) the ionizable amino lipid is a compound of Formula (I) or a salt thereof; the structural lipid is cholesterol; the phospholipid is 1,2-distearoyl-sn-glycero-3-phosphocholine or 1, 2-dioleoyl-sn-glycero-3-phosphoethanolamine (DOPE); and the PEG lipid is PEG 2000 dimyristoyl glycerol or 134-hydroxy-3,6,9,12,15,18,21,24,27,30,33,36,39,42,45,48,51,54,57,60,63,66,69,72,75,78,81,84,87 ,90,93,96,99,102,105,108,111.114,117,120,123.126,129,132-tetratetracontaoxatetratriacontahectyl stearate or a salt thereof: or (ii) the ionizable amino lipid is a compound of Formula I-1 or a salt thereof, the structural lipid is cholesterol, the phospholipid is DSPC, and the PEG lipid is 134-hydroxy-3,6,9,12,15,18,21,24,27,30,33,36,39,42,45,48,51,54,57,60,63,66,69,72,75,78,81,84,87 ,90,93,96,99,102,105,108,111,114,117,120,123,126,129,132-tetratetracontaoxatetratriacontahectyl stearate, or PEG-DMG, or a salt thereof.

26. The lipid nanoparticle of claim 25, wherein the lipid nanoparticle comprises about 47.5 mol % of the ionizable amino lipid: about 39 mol % of cholesterol; about 10.5 mol % of DSPC; and about 3 mol % of the PEG-lipid; optionally wherein the lipid nanoparticle comprises 47.5 mol % of the ionizable amino lipid; 39 mol % of cholesterol: 10.5 mol % of DSPC; and 3 mol % of the PEG-lipid.

27. A kit comprising (i) the binding molecule of any one of claims 1 to 6; the bispecific antibody of claim 7; the T or NK cell of claim 9, the pharmaceutical composition of claim 10 or 23; the mRNA or mRNAs of claim 16; the polynucleotide of any one of claims 17 to 22; or the lipid nanoparticle of claim 24, 25, or 26, and (ii) a package insert instructing a user of the kit to administer the binding molecule, the bispecific antibody, the T or NK cell, the pharmaceutical composition, the polynucleotide, or the lipid nanoparticle to a human subject in need thereof.

28. A method of treating a cancer in a human subject in need thereof, the method comprising administering to the human subject a therapeutically effective amount of the mRNA or mRNAs of claim 16; the polynucleotide of any one of claims 17 to 22; the pharmaceutical composition of claim 23; or the lipid nanoparticle of claim 24, 25, or 26, optionally wherein the cancer is multiple myeloma (MM), further optionally wherein the MM is Stage I, Stage II, Stage III MM, MGUS, or SMM.
